(19) United States
(12) Patent Application Publication
(10) Pub. No.: US 2025/0282837 A1
(43) Pub. Date: Sep. 11, 2025

(54) METHOD AND COMPOSITIONS FOR NEURONAL REPROGRAMMING

(71) Applicants: SUNNYBROOK RESEARCH INSTITUTE, Toronto, Ontario (CA); THE GOVERNING COUNCIL OF THE UNIVERSITY OF TORONTO, Toronto, Ontario (CA)

(72) Inventors: Carol SCHUURMANS, Scarborough, Ontario (CA); Joanne MCLAURIN, Toronto, Ontario (CA); Cindy MORSHEAD, Toronto, Ontario (CA); Eunjee PARK, North York, Ontario (CA); Tarlan KEHTARI, Toronto, Ontario (CA); Lakshmy VASAN, Toronto, Ontario (CA); Maryam FAIZ, Toronto, Ontario (CA); Tom ENBAR, Thornhill, Ontario (CA)

(21) Appl. No.: 18/568,747

(22) PCT Filed: Jun. 9, 2022
(86) PCT No.: PCT/CA2022/050923

§ 371 Date: Dec. 8, 2023

Related U.S. Application Data
(60) Provisional application No. 63/208,627, filed on Jun. 9, 2021.

(51) Int. Cl. C07K 14/47; A61K 38/17; A61K 48/00; A61P 25/28; C12N 15/86

(52) U.S. Cl. CPC C07K 14/4705; A61K 38/1709; A61K 48/0058; A61P 25/28; C12N 15/86; C12N 2750/14143; C12N 2830/008

(57) ABSTRACT

The present application provides a mutant basic-helix-loop-helix (bHLH) transcription factor that comprises a mutation of one or more phosphoacceptor site for proline-directed serine-threonine kinases alone or together with a mutation in a conserved PKA site in the HLH domain, found in the corresponding wild-type bHLH transcription factor, and exhibits reduced phosphorylation by proline-directed serine-threonine kinases and PKA. Also provided are nucleic acids encoding the mutant bHLH transcription factor, and vectors comprising the encoding nucleic acids. Use of the mutant bHLH transcription factor or nucleic acid encoding the mutant bHLH transcription factor for therapeutic purposes can efficiently induce neuronal lineage conversion of glial cells, even in an inhibitory environment, and are useful in preventing or treating neurodegenerative diseases and disorders, and for treating CNS injuries. Further provided a use a ZBTB 18 transcription factor or a nucleic acid encoding the ZBTB 18 transcription factor for neuronal lineage conversion of glial cells.

### RELATED APPLICATION

This application is a 35 U.S.C. § 371 filing of International Patent Application No. PCT/CA2022/050923, filed Jun. 9, 2022, which claims priority to U.S. Patent Application Ser. No. 63/208,627, filed Jun. 9, 2021, the contents of which are incorporated herein by reference in their entirety.

### SEQUENCE LISTING

The instant application contains a Sequence Listing which has been submitted electronically in ASCII format and is hereby incorporated by reference in its entirety. Said ASCII file, created on Jul. 9, 2024, is named 748466_SBR9-001US_ST25.txt and is 155,920 bytes in size.

### FIELD OF THE INVENTION

The present application pertains to the field of neuronal reprogramming. More particularly, the present application relates to methods and compositions for use in neuronal reprogramming for treatment of central nervous system diseases, disorders, or injuries.

### INTRODUCTION

The vertebrate central nervous system (CNS) is comprised of a diverse array of neuronal and macroglial (astrocytes and oligodendrocytes) cell types that make up and connect functional brain regions in a complex manner to allow for higher order cognitive functioning and motor and sensory processing. The basic-helix-loop-helix (bHLH) family of transcription factors are essential regulators of neural cell fate specification and differentiation during embryonic development. In particular, proneural and neuronal differentiation bHLH genes promote neuronal differentiation in the embryonic brain, and in some cellular contexts can also induce oligodendrocyte fate specification.

Neural-specific bHLH genes with proneural activity include Neurog1, Neurog2, Ascl1, Neurod4, Atoh1 and Atoh7. These bHLH genes act in transcriptional cascades, turning on other bHLH genes, which function at later developmental stages to control neuronal differentiation. In particular, several bHLH genes are expressed and function later in the development of neural lineages; either in committed neuronal/glial precursors or in post mitotic neuronal and glial cells. Among these are bHLH genes that play important roles in neuronal and glial differentiation and maturation, including members of the NeuroD (Neurod1, Neurod2/Ndrf, Neurod6/Math2), Nscl (Nhlh1/Nscl1, Nhlh2/Nscl2), and Olig (Olig1, Olig2, Olig3, Bhlhe22) families. However, bHLH genes are not active in all cellular contexts, and can be inhibited by environmental signals.

Recently bHLH neuronal differentiation (Neurod1) and proneural (Neurog2, Ascl1) genes have been used to promote the conversion of astrocytes to neurons in vitro and in vivo. Gene therapy methods of neuronal conversion using expression vectors expressing these bHLH neuronal differentiation and proneural genes have been proposed for treatment of neurological conditions such as brain injury and Huntington's disease (See, for example, U.S. Patent Publication Nos. 2019/0117797, 2020/0405801 and 2021/0032300). Researchers have also shown that Neurod1 reprogramming can provide functional recovery in a stroke model (see, for example, Livingston et al., 2020, BioRx, doi: https://doi.org/10.1101/2020.02.02.929091). These methods are intended to target generation of healthy neurons at the site of injury or disease to treat the condition.

Although neuronal programming/reprogramming is a promising approach for treatment, to date, the efficiency of reprogramming is often low and reported conversion rates are highly variable from different groups. The ability of each of these bHLH genes to promote neurogenesis is highly dependent on the environment; neuronal conversion efficiencies vary at different developmental times, in different brain regions and in the diseased or injured brain depending on inhibitory mechanisms present.

A need remains for effective treatments for neurological diseases and disorders, including injury.

The above information is provided for the purpose of making known information believed by the applicant to be of possible relevance to the present invention. No admission is necessarily intended, nor should be construed, that any of the preceding information constitutes prior art against the present invention.

### SUMMARY OF THE INVENTION

An object of the present application is to provide compositions and methods of use thereof in neuronal reprogramming for the treatment or prevention of a neurodegenerative disease or disorder, or for the treatment of neurological (e.g., brain) injury.

In accordance with an aspect of the present application, there is provided a mutant basic-helix-loop-helix (bHLH) transcription factor, wherein the mutant bHLH transcription factor comprises a mutation of one or more phosphoacceptor sites for proline-directed serine-threonine kinases found in the corresponding wild-type bHLH transcription factor, and wherein the mutant bHLH transcription factor exhibits reduced inhibition of function from phosphorylation by proline-directed serine-threonine kinases than the corresponding wild-type bHLH transcription factor. In some embodiments, the mutant comprises a mutation of two or more, or three or more, or four or more, phosphoacceptor sites for proline-directed serine-threonine kinases found in the corresponding wild-type bHLH transcription factor. Alternatively, all of the phosphoacceptor sites for proline-directed serine-threonine kinases found in the corresponding wild-type bHLH transcription factor are mutated. In some embodiments, an additional conserved serine or threonine residue in the conserved HLH domain that is a target of protein kinase A (PKA) phosphorylation, an evolutionarily conserved and inhibitory on-off switch, is also mutated.

In accordance with some embodiments, the mutant bHLH transcription factor is in an isolated and/or purified form that is, for example, suitable for administration to a subject.

In accordance with some embodiments, each mutation of a phosphoacceptor site for proline-directed serine-threonine kinases and/or PKA comprises substitution of a serine or threonine within the phosphoacceptor site with another amino acid selected from the group consisting of alanine, glycine, valine, leucine, isoleucine, D-serine, D-threonine, D-alanine, D-glycine, D-valine, D-leucine and D-isoleucine.

In accordance with one embodiment, the mutant bHLH transcription factor is a mutant of a proneural bHLH transcription factor selected from the group consisting of Neurog2, Ascl1, and Neurod4, which is, optionally, (a) at least 80%, 85%, 90%, 95%, or 97% identical to SEQ ID NO:1 or SEQ ID NO:2; (b) at least 75%, 80%, 85%, 90%, 95%, or 97% identical to SEQ ID NO:13 or SEQ ID NO:14; or (c) at least 70%, 75%, 80%, 85%, 90%, 95%, or 97% identical to SEQ ID NO:19 or SEQ ID NO:20. Examples of such mutant bHLH transcription factors are human ASCL1-SA5, mouse Ascl1-SA6, human ASCL1-SA6 (optionally with additional PKA site mutation), mouse Ascl1-SA7 (optionally with additional PKA site mutation), human NEUROD4-SA4TA6, mouse Neurod4-SA3TA4, human NEUROD4-SA5TA6 (optionally with additional PKA site mutation), mouse Neurod4-SA4TA4 (optionally with additional PKA site mutation), human NEUROG2-SA8TA1, mouse Neurog2-SA9, human NEUROG 2-SA8TA2 (optionally with additional PKA site mutation), and mouse Neurog2-SA9TA1 (optionally with additional PKA site mutation).

In accordance with another embodiment, the mutant bHLH transcription factor is a mutant of a Neurod1 neuronal differentiation transcription factor, which is, optionally, at least 80%, 85%, 90%, 95%, or 97% identical to SEQ ID NO:7 or SEQ ID NO:8. Examples of such mutant bHLH transcription factors are human NEUROD1-SA6, mouse Neurod1-SA6, human NEUROD1-SA7 (optionally with additional PKA site mutation), and mouse Neurod1-SA7 (optionally with additional PKA site mutation).

In accordance with another aspect of the present application, there is provided nucleic acids that encode a mutant bHLH transcription factor as described herein, which can be operably associated with one or more regulatory elements. The nucleic acid can be in a viral (e.g., an AAV, lentiviral or retroviral vector) or non-viral vector.

In accordance with another aspect, there is provided a pharmaceutical composition comprising the mutant bHLH transcription factor as described herein or the nucleic acid encoding the mutant bHLH transcription factor, and a pharmaceutically acceptable carrier or excipient. The composition is optionally formulated for direct intracranial administration, intracranial injection, intracerebroventricular injection, intracisternal injection, intrathecal injection, intravenous injection, intraperitoneal injection, delivery via nanoparticles, delivery via extracellular vesicles, and/or administration using focused ultrasound.

In accordance with another aspect, there is provided a use of the mutant bHLH transcription factor as described herein or the nucleic acid encoding the mutant bHLH transcription factor for neuronal transformation of glial cells in a subject in need thereof. This use can be for prevention or treatment of a neurodegenerative disease or disorder, or for treatment of a brain injury, such as stroke, in the subject.

In accordance with another aspect, there is provided a method for neuronal transformation of glial cells in a subject, said method comprising administering to the subject the mutant bHLH transcription factor as described herein or the nucleic acid encoding the mutant bHLH transcription factor or the pharmaceutical composition containing the mutant bHLH transcription factor or the encoding nucleic acid. This method is useful for preventing or treating a neurodegenerative disease or disorder or for treating of a CNS injury, such as a brain injury, in the subject.

In accordance with some embodiments, the neurodegenerative disease or disorder is selected from the group consisting of amyotrophic lateral sclerosis (ALS), Alzheimer's disease (AD), Parkinson's disease (PD), down's syndrome, dementia pugilistica, multiple system atrophy, frontal temporal dementia, progressive supranuclear palsy, prion diseases, Huntington's disease, motor neuron diseases and Lewy body diseases.

In accordance with another aspect, there is provided a use of a ZBTB18 transcription factor or a nucleic acid encoding the ZBTB18 transcription factor for neuronal transformation of glial cells in a subject in need thereof. In some embodiments, the ZBTB18 transcription factor has an amino acid sequence that is at least 80%, 85%, 90%, 95%, or 97% identical to one of SEQ ID NOs:36, 38, 40, 42, 44, 46, 48, 50, 52 or 54.

In accordance with another aspect, there is provided a method for neuronal transformation of glial cells in a subject, said method comprising administering to the subject a ZBTB18 transcription factor or a nucleic acid encoding the ZBTB18 transcription factor.

In accordance with another aspect, there is provided a vector comprising a nucleic acid encoding a ZBTB18 transcription factor comprising an amino acid sequence that is at least 80%, 85%, 90%, 95%, or 97% identical to one of SEQ ID NOs:36, 38, 40, 42, 44, 46, 48, 50, 52 or 54, wherein the vector is a non-viral vector or a viral vector.

In accordance with another aspect, there is provided a pharmaceutical composition comprising the ZBTB18 transcription factor as described herein or the nucleic acid encoding the ZBTB18 transcription factor, and a pharmaceutically acceptable carrier or excipient. The composition is optionally formulated for direct intracranial administration, intracranial injection, intracerebroventricular injection, intracisternal injection, intrathecal injection, intravenous injection, intraperitoneal injection, delivery via nanoparticles, delivery via extracellular vesicles, and/or administration using focused ultrasound.

### BRIEF DESCRIPTION OF TABLES AND FIGURES

For a better understanding of the application as described herein, as well as other aspects and further features thereof, reference is made to the following description which is to be used in conjunction with the accompanying drawings, where:

FIG. 1: Images (A) of brain sections from mice treated with Ascl1-t2a-iCre and Ascl1-SA6-t2a-iCre vectors immunostained with tdtomato and NeuN antibodies and quantified (B) to show expression of Ascl1-SA6 was more efficient than Ascl1 at promoting conversion of cortical astrocytes to neurons (bars represent mean+/−SEM. p-values: ns—not significant, <0.05*, <0.01**, <0.001***).

FIG. 2: Immunostaining and quantified expression results from stereotactic injections of AAV5-GFAP-iCre (control) and AAV5-GFAP-Ascl1-SA6-t2a-iCre into 16 weeks old hSOD^{G93A} transgenic ALS mice, demonstrating efficient induction of neuronal lineage conversion by Ascl1-SA6 expression (bars represent mean+/−SEM. p-values: ns—not significant, <0.05*, <0.01**, <0.001***).

FIG. 3: Graphical representation of body weight measurements taken from mice following treatment with AAV5-GFAP-iCre (control) and AAV5-GFAP-Ascl1-SA6-t2a-iCre showing better maintenance of body weight in Ascl1-SA6 vs control injected animals (Mann-Whitney test, P=0.0238), significant at 22 weeks (bars represent mean+/−SEM. p-values: ns—not significant, <0.05*, <0.01**, <0.001***).

FIG. 4: Graphical representation of neuronal density measurements taken from mice following treatment with AAV5-GFAP-iCre (control) and AAV5-GFAP-Ascl1-SA6-t2a-iCre showing trend towards increased neuronal density in Ascl1-SA6 vs control injected animals (bars represent mean+/−SEM. p-values: ns—not significant, <0.05*, <0.01**, <0.001***).

FIG. 5: Graphical representation of neurological score (NS) measurements captured by ALSTDI-Neuroscore, demonstrating improved health (the score gets higher with disease progression) in longer surviving animals following treatment with Ascl1-SA6 vs control injections (bars represent mean+/−SEM. p-values: ns—not significant, <0.05*, <0.01**, <0.001***).

FIG. 6: Graphical representation of rotarod results demonstrating improved motor behaviour in animals treated with Ascl1-SA6 vs control injections showing significance at 20 weeks (Mann-Whitney test, P=0.0952; bars represent mean+/−SEM. p-values: ns—not significant, <0.05*, <0.01**, <0.001***).

FIG. 7: Characterization of Aβ plaques and tau hyperphosphorylation. (a) Representative micrographs of cerebrovascular and parenchymal plaques labeled with 6F3D. (b) Representative immunoblots showing that various phosphorylation sites of tau have increased levels in TgF344-AD rats compared to non-transgenic (nTg) controls (c) Significant deficits in burrowing and Barnes Maze task were detected between TgF344 AD and nTg rats. Bars represent mean+SEM. *p<0.05.

FIG. 8: Qualitative analysis of NeuN immunohistochemical staining in the hippocampus of 12-month old TgF344 AD rats and their nTg littermates, with no differences detected in the number of NeuN+ neurons or synaptic markers in TgF344 AD rats in comparison to nTg littermates.

FIG. 9: GABAergic neuronal deficits in TgF344 AD rats. Analyses of the Western blot (Ai) showed an increase in GAD65/GAD2 (Aii) and no change in GAD67/GAD1 (Aiii) protein levels. In contrast, cell counts (Bi) demonstrated a loss of GAD67+ cells that expressed SST or NPY. *p<0.05 and bars are the mean±SEM.

FIG. 10: Analyses of the Western blot (Ai) demonstrated an increase in α-5 (Aiii) and δ (Aiv) but not α-1 (Aii) GABA_{A} receptor subunits when comparing TgF344 AD to nTg rats. Bars are mean±SEM. *p<0.05.

FIG. 11: Representative traces of raw (black) and filtered (theta in the lower end of the modulation index, high-gamma in higher end of the modulation index) recordings from the HPC in an nTg rat (A). Mean co-modulation maps of an nTg (left) and a TgAD (right) rat in the HPC. Theta to high-gamma modulation index was reduced by 78.9+/−0.8%, p=0.05 in the HPC of TgAD vs. that in nTg cohort (B). No difference in modulation index or comodulation maps between nTg and TgAD rats at 2 months of age.

FIG. 12: AAV2/8-Ascl1-mCherry transduction of 12 month old TgF344 AD rat hippocampus is illustrated by the colocalization of GFAP immunoreactivity (light grey) with mCherry expression (dark grey). All infected cells have lost the reactive astrocytic morphology.

FIG. 13: Transdifferentiation of reactive astrocytes into neurons was confirmed by the presence of NeuN+mCherry+ cells without the presence of GFAP+mCherry+ cells within the hilus of the hippocampus. Panel A is the control vector, AAV-mCherry, injected hemisphere while panel B is the AAV-Ascl1-mCherry injected hemisphere, with panel C being a magnification of the AAV-Ascl1-mCherry hemisphere. Scale bars=250 μm (A&B), 100 μm (C).

FIG. 14: The differentiation of new neurons into GABAergic interneurons was confirmed by the presence of GAD67+mCherry+ neurons in the hilus of the hippocampus. Following intracranial stimulation in the hippocampus of Tg and nTg subjects (black arrow) nTg rat showed comparable responses between the mCherry transfected and the Ascl1-mCherry transfected hippocampi (A). On the other hand, TgF344 AD rat showed larger responses in the Ascl1-mCherry hippocampus than in the mCherry transfected (B). These results suggest improved neuronal network function after transduction of new neurons. Scale bars=100 μm

FIG. 15: Ascl1, Ascl1-SA6 and Neurod1 effectively increase the number of NeuN+ neurons in the hilus of the hippocampus in comparison to the ipsilateral hemisphere.

FIG. 16: Direct lineage reprogramming using Ascl1-SA6 leads to a decrease in GFAP+ cells. (A) Respective immunofluorescence images of GFAP+ cells in the hippocampal hilus (i: 10×, ii-vi: 20×). (B) Quantification of GFAP+ cells per mm² (n=5, 2 way ANOVA, Bonferroni test). (C) Fold change in GFAP+ cells in the hippocampal hilus between injected hemispheres and naïve rats (n=5, One-way ANOVA, Bonferroni test).

FIG. 17: Reactive astrocytes are decreased in the Ascl1-SA6 infected hilus (right panel) in comparison to the uninfected hemisphere (left panel). Reactive astrocytes are circled to illustrate the difference from non-reactive cells. Scale bars=100 m

FIG. 18: Ascl1-SA6 leads to a significant decrease in plaque coverage in the hippocampal hilus in vivo. (A) Immunofluorescence images using anti-MOAB2 antibody in naïve and treated TgF344 AD rats (20×). (B) Quantitative analysis of plaque coverage, as a percent of the total area (n=5, 2 way ANOVA, Bonferroni test). (C) Fold change in plaque coverage between injected hemisphere of treated versus naïve rats (n=5, One-way ANOVA, Bonferroni test).

FIG. 19: Viral delivery of Ascl1 and Ascl1-SA6 led to significant changes in plaque-associated microglia and mean plaque size in vivo. (A) Immunofluorescence of IBA-1+4G8+ cells in the hippocampal hilus (20×). (B) Quantitative analysis of IBA-1+4G8+ cells (n=5, One-way ANOVA, Bonferroni test). (C) Quantitative analysis of mean plaque size, using an anti-4G8 antibody (n=5, One-way ANOVA, Bonferroni test).

FIG. 20: Viral delivery of Ascl1-SA6 leads to a decrease in activated and increase in ramified IBA-1+ cells in vivo. (A) Immunofluorescence of IBA+ cells in the hippocampal hilus (20×). (B) High magnification of a (i) activated and (ii) ramified IBA-1+ cells. (C) Quantitative analysis of total IBA-1+ cells in the hippocampal hilus (n=5, One-way ANOVA, Bonferroni test). (D) Quantitative analysis of activated and ramified cells as a percent of total IBA-1+ cells (n=5, 2 way ANOVA, Bonferroni test).

FIG. 21: Transient expression of Ascl1-SA6 leads to a decrease in hypertrophic and increase in physiologic astrocytes in vivo. (A) Immunofluorescence of GFAP+ cells in the hippocampal hilus (20×). (B) High magnification of a (i) hypertrophic and (ii) physiologic astrocyte. (C) Quantitative analysis of hypertrophic and physiologic astrocytes as a percent of total GFAP+ cells (n=5, 2 way ANOVA, Bonferroni test).

FIG. 22: Viral delivery of Ascl1-SA6 results in decreased abundance of markers indicative of reactive astrocytes. (A) Immunofluorescence of S1003+ cells in the hippocampal hilus (20×). (B) Quantitative analysis of S1003+ cells (n=5, One-way ANOVA, Bonferroni test). (C) In situ hybridization for C3 and immunofluorescence staining for S1003 in AD. Modified from Liddelow et al, Nature 541, 481-487 (2017). (D) Quantitative analysis of immunofluorescence of C- and S100β-positive cells in the hippocampal hilus (n=5, One-way ANOVA, Bonferroni test).

FIG. 23: Direct lineage reprogramming leads to decreases in necrotic neurons in vivo. (A) Immunofluorescence of @111-tubulin+ and RIPK1+ cells in naïve and treated TgF344 AD rats (20×). (B) Quantitative analysis of necrotic neurons (βIII-tubulin+RIPK1+) as a percent of total β111-tubulin+ cells (n=5, One-way ANOVA, Bonferroni test).

FIG. 24: Direct lineage reprogramming leads to increased interneurons in vivo. (A) Quantification of GAD67+ cells in naïve and Ascl1 or Ascl1-SA6 treated TgF344 AD rats. (B) Quantitative analysis of fold changes in interneurons between the two hemispheres of TgF344 AD rat treated with Ascl1-SA6 in comparison to between treated versus untreated TgF344 AD rats (n=5, One-way ANOVA, with correction for multiple groups).

FIG. 25: Direct lineage reprogramming leads to improved cognitive function. (A) Comparison of spatial memory between naïve, Ascl1- and Ascl1-SA6 treated NTg rats showed no significant difference whereas TgF344 AD rats treated with Ascl1 and significantly with Ascl1-SA6 showed improved memory in comparison to untreated rats. (B) Untreated TgF344 AD rats learned the Barnes maze task significantly slower than TgF344 AD rats treated with Ascl1-SA6 and NTg rats both treated and untreated. TgF344 AD rats treated with Ascl1-SA6 were not significantly different from NTg rats both treated and untreated. (C) Examination of executive function or the ability to problem solve was significantly better in TgF344 AD rat treated with Ascl1-SA6 in comparison to between untreated TgF344 rats and were not significantly different from treated and untreated NTg rats. (n=5, One-way ANOVA, with correction for multiple groups).

FIG. 26: Ascl1-SA6 is more efficient at astrocyte to neuron reprogramming following stroke. (A) Schematic of experimental paradigm. Mice received ET-1 stroke (single) on day 0 and reprogramming (AAV-Cre; AAV-Ascl1; AAV-Ascl1-SA6) and were sacrificed on PSD28 for tissue analysis. (B) The percent reprogramming (TdTom+NeuN+/TdTom+ cells) reveals significantly greater astrocyte to neuron reprogramming in AAV-Ascl1 and AAV-Ascl1-SA6 treated mice relative to controls (AAV-Cre). Ascl1-SA6 treated mice had a greater percentage of reprogrammed neurons compared to AAV-Ascl1 treated mice. (C) Representative images from stroke injured cortices of (i) AAV-Cre (ii) AAV-Ascl1 and (iii) AAV-Ascl1-SA6 brains on PSD28. White arrows indicate examples of TdTom+(AAV transduced), NeuN+. Scale bar=100 um. Data represents means+/−SEM. N=6-12 mice/group; 1-way ANOVA; *p<0.05, **p<0.01, ***p<0.001.

FIG. 27: Ascl1-SA6 overexpression results in improved motor function in a subacute model of stroke at 3 weeks post-AAV transduction. (A) Schematic of experimental timeline. Mice received ET-1 stroke (single injection) on day 0 and reprogramming on day 7 in the subacute phase (AAV-Cre (control, light grey bars, n=4,7); AAV-Ascl1 (black bars, n=7,8) or AAV-Ascl1-SA6, (dark grey bars, n=9,10)). Percent slippage of the Hindpaw (B) and Forepaw (C) was assessed at baseline (prior to stroke, PSD4 (prior to reprogramming) and PSD29. Mice that received Ascl1-Sa6 were has significantly reduced % slippage at 21 days post-reprogramming. Data represents means+/−SEM. N=4-10 mice/group; 2 way ANOVA Tukey's multiple comparisons, *p<0.05, **p<0.01, ***p<0.001, ****p<0.0001

FIG. 28: Ectopic expression of Ascl1-SA6 results in improved gait outcomes at 3 weeks post-reprogramming: At 28 days post-stroke and treatment, the impaired (left) hindpaw print area and max intensity are significantly impaired in control (AAV-Cre, light grey bars) treated mice. Mice that received stroke and AAV-Ascl1-SA6 performed significantly better in these gait parameters. Data represents means+/−SEM. N=5-7 mice/group; 1 way ANOVA, Tukey's multiple comparisons, *p<0.05, **p<0.01.

FIG. 29: Ectopic expression of Ascl1-SA6 improves skilled motor behaviour in the horizontal ladder task: (A) Schematic of experimental design to assess skilled motor function in the horizontal ladder task in mice that receive a triple ET-1 stroke. (B) The average percent slippage of the impaired forepaw (left slips/left steps*100) reveals that AAV-Ascl1-SA6 treated mice are significantly improved by PSD29 compared to their PSD4 performance, whereas AAV-Cre and AAV-Ascl1 treated mice remain impaired at PSD29. N=6-11 mice/group; 2 way ANOVA Tukey's multiple comparisons, *p<0.05, **p<0.01, ****p<0.0001, ns=not significant.

FIG. 30: Ascl1 and Ascl1-SA6 are able to promote functional recovery in the grip test at PSD28 following ET-1 stroke: (A) Schematic of the experimental design to assess forepaw strength (g) following a single ET-1 stroke. (B) Stroke_AAV-Cre mice did not demonstrate grip strength improvement at any time examined. Mice that received Ascl1 or Ascl1-SA6 show significant improvement by PSD28 (not significantly different from baseline performance (100%) pre-stroke). Data represents means+/−SEM. N=12-26 mice/group; 2 way ANOVA Tukey's multiple comparisons, ****p<0.0001, ns=not significant.

FIG. 31: Ectopic expression of Ascl1-SA6 promotes functional recovery in the grip strength task at a faster rate than Ascl1 in a more severe model of stroke: (A) Schematic of the experimental design to assess forepaw strength (g) following a triple ET-1 stroke. (B) Stroke_AAV-Cre mice were significantly impaired from baseline (pre-stroke) performance at all times examined. Mice that received Ascl1 or Ascl1-SA6 show significant improvement by PSD120 (not significantly different from baseline performance pre-stroke). Mice that received AAV-Ascl1-SA6 recovered grip strength by PSD59 and the recovery was maintained to PSD120 (the longest time examined). Data represents means+/−SEM. N=12-26 mice/group; 2 way ANOVA Tukey's multiple comparisons, 2 way ANOVA Tukey's multiple comparisons, *p<0.05, **p<0.01, ***p<0.001, ****p<0.0001, ns=not significant.

FIG. 32: Schematic of the experimental workflow for cerebral organoid (CO) culture.

FIG. 33: In COs injected with AAV2/8-GFAP-mCherry (left panel), GFAP expression (astrocyte marker) was co-localized with mCherry expressed under GFAP promoter. Conversely, the expression of GFAP was reduced in COs injected with AAV2/8-GFAP-Ascl1-mCherry (right panel) and the morphology of cells expressing mCherry was changed from astrocyte-like structure into neuronal-like structure.

FIG. 34: In COs injected with AAV2/8-GFAP-mCherry (left panel), Doublecortin (DCX; early neuron marker) expression was not co-localized with mCherry expressed under the GFAP promoter. However, the expression of DCX was co-localized with mCherry (bright arrows) in COs injected with AAV2/8-GFAP-Ascl1-mCherry (right panel).

FIG. 35: (A) In human CO's (90 days) injected with AAV2/8-GFAP-Ascl1-mCherry or AAV2/8-mCherry showed a decreased proportion of astrocytes (GFAP) with a concomitant increase in neuronal markers (NeuN, mature neurons; DCX, immature neurons at 21 days following transduction; (B) Photomicrograph showing transduced astrocytes co-labeled with NeuN (arrows) at 21 days (i); (II) mCherry transduced cells (arrows) express NeuN (iii) and not GFAP (iv); DAPI (v).

FIG. 36: Establishing a lineage tracing system to follow the fate of cortical astrocytes transduced in vivo. (A) Schematic illustration of AAV8-GFAPlong-mCherry vectors and injection strategy into the rostral cerebral cortex in vivo. (B) mCherry co-expression with GFAP or Dcx one-month post-transduction, and with GFAP or NeuN two-months post-transduction. Blue is DAPI counterstain. (C) Quantification of the percentage of mCherry⁺ transduced cells expressing NeuN after transduction with AAV8-GFAPlong-Ascl1-mCherry. (D) Schematic illustration of AAV5-GFAPshort-iCre vectors and injection strategy into the cortex in vivo. (E) zsGreen expression in the cortex of Rosa-zsGreen mice injected with AAV5-GFAPshort-Ascl1-iCre at two-months post-transduction. Blue is DAPI counterstain. (F,G) Colorimetric RNA in situ hybridization (F) and fluorescent RNAscope analysis of Ascl1 transcript distribution in cortices transduced with AAV5-GFAPshort-iCre (G) or AAV5-GFAPshort-Ascl1-iCre (F,G) at two-months post-transduction. The boxed area in G is magnified in the panel to the right. (H) Schematic illustration of the sequence of wild-type (wt) Ascl1 with the SP sites designated, and Ascl1-SA6, with the SA mutations indicated. Scale bars in B=25 μm, in E=200 μm, and in G=75 μm.

FIG. 37: Ascl1-SA6 induces more transduced cortical cells that express NeuN, a mature neuronal marker, than Ascl1. (A) Schematic illustration of Cre-based lineage tracing strategy, using AAV5-GFAPshort vectors and Rosa-tdtomato or Rosa-zsGreen transgenic animals. (B) Low magnification image of Rosa-zsGreen cortex transduced with AAV5-GFAPshort-iCre at 21 days post-transduction, showing zsGreen epifluorescence and NeuN expression. (C) Rosa-tdtomato cortex transduced with AAV5-GFAPshort-iCre, AAV5-GFAPshort-Ascl1-t2a-iCre, and AAV5-GFAPshort-Ascl1-SA6-t2a-iCre at 21 days post-transduction, showing tdtomato epifluorescence and NeuN expression. (D) Quantification of the percentage of tdtomato⁺ cells that co-express NeuN. (E) Rosa-zsGreen cortex transduced with AAV5-GFAPlong-iCre, AAV5-GFAPlong-Ascl1-t2a-iCre, and AAV5-GFAPlong-Ascl1-SA6-t2a-iCre at 21 days post-transduction, showing zsGreen epifluorescence and NeuN expression. (F) Quantification of the percentage of zsGreen⁺ cells that co-express NeuN. (G) Rosa-zsGreen cortex transduced with AAV8-GFAPshort-iCre, AAV8-GFAPshort-Ascl1-t2a-iCre, and AAV8-GFAPshort-Ascl1-SA6-t2a-iCre at 21 days post-transduction, showing zsGreen epifluorescence and NeuN expression. (H) Quantification of the percentage of zsGreen⁺ cells that co-express NeuN. Scale bars in B=200 μm, and C,E,G=100 μm.

FIG. 38. Ascl1-SA6 more efficiently represses Sox9 expression, a glioblast marker, in transduced cortical cells than Ascl1. (A) Rosa-zsGreen cortex transduced with AAV5-GFAPlong-iCre, AAV5-GFAPlong-Ascl1-t2a-iCre, and AAV5-GFAPlong-Ascl1-SA6-t2a-iCre at 21 days post-transduction, showing zsGreen epifluorescence, Sox9 (red) and GFAP (white) expression. Blue is a DAPI counterstain. Shown are merged images and separated Sox9 (red) and GFAP (white) channels. (B) Rosa-zsGreen cortex transduced with AAV8-GFAPshort-iCre, AAV8-GFAPshort-Ascl1-t2a-iCre, and AAV8-GFAPshort-Ascl1-SA6-t2a-iCre at 21 days post-transduction, showing zsGreen epifluorescence Sox9 (red) and GFAP (white) expression. Blue is a DAPI counterstain. Shown are merged images and separated Sox9 (red) and GFAP (white) channels. (C,D) Quantification of the percentage of zsGreen⁺ cells that co-express Sox9 or GFAP, and the percentage of zsGreen⁺Sox9⁺ cells that co-express GFAP for both the AAV5-GFAPlong series (C) and the AAV8-GFAPshort series (D). Scale bars=100 μm.

FIG. 39. Both Ascl1 and Ascl1-SA6 induce Dcx expression in GFAP⁺ transduced astrocytes. (A-C) Rosa-zsGreen cortex transduced with AAV5-GFAPlong-iCre (A), AAV5-GFAPlong-Ascl1-t2a-iCre (B), and AAV5-GFAPlong-Ascl1-SA6-t2a-iCre (C) at 21 days post-transduction, showing zsGreen epifluorescence, Dcx (red) and GFAP (white) expression. Blue is a DAPI counterstain. (D) Quantification of the percentage of zsGreen⁺ cells that co-express Dcx alone, or Dcx and GFAP. Scale bars=100 μm.

FIG. 40. Optogenetic stimulation of ChR2 actuator reveals that Ascl1-SA6 transduced cells have a shorter decay constant. (A) Schematic illustration of optogenetic experiment in which we injected AAVs carrying ChR2-(H134R)-YFP, an optogenetic actuator, with Cre-based AAVs into the cortex of Rosa-zsGreen mice. Four weeks later, photostimulation experiments were performed. (B) Craniotomy and cortical window used for simultaneous photostimulation and electrophysiological recordings. Also shown is a two-photon z-stack projection of the cortical tissue showing Zs-green positive cells (green channel) and the tungsten electrode tip (red channel); the circular yellow area represents the site of stimulation near the electrode; scalebar=100 m. (C) Representative raw voltage traces showing the changes in the LFP band following 3 seconds photostimulation at 4 Hz for both ASCL1-SA6 (purple trace) and iCre (black trace) treatments. Shaded areas indicate the voltage standard deviation measured across different repetitions, within the same photostimulated area. (D) Measurement of the decay constant of photostimulated cells in iCre and Ascl1-SA6 transduced brains. Dots in the boxplots indicate individual measures and “N” indicates the number of mice used in each group.

FIG. 41: Reduction in Ctip2⁺ upper motor neurons without reactive microgliosis in hSod1^{G93A} brains at 3 and 5 months of age. (A) Schematic representation of the rostrocaudal positions of analysis. (B) DAPI staining of cortical sections at Bregma 2.15, 1.85 and 1.34. (C-F) Coronal sections through C57Bl/6 control (C,E) and hSod1^{G93A} (D,F) motor cortices immunostained with Ctip2 (green) and Iba1 (red) at 3 mo (C,D) and 5 mo (E,F) of age. Blue is DAPI counterstain. (G-1) Quantification of the % Ctip2⁺ cells/DAPI⁺ nuclei at 3 mo and 5 mo of age, showing counts at Bregma 2.15 (G), 1.85 (H) and 1.34 (J). (J-O) Analysis of microgliosis, showing the % Iba1⁺ cells/DAPI⁺ nuclei in C57Bl/6 control and hSod1^{G93A} motor cortices at 3- and 5-mo of age (J). Characterization of Iba1⁺ cell density (K), mean soma size (L) and branches per cell (M) in 3 mo and 5 mo C57Bl/6 control and hSOD1^{G93A} motor cortices. (N,O) Identification of activated microglia using MORPHIOUS, showing the tuning process used to optimally identify activated cells. Red in the heatrmap indicates larger values, and black denotes 0, shown at 3 mo (N) and 5 mo (O) of age. Scale bars in B-F=200 μm.

FIG. 42: Astrogliosis is not evident in hSod1^{G93A} brains at 3 and 5 months of age. (A) Schematic representation of the rostrocaudal positions of analysis. (B-E) Coronal sections through C57Bl/6 control (B,D) and hSod1^{G93A} (C,E) motor cortices immunostained with GFAP (green) and Sox9 (red) at 3 mo (B,C) and 5 mo (D,E) of age. Blue is DAPI counterstain. (F-1) Quantification of the % Sox9⁺ cells/DAPI⁺ nuclei at 3 mo and 5 mo of age, showing counts at Bregma 2.15 (F), 1.85 (G) and 1.34 (H). (I-K) Analysis of astrogliosis, using MORPHIOUS to characterize GFAP expression in C57Bl/6 control and hSod1^{G93A} motor cortices at 3- and 5-mo of age (I). (N,O) The tuning process used to optimally identify activated cells is shown, with red in the heatmap indicating larger values, and black denoting 0, shown at 3 mo (J) and 5 mo (K) of age. Scale bars in B-E=200 μm.

FIG. 43: Cluster analysis and assignment in uninjected, iCre, Ascl1 and Ascl1^{SA6} injected hSod1^{G93A} motor cortices. (A,B) Experimental protocol, showing the injections of the 3 AAVs into the motor cortex of 16-week-old hSod1^{G93A}; Rosa-zsGreen mice using a stereotax (A). After 28 dpi, the brains were sectioned onto the Visium capture spots, the tissue was permeabilized to capture mRNA on barcoded primers (A). Primer sequences include spatial barcodes, a UMI and poly dT tail (B). The Fiducial frame and capture area of each ‘spot’ are shown (B). (C,D) ST analysis of uninjected hSod1^{G93A} motor cortex showing H&E-stained section in the Fiducial frame, spatial map, and UMAP (C). Heatmap of the top 50 variably expressed genes across the tissue (D). (E,F) ST analysis of hSod1^{G93A} motor cortex injected with iCre, showing H&E-stained section in the Fiducial frame, spatial map, and UMAP (E). Heatmap of the top 50 variably expressed genes across the tissue (F). (G,H) ST analysis of hSod1^{G93A} motor cortex injected with Ascl1, showing H&E-stained section in the Fiducial frame, spatial map, and UMAP (G). Heatmap of the top 50 variably expressed genes across the tissue (H). (I,J) ST analysis of hSod1^{G93A} motor cortex injected with Ascl1^{SA6}, showing H&E-stained section in the Fiducial frame, spatial map, and UMAP (I). Heatmap of the top 50 variably expressed genes across the tissue (J). Cluster assignments in D,F, H,J) were made using the Allen Brain Atlas and comparing to a brain palette of 266 genes in a molecular brain atlas³⁹.

FIG. 44: iCre transduced cells are associated with an inflammatory transcriptional signature. (A-E) Mapping iCre transcript distribution onto the spatial maps for the uninjected, iCre, Ascl1 and Ascl1^{SA6} transduced brains (A). Schematic of Rosa-zsGreen locus that is recombined by iCre (B). Confirmation of zsGreen expression in the iCre, Ascl1 and Ascl1^{SA6} transduced brains (C). Model of v2a-triggered ribosome skipping, resulting in the generation of bicistronic mRNA carrying coding sequences for Ascl1 and iCre, both of which are translated as separate proteins by ribosome skipping (D). Normalization and scaling of iCre expression in the iCre, Ascl1 and Ascl1^{SA6} transduced brains to allow for DEG analysis (E). (F-J) Venn diagrams showing the distributions of DEGs that are upregulated and downregulated when comparing Ascl1 to iCre or Ascl1^{SA6} to iCre (F). Heatmap of DEGs between the iCre positive and iCre negative cells within each tissue (G). Comparison of GO terms separated in semantic space that relate to the DEG in iCre transduced cells, including GO terms associated with upregulated DEGs (H). Spatial mapping of transcript distribution for Gfap, Vim, Tyrobp, Mpeg1, Cd52, and C4b on uninjected, iCre, Ascl1 and Ascl1^{SA6} brains (1). Log 2FC in transcript reads for a set of DEGs, showing values for iCre (black circles), Ascl1 (blue circles) and Ascl1^{SA6} (green circles) (J).

FIG. 45: Quality control of spatial transcriptomics (ST) data. (A-A″) ST analysis of uninjected hSod1^{G93A} motor cortex, showing H&E-stained section in the Fiducial frame (A), a tSNE plot with UMI counts in each cluster (A), a spatial map (A′) and a corresponding, color-coded tSNE plot (A′). Sequencing saturation is also indicated (A″). (B-B″) ST analysis of hSod1^{G93A} motor cortex injected with iCre, showing H&E-stained section in the Fiducial frame (B), a tSNE plot with UMI counts in each cluster (B), a spatial map (B′) and a corresponding, color-coded tSNE plot (B′). Sequencing saturation is also indicated (B″). (C-C″) ST analysis of hSod1^{G93A} motor cortex injected with Ascl1, showing H&E-stained section in the Fiducial frame (C), a tSNE plot with UMI counts in each cluster (C), a spatial map (C′) and a corresponding, color-coded tSNE plot (C′). Sequencing saturation is also indicated (C″). (D-D″) ST analysis of hSod1^{G93A} motor cortex injected with Ascl1^{SA6}, showing H&E-stained section in the Fiducial frame (D), a tSNE plot with UMI counts in each cluster (D), a spatial map (D′) and a corresponding, color-coded tSNE plot (D′). Sequencing saturation is also indicated (D″).

FIG. 46: Neuron-associated gene expression is enriched in Ascl1 and Ascl1^{SA6} transduced cells. (A-C) Transcript distribution of DEGs in the iCre positive cells in uninjected, iCre, Ascl1 and Ascl1^{SA6} transduced cells, including Ptgds, Pvalb, Gad1, and Sst (A). Transcript distribution of DEGs identified in an Ascl1^{ERT2} overexpression experiment in postnatal astrocytes in vitro¹⁰ in uninjected, iCre, Ascl1 and Ascl1^{SA6} transduced cells, including Id3, C1qb, and C1qa (B). Log 2FC in transcript reads for a set of DEGs, showing values for iCre (black circles), Ascl1 (blue circles) and Ascl1^{SA6} (green circles) (C). (D-G) Comparison of GO terms separated in semantic space that relate to DEGs upregulated (D,F) and downregulated (E,G) in Ascl1 and Ascl1^{SA6}-transduced cells, respectively.

FIG. 47: Identification of a GRN involving Zbtb18 and Id TFs activated by Ascl1 and Ascl1^{SA6} in vivo. (A-C) GRNs comprised of TFs were built from the ST data for iCre (A), Ascl1 (B) and Ascl1^{SA6} (C) transduced brains. The darkest grey boxes (originally red) surround DEGs specifically upregulated either in iCre alone, or in Ascl1 and/or Ascl1^{SA6} transduced brains. The dark grey boxes (originally orange) surround DEGs upregulated in iCre and either Ascl1 and/or Ascl1^{SA6} transduced brains. Lightest grey boxes (originally blue) surround DEGs specifically downregulated either in iCre alone, or in Ascl1 and/or Ascl1^{SA6} transduced brains. Medium grey boxes (originally purple) surround DEGs specifically downregulated in iCre and either Ascl1 and/or Ascl1^{SA6} transduced brains. (D-1) Comparative analysis of upregulated DEGs from the in vivo ST data in this study to upregulated DEGs from in vitro reprogramming of glioblastoma cells using native ASCL1 (WT 51) (D-F) or a mutated version of ASCL1^{SA5} (G-1) (GSE153823; ²⁵). Shown are comparisons to iCre (D,G), Ascl1 (E,H) and Ascl1^{SA6} ST data, focusing on upregulated DEGs (F,I). (J,K) Log 2FC in transcript reads for upregulated (J) and downregulated (K) DEGs, showing values for iCre (black circles), Ascl1 (blue circles) and Ascl1^{SA6} (green circles). (L,M) Generation of an embryonic cortical GRN inferred from a published scRNAseq dataset⁹⁶, showing TFs that are upregulated (dark grey squares) or downregulated (light grey squares) in response to Ascl1^{SA6} overexpression in glioblastoma cells (GSE153823; ²⁵), either in an unperturbed state (L) or following an in silico knock-out (‘KO’) of Zbtb18 (M). (N-Q) Comparative analysis of Neurog2 (designated ‘N’), Ascl1 (designated ‘A’) and Zbtb18 (designated ‘Z’) in E12.5 cortical NPCs, first demonstrating that all three genes are part of the same GRN built from RNAseq data of Neurog2+Ascl1+NPCs 97. Lineage-trajectory analysis of Ascl1, Neurog2 and Zbtb18, shown on SPRING plots of E12.5 cortical scRNAseq data⁹⁸, showing a bifurcation of glutamatergic and GABAergic neuronal lineages (O). Also shown are pseudoDV scores, depicting regional biases of Ascl1, Neurog2 and Zbtb18 expression (P) and a pseudotime analysis of the three genes (Q).

FIG. 48: Comparison of in vivo (ST data) to published in vitro neuronal reprogramming datasets. (A-C) Comparative analysis of upregulated DEGs from the in vivo ST data in this study to upregulated DEGs from in vitro reprogramming of postnatal astrocytes using ASCL1 (GSE06389; ¹⁰). Shown are comparisons to iCre (A), Ascl1 (B) and Ascl1^{SA6} (C) ST data, focusing on upregulated DEGs. (D-F) Comparative analysis of downregulated DEGs from the in vivo ST data in this study to downregulated DEGs from in vitro reprogramming of postnatal astrocytes using ASCL1 (GSE06389; ¹⁰). Shown are comparisons to iCre (D), Ascl1 (E) and Ascl1^{SA6} (F) ST data, focusing on downregulated DEGs. (G-J) Comparative analysis of downregulated DEGs from the in vivo ST data in this study to downregulated DEGs from in vitro reprogramming of glioblastoma cells using native ASCL1 (WT 51) (G-1) or a mutated version of ASCL1^{SA5} (J-L) (GSE153823; ²⁵). Shown are comparisons to iCre (G,J), Ascl1 (H,K) and Ascl1^{SA6} (I,L) ST data, focusing on downregulated DEGs.

FIG. 49: Experimental outline for iCre and Ascl1^{SA6} injections at 16 weeks of age in hSOD1^{G93A} and control mice and body weight measurements. (A) AAV injections and behavior test timeline. iCre and Ascl1^{SA6} were injected bilaterally into 16-week-old Rosa-zsGreen (control) and 16-week-old (symptomatic) hSOD1^{G93A}; Rosa-zsGreen mice, both male and female. Body weight and motor behavioural tests were performed weekly and compared to 15-week baseline measures. (B,C) Change in body weight over the experimental timeline for male (B) and female (C) mice. Two-way ANOVA showed significant differences at 19 and 22 weeks in hSOD1^{G93A} mice (p=0.0470 and p=0.0370, respectively).

FIG. 50: Neurological scores and survival curves for iCre and Ascl1^{SA6} injection at 16 weeks in male and female hSOD1^{G93A} mice. Behavioural tests were performed weekly and continued until humane endpoint. (A,B) NS assignments for male (A) and female (B) hSOD1^{G93A} mice. (C) Kaplan-Meier survival curves for iCre and Ascl1^{SA6}_injected male (C) and female (D) mice.

FIG. 51: Rotarod and grip strength for iCre and Ascl1^{SA6} injection at 16 weeks in hSOD1^{G93A} and control mice. (A,B) Rotarod assays were performed at 15-weeks (baseline) and weekly from 17 weeks after AAV injections at 16 weeks-of-age in male (A) and female (B) hSOD1^{G93A} and control mice. hSOD1^{G93A} male treated with Ascl1^{SA6} increased their ability to stay on the rotarod and showed significant differences at 19, 20, and 21 weeks of age compared to the iCre injected cohort (A). Statistical tests could not be performed at 24 weeks and beyond because control animals did not survive. (Šídák's multiple comparisons test, p=0.0471, p=0.0379, p=0.0329 at 19, 20, and 21 weeks, respectively). hSOD1^{G93A} female mice injected with Ascl1^{SA6} had an increased ability to stay on the rotarod and showed significant differences at 23 and 25 weeks-of-age. (Šídák's multiple comparisons test, p=0.0359 and p<0.0001, respectively) (C,D) Grip strength measurements of neuromuscular function were performed at 15-weeks (baseline) and weekly from 17 weeks after AAV injections at 16 weeks-of-age in male (C) and female (D) hSOD1^{G93A} and control mice. hSOD1^{G93A} male mice injected with Ascl1^{SA6} showed a trend towards increased grip strength.

FIG. 52: Gait analysis for iCre and Ascl1^{SA6} treated control and hSOD1^{G93A} mice at 16 weeks of age. Gait analysis of stride length distance based on measurement of fore and hindlimb paw prints in male (A) and female (B) control and hSOD^{G93A} mice. (A) hSOD1^{G93A} male mice injected with Ascl1^{SA6} displayed a trend towards increased stride length but did not show significant differences. (B) hSOD1^{G93A} female mice injected with iCre had a significantly longer stride length at 15 and 21 weeks of age compared to Ascl1^{SA6} treated animals (Šídák's multiple comparisons test, p=0.0319 and p=0.0057, respectively).

FIG. 53: CatWalk XT® gait analysis of right (RH) and left (LH) hindlimb stride distances after iCre and Ascl1^{SA6} injections at 16 weeks. (A,B) RH (A) and LH (B) stride lengths in control and hSOD1^{G93A} male mice. (C,D) RH (C) and LH (D) stride lengths in control and hSOD1^{G93A} female mice.

FIG. 54: Body weight measures after iCre and Ascl1^{SA6} injection at 19 weeks-of-age in hSOD1^{G93A} and control mice. Body weight was measured weekly and compared to 18-week baseline measures in male (A) and female (B) mice.

FIG. 55: Neuroscores and Kaplan-Meier survival curves for iCre and Ascl1^{SA6} injected hSOD1^{G93A} mice at 19 weeks of age. Behavioural tests were performed weekly and continued until humane endpoint. (A,B) NS assignments for male (A) and female (B) hSOD1^{G93A} mice. (C) Kaplan-Meier survival curves for iCre and Ascl1^{SA6}_injected male and female mice. Ascl1^{SA6} injected male mice showed 60% survival probability until 28 weeks compared to 0% for iCre injected male mice. Ascl1^{SA6} injected female mice showed 35% survival probability until 26 weeks compared to 0% for iCre injected female mice.

FIG. 56: Rotarod and grip strength for iCre and Ascl1^{SA6} injection at 19 weeks in hSOD1^{G93A} and control mice. (A,B) Rotarod assays were performed at 18-weeks (baseline) and weekly from 20 weeks after AAV injections at 19 weeks-of-age in male (A) and female (B) hSOD1^{G93A} and control mice. hSOD1^{G93A} male treated with Ascl1SA6 trended towards an increase in their ability to stay on the rotarod (A). hSOD1^{G93A} female mice injected with Ascl1^{SA6} or iCre did not show a difference in latency to fall on the rotarod (B) (C,D) Grip strength measurements of neuromuscular function were performed at 18-weeks (baseline) and weekly from 20 weeks after AAV injections at 19 weeks-of-age in male (C) and female (D) hSOD1^{G93A} and control mice. hSOD1^{G93A} male mice injected with Ascl1^{SA6} showed a trend towards increased grip strength.

FIG. 57: CatWalk XT® gait analysis of right (RH) and left (LH) hindlimb stride distances after iCre and Ascl1^{SA6} injections at 19 weeks. (A,B) RH (A) and LH (B) stride lengths in control and hSOD1^{G93A} male mice. (C,D) RH (C) and LH (D) stride lengths in control and hSOD1^{G93A} female mice.

FIG. 58: Ascl1-SA7 represses Sox9 expression, a glioblast marker, in transduced cortical cells more efficiently than Ascl1 and to the same extent as Ascl1-SA6. (A) Rosa-zsGreen cortex transduced with AAV8-GFAPshort-iCre, AAV8-GFAPshort-Ascl1-t2a-iCre, AAV8-GFAPshort-Ascl1-SA6-t2a-iCre and AAV8-GFAPshort-Ascl1-SA7-t2a-iCre at 21 days post-transduction, showing zsGreen epifluorescence, Sox9 and GFAP (white) expression. DAPI was used as a counterstain. Shown are merged images (top panel) and separated Sox9 (second panel) and GFAP (bottom pannel) channels. (B) The graphs show quantification of the percentage of zsGreen⁺ cells that co-express Sox9 or GFAP, and the percentage of zsGreen⁺Sox9⁺ cells that co-express GFAP. Scalebars=100 μm.

FIG. 59: Ascl1-SA7 and Ascl1-SA6 induce more transduced cortical cells to express NeuN, a mature neuronal marker, than Ascl1. (A) Rosa-zsGreen cortex transduced with AAV8-GFAPshort-iCre, AAV8-GFAPshort-Ascl1-t2a-iCre, AAV8-GFAPshort-Ascl1-SA6-t2a-iCre and AAV8-GFAPshort-Ascl1-SA7-t2a-iCre at 21 days post-transduction, showing zsGreen epifluorescence and NeuN expression. (B) Quantification of the percentage of zsGreen+ cells that co-express NeuN. Scalebars in 100 μm.

### DETAILED DESCRIPTION

### Definitions

Unless defined otherwise, all technical and scientific terms used herein have the same meaning as commonly understood by one of ordinary skill in the art to which this invention belongs.

As used in the specification and claims, the singular forms “a”, “an” and “the” include plural references unless the context clearly dictates otherwise.

The term “comprising” as used herein will be understood to mean that the list following is non-exhaustive and may or may not include any other additional suitable items, for example one or more further feature(s), component(s) and/or ingredient(s) as appropriate.

Reference throughout this specification to “one embodiment,” “an embodiment,” “another embodiment,” “a particular embodiment,” “a related embodiment,” “a certain embodiment,” “an additional embodiment,” or “a further embodiment” or combinations thereof means that a particular feature, structure or characteristic described in connection with the embodiment is included in at least one embodiment. Thus, the appearances of the foregoing phrases in various places throughout this specification are not necessarily all referring to the same embodiment. Furthermore, the particular features, structures, or characteristics may be combined in any suitable manner in one or more embodiments.

As used herein, the term “effective amount” of a substance is an amount sufficient to produce a desired effect. In some embodiments, an effective amount of a transcription factor, or a nucleic acid encoding a transcription factor, is an amount sufficient to transdifferentiate a sufficient number of target cells of a target tissue of a subject. In some embodiments, a target tissue is neuronal tissue (e.g., brain tissue, such as, but not limited to, cerebral cortex (CCTX), hippocampus (HIPPO), entorhinal cortex (EC), pre-frontal cortex (PFC), posterior medial cortex (PMC), locus coeruleus (LC), thalamus (TH), inferior colliculus (IC), olfactory bulb (OB), anterior olfactory nucleus (AON), hypothalamus (HT), cerebellum (CBL), striatum, basal ganglia, limbic system (LC), default mode network (DMN), medial temporal lobes (MTL), brain stem, cerebellum, and spinal cord). In some embodiments, an effective amount of a composition or vector may be an amount sufficient to have a therapeutic benefit in a subject, e.g., to provide neuronal reprogramming of astrocytes to neurons, to extend the lifespan of a subject, to delay, prevent or treat a neurodegenerative disease or disorder or injury in the subject, or to treat a central nervous system disease, disorder or injury in the subject, etc. The effective amount will depend on a variety of factors such as, for example, the species, sex, age, weight, health of the subject, and the mode of administration or site of administration, and may thus vary among subjects and administrations.

As used herein, a “subject” or “patient” or “individual” to be treated by the method of the invention is meant to refer to either a human or non-human animal. A “non-human animal” includes any vertebrate or invertebrate organism, but is preferably a mammal. A human subject can be of any age, gender, race or ethnic group, e.g., Caucasian (white), Asian, African, black, African American, African European, Hispanic, Middle Eastern, etc. In some embodiments, the subject can be a patient or other subject in a clinical setting. In some embodiments, the subject is already undergoing treatment. In some embodiments, the subject is a neonate, infant, child, adolescent, adult, or an elderly adult.

As used herein, the terms “protein” and “polypeptide” are used interchangeably and thus the term polypeptide may be used to refer to a full-length protein and may also be used to refer to a fragment of a full-length protein, and/or functional variants thereof.

As used herein, the terms “polynucleotide” and “nucleic acid sequence” may be used interchangeably and may comprise genetic material including, but not limited to: RNA, DNA, mRNA, cDNA, etc., which may include full length sequences, functional variants, and/or fragments thereof.

As used herein, the term “vector” is meant to refer to a vehicle to artificially carry foreign genetic material into a host cell, such as a recombinant plasmid or virus that comprises a nucleic acid to be delivered into the host cell, either In vitro or in vivo.

As used herein, the term “expression vector” refers to a vector that directs expression of an RNA or polypeptide from sequences linked to transcriptional regulatory sequences on the vector. The sequences expressed will often, but not necessarily, be heterologous to the host cell. An expression vector may comprise additional elements, for example, the expression vector may have two replication systems, thus allowing it to be maintained in two organisms, for example in human cells for expression and in a prokaryotic host for cloning and amplification. The term “expression” refers to the cellular processes involved in producing RNA and proteins and, as appropriate, secreting proteins, including where applicable, but not limited to, for example, transcription, transcript processing, translation and protein folding, modification and processing. Expression products include RNA transcribed from a gene, and polypeptides obtained by translation of mRNA transcribed from a gene. The term “gene” means the nucleic acid sequence which is transcribed (DNA) to RNA in vitro or in vivo when operably linked to appropriate regulatory sequences. The gene may be a mutant gene and may or may not include regions preceding and following the coding region, e.g., 5′ untranslated (5′UTR) and Kozak sequence, or “leader” sequences and 3′ UTR or “trailer” sequences, as well as intervening sequences (introns) between individual coding segments (exons).

The present invention relates to products and methods of their use in direct neuronal reprogramming, for example, for the treatment of central nervous system (e.g., neurodegenerative) diseases or disorders or central nervous system injury (e.g., stroke). The product of the invention comprises a proneural or neuronal differentiation basic-helix-loop-helix (bHLH) transcription factor (e.g., Neurog2, Ascl1, Neurod1, Neurod4) that includes one or more mutations to de-sensitize the transcription factor to inhibitory environmental controls, or a nucleic acid encoding the mutant bHLH transcription factor.

The present inventors have surprisingly found that incorporation of phosphoacceptor site mutations allow the bHLH transcription factor Ascl1 to maintain its activity even in cellular contexts in which it is not normally active. This indicates that similar mutations of phosphoacceptor sites in other bHLH transcription factors will also improve the ability of these transcription factors to maintain activity even in cellular contexts in which they are not normally active. Examples of such mutated forms of bHLH transcription factors, include, for example, Neurog2-SA9, Ascl1-SA6, Neurod1-SA6, Neurod4-SA3/TA4. Expression of these mutated forms of the bHLH transcription factors, can induce neuronal lineage conversion of glial cells (e.g., astrocytes, oligodendrocytes and microglia) in a treated subject, even in an inhibitory environment, such as is present in neurodegenerative disease or following injury.

bHLH Transcription Factor Mutants

As noted above, bHLH transcription factors can be classified based on functional properties into proneural and neuronal differentiation genes (D. J. Dennis et al./Brain Research 1705 (2019) 48-6549). Proneural bHLH genes with proneural activity include Neurog1, Neurog2, Ascl1, Neurod4, Atoh1 and Atoh7. Neural differentiation bHLH transcription factors, which function later in the development of neural lineages, include NeuroD (Neurod1, Neurod2/Ndrf, Neurod6/Math2), Nscl (Nhlh1/Nscl1, Nhlh2/Nscl2), and, in some contexts, Olig (Olig1, Olig2, Olig3, Bhlhe22) families.

During neurodevelopment, bHLH transcription factors are regulated, at least in part, by phosphorylation by various kinases such that upon phosphorylation they lose activity. bHLH transcription factors comprise nucleotides encoding serines (S) or threonines (T) adjacent to proline (P) residues (designated SP or TP), which are phosphoacceptor sites for proline-directed serine-threonine kinases. Phosphorylation of these bHLH proteins by proline-directed serine threonine kinases (e.g., GSK3, ERK, CDK1) inhibits the ability of these transcription factors to bind DNA and induce neuronal differentiation. Such inhibition is an environmental inhibitory control.

The present inventors have now found that mutants of bHLH transcription factors that do not include phosphoacceptor sites for proline-directed serine-threonine kinases, or that have a reduced number of phosphoacceptor sites for proline-directed serine-threonine kinases have a surprising ability to efficiently reprogram astrocytes within the CNS, for example, the brain, into functional neurons, in a manner that is not susceptible to environmental inhibitory controls.

The ability of bHLH genes to promote neurogenesis has been found to be highly dependent on the environment; neuronal conversion efficiencies vary at different developmental times, in different brain regions and in the diseased or injured brain depending on inhibitory mechanisms present. This is demonstrated by the summary provided in the table below. Also, shown is how reducing phosphorylation of a bHLH transcription factor can significantly improve and stabilize neuronal conversion efficiencies.

| SOURCE | | | | FUNCTIONAL | |
|---|---|---|---|---|---|
| CELL | FACTORS | SUBTYPE | EFFICIENCY | OUTCOME | REFERENCE |
| Hippocampal | Neurod1, Ascl1 | Not tested | NeuN+ | Neurod1- enhanced | Present |
| Astrocytes | or Ascl1-SA6 | | neuronal density: | neural network | application |
| (AD rats) | | | Ascl1-SA6: | activity | |
| | | | 4.5x↑ | | |
| | | | Ascl1: 4.0 x ↑ | | |
| | | | Neurod1: 2.5 x ↑ | | |
| Motor cortex | Ascl1-SA6, Ascl1 | Not tested | Ascl1-SA6: 68% | Not tested | Present |
| Astrocytes | | | Ascl1: 45% | | application |
| (wild-type | | | | | |
| mice) | | | | | |
| Motor cortex | Ascl1-SA6 | Not tested | Ascl1-SA6: 80% | Not tested | Present |
| Astrocytes | | | | | application |
| (ALS) | | | | | |
| Cortical | Ascl1-SA6, Ascl1 | Not tested | Ascl1-SA6: 60% | | Present |
| Astrocytes | | | Ascl1 - 47% | | application |
| (stroke) | | | | | |
| Proliferative | Neurog2, FGF, | Glutamatergic | 72 +/− 5% | Not tested | (Grande, |
| Glial cells | EGF | | | | Sumiyoshi et |
| | | | | | al. 2013) |
| Astrocytes | Neurog2, DAPT | Glutamatergic | 50% (cortex), | Not tested | (Hu, Qin et al. |
| | | and GABAergic | 30% | | 2019) |
| | | | (cerebellum), | | |
| | | | 20% (SC) | | |
| Astrocytes | Neurog2, Nurr1 | Glutamatergic | 80% | Electrophysiologically | (Mattugini, |
| | | and GABAergic | | active, generate action | Bocchi et al. |
| | | | | potentials, form | 2019) |
| | | | | synapses, long | |
| | | | | distance axonal | |
| | | | | projections | |
| Striatal | Ascl1, Neurod1, | DA | 16% | Electrophysiologically | (Rivetti di Val |
| astrocytes | Nurr1 | | | active, generate action | Cervo, |
| | | | | potentials, Motor | Romanov et al. |
| | | | | behavioral recovery | 2017) |
| Striatal | Sox2, Nurr1, | DA | 79% | Electrophysiologically | (Niu, Zang et |
| Neurons | Lmx1a and Foxa1 | | | active, generate action | al. 2018) |
| | TFs and Valproic | | | potentials, dopamine | |
| | acid, | | | restoration | |
| Cortical | Neurod1 | Glutamatergic | 92.8% | Electrophysiologically | (Guo, Zhang et |
| astrocytes | | neurons | | active, generate action | al. 2014) |
| | | | | potentials | |
| NG2 glia | Neurod1 | Glutamatergic | 42.5% | Electrophysiologically | (Guo, Zhang et |
| | | and GABAergic | | active, generate action | al. 2014) |
| | | | | potentials | |
| Striatal | Neurod1 | Glutamatergic | 25-35% | Electrophysiologically | (Matsuda, Irie |
| microglia | | and GABAergic | | active, generate action | et al. 2019) |
| | | | | potentials and | |
| | | | | spontaneous synaptic | |
| | | | | currents | |
| Reactive | Neurod1 | Glutamatergic | 70% | Electrophysiologically | (Chen, Ma et |
| astrocytes | | and GABAergic | | active, generate action | al. 2020) |
| | | | | potentials, Motor | |
| | | | | behavior | |
| | | | | improvement | |
| Striatal non- | Neurod1 | Glutamatergic | 80% | Not tested | (Brulet, |
| reactive | | neurons | | | Matsuda et al. |
| astrocytes | | | | | 2017) |
| Cortical | Neurod1 | Glutamatergic | 20-27% | Improved locomotor | (Livingston, |
| Astrocytes | | | | activities and | Lee et al. |
| (stroke) | | | | functional recovery | 2020) |
| Striatal | Neurod1, Dlx2 | GABAergic | 80% | Electrophysiologically | (Wu, Parry et |
| astrocytes | | medium spiny | | active, generate action | al. 2020) |
| | | neurons | | potentials, Extension | |
| | | | | of life span and | |
| | | | | improved motor | |
| | | | | functions | |

The transcription factor mutants provided herein comprise a substitution mutation, such as a serine to alanine or threonine to alanine, at one or more proline-directed serine-threonine kinase phosphoacceptor sites. In each case the substitution mutation functions to stop phosphorylation at the mutated site without affecting the transcription factor activity. Although the examples provided herein include substitutions of serine or threonine to alanine, other substitutions are possible. For example, the serine or threonine of the targeted proline-directed serine-threonine kinase phosphorylation site(s) can be replaced with an amino acid having biochemical properties (e.g., size, charge and hydrophobicity) that are similar to alanine; such as, glycine, valine, leucine or isoleucine. Alternatively, the mutants can contain substitutions of serine or threonine to a D-amino acid, such as D-serine, D-threonine, D-alanine, D-glycine, D-valine, D-leucine or D-isoleucine.

Mutation of all the proline-directed serine-threonine kinase phosphoacceptor sites in a bHLH transcription factor will provide the maximum effect in terms reducing or eliminating inhibition of the ability of the transcription factor to induce neuronal differentiation. However, these bHLH transcription factors are regulated in a rheostat-like fashion, whereby the degree of phosphorylation provides variable control of activity. Consequently, it is not always necessary to mutate all of the proline-directed serine-threonine kinase phosphoacceptor sites present in the naturally occurring sequence. In particular, in some embodiments, a maximum effect is not necessary or beneficial and only a partial reduction of the inhibitory control is required. In such instances less than all of the proline-directed serine-threonine kinase phosphoacceptor sites are mutated. In these embodiments, since the bHLH transcription factors are controlled like a rheostat, it is the number of mutations rather than the location of the mutations that allows variation in the inhibitory control.

In accordance with some embodiments, the bHLH transcription factor mutants provided herein comprise a substitution of the serine or threonine at one or more proline-directed serine-threonine kinase phosphoacceptor sites. Alternatively, the mutant bHLH transcription factor comprises a substitution of serine or threonine residues at two or more proline-directed serine-threonine kinase phosphoacceptor sites, or at three or more proline-directed serine-threonine kinase phosphoacceptor sites, or at four or more proline-directed serine-threonine kinase phosphoacceptor sites, or at five or more proline-directed serine-threonine kinase phosphoacceptor sites. In other embodiments, the bHLH transcription factor mutant comprises a substitution of the serine or threonine at all of the proline-directed serine-threonine kinase phosphoacceptor sites present in the naturally occurring sequence.

The genes encoding mammalian bHLH transcription factors are highly conserved. This conservation is illustrated from a BLAST alignment of mouse vs. human coding sequences (using the NCBI reference sequences), which provides the following percent identities: Ascl1 89.73%; Neurod1 90.78%; Neurod4 87.41%; Neurog2 82.54%. In some embodiments, the mutant bHLH transcription factor is based on a human or mouse sequence. However, given the sequence similarity, a mutant based on a non-human mammalian sequence, such as mouse sequence, is useful in humans.

The mutant bHLH transcription factor protein can comprise a polypeptide sequence that is at least 90%, or more preferably at least 93%, at least 95% or at least 97% identical to the corresponding sequence of the naturally occurring protein, and which comprises a mutation of at least one proline-directed serine-threonine kinase phosphorylation site, as described above.

Percent sequence identity is calculated by determining the number of matched positions in aligned amino acid sequences, dividing the number of matched positions by the total number of aligned amino acids, and multiplying by 100. A matched position refers to a position in which identical amino acids occur at the same position in aligned amino acid sequences. Percent sequence identity also can be determined for any nucleic acid sequence.

In accordance with some embodiments, the mutant bHLH transcription factor is a mutant proneural bHLH transcription factor. In particular embodiments, the mutant transcription factor is based on the human or mouse proneural bHLH transcription factor. In accordance with other embodiments, the mutant bHLH transcription factor is a mutant neuronal differentiation bHLH transcription factor. In particular embodiments, the mutant transcription factor is based on the human or mouse neuronal bHLH transcription factor. To the extent that bHLH transcription factor sequences originating from other organisms are to be used for the design of corresponding mutants, corresponding amino acids positions can be readily determined, for example by means of pairwise or multiple sequence alignment.

In some embodiments, the proline-directed serine-threonine kinase phosphoacceptor site mutations are incorporated in combination with a mutation of a single conserved S/T residue at the Loop/Helix 2 (L-H2) junction, which is phosphorylated by PKA, and acts as a binary ‘off’ switch for both vertebrate and invertebrate proneural TFs (Quan et al. Cell 2016 Jan. 28:164(3):460-75. Doi: 10.1016/j.cell.2015.12.048).

In accordance with some embodiments, there is provided Neurog2, Ascl1, Neurod4, and Neurod1 transcription factor mutants. Table 1 lists the number of proline-directed serine-threonine kinase phosphorylation sites present in these transcription factors, of which those including a serine are identified as “SP” and those including a threonine are identified as “TP”.

TABLE 1

| | |
|---|---|
| Human | Mouse |

| Gene | SP | TP | SP | TP |
|---|---|---|---|---|
| ASCL1 | 5 | | 6 | |
| NEUROD1 | 6 | | 6 | |
| NEUROD4 | 4 | 6 | 3 | 4 |
| NEUROG2 | 8 | 1 | 9 | 1 |

Exemplary amino acid sequences of a human and a mouse wild-type ASCL1 are shown below, where the phosphorylation sites are shaded and the residue shown in a box is serine 155/150, the conserved serine residue at the L-H2 junction that is phosphorylated by PKA:

Human (SEQ ID NO: 1):
MESSAKMESGGAGOQPQPOPQQPFLPPAACFFATAAAAAAAAAAAAAQSAQQQQQQQ

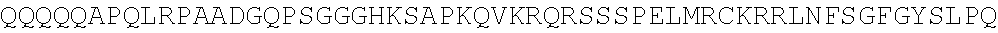

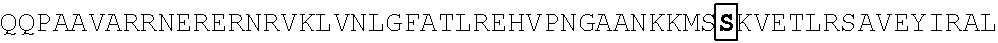

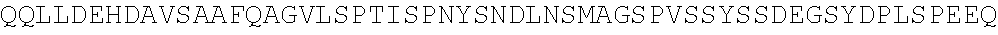

ELLDFTNWF
Mouse (SEQ ID NO: 2):
MESSGKMESGAGQQPOPPQPFLPPAACFFATAAAAAAAAAAAAQSAQQQQPQAPPQQ

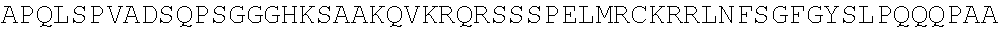

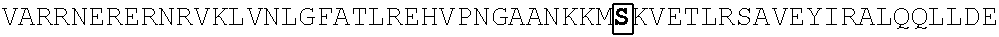

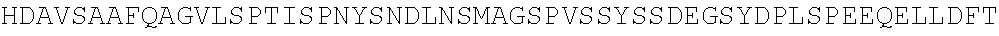

NWF

In accordance with some embodiments, there is provided an ASCL1 transcription factor mutant that is at least 80% identical to SEQ ID NO:1 or SEQ ID NO:2 and comprises a mutation of at least one, at least two, at least three or at least four phosphoacceptor sites, where each mutation eliminates phosphorylation at the corresponding mutated phosphoacceptor site. More preferably the ASCL1 transcription factor mutant is at least 85%, 90%, 95%, 97% identical to SEQ ID NO:1 or SEQ ID NO:2. Also provided herein are nucleic acids encoding an ASCL1 transcription factor mutant that is at least 80%, 85%, 90%, 95%, or 97% identical to SEQ ID NO:1 or SEQ ID NO:2 and comprises a mutation of at least one, at least two, at least three or at least four phosphoacceptor sites, where each mutation eliminates phosphorylation at the corresponding mutated phosphoacceptor site. These mutants retain transcription factor activity.

In accordance with one example, the bHLH transcription factor mutant is human Ascl1-SA5 or mouse Ascl1-SA6 (S62/88/185/189/202/218A; SEQ ID NO:3). Alternatively, the bHLH transcription factor mutant is human Ascl1-SA6 or mouse Ascl1-SA7 (S62/88/150/185/189/202/218A; SEQ ID NO:5), in which the serine at the L-H2 junction is also substituted.

The nucleic acid coding sequence (SEQ ID NO:4) for the mouse Ascl1-SA6 mutant is provided below, where the mutant codons are shaded:

ATGGAGAGCTCTGGCAAGATGGAGAGTGGAGCCGGCCAGCAGCCGCAGCCCCCGCAG
CCCTTCCTGCCTCCCGCAGCCTGCTTCTTTGCGACCGCGGCGGCGGCGGCAGCGGCG
GCGGCCGCGGCAGCTCAGAGCGCGCAGCAGCAACAGCCGCAGGCGCCGCCGCAGCAG

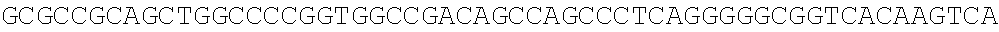

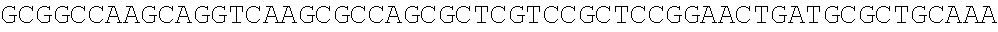

CGCCGGCTCAACTTCAGCGGCTTCGGCTACAGCCTGCCACAGCAGCAGCCGGCCGCC
GTGGCGCGCCGCAACGAGCGCGAGCGCAACCGGGTCAAGTTGGTCAACCTGGGCTTT
GCCACCCTCCGGGAGCATGTCCCCAACGGCGCGGCCAACAAGAAGATGGCCAAGGTG
GAGACGCTGCGCTCGGCGGTCGAGTACATCCGCGCGCTGCAGCAGCTGCTGGACGAG

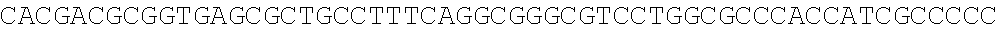

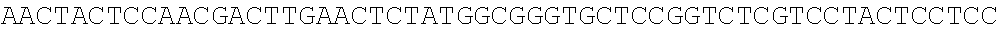

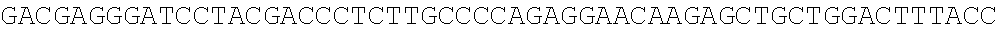

AACTGGTTC (TGA)

The nucleic acid coding sequence (SEQ ID NO:6) for the mouse Ascl1-SA7 mutant is provided below, where the mutant codons are shaded:

ATGGAGAGCTCTGGCAAGATGGAGAGTGGAGCCGGCCAGCAGCCGCAGCCCCCGCAG
CCCTTCCTGCCTCCCGCAGCCTGCTTCTTTGCGACCGCGGCGGCGGCGGCAGCGGCG
GCGGCCGCGGCAGCTCAGAGCGCGCAGCAGCAACAGCCGCAGGCGCCGCCGCAGCAG

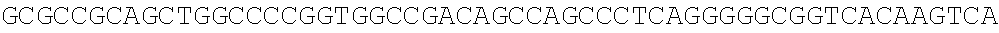

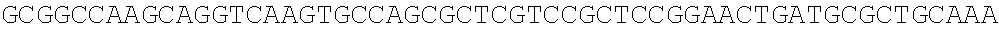

CGCCGGCTCAACTTCAGCGGCTTCGGCTACAGCCTGCCACAGCAGCAGCCGGCCGCC
GTGGCGCGCCGCAACGAGCGCGAGCGCAACCGGGTCAAGTTGGTCAACCTGGGCTTT

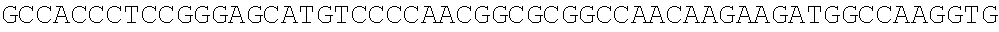

GAGACGCTGCGCTCGGCGGTCGAGTACATCCGCGCGCTGCAGCAGCTGCTGGACGAG

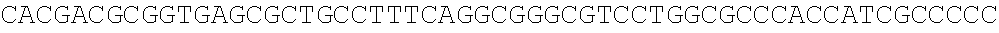

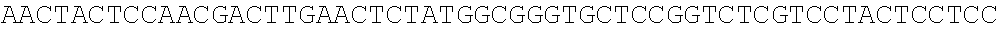

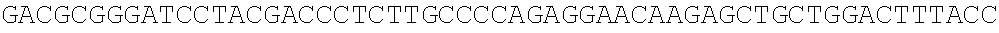

AACTGGTTC (TGA)

Exemplary amino acid sequences of a human and a mouse wild-type Neurod1 are shown below, where the phosphoacceptor sites are shaded (the residue in the box is serine 138/138, the conserved serine residue at the L-H2 junction that is phosphorylated by PKA:

Human (SEQ ID NO: 7)
MTKSYSESGLMGEPQPQGPPSWTDECLSSQDEEHEADKKEDDLETMNAEEDSLRNGG
EEEDEDEDLEEEEEEEEEDDDQKPKRRGPKKKKMTKARLERFKLRRMKANARERNRM

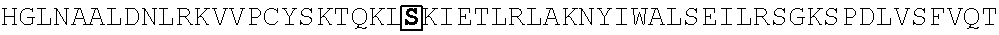

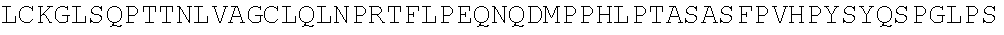

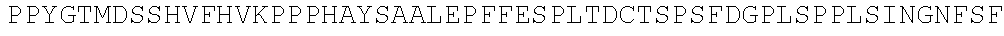

KHEPSAEFEKNYAFTMHYPAATLAGAQSHGSIFSGTAAPRCEIPIDNIMSEDSHSHH
ERVMSAQLNAIFHD
Mouse (SEQ ID NO: 8)
MTKSYSESGLMGEPQPQGPPSWTDECLSSQDEEHEADKKEDELEAMNAEEDSLRNGG
EEEEEDEDLEEEEEEEEEEEDQKPKRRGPKKKKMTKARLERFKLRRMKANARERNRM

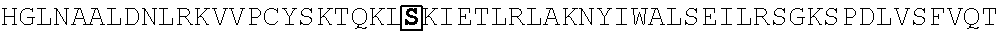

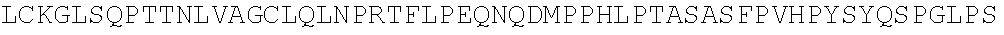

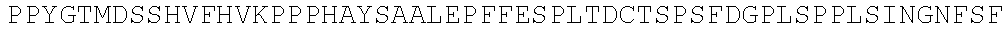

KHEPSAEFEKNYAFTMHYPAATLAGPQSHGSIFSSGAAAPRCEIPIDNIMSFDSHSH
HERVMSAQLNAIFHD

In accordance with some embodiments, there is provided a Neurod1 transcription factor mutant that is at least 80% identical to SEQ ID NO:7 or SEQ ID NO:8 and comprises a mutation of at least one, at least two, at least three or at least four phosphoacceptor sites, where each mutation eliminates phosphorylation at the corresponding mutated phosphoacceptor site. More preferably the Neurod1 transcription factor mutant is at least 85%, 90%, 95%, 97% identical to SEQ ID NO:7 or SEQ ID NO:8. Also provided herein are nucleic acids encoding a Neurod1 transcription factor mutant that is at least 80%, 85%, 90%, 95%, or 97% identical to SEQ ID NO:7 or SEQ ID NO:8 and comprises a mutation of at least one, at least two, at least three or at least four phosphoacceptor sites, where each mutation eliminates phosphorylation at the corresponding mutated phosphoacceptor site.

In accordance with one example, the bHLH transcription factor mutant is human Neurod1-SA6 or mouse Neurod1-SA6 (S162/223/228/259/266/274A; SEQ ID NO:9). Alternatively, the bHLH transcription factor mutant is human Neurod1-SA7 or mouse Neurod1-SA7 (S138/162/223/228/259/266/274A; SEQ ID NO:11), in which the serine at the L-H2 junction is also substituted.

The nucleic acid coding sequence (SEQ ID NO:10) for the mouse Neurod1-SA6 mutant is provided below, where the mutant codons are shaded:

ATGACCAAATCATACAGCGAGAGCGGGCTGATGGGCGAGCCTCAGCCCCAAGGTCCC
CCAAGCTGGACAGATGAGTGTCTCAGTTCTCAGGACGAGGAACACGAGGCAGACAAG
AAAGAGGACGAGCTTGAAGCCATGAATGCAGAGGAGGACTCTCTGAGAAACGGGGGA
GAGGAGGAGGAGGAAGATGAGGATCTAGAGGAAGAGGAGGAAGAAGAAGAGGAGGAG
GAGGATCAAAAGCCCAAGAGACGGGGTCCCAAAAAGAAAAAGATGACCAAGGCGCGC
CTAGAACGTTTTAAATTAAGGCGCATGAAGGCCAACGCCCGCGAGCGGAACCGCATG
CACGGGCTGAACGCGGCGCTGGACAACCTGCGCAAGGTGGTACCTTGCTACTCCAAG
ACCCAGAAACTGTCTAAAATAGAGACACTGCGCTTGGCCAAGAACTACATCTGGGCT

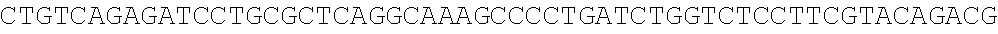

CTCTGCAAAGGTTTGTCCCAGCCCACTACCAATTTGGTCGCCGGCTGCCTGCAGCTC
AACCCTCGGACTTTCTTGCCTGAGCAGAACCCGGACATGCCCCCGCATCTGCCAACC

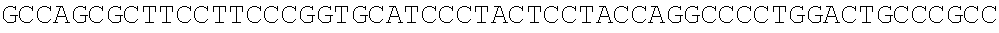

CCGCCCTACGGCACCATGGACAGCTCCCACGTCTTCCACGTCAAGCCGCCGCCACAC

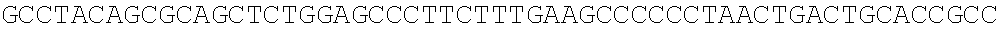

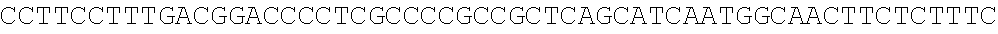

AAACACGAACCATCCGCCGAGTTTGAAAAAAATTATGCCTTTACCATGCACTACCCT
GCAGCGACGCTGGCAGGGCCCCAAAGCCACGGATCAATCTTCTCTTCCGGTGCCGCT
GCCCCTCGCTGCGAGATCCCCATAGACAACATTATGTCTTTCGATAGCCATTCGCAT
CATGAGCGAGTCATGAGTGCCCAGCTTAATGCCATCTTTCACGAT (TAG)

The nucleic acid coding sequence (SEQ ID NO:12) for the mouse Neurod1-SA7 mutant is provided below, where the mutant codons are shaded:

ATGACCAAATCATACAGCGAGAGCGGGCTGATGGGCGAGCCTCAGCCCCAAGGTCCC
CCAAGCTGGACAGATGAGTGTCTCAGTTCTCAGGACGAGGAACACGAGGCAGACAAG
AAAGAGGACGAGCTTGAAGCCATGAATGCAGAGGAGGACTCTCTGAGAAACGGGGGA
GAGGAGGAGGAGGAAGATGAGGATCTAGAGGAAGAGGAGGAAGAAGAAGAGGAGGAG
GAGGATCAAAAGCCCAAGAGACGGGGTCCCAAAAAGAAAAAGATGACCAAGGCGCGC
CTAGAACGTTTTAAATTAAGGCGCATGAAGGCCAACGCCCGCGAGCGGAACCGCATG
CACGGGCTGAACGCGGCGCTGGACAACCTGCGCAAGGTGGTACCTTGCTACTCCAAG

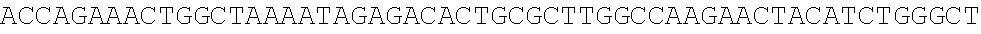

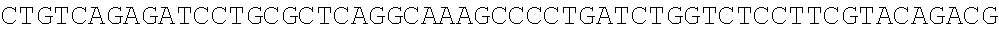

CTCTGCAAAGGTTTGTCCCAGCCCACTACCAATTTGGTCGCCGGCTGCCTGCAGCTC
AACCCTCGGACTTTCTTGCCTGAGCAGAACCCGGACATGCCCCCGCATCTGCCAACC

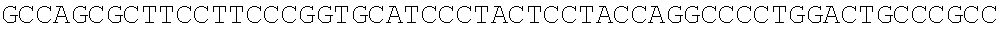

CCGCCCTACGGCACCATGGACAGCTCCCACGTCTTCCACGTCAAGCCGCCGCCACAC

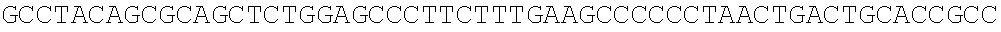

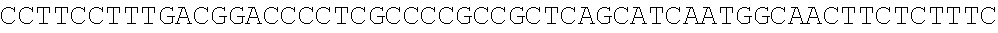

AAACACGAACCATCCGCCGAGTTTGAAAAAAATTATGCCTTTACCATGCACTACCCT
GCAGCGACGCTGGCAGGGCCCCAAAGCCACGGATCAATCTTCTCTTCCGGTGCCGCT
GCCCCTCGCTGCGAGATCCCCATAGACAACATTATGTCTTTCGATAGCCATTCGCAT
CATGAGCGAGTCATGAGTGCCCAGCTTAATGCCATCTTTCACGAT (TAG)

Exemplary amino acid sequences of a human and a mouse wild-type Neurod4 are shown below, where the phosphorylation sites are shaded (the residue in the box is serine 124/124, the conserved serine residue at the L-H2 junction that is phosphorylated by PKA:

Human (SEQ ID NO: 13)

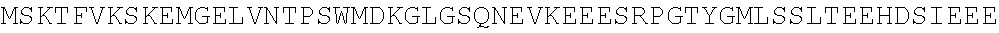

EEEEEDGEKPKRRGPKKKKMTKARLERFRARRVKANARERTRMHGLNDALDNLRRVM

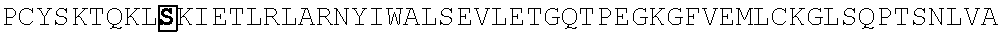

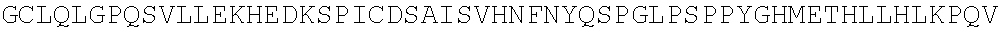

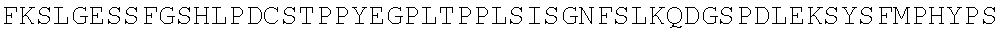

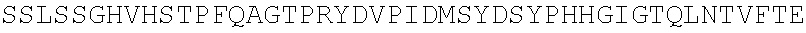

Mouse (SEQ ID NO: 14)
MAKMYMKSKDMVELVNTQSWMDKGLSSQNEMKEQERRPGSYGMLGTLTEEHDSIEED
EEEEEDGDKPKRRGPKKKKMTKARLERFRARRVKANARERTRMHGLNDALDNLRRVM

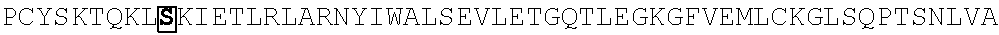

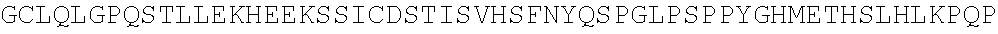

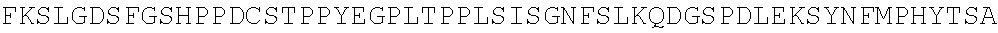

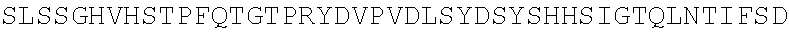

In accordance with some embodiments, there is provided a Neurod4 transcription factor mutant that is at least 75% identical to SEQ ID NO:13 or SEQ ID NO:14 and comprises a mutation of at least one, at least two, at least three or at least four phosphoacceptor sites, where each mutation eliminates phosphorylation at the corresponding mutated phosphoacceptor site. More preferably the Neurod4 transcription factor mutant is at least 80%, 85%, 90%, 95%, or 97% identical to SEQ ID NO:13 or SEQ ID NO:14. Also provided herein are nucleic acids encoding a Neurod4 transcription factor mutant that is at least 75%, 80%, 85%, 90%, 95%, or 97% identical to SEQ ID NO:13 or SEQ ID NO:14 and comprises a mutation of at least one, at least two, at least three or at least four phosphoacceptor sites, where each mutation eliminates phosphorylation at the corresponding mutated phosphoacceptor site.

In accordance with one example, the bHLH transcription factor mutant is human Neurod4-SA4TA6 or mouse Neurod4-SA3TA4 (S206/211/269A; T245/253/295/301A; SEQ ID NO:15). Alternatively, the bHLH transcription factor mutant is human Neurod4-SA5TA6 or mouse Neurod4-SA4TA4 (S124/206/211/269A; T245/253/295/301A; SEQ ID NO:17), in which the serine at the L-H2 junction is also substituted.

The nucleic acid coding sequence (SEQ ID NO:16) for the mouse Neurod4-SA3TA4 mutant is provided below, where the mutant codons are shaded:

ATGGCAAAAATGTATATGAAATCCAAGGACATGGTGGAGCTGGTCAACACACAATCC
TGGATGGACAAAGGTCTGAGCTCTCAAAATGAGATGAAGGAGCAAGAGAGAAGACCG
GGCTCTTATGGAATGCTCGGAACCTTAACTGAAGAGCATGACAGTATTGAGGAGGAT
GAAGAAGAGGAAGAAGATGGAGATAAACCTAAAAGAAGAGGTCCCAAGAAAAAGAAG
ATGACTAAAGCTCGCCTTGAAAGATTCAGGGCTCGAAGAGTCAAGGCCAATGCTAGA
GAACGGACCCGGATGCATGGCCTGAATGATGCCTTGGATAATCTTAGGAGAGTCATG
CCATGTTACTCTAAAACTCAAAAGCTTTCCAAGATAGAGACTCTTCGACTGGCAAGG
AACTACATCTGGGCCTTGTCTGAAGTCCTGGAGACTGGTCAGACACTTGAAGGGAAG
GGATTTGTAGAGATGCTATGTAAAGGTCTCTCTCAACCCACAAGCAACCTGGTTGCT
GGATGCCTCCAACTGGGGCCTCAATCTACCCTCCTGGAGAAGCATGAGGAAAAATCT

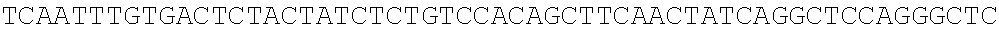

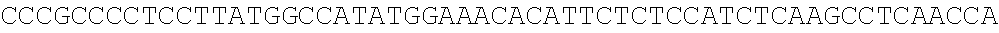

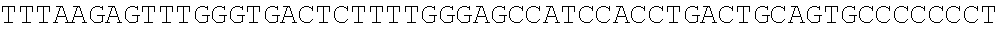

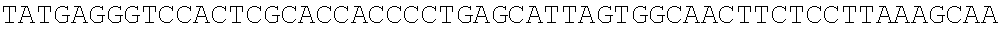

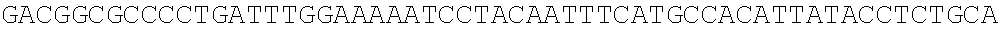

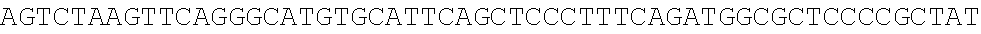

GATGTTCCTGTAGACCTGAGCTATGATTCCTACTCCCACCATAGCATTGGAACTCAG
CTCAATACGATCTTCTCTGAT (TAG)

The nucleic acid coding sequence (SEQ ID NO:18) for the mouse Neurod4-SA4TA4 mutant is provided below, where the mutant codons are shaded:

ATGGCAAAAATGTATATGAAATCCAAGGACATGGTGGAGCTGGTCAACACACAATCC
TGGATGGACAAAGGTCTGAGCTCTCAAAATGAGATGAAGGAGCAAGAGAGAAGACCG
GGCTCTTATGGAATGCTCGGAACCTTAACTGAAGAGCATGACAGTATTGAGGAGGAT
GAAGAAGAGGAAGAAGATGGAGATAAACCTAAAAGAAGAGGTCCCAAGAAAAAGAAG
ATGACTAAAGCTCGCCTTGAAAGATTCAGGGCTCGAAGAGTCAAGGCCAATGCTAGA
GAACGGACCCGGATGCATGGCCTGAATGATGCCTTGGATAATCTTAGGAGAGTCATG

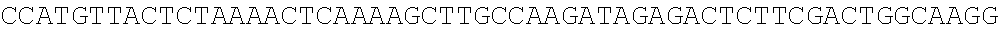

AACTACATCTGGGCCTTGTCTGAAGTCCTGGAGACTGGTCAGACACTTGAAGGGAAG
GGATTTGTAGAGATGCTATGTAAAGGTCTCTCTCAACCCACAAGCAACCTGGTTGCT
GGATGCCTCCAACTGGGGCCTCAATCTACCCTCCTGGAGAAGCATGAGGAAAAATCT

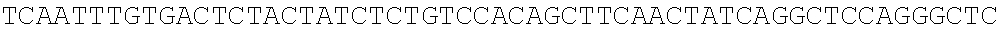

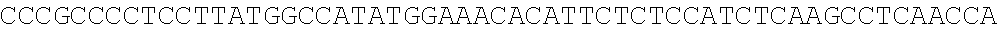

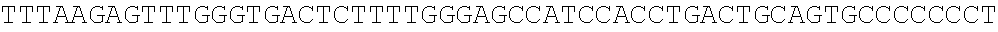

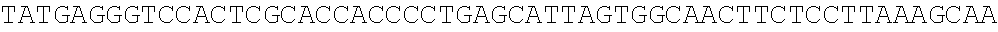

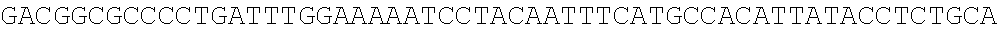

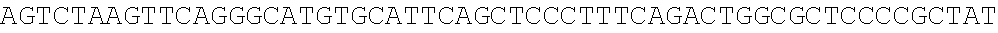

GATGTTCCTGTAGACCTGAGCTATGATTCCTACTCCCACCATAGCATTGGAACTCAG
CTCAATACGATCTTCTCTGAT (TAG)

Exemplary amino acid sequences of a human and a mouse wild-type Neurog2 are shown below, where the phosphoacceptor sites are shaded (the residue in the box is threonine 149/149, the conserved threonine residue at the L-H2 junction that is phosphorylated by PKA):

Human (SEQ ID NO: 19)

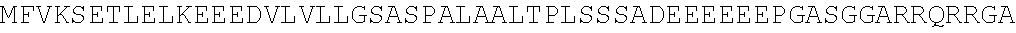

EAGQGARGGVAAGAEGCRPARLLGLVHDCKRRPSRARAVSRGAKTAETVQRIKKTRR

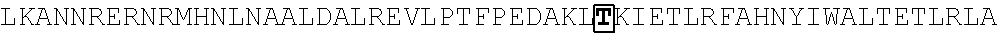

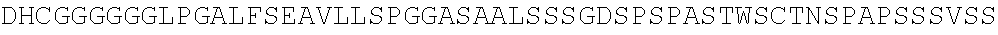

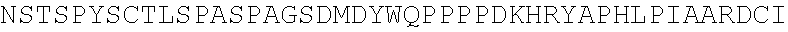

Mouse (SEQ ID NO: 20)

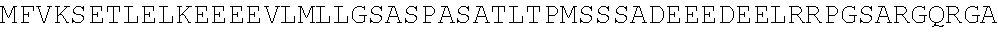

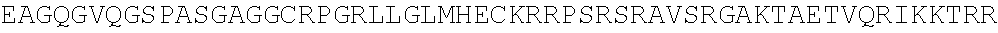

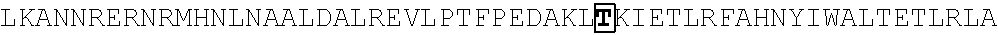

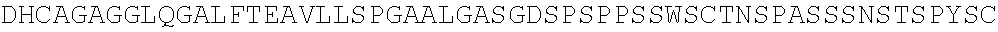

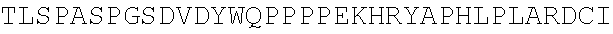

In accordance with some embodiments, there is provided a Neurog2 transcription factor mutant that is at least 70% identical to SEQ ID NO:19 or SEQ ID NO:20 and comprises a mutation of at least one, at least two, at least three or at least four phosphoacceptor sites, where each mutation eliminates phosphorylation at the corresponding mutated phosphoacceptor site. More preferably the Neurog2 transcription factor mutant is at least 75%, 80%, 85%, 90%, 95%, or 97% identical to SEQ ID NO:19 or SEQ ID NO:20. Also provided herein are nucleic acids encoding a Neurod4 transcription factor mutant that is at least 70%, 75%, 80%, 85%, 90%, 95%, or 97% identical to SEQ ID NO:19 or SEQ ID NO:20 and comprises a mutation of at least one, at least two, at least three or at least four phosphoacceptor sites, where each mutation eliminates phosphorylation at the corresponding mutated phosphoacceptor site.

In accordance with one example, the bHLH transcription factor mutant is human Neurog2-SA8TA1 or mouse Neurog2-SA9TA1 (S24/66/192/203/205/215/224/231/234A;T31A; SEQ ID NO:21). Alternatively, the bHLH transcription factor mutant is human Neurog2-SA8TA2 or mouse Neurog2-SA9TA2 (S24/66/192/203/205/215/224/231/234A;T31/149A; SEQ ID NO:23), in which the serine at the L-H2 junction is also substituted. In other embodiments, the bHLH transcription factor mutant is mouse Neurog2-SA9 (S24/66/192/203/205/215/224/231/234A; SEQ ID NO:25).

The nucleic acid coding sequence (SEQ ID NO:22) for the mouse Neurog2-SA9TA1 mutant is provided below, where the mutant codons are shaded:

ATGTTCGTCAAATCTGAGACTCTGGAGTTGAAGGAGGAAGAGGAGGTACTGATGCTG

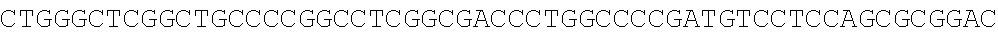

GAGGAGGAGGACGAGGAGCTGCGCCGGCCGGGCTCCGCGCGTGGGCAGCGTGGAGCG

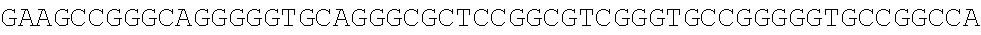

GGGCGGCTGCTGGGCCTGATGCACGAGTGCAAGCGTCGCCCGTCGCGCTCACGGGCC
GTCTCCCGAGGTGCCAAGACGGCGGAGACGGTGCAGCGCATCAAGAAGACCCGCAGG
CTCAAGGCCAACAACCGCGAGCGCAACCGCATGCACAACCTAAACGCCGCGCTGGAC
GCGCTGCGCGAGGTGCTGCCCACCTTCCCCGAGGATGCCAAGCTCACGAAGATCGAG
ACGCTGCGCTTCGCCCACAATTACATCTGGGCGCTCACCGAGACTCTGCGCCTGGCG
GACCACTGCGCCGGCGCCGGTGGCCTCCAGGGGGCGCTCTTCACGGAGGCGGTGCTC

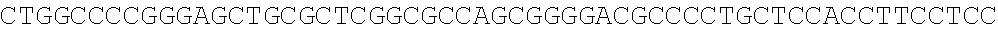

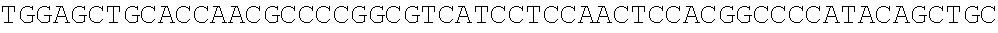

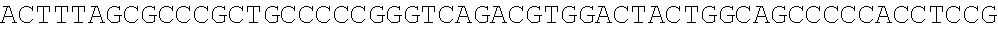

GAGAAGCATCGTTATGCGCCTCACCTGCCCCTCGCCAGGGACTGTATC (TAG)

The nucleic acid coding sequence (SEQ ID NO:24) for the mouse Neurog2-SA9TA2 mutant is provided below, where the mutant codons are shaded:

ATGTTCGTCAAATCTGAGACTCTGGAGTTGAAGGAGGAAGAGGAGGTACTGATGCTG

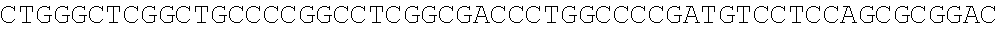

GAGGAGGAGGACGAGGAGCTGCGCCGGCCGGGCTCCGCGCGTGGGCAGCGTGGAGCG

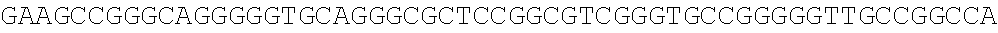

GGGCGGCTGCTGGGCCTGATGCACGAGTGCAAGCGTCGCCCGTCGCGCTCACGGGCC
GTCTCCCGAGGTGCCAAGACGGCGGAGACGGTGCAGCGCATCAAGAAGACCCGCAGG
CTCAAGGCCAACAACCGCGAGCGCAACCGCATGCACAACCTAAACGCCGCGCTGGAC

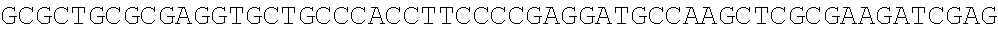

ACGCTGCGCTTCGCCCACAATTACATCTGGGCGCTCACCGAGACTCTGCGCCTGGCG
GACCACTGCGCCGGCGCCGGTGGCCTCCAGGGGGCGCTCTTCACGGAGGCGGTGCTC

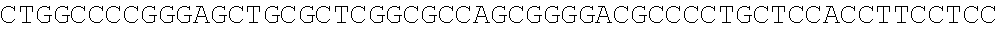

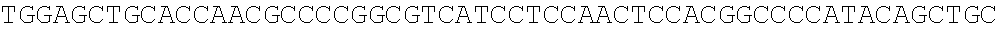

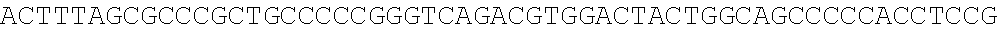

GAGAAGCATCGTTATGCGCCTCACCTGCCCCTCGCCAGGGACTGTATC (TAG)

In some instances, the mutant bHLH transcription factor protein can include additional substitutions, beyond those introduced to limit or prevent phosphorylation. Such additional substitutions would introduce one or more conservative changes, which replace amino acids with other amino acids of similar chemical structure, similar chemical properties or similar side-chain volume. The amino acids introduced will have similar polarity, hydrophilicity, hydrophobicity, basicity, acidity, neutrality or charge to the amino acids they replace. Alternatively, the conservative change may introduce another amino acid that is aromatic or aliphatic in the place of a pre-existing aromatic or aliphatic amino acid. Conservative amino acid changes are well known in the art and may be selected in accordance with the properties of the 20 main amino acids. Conservative substitutions do not affect the function of the protein or polypeptide. Proteins including such conservative substitutions are referenced herein as “conservative variants.”

### Therapeutic Use and Compositions

As described herein, a mammal (e.g., a mammal having a neurodegenerative disease or disorder, or a CNS or brain injury) can be treated by delivering an isolated mutant bHLH transcription factor as described above, to glial cells, preferably astrocytes, within the mammalian brain (e.g., cerebral cortex) in a manner that triggers the glial cells to form neurons. The formed neurons may or may not be electrophysiologically functional. In some embodiments the formed neurons function to enhance neuroplasticity, support growth and development of other cell types, and/or modify their microenvironment (e.g., reduce toxicity of their microenvironment). The mutant bHLH transcription factor can be delivered either directly as an isolated or purified protein, or via expression of a nucleic acid encoding the mutant bHLH transcription factor.

In accordance with some embodiments, two or more mutant bHLH transcription factors are used in the treatment of a subject. The two or more mutants can be used simultaneously or in sequence.

The neurodegenerative disease or disorder can be, for example, Alzheimer's disease (AD), presenile and senile forms; mild cognitive impairment; Alzheimer's disease-related dementia; tauopathy; α-synucleinopathy; Parkinson's disease; Amyotrophic Lateral Sclerosis (ALS); motor neuron Disease; Spastic paraplegia; Huntington's Disease, spinocerebellar ataxia, Freidrich's Ataxia; neurodegenerative diseases associated with intracellular and/or intraneuronal aggregates of proteins with polyglutamine, polyalanine or other repeats arising from pathological expansions of tri- or tetra-nucleotide elements within corresponding genes; Down's syndrome; Prion related disease; Familial British Dementia; Familial Danish Dementia; Presenile Dementia with Spastic Ataxia; Cerebral Amyloid Angiopathy, British Type; Presenile Dementia With Spastic Ataxia Cerebral Amyloid Angiopathy, Danish Type; grain dementia, corticobasal degeneration, dementia pugilistica, diffuse neurofibrillary tangles with calcification, frontotemporal dementia with parkinsonism, Prion-related disease, Hallervorden-Spatz disease, myotonic dystrophy, Niemann-Pick disease type C, non-Guamanian Motor Neuron disease with neurofibrillary tangles, Pick's disease, postencephalitic parkinsonism, prion protein cerebral amyloid angiopathy, progressive subcortical gliosis, progressive supranuclear palsy, subacute sclerosing panencephalitis, and tangle only dementia; dementia with Lewy bodies, multiple system atrophy with glial cytoplasmic inclusions, Shy-Drager syndrome, striatonigral degeneration, olivopontocerebellar atrophy, neurodegeneration with brain iron accumulation type I olfactory dysfunction, and amyotrophic lateral sclerosis; the Spastic paraplegia and the spinocerebellar ataxia is DRPLA or Machado-Joseph Disease; the Prion related diseases Creutzfeldt-Jakob disease, Gerstmann-Straussler-Scheinker disease, and variant Creutzfeldt-Jakob disease. Preferably, the neurodegenerative disease or disorder is ALS, AD, Alzheimer's disease-related dementia, Parkinson's disease, Down's syndrome, dementia pugilistica, multiple system atrophy, frontal temporal dementia, progressive supranuclear palsy, prion diseases, Huntington's disease, a motor neuron disease or a Lewy body disease. A brain or CNS injury can be, for example, trauma, concussion, stroke, tumor, infection, substance abuse, hypoxia, anoxia, aneurysm, injury resulting from neurological illness, toxins, embolisms, hematomas, brain hemorrhaging, injury resulting from a genetic disease, or a coma.

The present application further provides compositions comprising a mutant bHLH transcription factor and one or more pharmaceutically acceptable diluent, excipient or carrier. The composition for delivery of one or more mutant bHLH transcription factor can be in the form of a nanocarriers or nanoparticles, such as, for example, liposomes, or biomaterials (for a review of such delivery formulations, see, Khait N L, Ho E, and Shoichet M S, Advanced Functional Materials, special issue on Advanced Materials for Drug Delivery and Theranostics. (2021) 2010674: 1-32). Such delivery formulations can additionally incorporate targeting elements to specifically direct the mutant bHLH transcription factor(s) to the target glial cells in the CNS.

In some embodiments, to improve the efficiency of neuronal lineage conversion, or to confer subtype identities, the mutated bHLH transcription factor is used together with one or more other transcription factors (TFs). Other TFs that act as fate determinants that may be expressed together with the mutant bHLH transcription factor described herein include, but are not limited to, homeodomain TFs that specify a regional identity (Dlx1, Dlx2, Pax6, Emx1, Lhx2), layer V determinants (Fezf2, Ctip2), Nurr1 (official gene name, Nr4a2, increases neuronal reprogramming efficiency), Brn2 (official gene name, Pou3f2, cooperates with Ascl1 to promote neurogenesis), Sox family TFs (specify a neural identity), and Myt1l, which is a multi-lineage repressor of alternative, non-neural cell fates. The use of several of these TFs in a method for transdifferentiation of differentiated cells has been described European Patent application No. EP 3 099 786, which is incorporated herein by reference.

Alternatively, as also described herein, a mammal (e.g., a mammal having a neurodegenerative disease or disorder, or a CNS or brain injury) can be treated by administering a nucleic acid designed to express a mutant bHLH transcription factor as described above to glial cells, preferably astrocytes, within the mammal's brain (e.g., cerebral cortex) in a manner that triggers the glial cells, preferably astrocytes, to form neurons. As described above, the formed neurons may or may not be electrophysiologically functional. In some embodiments the formed neurons function to enhance neuroplasticity, support growth and development of other cell types, and/or modify their microenvironment (e.g., reduce toxicity of their microenvironment).

Accordingly, the present application further provides a nucleic acid, or nucleic acid construct, designed to express a mutant bHLH transcription factor polypeptide. The nucleic acid comprises a polynucleotide encoding a mutant bHLH transcription factor as described herein operably linked to one or more control sequences that direct the expression of the coding sequence in a suitable host cell under conditions compatible with the control sequences.

In some embodiments, the nucleic acid is designed for expression target glial cells. Alternatively, the nucleic acid is designed for expression in an in vitro system, such as in a bacterial system, for manufacture of the mutant bHLH transcription factor. Also provided herein are compositions comprising such nucleic acids and one or more diluent, excipient or carrier, which is one or more pharmaceutically acceptable diluent, excipient or carrier when the composition is for administration to treat a subject.

In addition to the polynucleotide encoding a mutant bHLH transcription factor polypeptide, the nucleic acid can contain one or more regulatory elements operably linked to the coding sequence. As used herein, “operably linked” refers to positioning of a regulatory element in a vector relative to a nucleic acid in such a way as to permit or facilitate expression of the encoded polypeptide. Examples of regulatory elements can include, without limitation, translation initiation sequences (e.g., a Kozak consensus sequence or a tissue-specific Kozak sequence (e.g., McClements et al. Mol. Vis. 2021, 27:233-242) to avoid expression in non-target tissues), promoter sequences, enhancer sequences, response elements, signal peptides, internal ribosome entry sequences, 5′UTR, 3′UTR, polyadenylation signals, terminators, or inducible elements that modulate expression (e.g., transcription or translation) of a nucleic acid.

For example, a promoter can be included in the nucleic acid to facilitate transcription of the nucleic acid encoding a mutant bHLH transcription factor polypeptide. A promoter can be constitutive or inducible, and can affect the expression of a nucleic acid encoding a polypeptide in a general or tissue-specific manner. Examples of cell-specific and/or tissue-specific promoters that can be used to drive expression of a mutant bHLH transcription factor polypeptide in glial cells (e.g., astrocytes) include, without limitation, Nestin, Vimentin, NG2, GFAP, gfaABC₁D (minimal GFAP promoter), Olig2, CAG, EF1a, Aldh1L1, CMV and CBA (chimeric CMV-chicken 3-actin) promoters. In particular embodiments, a GFAP promoter can be included in a viral vector to facilitate transcription of a nucleic acid encoding a mutant bHLH transcription factor polypeptide in astrocytes.

Among the additional regulatory elements that can be included in the nucleic acid for expression of the mutant bHLH transcription factor are 3′UTR and/or 5′UTR sequences. In a particular example the nucleic acid incorporates the 3′UTR of wild-type Ascl1. The 3′UTR in wild-type Ascl1 includes an intron that targets the message for nonsense-mediated decay (NMD). Addition of the Ascl1 3′UTR to the nucleic acid encoding a mutant bHLH transcription factor may make the transcript less stable so that it is degraded following cell conversion to a neuron and, thereby avoids continued expression after neuronal reprogramming has been achieved. In some instances, bHLH proteins are able to convert P1 brain NSCs to neurons, but these neurons die over time if the bHLH protein expression is maintained (L. Cai, E. M. Morrow, and C. L. Cepko, Misexpression of basic helix-loop-helix genes in the murine cerebral cortex affects cell fate choices and neuronal survival. Development 127 (2000) 3021-30). Accordingly, it can be beneficial, in some embodiments, to incorporate such a regulatory sequence useful for turning off expression of the mutant bHLH transcription factor gene.

The sequence of the Ascl1 3′UTR sequence (SEQ ID NO: 38) is shown below (NM_008553.5). The shaded sequence is the first exon and the remainder of the sequence constitutes the second exon.

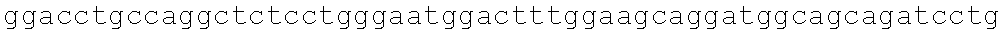

catctttagtgtttctcgccaacgacgtcaaatggggaggcagaaaaacaaggggaa
aaaagaagaagaaatgaaacaaacaaaccagacagccaacctacaggggcaccttca
ctaagatgcaatgttctcagcaaacaggggtgggctccaacagtgtctctgcattcc
aacatcatttccagacacgagaagagtgactggtgtctgaacctaagcccgaatcac
agatgggttcctttcctggagcaagagcgtcacacacacacacacacacacagacag
acactatattaactcccaaccactaacaggcagggctggaagcgcgcatgtgcaagt
gccttcacctcccactctctgtcagagctgtcttagccccctgaaactgggttgatg
tctttcctcagtcacccccattccagcgatctatggacatttgcctccattgaagca
acgtcagttctcggacagcctttccctctcctggtggcctcctccccaaaccccaca
tcgccctcccacggtctttgcttctgttttcttcatagaatgcttccaatctttgtg
aatttttttattataagaaaaaaatctatttgtatctatcctaaccagtttggggat
atattaagatatttttgtacataagaaaaagagagagaaaaaatttatagaagtttt
gtacaaatggtttaaaaatgtgtatatcttgatactttaacatgtaatgagattacc
tctgcgtactttagatatgtagttcatcttacaactgccatccccacccccatcccc
agtgtggttttggaaagaactctcctcataggtgagatctaaatgccaccagaatga
cttcagcaccaatgtgtcttacttcacagaaacgtggttaatgtattaatgatgtta
ttaaaaaaaactgttcaagaagaacaaaagtttatgcagctactgtccaaacgcaaa
gtggcagcccgttggttcccataggttgccttttggaagtttctaccattgccttcc
ccctcacacccaccccttactgttttattacaaacttacaaaaaaaagtgtataacc
ctgttttatacaaactagttttgtaataaaactttttccttttttatataaaaagaa
gagagagagagaatgaaaaaaaaaa

Provided below is the Ascl1 intron sequence (mouse; (SEQ ID NO: 39)) useful for turning off expression.

gtacgcttcatgtggggatgggcagagcctttcttgactaggtccctt
tttctttttcggtgtggaggggggagttcacggaaggtaggggctgtc
tccaggaacccaggggtgagaggaggttactatgttgttagtttgtcc
atctcaagtgctagtgtgttaacttcagtcttggcccctgagagtggc
tttgcccaagtttccaagagcagaaggtcgaggtgaggcatagagacc
ctgcgtattgagtcctgggactttgtccaaagagatgttttacctgac
agattctccaagcctgaatagcttctaccacaccccttcaagttcaca
ctaacctctcttgtttctgttacag

In some embodiments, to improve the efficiency of neuronal lineage conversion, or to confer subtype identities, the mutated bHLH gene is expressed together with one or more other transcription factors (TFs). The coding sequences are separated by a ribosome skipping sequence (e.g., v2A, t2A, p2A), to allow for stoichiometric expression of multiple genes from a polycistronic transcript. Other TFs that act as fate determinants that may be expressed together with the mutant bHLH transcription factor described herein include, but are not limited to, homeodomain TFs that specify a regional identity (Dlx1, Dlx2, Pax6, Emx1, Lhx2), layer V determinants (Fezf2, Ctip2), Nurr1 (official gene name, Nr4a2, increases neuronal reprogramming efficiency), Brn2 (official gene name, Pou3f2, cooperates with Ascl1 to promote neurogenesis), Sox family TFs (specify a neural identity), and Myt1l, which is a multi-lineage repressor of alternative, non-neural cell fates. As noted above, the use of several of these TFs in a method for transdifferentiation of differentiated cells has been described European Patent application No. EP 3 099 786.

As noted above, in some embodiments, two or more mutant bHLH transcription factors are used in the treatment of a subject. In one example of this embodiment, a nucleic acid can comprise coding sequences for two or more mutant bHLH transcription factors, which are separated by a ribosome skipping sequence (e.g., v2A, t2A, p2A), to allow for stoichiometric expression of multiple genes from a polycistronic transcript. For example, the nucleic acid can comprise sequences encoding a first mutant bHLH transcription factor and a second, different, mutant bHLH transcription factor, and an intervening t2a polypeptide sequence. Such a nucleic acid is useful for expression of two mutant bHLH transcription factors in tandem to increase neuronal reprogramming efficiency.

In some embodiments the nucleic acid encoding one or more mutant bHLH transcription factor polypeptide can be administered to a mammal using one or more vectors, such as viral vectors. Vectors for administering nucleic acids (e.g., nucleic acid encoding a mutant bHLH transcription factor polypeptide) can be prepared using appropriate materials (e.g., packaging cell lines, helper viruses, and vector constructs). See, for example, Gene Therapy Protocols (Methods in Molecular Medicine), edited by Jeffrey R. Morgan, Humana Press, Totowa, N.J. (2002) and Viral Vectors for Gene Therapy: Methods and Protocols, edited by Curtis A. Machida, Humana Press, Totowa, N.J. (2003). In some cases, virus-based vectors can be used to express nucleic acid in dividing cells. In some cases, virus-based vectors can be used to express nucleic acid in non-dividing cells. In some cases, virus-based vectors can be used to express nucleic acid in both dividing cells and non-dividing cells. Virus-based nucleic acid delivery vectors for delivering nucleic acid designed to express a mutant bHLH transcription factor polypeptide within the CNS of a living mammal can be derived from animal viruses, such as adenoviruses, adeno-associated viruses, retroviruses, lentiviruses, vaccinia viruses, herpes viruses, and papilloma viruses. In some cases, nucleic acid encoding a mutant bHLH transcription factor polypeptide can be delivered to astrocytes using adeno-associated virus (AAV) vectors (e.g., AAV serotype 2, AAV serotype 2/5, AAV serotype 2/8, AAV serotype 5, or AAV serotype 9 viral vector), lentiviral vectors, retroviral vectors, adenoviral vectors, herpes simplex virus vectors, or poxvirus vector. For example, nucleic acid encoding a mutant bHLH transcription factor polypeptide can be administered to a mammal using adeno-associated virus vectors to express the mutant bHLH transcription factor polypeptide in both dividing and non-dividing cells.

Examples of site-specific recombination elements that can be incorporated in the nucleic acid encoding the mutant bHLH transcription are, without limitation, recombination target sites (e.g., LoxP sites), or flip-excision (FLEx) switches that modulate site-specific recombination of a nucleic acid. The choice of element(s) that may be included in a viral vector depends on several factors, including, without limitation, inducibility, targeting, the level of expression desired, and the desired recombination site.

In some embodiments, the nucleic acid encoding one or more mutant bHLH transcription factor can be formulated in a non-viral vector. Examples of non-viral vectors include, but are not limited to, lipoplexes, inorganic nanoparticles and naked or plasmid DNA. Non-viral vectors typically do not rely on any viral component. In some embodiments, the non-viral vector is a transposon or mobile DNA element that can integrate the sequence encoding the one or more mutant bHLH transcription factor into target cell chromosomes.

The viral vector or non-viral vector nucleic acid for expression of one or more mutant bHLH transcription factor polypeptide can be formulated in nanocarriers or nanoparticles, such as, for example, DNA Trojan Horse, liposomes, or biomaterials (for a review of such delivery formulations, see, Khait N L, Ho E, and Shoichet M S, Advanced Functional Materials, special issue on Advanced Materials for Drug Delivery and Theranostics. (2021) 2010674: 1-32). Such formulations can additionally incorporate targeting elements to specifically direct the nucleic acid to the target glial cells in the CNS.

Nucleic acids encoding a mutant bHLH transcription factor polypeptide, including viral and non-viral vectors, can be produced by techniques including, without limitation, molecular cloning, polymerase chain reaction (PCR), chemical nucleic acid synthesis techniques, and combinations of such techniques. For example, PCR or RT-PCR can be used with oligonucleotide primers designed to amplify nucleic acid (e.g., genomic DNA or RNA) encoding a mutant bHLH transcription factor polypeptide. Subsequent molecular cloning techniques can then be used to insert the nucleic acid encoding a mutant bHLH transcription factor polypeptide into the appropriate location within a viral vector (e.g., AAV, such as AAV2/5, AAV2/8, AAV9) or a non-viral vector.

Delivery of the nucleic acid for expression of one or more mutant bHLH transcription factor polypeptides to glial cells in a subject results in efficient expression of the mutant bHLH transcription factor polypeptide and subsequent reprogramming of the glial cells, without any, or with minimal, effect from environmental inhibitory controls. Similarly, delivery of one or more mutant bHLH transcription factor polypeptides to glial cells in a subject results in reprogramming of the glial cells, without any, or with minimal, effect from environmental inhibitory controls.

Any appropriate method can be used to deliver one or more mutant bHLH transcription factor polypeptide, or nucleic acid designed to express one or more mutant bHLH transcription factor polypeptide, to glial cells within the CNS (e.g., astrocytes).

The compositions described herein that contain one or more mutant bHLH transcription factor polypeptide, or nucleic acid designed to express one or more mutant bHLH transcription factor polypeptide, can be administered to glial cells within the CNS (e.g., astrocytes), such as within the cerebral cortex via, for example, direct intracranial administration, intrastriatal injection, intracerebroventricular injection, intracisternal injection, intraparenchymal injection, intrathecal injection, intraperitoneal administration, intravenous administration, intra-arterial, intranasal administration, intramuscular administration, or oral administration in nanoparticles and/or drug tablets, capsules, or pills.

As described herein, a mutant bHLH transcription factor polypeptide, or nucleic acids designed to express a mutant bHLH transcription factor polypeptide, or any combination thereof, can be administered to a mammal (e.g., a human) having a neurodegenerative disease or disorder, or a CNS injury (e.g., stroke) to treat or prevent the disease or disorder or to treat the injury. The administered mutant bHLH transcription factor(s) or expressed mutant bHLH transcription factor(s) functions to treat or prevent the neurodegenerative disease or disorder, or to treat the injury, by converting glial cells (e.g., astrocytes in the CNS) into neurons, rebalancing neuron:glia ratios, rebalancing excitation:inhibition, repairing damaged brain tissue, reducing glial scar, reducing neuroinflammation, restoring the blood-brain-barrier, and/or reducing the amount of toxic microglia. Optionally, the method for treatment or prevention of the neurodegenerative disease or disorder, or for treatment of the injury, can comprise expression of two or more mutant bHLH transcription factor polypeptide, in parallel or in sequence.

In accordance with a particular embodiment, the mutant bHLH transcription factor polypeptide or the nucleic acid designed to express a mutant bHLH transcription factor polypeptide, or any combination thereof, can be administered to a mammal (e.g., a human) having, or at risk of developing, ALS. In this embodiment, the administered or expressed mutant bHLH transcription factor polypeptide(s) functions to treat or prevent ALS in the mammal, by converting glial cells (e.g., astrocytes) into functional neurons, rebalancing neuron:glia ratios, rebalancing excitation:inhibition, repairing damaged brain tissue (e.g., repairing neuronal degeneration in the motor cortex and/or spinal cord), reducing pathogenic TDP-43 protein aggregation, and/or reducing neuroinflammation. Optionally, the method for treatment or prevention of ALS can comprise administration or expression of two or more mutant bHLH transcription factor polypeptide, in parallel or in sequence.

In accordance with another particular embodiment, the mutant bHLH transcription factor polypeptide or the nucleic acid designed to express a mutant bHLH transcription factor polypeptide, or any combination thereof, can be administered to a mammal (e.g., a human) having or at risk of developing Alzheimer's Disease (AD). In this embodiment, the administered or expressed mutant bHLH transcription factor polypeptide functions to treat or prevent AD in the mammal, by converting glial cells (e.g., astrocytes) into neurons, in particular glutamatergic and GABAergic neurons, rebalancing excitation:inhibition, rebalancing neuron:glia ratios, reducing neuroinflammation, increasing hippocampal neuronal connectivity, increasing microglial association with plaques and/or stimulating amyloid plaque clearance. Optionally, the method for treatment or prevention of AD can comprise administration or expression of two or more mutant bHLH transcription factor polypeptide, in parallel or in sequence.

In accordance with another particular embodiment, the mutant bHLH transcription factor polypeptide or the nucleic acid designed to express a mutant bHLH transcription factor polypeptide, or any combination thereof, can be administered to a mammal (e.g., a human) following a brain injury, such as stroke. In this embodiment, the administered or expressed mutant bHLH transcription factor polypeptide functions to treat the injury in the mammal, by converting glial cells (e.g., astrocytes) into neurons, for example, glutamatergic and GABAergic neurons, rebalancing excitation:inhibition, rebalancing neuron:glia ratios, reducing scar formation, reducing neuroinflammation, increasing neuronal connectivity, increasing neuroplasticity. Optionally, the method for treatment of stroke can comprise administration or expression of two or more mutant bHLH transcription factor polypeptide, in parallel or in sequence.

Accordingly, further provided and to be used herein are composition(s) comprising the herein disclosed nucleic acid(s), vector(s), and/or protein(s). The composition may be a pharmaceutical composition that comprises one or more pharmaceutically acceptable diluents, excipients, carriers or the like.

In accordance with some embodiments, the brain (e.g., the cerebral cortex) within a mammal (e.g., a living mammal) can be monitored to evaluate the effectiveness of a treatment described herein. Any appropriate method can be used to determine whether or not a disease or disorder, or a brain injury, present within a mammal is being effectively treated. For example, imaging techniques and/or laboratory assays can be used to assess the number astrocytes and/or the number of neurons present within a mammal's brain. In some cases, imaging techniques and/or laboratory assays can be used to assess whether or not any mutant bHLH transcription factor-mediated effects (e.g., converting glial cells (e.g., astrocytes) into functional neurons, rebalancing neuron:glia ratios, repairing damaged brain tissue (e.g., reducing glial scar formation), reducing neuroinflammation, restoring the blood-brain-barrier, restoring the neurovascular unit, reducing the amount of toxic microglia, increasing hippocampal neuronal connectivity, increasing microglial association with plaques and/or stimulating amyloid plaque clearance) are observed. In some cases, imaging techniques and/or laboratory assays can be used to assess mutant bHLH transcription factor-mediated effects can be as described in the Examples. In treated subjects, the effect of treatment can be monitored, for example, using PET, MRI or CT scans.

Also provided herein are kits that include one or more mutant bHLH transcription factor polypeptide described herein (e.g., Neurog2-SA9, Ascl1-SA6, Neurod4-SA3/TA4, Neurod1-SA6 or any combination thereof) or nucleic acid encoding one or more mutant bHLH transcription factor polypeptide described herein. The kits also can include instructions for performing any of the methods described herein. In some cases, the kits can include at least one dose of any of the compositions described herein. In some embodiments, the kits can provide a means (e.g., a syringe) for administering any of the compositions described herein.

To gain a better understanding of the invention described herein, the following examples are set forth. It should be understood that these examples are for illustrative purposes only. Therefore, they should not limit the scope of this invention in any way.

### EXAMPLES

### Example 1: Neuronal Lineage Conversion for ALS Therapy

Amyotrophic lateral sclerosis (ALS) is a terminal neurodegenerative disease that results in a loss of motor neurons in the brain and spinal cord, leading to a deterioration of motor function and ultimately culminating in death. Astrocytes contribute to neuronal toxicity, and a dying forward model suggests that neuronal degeneration first occurs in the motor cortex, which triggers motor neuron death in the spinal cord. Here, neuronal lineage conversion of motor cortex astrocytes was studied as a means to delay disease progression using hSOD1^{G93A} transgenic mice, which express a human SOD1 transgene carrying a G93A mutation that is found in ALS patients. To promote astrocyte to neuron conversion, mis-expression of a modified form of the proneural gene Ascl1 in which six serines were converted to alanines (Ascl1-SA6), enhances neuronal conversion rates in the embryonic brain. Gene delivery was achieved using adeno-associated virus (AAV) 2/5 as a vector, under the control of a glial fibrillary acidic protein (GFAP) promoter. It was first confirmed that Ascl1-SA6 could convert cortical astrocytes to neurons more efficiently than Ascl1. Then the effects of mis-expressing Ascl1-SA6 in cortical astrocytes on disease progression were monitored, revealing that hSOD1^{G93A} transgenic mice were better able to maintain their body weight, retained a better (i.e., lower) neuroscore, and had signs of improved motor function. Taken together, these data demonstrate that neuronal lineage conversion targeted to motor cortex astrocytes can be used as a therapeutic strategy for the treatment of ALS.

### Introduction

ALS is associated with the death of upper motor neurons in the motor cortex and their innervation targets, lower motor neurons in the brainstem and spinal cord, which innervate skeletal, visceral and cardiac muscles^{1,2}. The loss of lower motor neurons causes muscle denervation, muscle atrophy, and ultimately death². Most studies of this disease focused on the pathology seen in spinal cord motor neurons, but recent evidence supports a dying forward model, in which neuronal degeneration begins in the motor cortex and then proceeds to the spinal cord. In support of this model, transcranial magnetic stimulation (TMS) revealed an increase in cortical hyperexcitability prior to clinical motor symptoms in patients with ALS 3. Similarly, in hSOD1^{G93A} transgenic mice, an animal model of ALS, dendritic regression, spine loss and hyperexcitability are observed in the motor cortex before symptom onset at postnatal day (P) 30⁴.

The dying-forward model also proposed that pathology in the motor cortex may not only precede spinal cord disease, but also drives the disease forward. As a corollary to this model, it has been suggested that by preventing the death of upper motor neurons, disease progression could be stalled. Consistent with this finding, knockdown of mutant SOD1 in the motor cortex of hSOD1^{G93A} rats prior to symptom onset delays disease progression, increases survival, and delays lower motor neuron and neuromuscular junction degeneration⁵. In addition, neural progenitor cells transplanted into the motor cortex of hSOD1^{G93A} transgenics, which differentiate into astrocytes secreting glial cell-derived neurotrophic factor (GDNF), delays both upper and lower motor neuron death⁶.

Astrocytes are a major contributor to neuronal cell death in an ALS animal model, as they can induce pathology when transplanted into a healthy host brain⁷. To prevent toxicity associated with ALS astrocytes, and to potentially replace lost upper motor neurons, a neuronal lineage conversion approach has now been developed. Proneural genes, including Neurog1, Neurog2, Ascl1 and Neurod4, encode bHLH transcription factors (TFs) that have emerged as critical architects of neurogenesis in the embryonic brain and neuronal reprogramming in the adult⁹. These bHLH genes act in transcriptional cascades, turning on other bHLH genes, such as Neurod1, which functions at later developmental stages to control neuronal differentiation. However, these bHLH genes are not active in all cellular contexts, and can be inhibited by environmental signals. For example, in the embryonic cortex, Neurog2 is only sufficient (by gain-of-function¹⁰) and necessary (by loss-of-function) to specify a glutamatergic neuronal fate between embryonic day (E) 11.5 to E14.5, despite continued expression at later stages during the neurogenic period, which ends at E17¹¹⁻¹³.

One way that proneural bHLH TF function is inhibited is by phosphorylation by proline-directed serine threonine kinases (e.g. GSK3, ERK1/2, Cdks), which act in a “rheostat-like fashion”; the more serine-proline (SP) or threonine-proline (TP) sites phosphorylated, the less capable these TFs are to bind DNA and transactivate their target genes to promote neuronal fate specification and differentiation^{10,14-16}. There are nine SP sites in mouse Neurog2 and six SPs in mouse Ascl1. Wild-type Ascl1 is phosphorylated by ERK¹⁶. Furthermore, mutation of six serines (SP) to alanines (SA) to generate Ascl1-SA6 reduced the pro-proliferative/gliogenic activity of Ascl1 so that it almost exclusively promotes neurogenesis¹⁶. It has also been found that Neurog2 is phosphorylated by GSK3 on SP sites, which reduces Neurog2 proneural activity. When Neurog2 phosphorylation is inhibited, Neurog2 has a more robust proneural activity, even at E14.5 of development.

The cell context-dependent activities of the proneural genes extends to the adult brain and neuronal reprogramming: Ascl1 can efficiently drive neuronal lineage conversion of fibroblasts⁷⁻²², hepatocytes²³, cardiomyocytes²⁴, astrocytes²⁵ and the differentiation of pluripotent cells²⁶, but Ascl1 is less efficient in the adult neocortex²⁷, hippocampus and spinal cord^{28,29}. Similarly, Neurog2 is used less often for neuronal reprogramming as it must be combined with other signals to become a potent lineage converter³⁰. Thus, by better understanding how proneural genes are regulated, they can be more efficiently used in regenerative medicine.

The present study was performed using a modified version of Ascl1, termed Ascl1-SA6, which was designed such that it would not be subject to environmental inhibitory controls, under the control of an astrocytic promoter (i.e., GFAP).

### Methods

Animals and genotyping. Animal procedures were approved by the Sunnybrook Research Institute (21-757) in compliance with the Guidelines of the Canadian Council of Animal Care. hSOD1^{G93A} (B6.Cg-Tg(SOD1*G93A)1Gur/J, JAX #008230) and Rosa-ZsGreen (JAX #007906) mice were purchased from Jackson Laboratory and maintained on a C57BL/6 background. Mice were housed in cages under 12:12 hour light/dark cycles with ad libitum access to food and water. PCR primers and conditions for genotyping were conducted using Jackson Laboratory protocols: hSOD1^{G93A} forward: CAT CAG CCC TAA TCC ATC TGA (SEQ ID NO: 26); reverse: CGC GAC TAA CAA TCA AAG TGA (SEQ ID NO:27). Rosa-ZsGreen: wild-type forward: CTG GCTTCT GAG GAC CG (SEQ ID NO: 28); wild-type reverse: AAT CTG TGG GAA GTC TTG TCC (SEQ ID 29); mutant forward: ACC AGA AGT GGC ACC TGA C (SEQ ID NO: 30); mutant reverse: CAA ATT TTG TAA TCC AGA GGT TGA (SEQ ID NO: 31).

hSOD1^{G93A} mice were monitored closely for symptoms, including weekly body weight measurements and daily behavioural assessment to determine disease progression. Disease onset and progression measures was monitored using an ALSTDI-Neuroscore. As detailed in Hatzipetros, T., Kidd, J. D., Moreno, A. J., Thompson, K., Gill, A., & Vieira, F. G. (2015). A Quick Phenotypic Neurological Scoring System for Evaluating Disease Progression in the SOD1-G93A Mouse Model of ALS. Journal of visualized experiments: JoVE, (104), 53257, the scoring was performed as follows:
1. Assign NS 0 (Pre-symptomatic) if the following is observed: When the mouse is suspended by the tail, the hindlimb presents a normal splay i.e., it is fully extended away from the lateral midline and it stays in this position for 2 sec or longer. When the mouse is allowed to walk, normal gait is observed.
2. Assign NS 1 (First symptoms) if the following is observed: When the mouse is suspended by the tail, the hindlimb presents an abnormal splay, i.e., it is collapsed or partially collapsed towards lateral midline OR it trembles during tail suspension OR it is retracted/clasped. When the mouse is allowed to walk, normal OR slightly slow gait is observed.
3. Assign NS 2 (Onset of paresis) if the following is observed: When the mouse is suspended by the tail, the hindlimb is partially OR completely collapsed, not extending much. (There might still be joint movement). When the mouse is allowed to walk, the hindlimb is used for forward motion however the toes curl downwards at least twice during a 90 cm walk OR any part of the foot is dragging along cage bottom/table. When the mouse is placed on its left AND right side, it is able to right itself within 10 sec from BOTH sides.
4. Assign NS 3 (Paralysis) if the following is observed: When the mouse is suspended by the tail, there is rigid paralysis in the hindlimb OR minimal joint movement. When the mouse is allowed to walk there is forward motion however the hindlimb is NOT being used for forward motion. When the mouse is placed on its left AND right side, it is able to right itself within 10 sec from BOTH sides. Note: Rarely, after the onset of paralysis, urine moisture might appear on the hindlimbs. Left untreated urine moisture might result to urine “burn” and skin lesions. Treat urine moisture by clipping the hair, apply warm water soaks to remove the urine, and gently blot dry. If skin lesions are present apply antibiotic ointment. 5. Assign NS 4 (Humane end-point) if the following is observed: When the mouse is suspended by the tail, there is rigid paralysis in the hindlimbs. When the mouse is allowed to walk, there is no forward motion. When the mouse is placed on its left AND right side it is NOT able to right itself within 10 sec from EITHER side. i.e., absence of righting reflex.

Disease end-stage is reached if accumulating paralysis leads to the animal no longer righting itself within 15 seconds, and/or a loss of 20% of their body weight compared to age-matched controls, unable to groom, and/or exhibiting signs of bladder dysfunction. These animals were sacrificed immediately.

AAV cloning. AAV2/5-GFAP-iCre and AAV2/5-GFAP-Ascl1-SA6-t2a-iCre were cloned by GenScript™ and packaged by VectorBuilder™ Inc.

Intracranial injection of AAVs. For intracranial injections, 16-week-old C57BL/6 (littermate control) or hSOD1^{G93A} mice were anaesthetized using isoflurane (2%, 1 L/min) and injected subcutaneously with buprenorphine (0.1 mg/kg), baytril (2.5 mg/kg), and saline (0.5 ml). A burr hole was drilled through the skull over the motor cortex and a stereotax was used to identify bregma and lambda levels for injection (AP: +2.15 mm, L/M: +1.7 mm, DV: −1.7 mm). AAV2/5-GFAP-iCre or AAV2/5-GFAP-Ascl1-SA6-2a-iCre were injected at 1.0×10¹²/ml in a 1 μL total volume at 0.1 μl/min over the span of 10 mins using a 5 μl Hamilton syringe with 30-gauge needle. 21 days later, animals were sacrificed and tissues were harvested as described. Disease progression was measured by checking their body weight, NS, motor behavior test (Rotarod, grip strength, and gait analysis) until the experimental endpoint. When mice were not able to right themselves within 30 sec after being placed on either side, they were sacrificed.

Tissue processing and sectioning. Mice were anesthetized with ketamine (75 mg/kg) and xylazine (10 mg/kg) prior to perfusion. Intracardial perfusion was performed with approximately 20× of their blood volume using a peristaltic pump at a flow rate of 10 ml/min with ice cold saline (0.9% NaCl), followed by 4% paraformaldehyde (PFA) in phosphate buffer saline (PBS) for approximately five minutes. Brains and spinal cords were collected and post-fixed overnight in 4% PFA in PBS, cryoprotected at 4° C. in 20% sucrose/1×PBS overnight, embedded in O.C.T. compound (Tissue-Tek® O.C.T. Compound, Sakura® Finetek, PA, USA) and stored at −80° C. Coronal brain sections were cut at 30 μm on a Leica CM3050™ cryostat (Leica Microsystems Canada Inc., Richmond Hill, ON, Canada) and collected on Fisherbrand™ Superfrost™ Plus Microscope Slides (Thermo Fisher Scientific, Ottawa, ON, Canada).

Immunostaining. Slides were washed in PBS, then incubated for 1 hour at room temperature in 10% horse serum (HS), 0.075% bovine serum albumin (BSA), and 0.3% Triton-X-100 in PBS. Blocking was followed by an overnight incubation at 4° C. with rabbit anti-GFAP (1:500, #G9260, Sigma Aldrich Canada, Mississauga, ON, Canada) in antibody solution (10% HS, 0.75% BSA, and 0.1% Triton-X-100 in PBS). Primary antibodies included: rabbit anti-zsGreen (1:500, Takara #632474), rabbit anti-NeuN (1:500, Abcam #ab177487), rabbit anti-GFAP (1:500, DakoCytomation #Z0334), rat anti-GFAP antibody (1/500, Thermo Fisher Scientific #13-0300), rabbit anti-Sox9 (1:500, Millipore #AB5535), rabbit anti-Tbr1 (1:500, Abcam #Ab31940), mouse anti-Satb2 (1:500, Abcam #ab51502), rat anti-Ctip2 (1:100, Abcam, ab18465), and rabbit anti-S100b (1:100, Dako/Agilent #Z031129-2). Slides were washed three times for 10 min each in 0.1% Triton-X-100 in PBS, and incubated with species-specific secondary antibodies conjugated to Alexa568 (1:500; Invitrogen Molecular Probes™, ThermoFisher Scientific, Markham, ON, Canada), Alexa488 (1:500; Molecular Probes) for 1 hour in room temperature. Slides were washed three times in PBS and counterstained with 4,6-Diamidino-2-pheylindole (DAPI) (Sigma Aldrich Canada, Mississauga, ON, Canada). Finally, the slides were washed three times and mounted in Aqua-polymount™ (Polysciences Inc., PA, USA).

RNA in situ hybridization. Colorimetric RNA-in situ hybridization (ISH) was performed using a digoxygenin-labeled Ascl1 riboprobe as previously described³¹ Fluorescent RNA-ISH was performed using an RNAscope® Multiplex Fluorescent Detection Kit v2 (ACD #323110) following the manufacturer's instructions. Briefly, brain sections were post-fixed (4% PFA/1×PBS) for 15 min at 4° C., and then, at room-temperature, dehydrated in 50%, 70% and 100% ethanol for 5 min each, and incubated in H₂O₂ solution for 10 min. Sections were then incubated in 1× target retrieval solution for 5 min at 95° C., washed in dH₂O, and then incubated in Protease Plus™ for 15 min at 40° C. before washing in washing buffer. RNA probes (Mm-Ascl1 #313291 and provided negative and positive control probes) were incubated on the sections for 2 hrs at 40° C. Amplification and staining steps were completed following the manufacturer's instructions, using an Opal™ 570 (Akoya #FP1488001KT; 1:1500) fluorophore.

Imaging. To calculate volume of brain targeted with the gene therapy, a Zeiss Axioscan™ Slide Scanner was used. For higher magnification images, a Leica DMI8™ fluorescent microscopy, a Zeiss Axiovert™ 200M confocal microscopy or a Zeiss observer Z1™ spinning disc confocal microscope was used. Immunolabeled cells were counted using ImageJ™ software.

Behaviour assays. Littermates were split between groups and gender balanced. The study was performed on 12 male and 12 female hSOD1^{G93A} animals per group for survival and behavioural measures based on a sample size calculation using a Cox proportional hazard model, with power 0.95, confidence 95% and 20% effect size.

Rotarod: Motor coordination, strength, and balance were assessed in rotarod apparatus. Mice were trained on the rotarod one week before recording the data. Mice were placed onto a cylinder and rotation started with 2 rpm. Within 180 sec the rotation speed increased up to 18 rpm over a min. with the occurrence of motor neuron symptoms animals get weaker and fall onto the sensing platforms. The time of drop is sensed by magnetic switches and shown on the display at the end of the experiment

Grip strength: A grip strength meter (Bioseb™) was used to study neuromuscular function. Mice were held by the tail over a 100×80 mm at an angle of 20° grid holder. Once mice were firmly grasping the grid, they were pulled along the axis of the force sensor until they released the grid. Grip strength tests were done in three rounds of one trial with 5 min in home cages between the rounds. All grip strength tests were normalized to body weight.

Gait abnormalities: The footprint pattern of mice was monitored as they walked along a 50 cm long and 10 cm wide runway. Forelimbs were painted with a red dye and hindlimbs were painted with a blue dye. Footprint patterns were analyzed for three step parameters: 1) stride length, to measure the average distance of forward movement between each stride; 2) sway distance, to measure the average distance between left and right hind footprints; 3) stance length, to measure the average distance from left or right front footprint to right or left hind footprint.

Quantification and statistics. Statistical analyses were conducted using GraphPad Prism™ Software. Mean values and error bars representing standard error of the mean (SEM) were plotted. Statistical tests used for each experiment are indicated in the figure legends. Quantification of immunostained cells was performed on at least three brains per condition and a minimum of three sections per brain.

### Results

### Mutating Serine Phosphoacceptor Sites in Ascl1 Removes Inhibitory Controls To Augment Neuronal Lineage Conversion in the Adult Motor Cortex

Inhibitory signals that block the neurogenesis-inducing activity of Neurog2 and Ascl1 in the embryonic brain have been identified^{10,16} This study was performed to demonstrate that these same signals block the ability of Ascl1 to induce neuronal lineage conversion in the adult brain. To make the proneural gene Ascl1 insensitive to inhibitory cues in the environment (especially in an injury or neurodegenerative disease), a mutant form of Ascl1 mutant was engineered in which the six nucleotides encoding serines (S) adjacent to prolines in Ascl1 were replaced with alanines (A), referred to herein as Ascl1-SA6. Ascl1 (wild-type, unmodified gene), Ascl1-SA6 or Neurod1, as a control, were cloned into an AAV2/5 vector, under the control of an astrocytic promoter (GFAP), that also carried a t2a-iCre cassette.

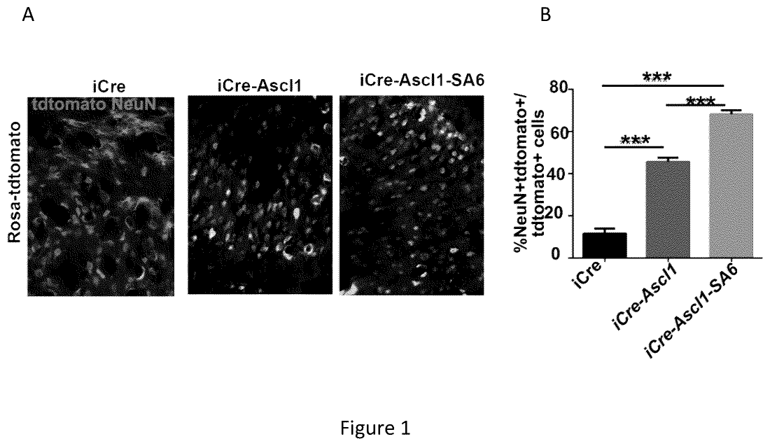

The AAV vectors were injected intracranially into the motor cortex of Rosa-tdtomato mice, a cre-reporter line, which allowed the fate of transduced neurons to be followed. Three weeks later, brains were dissected and sectioned and immunostained with tdtomato and NeuN antibodies. Strikingly, it was found that while Ascl1 and Neurod1 had similar conversion efficiencies of ˜50%, the mis-expression of Ascl1-SA6 nearly reached a 75% neuronal conversion efficiency (i.e., % NeuN⁺tdtomato⁺/tdtomato⁺ cells) (FIG. 1). All of these conversion rates were well above the ˜15% NeuN+zsGreen⁺/zsGreen⁺ cells observed after the expression of iCre alone, a control for transduction rates.

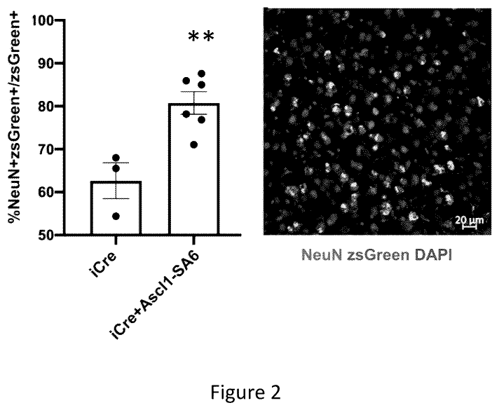

Stereotactic injections of AAV5-GFAP-iCre (control) and AAV5-GFAP-Ascl1-SA6-t2a-iCre were made into 16 weeks old hSOD^{G93A} transgenic ALS mice, this time carrying a Rosa-zsGreen transgene. The conversion of astrocytes into neurons was monitored using NeuN immunostaining. These animals were sacrificed at disease endpoint, so the assessment of conversion of efficiency included animals that were collected between 5-12 weeks (and one at 17 weeks) post-transduction. A significant increase in the number of NeuN+zsGreen+/zsGreen+ cells (neuronal conversion efficiency) was observed after the expression of Ascl1-SA6. Background levels of neuronal staining also increased in the iCre transduced brains. Note that there are many possible reasons for this increase; it is likely an artefact of zsGreen transferring between the primary transduced astrocytes and the neighboring neurons. Nonetheless, as shown in FIG. 2, there was a significant increase in neuronal conversion following expression of Ascl1-SA6 in comparison to the control.

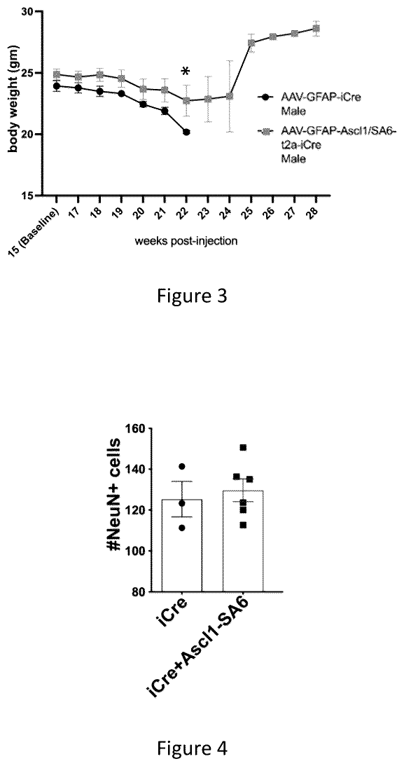

To model neurodegenerative disease progression, body weight was measured, since ALS is a wasting disease, as well as motor behaviour. Baseline measurements were set with 15 weeks old hSOD1^{G93A} and C57B16 (wild-type) male mice. 15 weeks is at the onset of a symptomatic phase in hSOD1^{G93A} mice. The results are shown in FIG. 3. It was observed that male mice injected with the Ascl1-SA6 vector were better able to sustain their body weight over time, an indirect measurement of enhanced neuronal survival and an indicator of disease treatment.

Neuronal density (total number of NeuN+ cells) was also studied. As shown in FIG. 4, there was a slight trend towards more neurons in the Ascl1-SA6 vs iCre control transduced brains.

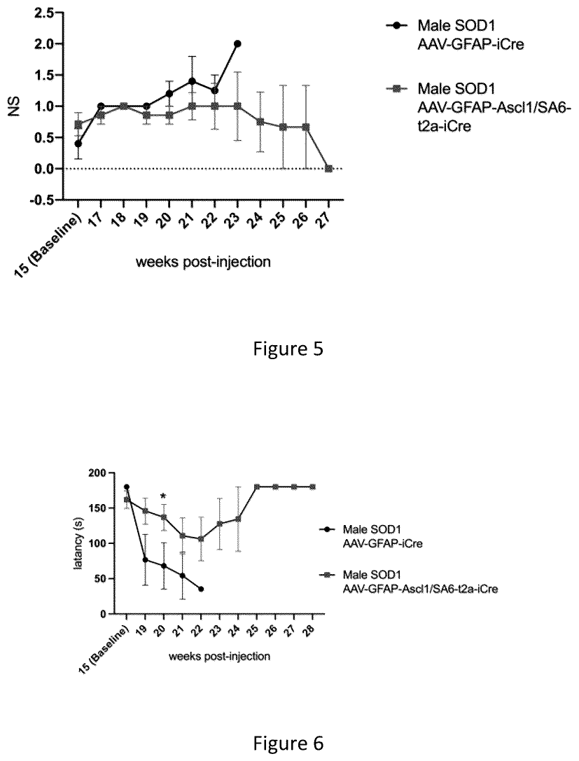

The neurological score (NS), captured by ALSTDI-Neuroscore (Score Sheet Appendix 1), was monitored in the treated mice. A lower score is indicative of better health (the score increases with disease progression), in longer surviving animals with Ascl1-SA6 vs control injections. The results are summarized in FIG. 5

Further, using the rotarod performance test to assess motor coordination, strength, and balance in the treated mice, a clear trend toward improved motor behaviour was observed after Ascl1-SA6 treatment in comparison to the control mice. The results are summarized in FIG. 6.

Overall, the results from this study confirm that use of the modified version of Ascl1, Ascl1-SA6, was more efficient than Ascl1 at inducing the conversion of motor cortex astrocytes to neurons and that by using Ascl1-SA6 to drive neuronal lineage conversion of motor cortex astrocytes in hSOD1^{G93A} transgenic mice, animals preserved a higher body weight and a better neuroscore for longer times than the control animals. These animals also had a trend towards improved motor behaviour. Taken together this data demonstrates that neuronal lineage conversion of motor cortex astrocytes is useful as a therapy for ALS.

### REFERENCES

1 de Carvalho, M. & Swash, M. Lower motor neuron dysfunction in ALS. Clin Neurophysiol 127, 2670-2681, doi:10.1016/j.clinph.2016.03.024 (2016).
2 Turner, M. R. et al. Controversies and priorities in amyotrophic lateral sclerosis. Lancet Neural 12, 310-322, doi:10.1016/S1474-4422(13)70036-X (2013).
3 Vucic, S., Nicholson, G. A. & Kiernan, M. C. Cortical hyperexcitability may precede the onset of familial amyotrophic lateral sclerosis. Brain 131, 1540-1550, doi:10.1093/brain/awn071 (2008).
4 Ozdinler, P. H. et al. Corticospinal motor neurons and related subcerebral projection neurons undergo early and specific neurodegeneration in hSOD1G(9)(3)A transgenic ALS mice. J Neurosci 31, 4166-4177, doi:10.1523/JNEUROSCI.4184-10.2011 (2011).
5 Thomsen, G. M. et al. Delayed disease onset and extended survival in the SOD1G93A rat model of amyotrophic lateral sclerosis after suppression of mutant SOD1 in the motor cortex. J Neurosci 34, 15587-15600, doi:10.1523/JNEUROSCI.2037-14.2014 (2014).
6 Thomsen, G. M. et al. Transplantation of Neural Progenitor Cells Expressing Glial Cell Line-Derived Neurotrophic Factor into the Motor Cortex as a Strategy to Treat Amyotrophic Lateral Sclerosis. Stem Cells, doi:10.1002/stem.2825 (2018).
7 Qian, K. et al. Sporadic ALS Astrocytes Induce Neuronal Degeneration In Vivo. Stem Cell Reports 8, 843-855, doi:10.1016/j.stemcr.2017.03.003 (2017).
8 Masserdotti, G., Gascon, S. & Gotz, M. Direct neuronal reprogramming: learning from and for development. Development 143, 2494-2510, doi:10.1242/dev.092163 (2016).
9 Oproescu, A. M., Han, S. & Schuurmans, C. New Insights Into the Intricacies of Proneural Gene Regulation in the Embryonic and Adult Cerebral Cortex. Front Mol Neurosci 14, 642016, doi:10.3389/fnmol.2021.642016 (2021).
10 Li, S. et al. GSK3 temporally regulates neurogenin 2 proneural activity in the neocortex. J Neurosci 32, 7791-7805, doi:10.1523/JNEUROSCI.1309-12.2012 (2012).
11 Britz, O. et al. A role for proneural genes in the maturation of cortical progenitor cells. Cerebral cortex 16 Suppl 1, i138-151, doi:10.1093/cercor/bhj168 (2006).
12 Schuurmans, C. et al. Sequential phases of cortical specification involve Neurogenin-dependent and -independent pathways. The EMBO journal 23, 2892-2902 (2004).
13 Fode, C. et al. A role for neural determination genes in specifying the dorsoventral identity of telencephalic neurons. Genes & development 14, 67-80 (2000).
14 Ali, F. et al. Cell cycle-regulated multi-site phosphorylation of Neurogenin 2 coordinates cell cycling with differentiation during neurogenesis. Development 138, 4267-4277, doi:10.1242/dev.067900 (2011).
15 Hindley, C. et al. Post-translational modification of Ngn2 differentially affects transcription of distinct targets to regulate the balance between progenitor maintenance and differentiation. Development 139, 1718-1723, doi:10.1242/dev.077552 (2012).
16 Li, S. et al. RAS/ERK signaling controls proneural genetic programs in cortical development and gliomagenesis. J Neurosci 34, 2169-2190, doi:10.1523/jneurosci.4077-13.2014 (2014).
17 Vierbuchen, T. et al. Direct conversion of fibroblasts to functional neurons by defined factors. Nature 463, 1035-1041, doi:10.1038/nature08797 (2010).
18 Pang, Z. P. et al. Induction of human neuronal cells by defined transcription factors. Nature 476, 220-223, doi:10.1038/nature10202 (2011).
19 Caiazzo, M. et al. Direct generation of functional dopaminergic neurons from mouse and human fibroblasts. Nature 476, 224-227, doi:10.1038/nature10284 (2011).
20 Son, E. Y. et al. Conversion of mouse and human fibroblasts into functional spinal motor neurons. Cell Stem Cell 9, 205-218, doi:10.1016/j.stem.2011.07.014 (2011).
21 Kim, J. et al. Functional integration of dopaminergic neurons directly converted from mouse fibroblasts. Cell Stem Cell 9, 413-419, doi:10.1016/j.stem.2011.09.011 (2011).
22 Pfisterer, U. et al. Direct conversion of human fibroblasts to dopaminergic neurons. Proceedings of the National Academy of Sciences of the United States of America 108, 10343-10348, doi:10.1073/pnas.1105135108 (2011).
23 Marro, S. et al. Direct lineage conversion of terminally differentiated hepatocytes to functional neurons. Cell Stem Cell 9, 374-382, doi:10.1016/j.stem.2011.09.002 (2011).
24 Chuang, W. et al. Partial Reprogramming of Pluripotent Stem Cell-Derived Cardiomyocytes into Neurons. Sci Rep 7, 44840, doi:10.1038/srep44840 (2017).
25 Rivetti di Val Cervo, P. et al. Induction of functional dopamine neurons from human astrocytes in vitro and mouse astrocytes in a Parkinson's disease model. Nat Biotechnol 35, 444-452, doi:10.1038/nbt.3835 (2017).
26 Yang, N. et al. Generation of pure GABAergic neurons by transcription factor programming. Nat Methods 14, 621-628, doi:10.1038/nmeth.4291 (2017).
27 Grande, A. et al. Environmental impact on direct neuronal reprogramming in vivo in the adult brain. Nat Commun 4, 2373, doi:10.1038/ncomms3373 (2013).
28 Jessberger, S., Toni, N., Clemenson, G. D., Jr., Ray, J. & Gage, F. H. Directed differentiation of hippocampal stem/progenitor cells in the adult brain. Nat Neurosci 11, 888-893, doi:10.1038/nn.2148 (2008).
29 Ohori, Y. et al. Growth factor treatment and genetic manipulation stimulate neurogenesis and oligodendrogenesis by endogenous neural progenitors in the injured adult spinal cord. J Neurosci 26, 11948-11960, doi:10.1523/JNEUROSCI.3127-06.2006 (2006).
30 Gascon, S. et al. Identification and Successful Negotiation of a Metabolic Checkpoint in Direct Neuronal Reprogramming. Cell Stem Cell 18, 396-409, doi:10.1016/j.stem.2015.12.003 (2016).
31 Touahri, Y. et al. Non-isotopic RNA In Situ Hybridization on Embryonic Sections. Curr Protoc Neurosci 70, 1 22 21-21 22 25, doi:10.1002/0471142301.ns0122s70 (2015).

### Example 2: Neuronal Lineage Conversion for Alzheimer's Disease Therapy

Alzheimer's Disease (AD) is a devastating neurodegenerative disorder that slowly robs individuals of their cognitive abilities. Progressive cognitive decline in AD is associated with several hallmark pathological features: deposition of amyloid beta peptide (Aβ), formation of neurofibrillary tangles, and neuronal loss¹. Synaptic loss is postulated to initiate symptoms of AD and has provided the best neurophysiological correlate of cognitive decline to date^{2,3}, suggesting that neuronal dysfunction is critical for disease progression. Cognitive decline, however, is not driven by disturbances in a few synapses in isolation, but by an aberration in the functioning of the entire neuronal network, which is assembled through interactions between excitatory, inhibitory and neuromodulatory cells. As disease progresses, loss of specific populations of cells exacerbates cognitive function and renders a point of no return for present therapeutic interventions. Neuropathological studies on TgAD mice that exhibit both Aβ and tau pathology showed accelerated rate of neuronal compromise and Aβ-independent neuronal loss, indicating significant role of tauopathy in AD progression,^{4,5} underscoring the significance of evaluating neuronal compromise in AD models exhibiting both Aβ accumulation and neurofibrillary tangle formation as both lead to toxicity⁶ and their complex interaction exacerbates degeneration.^{6,7}

Brain circuitry processes information by rapidly and selectively engaging specific subsets of neurons forming the neuronal networks. The dynamic formation of networks is often evident in rhythmically synchronized neuronal activity that tightly correlates with perceptual, cognitive, and motor performance². The spectral content of electrophysiological recordings is classically divided into frequency bands and oscillatory activities are related to global states or specific behaviors. Neural oscillations occur at various frequencies; important to AD are the theta waves (4-8 Hz), which are associated with motor behavior and memory formation, and gamma waves (30-200 Hz), which play a role in conscious thought. Importantly, theta and gamma oscillations interact via cross-frequency coupling during normal network communication. The impaired cognitive functions associated with AD in both humans and rodents are related to temporal modulation of theta oscillations and theta-gamma coupling^{9,10} which depends on the balance between excitatory and inhibitory neuronal activity.

Of particular interest here, the phase of theta oscillations affects memory processing through modulation of neuronal plasticity (i.e., neuronal changes in response to experience) and induction of long-term-potentiation in hippocampal and cortical areas (i.e., strengthening of neuronal synapses, or connections to other neurons, based on patterns of activity). A variety of cognitive processes, including working memory, modulates gamma oscillations^{11,12} which are in turn modulated by theta oscillations¹³: this modulation of gamma by theta is known to depend on an intact inhibitory network of gamma-aminobutyric acid (GABAergic) interneurons.

There are several types of GABAergic interneurons characterized by their expression of neuropeptides and calcium binding proteins. Memory acquisition requires changes in firing patterns of parvalbumin (PV) and somatostatin (SST) expressing hippocampal (GABAergic) interneurons^{14,15}. These inhibitory interneuronal sub-populations control the activity of hippocampal excitatory granule cells, with PV-expressing interneurons targeting granule cell soma and axon segments to control the action potential output of granule cells while SST-expressing cells target dendrites to control input to granule cells¹⁶. Recently it was shown that hippocampal granule cells activate SST-interneurons to regulate both the size of the memory engram, the units of memory storage, as well as the stability of the resultant memory¹⁷.

In AD patients and mouse models, the selective and early loss of PV and SST-expressing interneurons is mediated by Aβ or tau accumulation and ApoE4 expression¹⁸⁻²⁴. The loss of PV-GABAergic interneurons correlated with epileptic activity while SST-interneuron loss related to cognitive decline; modulation of GABA signaling prevented both the epileptic activity and memory deficits in mouse models^{23,25-27}.

Historically, the use of GABAergic targeting drugs as well as exogenous stem cell replacement have shown promise in rodent models of AD for improving memory, but they have resulted in severe side-effects in patients. GABAergic modulating drugs often induce sedative or anxiolytic side effects, and their chronic administration is associated with worsening of cognitive function with aging²¹. Furthermore, GABAergic drug interventions may have a limited temporal window of action as interneurons continue to deteriorate.

Although much recent research attention has been focused on cell therapy strategies, efficient and safe methods to generate a renewable source of specific interneuron populations are lacking²⁸⁻³⁰. The transplant of progenitor cells from the medial ganglionic eminence of the embryonic ventral telencephalon has demonstrated the potential to induce appropriate sub-type specific interneurons in various disease states, supporting the potential of cell replacement therapies^{28,29}. To move this approach into a more clinically relevant application, a more accessible and safer source of GABAergic neurons is required. A potentially safer alternative is to convert endogenous cells into neurons by direct neuronal reprogramming using supra-physiological expression of lineage-specific transcription factors³¹⁻³³. There have been a number of studies that have utilized this approach to convert fibroblasts, hepatocytes, pluripotent stem cells and other cells to neurons with combinations of up to 4 transcription factors³⁴⁻³⁷. Although these studies still required invasive cell transplantation, they demonstrated the potential to generate subtype specific GABAergic interneurons utilizing the transcription factor Ascl1 alone or in combination with Dlx2^{38,39}. Thus, the present treatment strategy is to rebalance the neuronal network by AAV-delivered transcription factor induction of transdifferentiation of endogenous activated astrocytes into fully integrated GABAergic neurons that respond appropriately to environment signals.

In both humans and rodents, spatial memory representations of the neuronal network are related to temporal modulation of theta oscillations and theta-gamma coupling. AD patients present a reduced coupling of resting state EEG rhythm; and coupling of cortical rhythms at specific frequency bands may be a specific marker of AD. Without wishing to be bound by theory, it is proposed that loss of GABAergic interneuron function and ultimately cell loss, results in neuronal network dysfunction and contributes to cognitive dysfunction. Thus, restoration of GABAergic interneuron function will rebalance excitatory-inhibitory neurotransmission leading to improved cognitive function.

To test demonstrate the efficacy of this approach in this study, the proneural transcription factors, Ascl1, Ascl1-SA6, Neurod1, were transiently expressed in reactive, GFAP+ astrocytes to induce transdifferentiation to fully functional and integrated GABAergic neurons within the hippocampal region.

To support this approach, studies to date have induced astrocyte-to-neuron transdifferentiation in early post-natal day animals or in AD mouse models that exhibit at best minimal neuronal loss: as a result, the newly formed neurons were required to integrate into a fully functional neuronal network, which is fundamentally unlike the conditions present in the AD brain^{29,38,40,41}. The present study distinguishes itself from previous work as it makes use of overexpression of a gene that specifies a GABAergic identity, as opposed to overexpression in glutamatergic neuronal lineage determinants (Neurod1).

Previous studies presented evidence that transdifferentiation is possible in old rodents⁴⁰. Another consideration for the present approach is the extent to which astrocytes are not only reactive but also proliferative; reactive astrocytes activated under different pathological conditions have been shown to exhibit different proliferation rates. In particular, stab injury reactive astrocytes can be highly proliferative, whereas reactive astrocytes in APPPS1 or CK/p25 mice have lower rates of proliferation⁴². This lower proliferation may actually be a benefit in AD, as the aim is to rebalance the network not produce an overpopulation of inhibitory cells that would change the balance from predominantly excitatory to inhibitory, which could also be detrimental.

### Methods

As AD affects both males and females, 4 sex-balanced groups of Tg344 AD and nTg (non-transgenic) rats were used: 1) AAV2/8-mCherry control virus infected and 2) AAV2/8-Ascl1-mCherry infected or Ascl1-SA6 or Neurod1. Although AAV2/8 prefers neurons in prenatal and post-natal rodents, it targets astrocytes in adult animals⁴⁰, which in conjunction with GFAP promoter increases vector specificity. As GABAergic neuronal loss occurs in the hippocampus and, in particular, in the hilus in early stages of disease, activated astrocytes were infected using stereotactic injections as dictated by disease state. Although invasive, this strategy allows for controlled delivery of a finite viral load within a defined location such that it is possible to determine the role of GABAergic loss in each hippocampal region individually and in concert. Some studies have examined astrocyte transdifferentiation to glutamatergic neurons at 3 weeks post viral infection⁴⁰; while other studies have suggested that maturation of GABAergic neurons occurred 28-42 days post infection⁵⁴: therefore, the present assays were performed at 4- and 7-weeks post-infection. The generation of new mature neurons was assessed via pathological analyses, structural and functional incorporation of new neurons into existing networks within the hippocampus utilizing in vivo electrophysiological readings. Viral infection was initiated at the stage following significant overt GABAergic neuronal loss, at 9 and 14 months of age.

Viral Administration: To induce lineage conversion in the transgenic AD rat model, AAV2/8-Ascl1-mCherry and AAV2/8-mCherry (control) vectors were used initially. These vectors were previously shown to promote the transdifferentiation of astrocytes into neurons in the dorsal midbrain⁴¹. The AAV2/5 vectors were then used for the comparison of the proneural transcription factors as this was subsequently shown to be more efficient within the CNS. Furthermore, due to the inclusion of the iCre, the mCherry tag was not necessary in the vectors.

Stereotaxic injections of AAV-GFAP-proneural transcription factor vectors were made into the hilus of each hemisphere of the hippocampus of anesthetized TgF344-AD rats and their nTg littermates through a burr hole drilled through the skull. Diffusion of AAV is limited by interstitial fluid flow changes as a function of the stereotactic injection and AAV has a short half-life in the extracellular space as it is removed by glymphatic drainage^{65,66}. Since the goal of this project was to rebalance the excitatory/inhibitory activity within the hippocampal network and GABAergic neurons account for only 10% of the neuronal population, the goal was to obtain sufficient but limited numbers of mCherry+NeuN+GAD67+ neurons. Viral titres of 4.0×10⁸ and 1.2×10⁹ were used and it was found that the lower viral titre was more conducive to a limited conversion rate and a benefit to the neuronal network.

In situ Electrophysiology: These experiments were performed on an independent set of rats to enable the most sensitive assay, namely the contrast between in vivo electrophysiological recordings in the ipsilateral and contralateral hemispheres within each animal. As such, the AAV2/8-mCherry and AAV2/8-Ascl1-mCherry vectors injection was randomized to right vs. left hemisphere across each rat within the cohort. The study followed previously established protocols for assessing hippocampal neuronal network functioning⁵³. Two cranial windows were opened, one per hemisphere, so that the centres of the craniotomies were situated over the dorsal hippocampus. A two-shank linear multielectrode array (LMA) was lowered into each window. Each shank was equipped with two platinum/iridium recording site (200 m in diameter, Microprobes™ for LifeScience). Resting state local field potentials were amplified between 0.3 Hz and 5 kHz, sampled at 20 kHz and stored for analysis.

The frequency bands of interest (Theta (3-9 Hz), Alpha (10-14 Hz), Beta (15-30 Hz), low Gamma (30-58) and high Gamma (62-120 Hz)), have been hypothesized to arise from dynamic motifs that are based on structural circuits and to dictate behavioural function after input-output transformation⁶⁷. High Gamma activity is modulated by sensory, motor, and cognitive events, is functionally distinct from low gamma, and has distinct physiological origins⁶⁸. The phenomenon by which one frequency band can modulate the activity of a different frequency band has been observed in both animal and human data and is termed cross-frequency coupling⁶⁹. Specifically, the phase of the theta wave can modulate the amplitude of gamma bands and this phenomenon, named phase-amplitude coupling, can be resolved with intracortical local field potential^{52,70,71}. To quantify the phase-amplitude coupling a Modulation Index (MI) was used, which combines the amplitude envelope of a high-frequency band time series with the phase time series of a low-frequency band into one composite, complex-valued signal.

Pathological Characterization: To evaluate GABAergic interneuronal loss/rescue, global GAD65/67, neuropeptide expression including SST, NPY, CCK, and VIP, as well as GABA_{A} (fast inhibition) and GABA_{B} (slow inhibition) receptor subunit expression was quantified using Western blot analyses²⁰. It was determined whether the GABAergic neuronal populations, GAD67+, are fully matured using co-expression with NeuN. The expression and colocalization of GABA to mCherry+ cells was examined immunohistochemically. Evaluation of AD-related outcomes and confirmation of the effect size immunohistochemical analyses of amyloid plaques, was done following previously described protocols^{53,63,86}. Changes in glial response after transdifferentiation were evaluated using GFAP antibody for astrocytes and Iba-1 antibody for microglial cells. Neurotoxin and neurodegeneration were evaluated using colocation of S100β and complement component 3 (C3) and colocalization of βIII tubilin and RIPK1, respectively. All immunohistochemistry was imaged on a Zeiss Observer™ Z1 microscope or Nikon A1 (Melville, New York, NY) laser scanning confocal microscope: Aβ, NeuN, GFAP, Iba-1 (10× analysis, 20× representative). A3 plaque analyses were conducted by subtracting background, binarizing images and analyzing for staining density (% area covered) in ImageJ™.

Barnes maze: All rats were naïve to behavioural assessment. Barnes maze (Maze Engineers) testing was conducted in a behavioural suite with spatial cues, and an aversive light in all trials except the training. EthoVision™ XT (Noldus, version 11.5) was utilized to collect and analyze video data. Following training to the location of the escape, rats learned the task across 3 days, with 2 trials per day. Spatial memory was assessed 3 days later, in 1 trial. Reversal learning (5 days, 2 trials per day) for executive function began the next day, in which the location of the escape hole was switched without re-training.

### Results

In light of previously published work, the F344 rat strain was used in the present study. The rat strain has been used as a model of normal aging for the last 40 years⁴³, thus having known temporal evolution of cognitive deficits^{44,45}. Six to eight month old F344 rats exhibit subtle executive function deficits, but no memory deficits⁴⁶. Furthermore, these rats show an age-dependent increase in rat Aβ accumulation, reduced Aβ brain-to-blood efflux, and decreased cholinergic synaptic function that has been correlated with cognitive dysfunction, similar to that reported in the aging human population⁴³⁻⁴⁶.

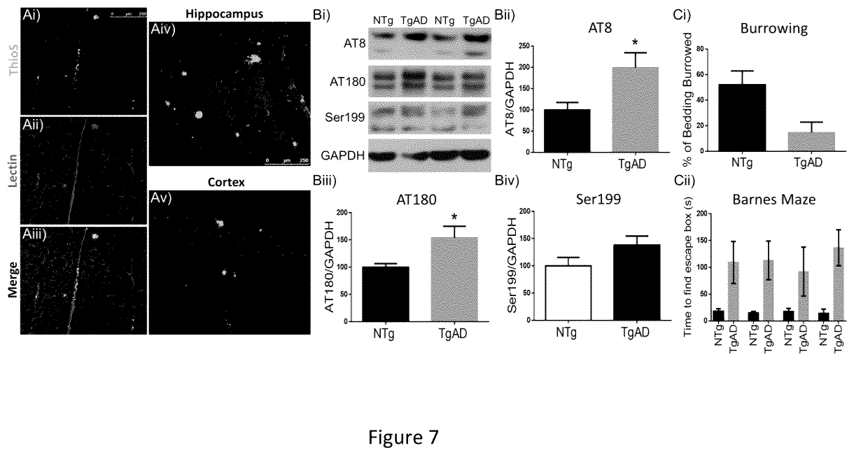

The TgR344AD rats, used herein, exhibit progressive amyloid and tau pathology as well as cognitive deficits, inflammation and frank neuronal loss, thus recapitulating the hallmark features of both the prodromal and symptomatic phases of human AD. The cognitive and amyloid phenotype in the colony used in the present study has been demonstrated and aligns well with the original publication on this model⁸. Specifically, the animals have no amyloid plaques at 3 months of age; some parenchymal plaques at 6 months of age; and cerebrovascular amyloid angiopathy, tau hyperphosphorylation, and amyloid tissue plaques by 9 months of age (FIG. 7). Notwithstanding, in the colony used in this study, the TgF344 AD rats exhibited normal activities of daily living and cognition up to 6 months of age. By 12 months of age, the TgAD animals showed deficits in open field test, in burrowing activity and in the Barnes Maze task (FIG. 7C).

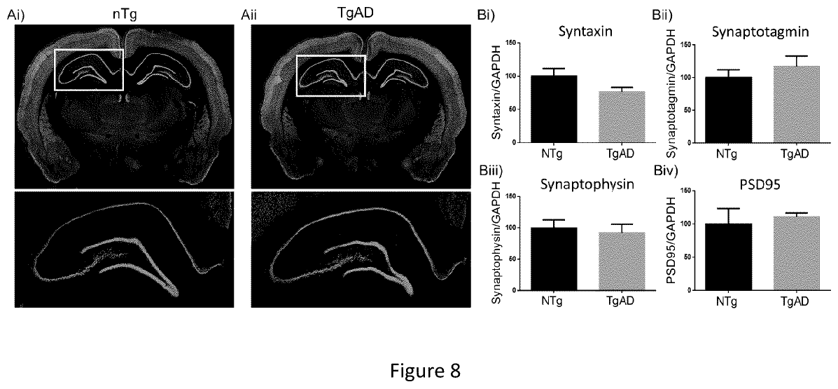

To evaluate neuronal survival in the hippocampus, the neuronal marker NeuN was used to label the nuclei of post-mitotic neurons⁴⁷. Qualitative analysis of NeuN immunohistochemical staining showed comparable number of NeuN+ cells in the hippocampus of 12-month old TgF344 AD rats and their non-transgenic (nTg) littermates, as shown previously (FIG. 8). Accordingly, the levels of pre-synaptic proteins, synaptophysin, syntaxin, and synaptotagmin as well as the post-synaptic protein PSD95 were indistinguishable between the two cohorts (FIG. 8). The majority of hippocampal neurons are excitatory, and about 10% are inhibitory GABAergic interneurons. Since interneurons account for only a small portion of neurons in the hippocampus, a NeuN count alone may mask differences in interneuronal populations and synaptic markers may be more indicative of excitatory than inhibitory neuronal function.

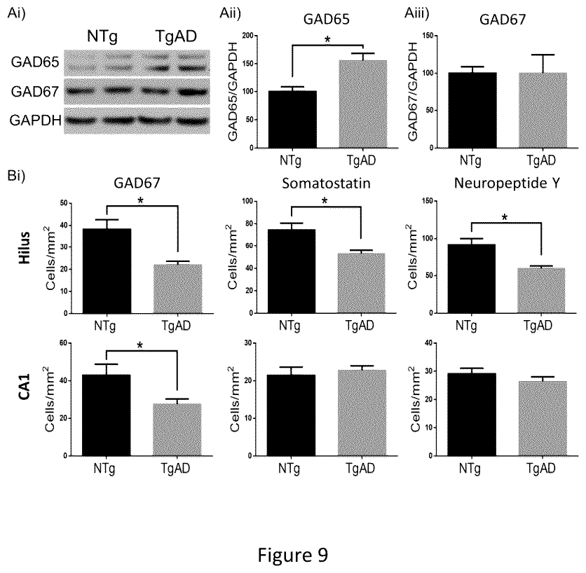

Glutamic acid decarboxylate (GAD) converts glutamic acid to GABA; its two isoforms, GAD67 and GAD65, are predominantly localized in GABAergic cell bodies and axon terminals, respectively. On postmortem histological analysis of hippocampal tissue from AD patients, GAD65 protein levels are decreased, while GAD67 is relatively preserved. In contrast, at the early stage of disease progression presently examined, an increase in the protein expression of GAD65 was found in TgF344 AD rats in comparison to that of nTg animals (FIG. 9A), while no change was detected in GAD67. These results indicated that synaptic GABA production, as illustrated by an increase in GAD65 protein, was increased in an attempt to maintain function.

The neuropeptides expressed by GABAergic interneurons include cholecystokinin (CCK), somatostatin (SST), vasoactive intestinal polypeptide (VIP), and neuropeptide Y (NPY). The calcium binding proteins are parvalbumin (PV), calbindin, and calretinin (CR). In contrast to global hippocampal protein levels of GAD67, a decrease in the number of interneurons was observed within all regions of the hippocampus that express GAD67, primarily comprising SST and NPY expressing cells (FIG. 9B). These combined results suggest that although the number of GAD67-expressing cells was decreased, the remaining cells increased GAD67 expression in an attempt to compensate for neuronal loss. The loss of SST-expressing GABAergic neurons in aging, AD and AD mouse models has been well characterized and shown to correlate with cognitive deficits¹⁸⁻²³. Importantly the magnitude of decline in SST-expressing GABAergic neurons in the hilus of the hippocampus strongly predicts the severity of memory deficits2^{8,48}. In contrast to mouse models of AD, a loss of PV-expressing GABAergic neurons across the hippocampus was not observed at the age examined in this study (data not shown).

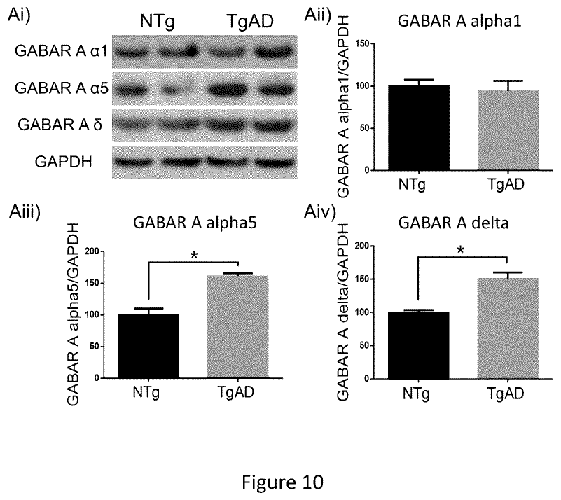

In the next part of the study, the levels of the two main classes of GABA receptors: GABA_{A} and GABA_{B} were examined. GABA_{A} receptors have been implicated in disease states including AD⁴⁹. GABA_{A} receptors are composed of multiple subunits with the most common type in the brain being a pentamer with combinations of 2αx, 2βx and 1γx subunits. GABA_{A} receptors are localized either intra-synaptically or extra-synaptically, mediating phasic and tonic inhibition, respectively. Subunit α-1 is a part of intra-synaptic receptors; α-5 can be either intra-synaptic or extra-synaptic; whereas δ subunit is exclusively extra-synaptic⁴⁹⁵⁰. Increased expression of α-5 and δ but not α-1 GABA_{A} receptor subunits was observed when comparing TgF344 AD to nTg rats suggesting an increase in tonic inhibition (FIG. 10). Although GABAergic interneuronal deficits have been previously reported in different AD rodent models, the sub-type level changes, as classified by neuronal neuropeptide and calcium binding protein expression, vary depending on transgene(s) expression. In toto, these data demonstrate dysfunction of the GABAergic system by 9-months of age in TgF344 AD rats.

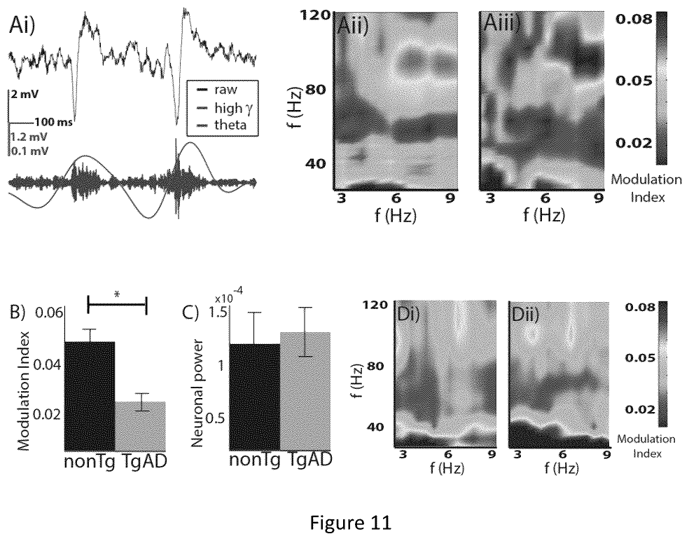

The interaction between oscillations in different frequency bands used in electrophysiology to characterize neuronal networks across or within brain regions is termed Cross Frequency Coupling (CFC). Fast-firing GABAergic interneurons are thought to play a key role in CFC because of their contribution to both gamma and theta rhythms^{52,52}. This makes CFC estimation a useful assay for investigating interneuronal activity. In vivo, 9-month old TgF344 AD rats showed significant impairments in hippocampal neuronal functioning⁵³, reflected in the attenuation of CFC in the hippocampus when compared to that of their age-matched nTg littermates (FIG. 11A-C). In particular, we estimated the Modulation Index (MI) of theta phase on high-gamma amplitude in the hippocampus was estimated and reduced MI in the TgAD cohort was observed (FIG. 11B, 11C) compared to that of nTg animals (FIG. 11B, 11C). Further, it was found that the carrying frequency of the modulation was centered at 6 Hz in the nTg cohort, in contrast to the TgF344 AD cohort (FIG. 11D). The power in the hippocampus of young (<2 months) animals was measured, which did not show a difference between genotypes (p>0.05). Likewise, the theta to high gamma modulation did not show any difference (p>0.05, data not shown). These data demonstrate that at 2 months of age, the TgF344 AD rats do not present evidence of hippocampal network dysfunction (FIG. 11D) suggesting, in contrast to many AD mouse models²⁶, neuronal networks in these rats develop normally and network deficits emerge with aging.

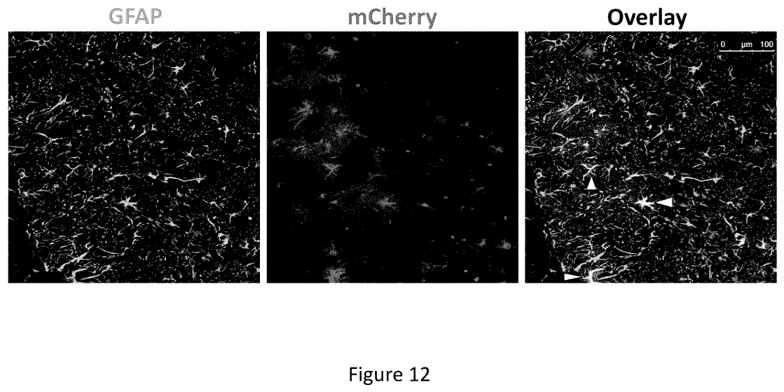

To establish that AAV2/8 driven Ascl1 transcription factor expression under the GFAP promoter⁴¹ in reactive astrocytes drives transdifferentiation into GABAergic neurons in TgF344 AD rats and their nTg littermates, the hilus of the hippocampus was infected in 6- and 12-month old TgF344 AD rats and nTg littermates by stereotactic injection of the virus. The AAV2/8-mCherry and AAV2/8-Ascl1-mCherry vectors were individually injected into the right or left hemisphere within the same rat. Effective infection of reactive astrocytes in 6-month old (data not shown) and 12-month old TgF344 AD rats (FIG. 6) was achieved, as evidenced by the colocalization of GFAP with the mCherry fluorescent marker expression in cells with an astrocytic morphology (FIG. 12).

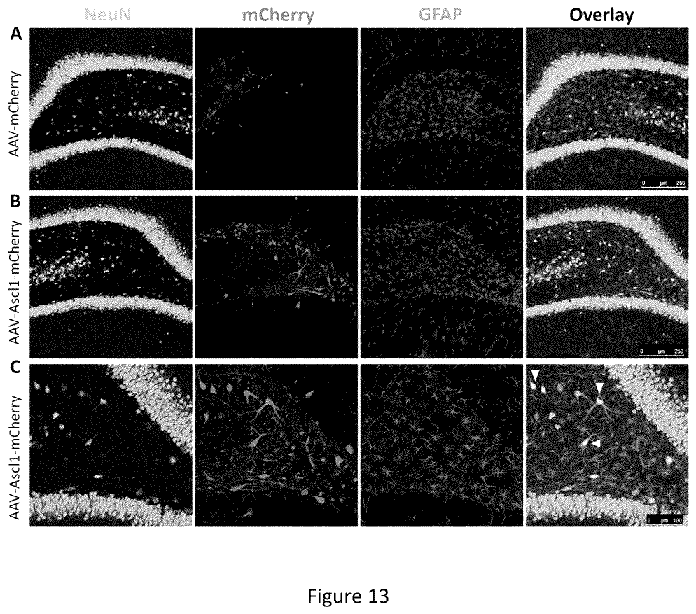

After 28 days, the AAV2/8-mCherry infected hemisphere had very little expression of mCherry and expression was not associated with NeuN expression (FIG. 13). This is not surprising as, although the AAV2/8-mCherry vector will infect proliferative astrocytes, the expression of the virus and hence mCherry expression will diminish over time as the cells end the proliferative cycle and GFAP expression stabilizes. GFAP is a stable structural protein with a slow turnover rate and since mCherry expression is governed by the GFAP promoter it will also decrease over time due to lack of translation. In contrast, the AAV2/8-Ascl1-mCherry infected hemisphere had extensive mCherry+NeuN+ double-labelled cells located in the region populated by reactive astrocytes. Based on the morphological characteristics and location of the mCherry+NeuN+ cells, it is proposed that these cells are immature and have not migrated and fully integrated into the neuronal network. There were also a number of mCherry+NeuN− cells present in the hilus: these cells are likely at an earlier stage of differentiation as not all astrocytes will transdifferentiate simultaneously (FIG. 13).

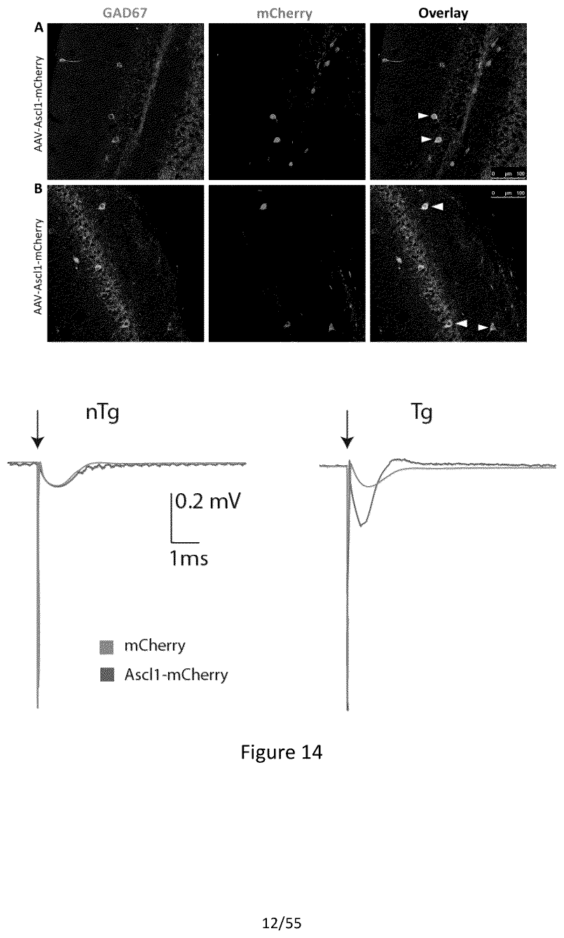

In both young and old TgF344 AD and nTg rats, GAD67+mCherry+ neurons were present in the hilus (FIG. 14). The number of GAD67+mCherry+ cells was low, likely due to fate specification using transdifferentiation taking 28-42 days⁵⁴, so that our results may represent an early stage of GABAergic neuronal maturation. More GABAergic neurons are thus expected to result from this intervention at a later time point. Most importantly to the objectives of this study, the potential benefit of neuronal specification on hippocampal network activity was investigated. In the nTg rats, the AAV2/8-mCherry infected hemisphere was not significantly different from the mCherry-Ascl1 infected hemisphere (FIG. 14). These results suggest that the specification of new neurons does not activate or inhibit a fully functional network. In TgF344 AD rats, on the other hand, hippocampal activity was enhanced in the AAV2/8-mCherry-Ascl1 infected hemisphere in contrast to the AAV2/8-mCherry-control virus infected hemisphere (FIG. 14B). These combined results suggest that transdifferentiation of reactive astrocytes to neurons exerts beneficial effects on functioning of a damaged neuronal network and represents a novel technique to address the mechanisms by which the loss of GABAergic neurons contributes to cognitive deficits in AD.

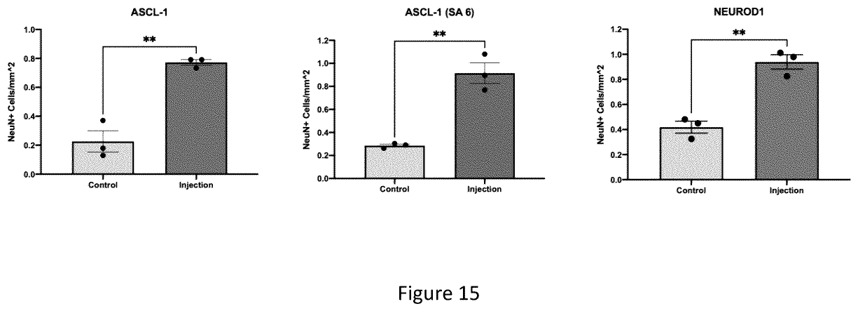

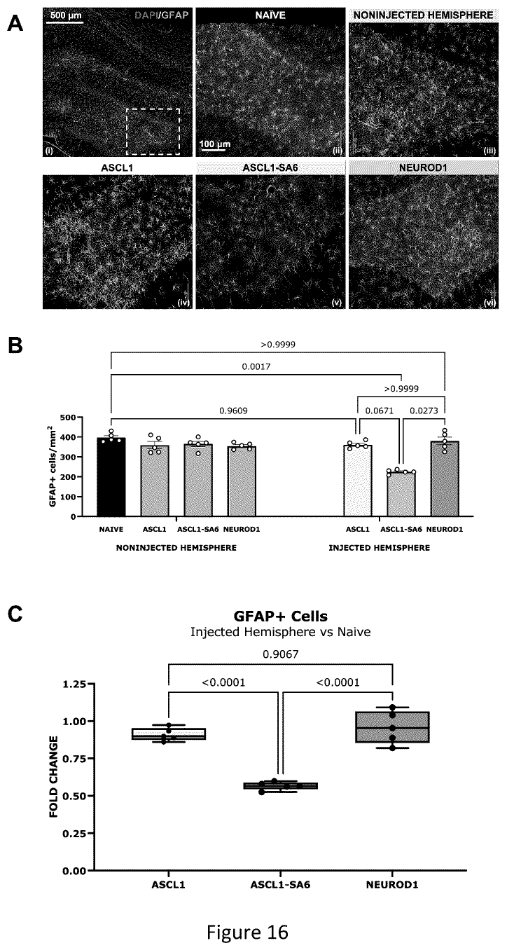

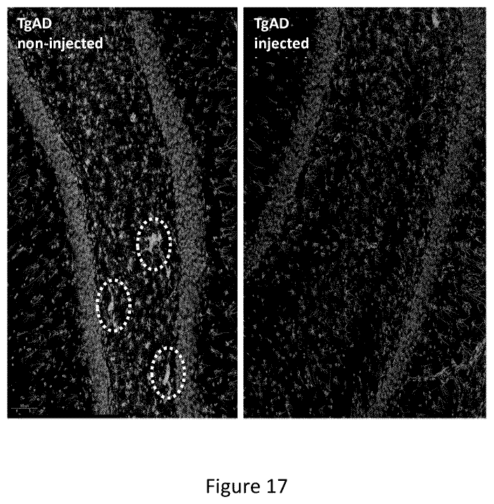

It has been previously shown that both GABAergic and glutamatergic neurons are lost in the TgF344AD rat and also in Alzheimer's disease patients. To illustrate that preferential production of GABAergic versus glutamatergic neurons is beneficial for AD pathology and hippocampal function, the efficacy of the different transcription factors, Ascl1 and Neurod1, was examined. As previously mentioned, Ascl1 preferentially promotes the differentiation of GABAergic and oligodendroglial cells depending on environmental cues whereas Neurod1 preferentially promotes glutamatergic neuronal fate. In development these transcription factors are controlled by the phosphorylation of serine/threonine residues throughout the sequence that results in inactivation of the transcription factors at precise developmental stages. To prevent the premature inactivation of Ascl1, alanine residues were incorporated in all 6 of the serine phosphorylation sites normally used to turn off Ascl1 activity (S to A point mutations). All three transcription factors effectively promoted the transdifferentiation of astrocytes to neurons, as measured by counting the number of NeuN+ cells within the hilus of the hippocampus, in the hemisphere infected with virus in comparison to the non-infected side (FIG. 15). However, Ascl1-SA6 mutant was 20% more effective than the parent Ascl1 with a 4.5 and 4.0 fold increase in NeuN+ neurons while Neurod1 was 2.5 fold effective at transdifferentiation. The increase in neurons was accompanied by a decrease in reactive astrocytes in the infected hemisphere (FIG. 16, 17). In agreement with the differential effects of the transcription factors on NeuN+ neurons, Ascl1-SA6 expression resulted in ˜50% reduction in astrocytes while Ascl1 reduced astrocytes by 15% and Neurod1 only reduced astrocytes by 5%. The loss of reactive astrocytes appeared to be of a lower fold change than that of the increase of neurons for Ascl1 and Neurod1 induced transdifferentiation, however, reactive astrocytes were decreased. Without wishing to be bound by theory, some of the reactive astrocytes may have transdifferentiated into neurons while others may represent quiescent phenotypes that do not contribute to AD pathology.

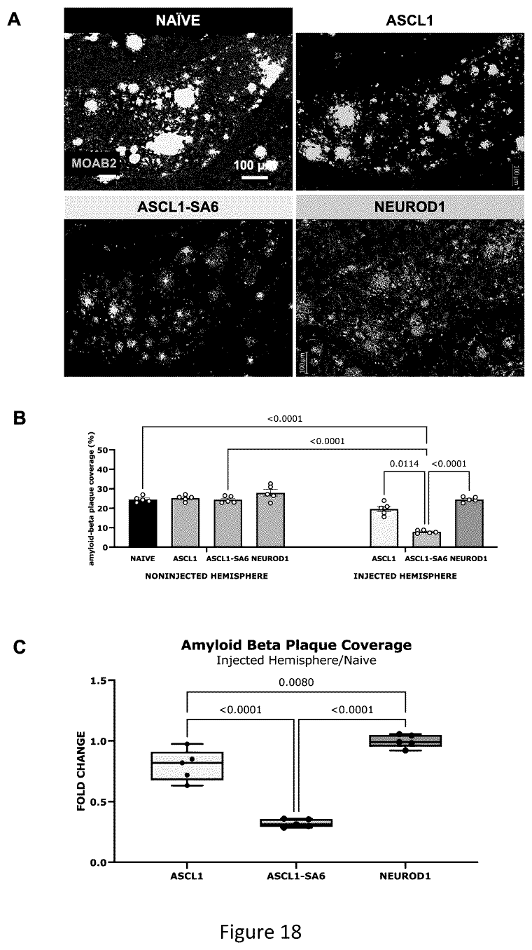

Since the reactive astrocytes have been decreased, it appeared that the toxic environment of the hilus may have altered, which was anticipated to ultimately result in positive downstream effects on AD-related pathology. To investigate this, the number of amyloid plaques and reactive microglia present in the infected hemispheres was counted and compared to uninfected hemispheres. After staining sections of the transfected hemispheres with MOAB2 (an antibody that recognizes the N-terminus of amyloid-beta peptide), it was immediately obvious that there was a decrease in amyloid plaques in the infected hilus in comparison to the uninfected ipsilateral hilus (FIGS. 18A and B). Quantification demonstrated that plaques were reduced by ˜70% after transdifferentiation of astrocytes to neurons by Ascl1 (SA6) while Ascl1 was reduced by ˜20% and Neurod1 was not reduced (FIG. 18C). A reduction in amyloid plaques may be the result of a decrease in production or an increase in degradation. However, as the experimental paradigm was only ˜45 days then the likelihood of a decrease in production resulting in a 50% loss of plaques between hemispheres is unlikely as it has been previously shown that plaques increase by 10% per month after the age of 9-months. In light of this, the reduction in amyloid plaques is likely due to an increase in amyloid degradation.

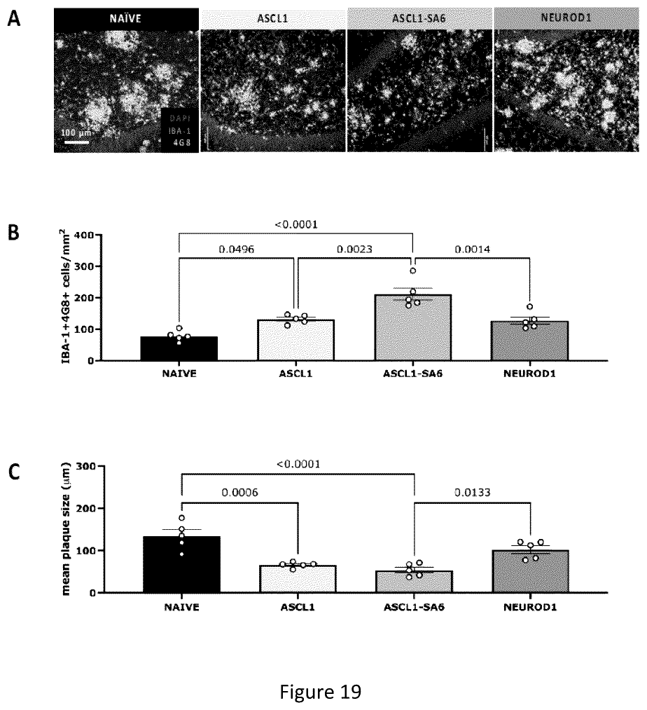

Amyloid is degraded by activated microglial cells surrounding plaques while soluble amyloid-beta peptide is degraded by non-plaque associated microglial cells. Using an anti-4G8 antibody, which is specific for amino acid residues 17-24 of Aβ peptides and an anti-IBA-1 antibody, pathological tissues were used to quantify IBA-1+4G8+ cells (FIG. 19). Although there was an increase of approximately 60% in IBA-1+4G8+ cells following lineage reprogramming with Neurod1, this was not significant from naïve transgenic rats. Rats that received Ascl1 exhibited significant increases in plaque-associated microglia with a fold-change of approximately 1.7×. As with other analyses, however, transdifferentiation with Ascl1-SA6 resulted in the greatest change, with an approximate 2.7× increase in IBA-1+4G8+ cells, in comparison to naïve transgenic rats. This increase in plaque-associated microglia was also significantly greater than both Ascl1 and Neurod1 (p<0.01), although Ascl1 and Neurod1 were not statistically distinct (p>1.00). Furthermore, mean plaque size was evaluated following transient expression of bHLH transcription factors. Although expression of Neurod1 resulted in an approximate 25% decrease in mean plaque size, these changes were not significant from naïve transgenic rats. Rats that received Ascl1, however, exhibited significant decreases in plaque size of ˜50%, whereas Ascl1-SA6, demonstrated a decrease of ˜60% (p<0.01). The decrease in plaque size following Ascl1-SA6 delivery was significantly different from Neurod1 (p=0.01). There was also a trend towards a decrease in mean plaque size with Ascl1 expression, in comparison to Neurod1 (p=0.09).

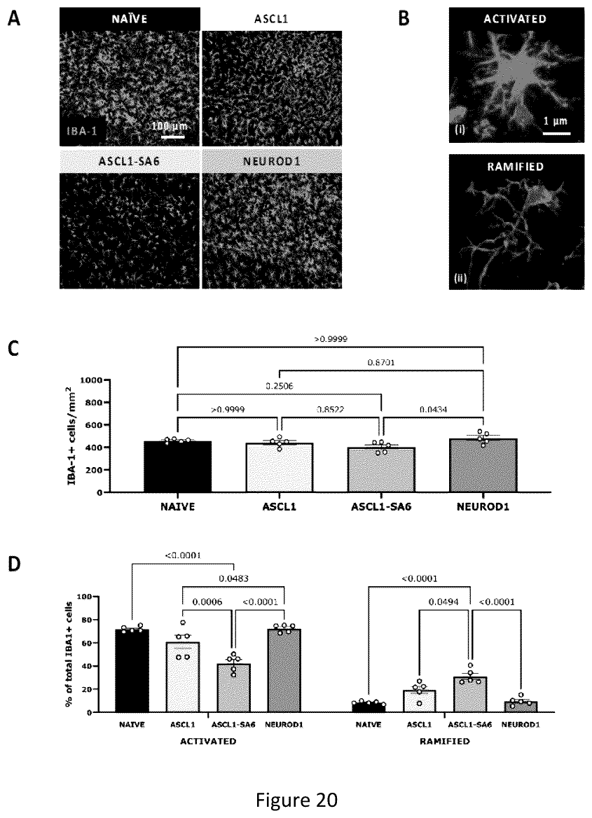

Since microglial demonstrated increased phagocytosis of amyloid plaques, this suggested that microglial phenotypes were reverting from a disease-phenotype to a more homeostatic phenotype. Therefore, immunofluorescence staining for IBA-1+ cells was used to examine microglial morphology (FIG. 20A). Direct lineage reprogramming with proneural bHLH transcription factors showed no significant changes in the total density of IBA-1+ cells in the hilus (FIG. 20B). Transient expression of Neurod1 also resulted in no significant difference from naïve rats (p>1.00), although there was a modest difference in comparison to Ascl1-SA6 (p=0.04; FIG. 20C). Naïve transgenic rats exhibited an activated morphology in approximately 72% of IBA-1+ cells, while only 9% were ramified (FIG. 20C). Following transdifferentiation with Ascl1, there was a trend towards a shift in IBA-1 morphology with 61% activated and 19% ramified (p=0.07 and p=0.09, respectively. However, rats that received Ascl1-SA6 exhibited significant changes in IBA-1 morphology; approximately 42% of cells exhibit activated morphology and 31% were ramified (FIG. 20D). Neurod1 resulted in no significant changes in microglial morphology in comparison to naïve rats (p>1.00). These data suggest that Ascl1-SA6 shift the environment towards homeostasis.

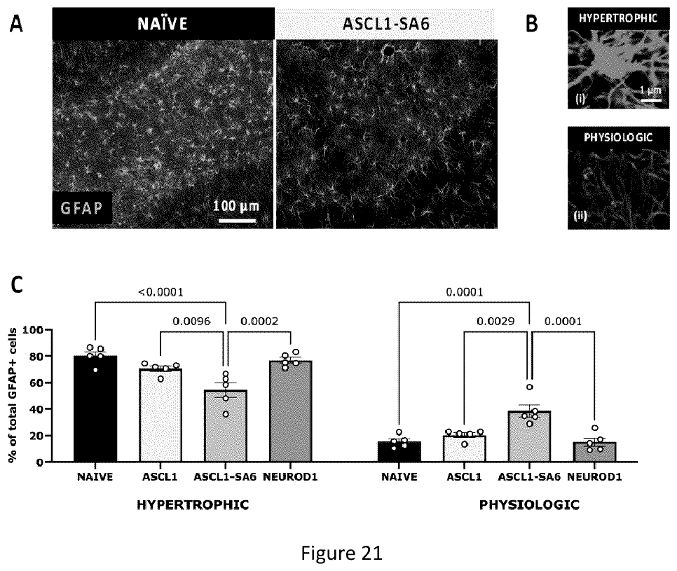

To test whether astrocyte phenotype was shifted towards a homeostasis, GFAP+ve astrocyte morphology was used and astrocytes were categorized as either hypertrophic or physiologic (FIG. 21). In addition to the decrease in total GFAP+ cells observed following Ascl1-SA6 expression, there was a decrease in hypertrophic astrocytes, concurrent with an increase in the physiologic state (FIG. 21C). In naïve, transgenic rats approximately 80% of astrocytes are hypertrophic, while only 10% are physiologic. Following transdifferentiation with Ascl1-SA6, however, there is a significant shift in astrocytic morphology with 54% hypertrophic and 39% physiologic. The abundance of hypertrophic and physiologic astrocytes, in comparison to Ascl1 and Neurod1, is significantly different following viral delivery of Ascl1-SA6 (p<0.01). Expression of Ascl1 led to an approximate 10% decrease in hypertrophic and 5% increase in physiologic GFAP+ cells, however these values were not significantly different from naïve transgenic rats (p=0.27 and p>1.00, respectively). Transient expression of Neurod1, on the other hand, led to no changes in astrocyte morphology with comparable levels of hypertrophic and physiologic GFAP+ cells and p values greater than 1.00 (FIG. 21C).

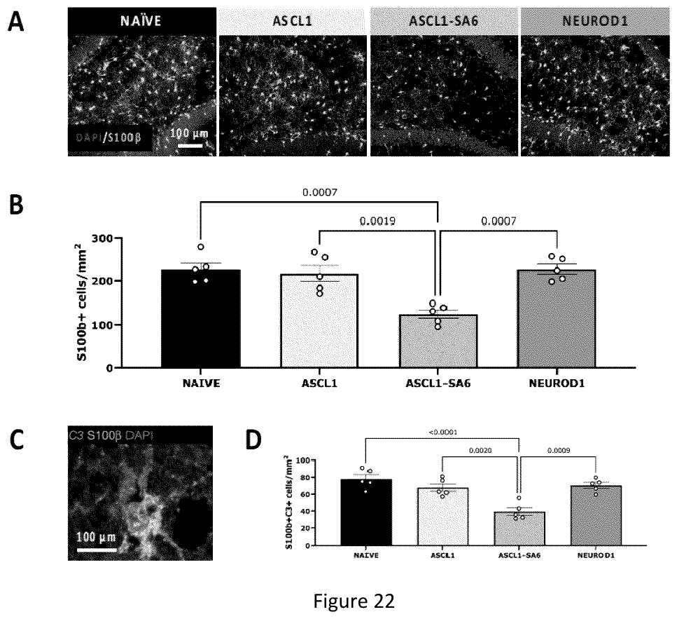

In order to further characterize astrogliosis, immunofluorescence analysis of S1003+ cells was performed at the injection site. In line with the quantitative analysis of GFAP+ cells, as well as astrocytic morphology, viral delivery of Ascl1-SA6 lead to significant decreases in S1003+ cells at the injection site (FIG. 22A,B). In comparison to naïve transgenic rats, rats that received Ascl1-SA6 exhibited an approximate 45% decrease in S1003+ cells. Ascl1 and Neurod1 resulted in minimal differences that were not significant from naïve Tg rats (p>1.00) but distinct from Ascl1-SA6 (p<0.01). In light of studies suggesting quantification of C3 for the characterization of astrogliosis, an immunofluorescence analysis to quantify S1003+C3+ cells in the injection site was performed (FIG. 22C,D). Although Ascl1 and Neurod1 led to an approximate 10% reduction in S1003+C3+ cells in the hippocampal hilus, these differences were not significant (p>0.73; FIG. 22D). Expression of Ascl1-SA6 resulted in ˜50% decrease in S1003+C3+ cells in comparison to untreated Tg rats (p<0.01) that was also significantly different from Ascl1 and Neurod1 (p<0.01). Therefore, Ascl1-SA6 resulted in decreases in four factors indicative of astrogliosis-reductions in GFAP+ cells, S1003+ cells, C3+ astrocytes and changes in astrocytic morphology.

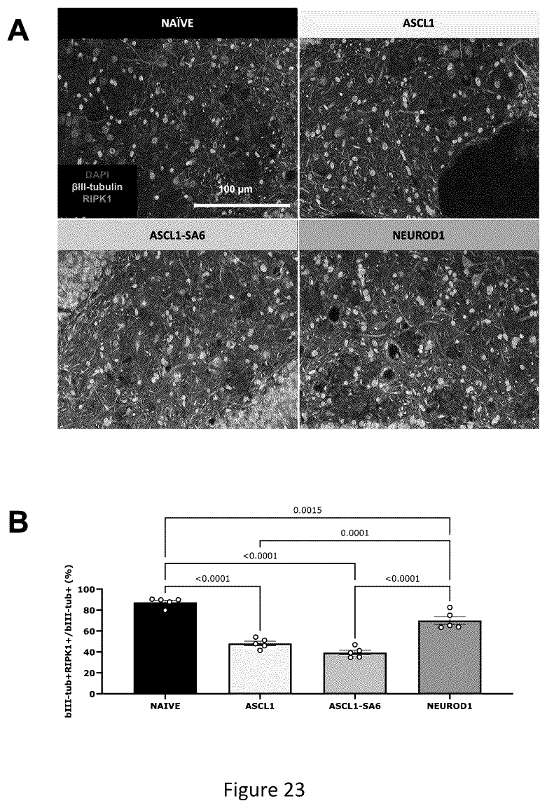

While the best correlate of cognitive decline in AD patients and animal models of the disease is synaptic dysfunction, neuronal loss and neurodegeneration play a role in synapse loss. Previous studies have shown that in CNS diseases, astrocytic and microglial activation results in an upregulation of an inflammatory gene signature. This results in RIPK1 activation in neurons and eventual cell death. In fact, RIPK1 inhibition results in abatement of AD pathology and prevents neuronal cell death in mouse models. It was therefore determined whether direct lineage reprogramming altered the number of necrotic neurons (FIG. 23). In order to quantify necrotic neurons, immunofluorescence staining was used, with anti-βIII-tubulin and anti-RIPK1 antibodies (FIG. 23A). The results revealed that transdifferentiation with Ascl1, Ascl1-SA6 and Neurod1 leads to significant reduction in βIII-tubulin+RIPK1+ cells in the hilus (FIG. 23B). Rats that received Ascl1-SA6 exhibited the greatest decrease in necrotic neurons with over 50% reduction in βIII-tubulin+RIPK1+ cells, compared to their naïve counterparts (p<0.01).

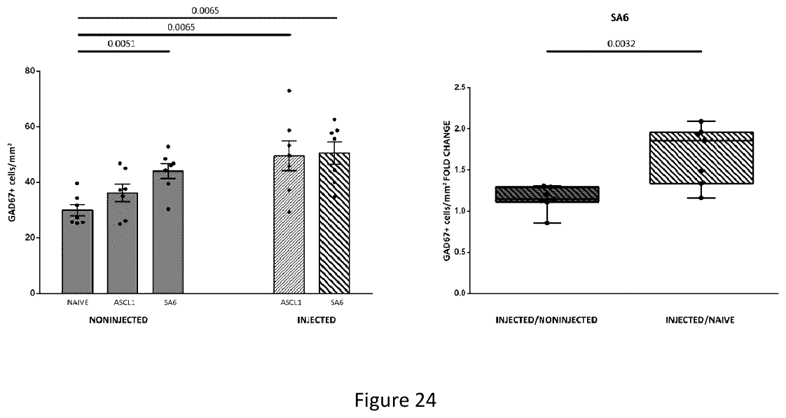

It was then hypothesized that the decrease in necrotic neurons and rebalancing of glial homeostasis would lead to an overall increase in GABAergic neurons not only within the treated hemisphere that but also the contralateral hemisphere. As Neurod1 did not exhibit these effects, analysis was focused on Ascl1 and Ascl1-SA6. Although both Ascl1 and Ascl1-SA6 significantly increased the number of GAD67+ cells in the transcription factor injected hemisphere (FIG. 24A), only Ascl1-SA6 significantly increased the number of GABAergic neurons in the contralateral side in comparison to untreated Tg rats. Comparison of the fold changes between ipsilateral to contralateral versus injected versus untreated hemispheres demonstrates the significant increase in overall GABAergic neurons in Ascl1-SA6 treated rats (FIG. 24B).

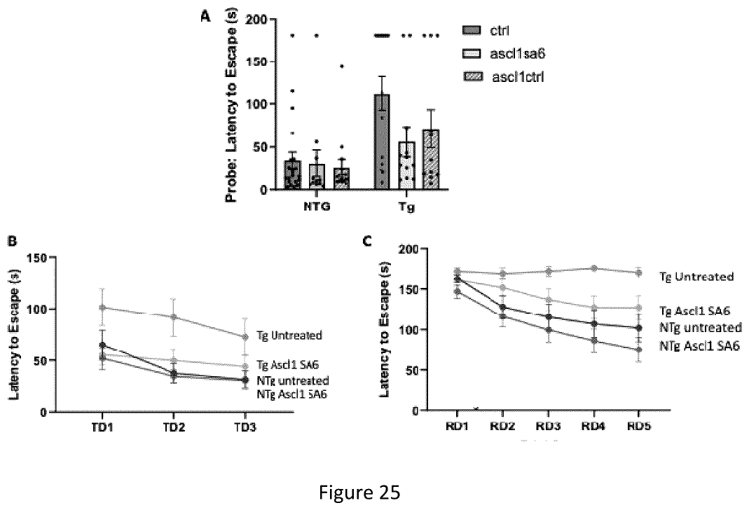

Lastly, to determine whether the direct lineage transdifferentiation of reactive astrocytes to neurons and resultant downstream homeostatic benefits lead to improved cognitive function in the TgF344 AD rats, the Barnes Maze task was performed on Ascl1-SA6 versus Ascl1 transdifferentiated rats in comparison to untreated rats (FIG. 25). Comparison of spatial memory between naïve, Ascl1- and Ascl1-SA6-treated NTg rats showed no significant difference, whereas TgF344 AD rats treated with Ascl1 and, significantly, with Ascl1-SA6 showed improved memory in comparison to untreated TgF344 AD rats (FIG. 25A). Ascl1-SA6 TgF344 AD rats showed a trend to increased memory in comparison to Ascl1 treated rats. To further probe the improved performance of TgF344 AD rats treated with Ascl1-SA6, the learning and reversal phase of the Barnes Maze task was examined. Untreated TgF344 AD rats learned the Barnes maze task significantly slower than TgF344 AD rats treated with Ascl1-SA6 and NTg rats both treated and untreated (FIG. 25B). Importantly, the TgF344 AD rats treated with Ascl1-SA6 were not significantly different from NTg rats both treated and untreated. Examination of executive function or the ability to problem solve was significantly better in TgF344 AD rats treated with Ascl1-SA6 in comparison to untreated TgF344 rats and were not significantly different from treated and untreated NTg rats (FIG. 25C).

### Conclusions

The present study demonstrates that proneural transcription factors, Ascl1, Ascl1-SA6 and Neurod1, effectively transdifferentiate reactive astrocytes into neurons in the TgF344 AD rat model. Ascl1 expression in the hippocampus increased the number of GABAergic neurons and Ascl1 expression increased hippocampal neuronal connectivity, illustrating that the new neurons produced integrate into the circuit and are beneficial. In addition, growth factors that are generated as a result of new born neurons also contribute to the benefit.

The mutant Ascl1-SA6 transcription factor was the most effective factor at transdifferentiation of astrocytes into neurons. Unexpectedly, Ascl1-SA6 reduction in reactive astrocytes, after transdifferentiation, altered the hippocampal environment such that amyloid plaque load clearance was endogenously stimulated. The decrease in amyloid plaques was accompanied by increased microglial association with plaques, which is one mechanism to decrease plaque load within the brain. After transdifferentiation with Ascl1-SA6, the parenchymal environment in the hippocampus was shifted towards a homeostatic phenotype as illustrated by the morphological changes in astrocytes and microglial cells, decrease in inflammatory mediators, S1003 and C3, and reduced necrotic neurons. The overall beneficial outcomes after transdifferentiation with Ascl1-SA6 resulted in improved spatial learning, memory and executive function in treated TgF344 AD rats in comparison to untreated TgF344 rats.

### REFERENCES

1. Querfurth H W, LaFerla F M. Alzheimer's disease. N Engl J Med. 2010 Jan. 28; 362(4):329-344.
2. Terry R D, et al. Physical basis of cognitive alterations in Alzheimer's disease: synapse loss is the major correlate of cognitive impairment. Ann. Neurol. 1991, 30, 572-580.
3. DeKosky S T, Scheff S W. Synapse loss in frontal cortex biopsies in Alzheimer's disease: correlation with cognitive severity. Ann. Neurol. 1990, 27, 457-464.
4. Oddo, S. et al. Triple-Transgenic Model of Alzheimer's Disease with Plaques and Tangles: Intracellular Aβ and Synaptic Dysfunction. Neuron 2003, 39, 409-421.
5. Bittner, T. et al. Multiple Events Lead to Dendritic Spine Loss in Triple Transgenic Alzheimer's Disease Mice. PLoS One 2010, 5, e15477.
6. Ittner, L. M. & Götz, J. Amyloid-β and tau—a toxic pas de deux in Alzheimer's disease. Nat Rev Neurosci 2011, 12, 65-72.
7. Rhein, V. et al. Amyloid-β and tau synergistically impair the oxidative phosphorylation system in triple transgenic Alzheimer's disease mice. Proc Natl Acad Sci 2009, 106, 20057-20062.
8. Cohen, R M et al., A transgenic Alzheimer's rat with plaques, tau pathology, behavioural impairment, oligomeric A [symbol] and frank neuronal loss. J. Neurosci. 2013, 33, 6245-6256.
9. Bott J-B, et al., Spatial Reference Memory is Associated with Modulation of Theta-Gamma Coupling in the Dentate Gyrus. Cereb Cortex. 2016, 26, 3744-3753.
10. HuxterJ, Burgess N, O'Keefe J. Independent rate and temporal coding in hippocampal pyramidal cells. Nature. 2003, 425, 828-832.
11. Cardin J A, et al., Driving fast-spiking cells induces gamma rhythm and controls sensory responses. Nature. 2009, 459, 663-667.
12. Onslow ACE, Bogacz R, Jones M W. Quantifying phase-amplitude coupling in neuronal network oscillations. Prog Biophys Mol Biol. 2011 105, 49-57.
13. Canolty R T, et al., High gamma power is phase-locked to theta oscillations in human neocortex. Science. 2006, 313, 1626-1628.
14. Lee H, et al., Phase locking of single neuron activity to theta oscillations during working memory in monkey extrastriate visual cortex. Neuron. 2005, 45, 147-156.
15. Sohal V S, et al., Parvalbumin neurons and gamma rhythms enhance cortical circuit performance. Nature. 2009, 459, 698-702.
16. Liguz-Leczinar M, Urban-Clecko J, Kossut M. Somatostatin and somatostatin containing neurons in shaping neuronal activity and plasticity. Frontiers in Neural Circuits 2016, 10, article 48, 1-15.
17. Stefanelli T., et al., Hippocampal somatostatin interneurons control the size of the neuronal memory ensembles. Neuron 2016, 89, 1074-1085.
18. Ramos B, et al., Early neuropathology of somatostatin/NPY GABAergic cells in the hippocampus of a PS1xAPP transgenic model of Alzheimer's disease. Neurobiol Aging 2006, 27, 1658-1672.
19. Krantic S, Isorce N, Mechawar N, Davoli M A, Vignault, E., Albuguergue, M., Chabot J-G, Chauvin J-P, Aubert I. McLaurin J, Quirion R, Hippocampal GABAergic neurons are susceptible to amyloid beta toxicity in vitro and are decreased in number in the Alzheimer's disease TgCRND8 mouse model. J. Alz. Disease 2012, 29, 293-308.
20. Ma K, McLaurin J. [symbol] -Melanocyte stimulating hormone prevents GABAergic cell loss to improve cognition in Alzheimer's disease. J. Neuroscience 2014, 34, 6736-6745.
21. Li Y, uet al., Implications of GABAergic neurotransmission in Alzheimer's disease. Frontiers in Aging Neuroscience 2016, 8, article 31, 1-12.
22. Tong L M, et al., Enhancing GABA signaling during middle adulthood prevents age-dependent GABAergic interneuron decline and learning and memory deficits in ApoE4 mice. J. Neuroscience 2016, 36, 2316-2322.
23. Schmid L C, et al., Dysfunction of somatostatin-positive interneurons associated with memory deficits in an Alzheimer's disease model. Neuron 2016, 92, 114-125.
24. Soler H, et al., The GABAergic septohipocampal connection is impaired in a mouse model of tauopathy. Neurobiol Aging 2017, 49, 40-51.
25. Verret L, et al., Inhibitory interneuron deficit links altered network activity and cognitive dysfunction in Alzheimer model. Cell 2012, 149, 708-721.
26. Palop J J, Mucke L. Network abnormalities and interneuron dysfunction in Alzheimer disease. Nat Rev Neurosci 2016, 17, 777-792.
27. Sanchez P E, et al., Levetiracetam suppresses neuronal network dysfunction and reverses synaptic and cognitive deficits in an Alzheimer's disease model. PNAS 2012, 109, E2895-2903.
28. Shettey A K, Bates A. Potential of GABAergic cell therapy for schizophrenia, neuropathic pain and Alzheimer's and Parkinson's disease. Brain Res. 2016, 1638, 74-87.
29. Colasante G, et al., Rapid conversion of fibroblasts into functional forebrain GABAergic interneurons by direct genetic reprogramming. Cell Stem Cell 2015, 17,719-734.
30. Hunt R F, et al., GABA progenitors grafted into the adult epileptic brain control seizures and abnormal behaviour. Nat Neurosci 2013, 16, 692-697.
31. Vierbuchen, T. et al. Direct conversion of fibroblasts to functional neurons by defined factors. Nature 2010, 463, 1035-1041.
32. Pang, Z. P. et al. Induction of human neuronal cells by defined transcription factors. Nature 2011, 476, 220-223.
33. Son, E. Y. et al. Conversion of mouse and human fibroblasts into functional spinal motor neurons. Cell stem cell 2011, 9, 205-218.
34. Pfisterer, U. et al. Direct conversion of human fibroblasts to dopaminergic neurons. Proc Nat Acad Sci USA 2011, 108, 10343-10348.
35. Marro, S. et al. Direct lineage conversion of terminally differentiated hepatocytes to functional neurons. Cell stem cell 2011, 9, 374-382.
36. Chuang, W. et al. Partial Reprogramming of Pluripotent Stem Cell-Derived Cardiomyocytes into Neurons. Scientific reports 2017, 7, 44840.
37. Rivetti di Val Cervo, P. et al. Induction of functional dopamine neurons from human astrocytes in vitro and mouse astrocytes in a Parkinson's disease model. Nature biotechnology 2017, 35, 444-452.
38. Yang, N. et al. Generation of pure GABAergic neurons by transcription factor programming. Nature methods 2017, 14, 621-628.
39. Dennis D J, Wilkinson G, Li S, Dixit R, Adnani L, Balakrishman A, Han S, Kovach C, Gruenig N, Kurrasch D M, Dyck R H, Schuurrmans C. Neurog2 and Ascl1 together regulate a postmitotic derepression circuit to govern laminar fact specification in the murine neocortex. PNAS 2017, 114, E4934-E4943.
40. Guo Z, et al., In vivo direct reprogramming of reactive glial cells into functional neurons after brain injury and in an Alzheimer's disease model. Cell Stem Cell. 2014, 14,188-202.
41. Liu Y, Miao Q, Yuan J, Han S'e, Zhang P, Li S, Rao Z, Zhao W, Ye Q, Geng J, Zhang X, Cheng L. Ascl1 Converts Dorsal Midbrain Astrocytes into Functional Neurons In Vivo. J Neurosci. 2015, 35, 9336-9355.
42. Sirko S, et al., Reactive glia in the injured brain acquire stem cell properties in response to sonic hedgehog. Cell Stem Cell. 2013, 12, 426-439.
43. Sprott, R L & Ramirez, L. Current inbred and hybrid rat and mouse models for gerontological research. ILAR J. 1997, 38:104-109.
44. Beas, B S et al., Distinct manifestations of executive dysfunction in aged rats. Neurbiol Aging 2013, 34: 2164-2174.
45. Pancani, T et al., Effect of high-fat diet on metabolic indices, cognition and neuronal physiology in aging F344 rats. Neurobiol. Aging 2013, 34: 1977-1987.
46. Guidi, M et al., Assessing the emergence and reliability of cognitive decline over the life span in Fisher 344 rats using the spatial water maze. Frontiers in Aging Neurosci 2014, 6:1-11.
47. Gusel'nikova V V, Korzhevskiy D E. NeuN as a neuronal nuclear antigen and neuron differentiation marker. Acta Naturae 2015, 7, 42-47.
48. McGail J A, Frazier C J, Bizon J L. Molecular aspects of age-related decline: the role of GABA signaling. Mol Med 2015, 21, 450-460.
49. Farrant M, Nusser Z. Variations on an inhibitory theme: phasic and tonic inhibition of GABA_{A} receptors. Nat Rev Neurosci 2005, 6, 215-229.
50. Tong X, et al., Ectopic expression of [symbol] 6 and [symbol] GABA_{A} receptor subunits in hilar somatostatin neurons increases tonic inhibition and alters network activity in the dentate gyrus. J. Neurosci 2015, 35, 16142-16158.
51. Buzsáki G, Leung L W, Vanderwolf C H. Cellular bases of hippocampal EEG in the behaving rat. Brain Res. 1983, 287, 139-171.
52. Bragin A, et al., Gamma (40-100 Hz) oscillation in the hippocampus of the behaving rat. J Neurosci. 1995, 15, 47-60.
53. Bazzigaluppi P, Beckett T L, Koletar M M, Lai A Y, Joo I L, Brown M E, Carlen P L, McLaurin J, Stefanovic B. Early stage attenuation of phase amplitude coupling in the hippocampus and medial prefrontal cortex in a transgenic rat model of AD. J Neurochem. 2017 doi: 10.1111/jnc.14136.
54. Christian K M, Song H, Ming G-I. Functions and dysfunctions of adult hippocampal neurogenesis. Ann Rev Neurosci 2014, 37, 243-262.
55. Revett T J, Baker G B, Jhamandas J, Kar S. Glutamate system, amyloid 0 peptides and tau protein: functional interrelationships and relevance to Alzheimer disease pathology. J. Psychiatry Neurosci 2013, 38, 6-23.
56. Berninger B, Guillemot F, Götz M. Directing neurotransmitter identity of neurones derived from expanded adult neural stem cells. Eur J Neurosci. 2007, 25, 2581-2590.
57. Britz O, Mattar P, Nguyen L, Langevin L-M, Zimmer C, Alam S, Guillemot F, Schuurmans C. A role for proneural genes in the maturation of cortical progenitor cells. Cereb Cortex. 2006, 16 Suppl 1:J138-151.
58. Casarosa S, Fode C, Guillemot F. Mash1 regulates neurogenesis in the ventral telencephalon. Development. 1999, 126, 525-534.
59. Li S, Mattar P, Dixit R, Lawn S O, Wilkinson G, Kinch C, Eisenstat D, Kurrasch D M, Chan J A, Schuurmans C. RAS/ERK signaling controls proneural genetic programs in cortical development and gliomagenesis. J Neurosci. 2014, 34, 2169-2190.
60. Masserdotti G, et al., Transcriptional Mechanisms of Proneural Factors and REST in Regulating Neuronal Reprogramming of Astrocytes. Cell Stem Cell. 2015, 17, 74-88.
61. Pang Z P, et al., Induction of human neuronal cells by defined transcription factors. Nature. 2011, 476, 220-223.
62. Treutlein B, et al., Dissecting direct reprogramming from fibroblast to neuron using single-cell RNA-seq. Nature. 2016, 534, 391-395.
63. McLaurin J, Kierstead M E, Brown M E, Hawkes C A, Lambermon M H L, Phinney A L, Darabie A A, Cousins J E, French J E, Lan M F, Chen F, Wong S S N, Mount H T J, Fraser P E, Westaway D, St George-Hyslop P. Cyclohexanehexol inhibitors of Abeta aggregation prevent and reverse Alzheimer phenotype in a mouse model. Nat Med. 2006, 12, 801-808.
64. Fenili D, Brown M, Rappaport R, McLaurin J. Properties of scyllo-inositol as a therapeutic treatment of AD-like pathology. J Mol Med. 2007, 85, 603-611.
65. Murlidharan G, Samluski R J, Asokan A. Biology of adeno-associated viral vectors in the central nervous system. Frontiers in Mol Neurosci 2014, 7, article 76, 1-9.
66. Murlidharan G, et al., Glymphatic fluid transport controls paravascular clearance of AAV vectors from the brain. J. Clin. Invest. Insight 2016, 1, e88034.
67. Womelsdorf T, et al., Dynamic circuit motifs underlying rhythmic gain control, gating and integration. Nature Neurosci 2014, 17, 1031-1039
68. Edwards E, et al., High gamma activity in response to deviant auditory stimuli recorded directly from human cortex. Journal of neurophysiology 2015, 94, 4269-4280.
69. Canolty R T, Knight R T. The functional role of cross-frequency coupling. Trends in cognitive sciences, 2010, 14, 506-515.
70. Lee H, et al., Phase locking of single neuron activity to theta oscillations during working memory in monkey extrastriate visual cortex. Neuron, 2005, 45, 147-156.
71. Chrobak J J, Buzsaki G. Gamma oscillations in the entorhinal cortex of the freely behaving rat. Journal of Neurosci 1998, 18, 388-398.
72. Turner K M, Burne T H J. Comprehensive behavioural analysis of Long Evans and Sprague-Dawley rats reveals differential effects of housing conditions on tests relevant to neuropsychiatric disorders. PLoS ONE. 2014, 9, e93411.
73. Deacon R M J. Burrowing: a sensitive behavioural assay, tested in five species of laboratory rodents. Behav Brain Res. 2009 200, 128-133.
74. Birrell J M, Brown V J. Medial frontal cortex mediates perceptual attentional set shifting in the rat. J Neurosci. 2000, 20, 4320-4324.
75. Barnes C A. Memory deficits associated with senescence: a neurophysiological and behavioral study in the rat. J Comp Physiol Psychol. 1979, 93, 74-104.
76. Hof P R, Bouras C, Object recognition deficit in Alzheimer's disease: possible disconnection of the occipito-temporal component of the visual system. Neurosci. Lett. 1991, 122, 53-56.
77. Viggiano, M., et al., Visual recognition memory in Alzheimer's disease: Repetition-lag effects. Experimental Aging Research 2008, 34, 267-281.
78. Francis, B. M., Kim J., Barakat, M. E., Fraenkl, S., Yucel, Y. H., Peng, S., Michalski, B., Fahnestock, M., McLaurin, J., Mount, H. T. J. Object recognition memory and BDNF expression are reduced in young TgCRND8 mice. Neurobiol. Aging 2012, 33, 555-563.
79. Barker G R I, et al., Recognition memory for object, place, and temporal order: A disconnection analysis of the role of the medial prefrontal cortex and perirhinal cortex. J. Neurosci 2007, 27, 2948-2957.
80. Harrison F E, Hosseini A H, McDonald M P. Endogenous anxiety and stress responses in water maze and Barnes maze spatial memory tasks. Behav Brain Res. 2009 247-251.
81. Cordova C A, et al., Impaired executive function following ischemic stroke in the rat medial prefrontal cortex. Behav Brain Res. 2014, 258, 106-111.
82. Caroni P. Inhibitory microcircuit modules in hippocampal learning. Current Opinion in Neurobiol 2015, 35, 66-73.
83. Yuan M, et al., Somatostatin-positive interneurons in the dentate gyrus of mice provide local- and long-range septal synaptic inhibition. eLife 2017, 6, e21105.
84. Ko J, Choii G, Lim J W. The balancing act of GABAergic synapse organizers. Trends in Molecular Medicine 2015, 21, 256-268.
85. Krueger-Burg D, Papadopoulos T, Brose N. Organizers of inhibitory synapses come of age. Current Opinion in Neurobiology 2017, 45, 66-77.
86. Joo, I L, Lai, A Y, Bazzigaluppi P, Koletar, M M, Dorr A, Brown M E, Thomason L A M, Sled J G, McLaurin J, Stefanovic B. Early neurovascular dysfunction in a transgenic rat model of Alzheimer's disease. Scientific Report, 2017, 7, Article number: 46427.
87. Busche M A, et al., Rescue of long-range circuit dysfunction in Alzheimer's disease models. Nat Neurosci 2016, 18, 1623-1630.

### Example 3: Neuronal Lineage Conversion to Treat Stroke

Stroke is a leading cause of death and disability worldwide. More than ⅔ of stroke survivors have cognitive and/or motor deficits that interfere with daily living. The neurological dysfunction represents a major contributor to the Global Burden of Disease¹⁻⁴. There are no cures and limited treatment options for stroke survivors. Stroke is an ischemic injury that leads to a loss of neurons and glial cells, concomitant with gliosis at the site of injury and in the perilesional parenchyma. Stroke also results in mechanical and cellular alterations, as well as global changes in excitatory-inhibitory balance at the neuronal network level due to the loss, damage, and ectopic proliferation of neural cells.

### Introduction

Stroke can happen at any age but it more prominent in the adult and aging population. Stroke is one of the major contributors to the global burden of disease⁴. There are presently no cures and limited treatment options to protect or repair the brain after an ischemic insult, the most common form of stroke. As a leading cause of disability, with over 15 million stroke sufferers worldwide per year, the estimated global costs (direct and indirect) are $750 billion in 20204 and these costs are expected to double in 2030.

A stroke occurs when blood flow to the brain is interrupted. This can happen through a hemorrhagic stroke (˜15% of stroke cases), which occurs when a blood vessel in the brain ruptures, or through an ischemic stroke, which occurs when blood flow to the brain is occluded. Hemorrhagic strokes lead to increased pressure on the brain due to leaked blood and are associated with increased risk of mortality.⁵ Ischemic strokes are the most prevalent type of stroke, accounting for ˜85% of total cases.⁶ Ischemic stroke results in decreased glucose and oxygen delivery to the brain, resulting in rapid cell neuronal cell death and leading to impaired neural function.

Ischemic stroke results in a loss of cells within minutes following stroke through mechanisms of excitotoxicity, calcium dysregulation, oxidative and nitrosative stress, cortical spreading depolarization, disruption of the blood-brain barrier (BBB), edema, and inflammation.^{7,8} These mechanisms propagate the rapid death of all cells within the central core of the ischemic insult, with a slower and more progressive cell death occurring in the surrounding penumbra region, which can remain viable for hours after the initial insult. The progression of stroke injury occurs in overlapping phases. In the acute phase (lasting up to 4 days) necrotic cell death in the ischemic core is a result of anoxic depolarization and lack of adenosine triphosphate (ATP), causing irreversible damage within minutes of stroke onset. The surrounding peri-infarct begins to undergo apoptotic cell death within days and for weeks post-stroke. Further, the peri-infarct environment and extra-cellular milieu are exposed to pro and anti-inflammatory signals that lead to activation of astrocytes (“reactive” astrocytes), activated microglia and infiltrating immune cells that contribute to the formation of the glial scar.^{9,11} Reactive astrocytes upregulate the intermediate filament protein GFAP (Glial Fibrillary Acidic Protein) and along with proteoglycans and inflammatory cells (e.g., macrophages, microglia), form a dense barrier that surrounds the injured tissue.^{11,12}

Therapeutic interventions to treat stroke have largely focused on blood flow restoration and neuroprotection to prevent cells from dying following the initial insult. These interventions have limited success and those that have been successful are time sensitive, requiring their administration within a short time window following stroke onset. In this regard, tissue plasminogen activator (tPA) is an FDA approved treatment for stroke that breaks down blood clots to restore flow and in turn, prevent further cell loss after the initial insult. However, this approach is limited by the short therapeutic window of efficacy (within 4.5 hours post-stroke), as well as patient candidacy for the treatment, precluding it's availability and usefulness for most stroke patients.¹³ The standard of care for stroke patients is focused on rehabilitation, although the benefits are limited and there is no standardized approach for implementation and evaluation.¹⁴

Stroke is an ideal target for cell-based therapies due to the need for therapeutic interventions to replace lost cells, expand the therapeutic window, and ultimately promote functional recovery. The conversion of resident brain cells to new neurons, to replace those lost to injury, is an attractive approach for brain repair. Further, the non-progressive nature of the stroke insult likely affords cellular reprogramming the greatest potential for a single dose gene therapy. Once the injured brain has been resolved, treatment ensues and neurons are replaced, leading to functional recovery and the opportunity of “regression” is less likely. Further, recent studies have highlighted astrocyte heterogeneity following stroke¹⁵, and have identified subpopulations of “toxic” astrocytes within the glial scar which can contribute to neuronal cell death. Direct cellular reprogramming to remove toxic astrocytes that contribute to neuronal cell death affords an additional benefit to the astrocyte to neuron reprogramming strategy.

Animal models that mimic the human condition are needed to study therapeutic interventions. With the knowledge that stroke results in heterogeneous outcomes, the advantages and disadvantages of each model must be considered along with its respective appropriateness for the type of study (e.g., regenerative versus neuroprotective) and the outcome measures to be used.¹⁶⁻²² For preclinical therapeutic recovery research, a model that results in predictable, reproducible tissue damage and functional deficits is essential.²³

A number of studies to date have induced astrocyte-to-neuron conversion in mouse and non-human primate models of neurological disease and injury using single neural-specific bHLH TFs, including Ascl1 and Neurod1, or a cocktail of TF's with varying degrees of reprogramming efficiency and functional improvement reported³⁰⁻³⁴. Improved functional outcomes were recently reported using the native transcription factor Neurod1, important for neuronal differentiation during development, in a rodent model of ischemia.^{31,32} While these studies highlight the promising potential of gene therapy for neurorepair and functional recovery after stroke, many questions remain in terms of the efficacy of reprogramming and the impact on functional outcomes.

The present study uses a modified version of the proneural gene Ascl1, termed Ascl1-SA6, which has been designed such that it will not be subject to environmental inhibitory controls that would be found in the stroke injured brain. The efficacy of astrocyte to neuronal conversion was examined and compared using the native Ascl1 gene (wild type) and Ascl1-SA6 (mutant) in the stroke injured brain in a subacute model of ischemic stroke. The functional outcomes following cellular reprogramming was compared in two models of focal ischemic stroke using Ascl1 and Ascl1-SA6 under the control of an astrocytic promoter (i.e., GFAP).

### Methods

Stroke model. Stroke is an ischemic injury that leads to a loss of neurons and glial cells, with astrogliosis at the site of injury and in the perilesional parenchyma. We will use the well-established and reproducible endothelin-1 (ET-1) model to induce motor cortical stroke. ET-1 is a vasoconstricting peptide that induces a focal ischemic injury, astrocyte reactivity and persistent motor deficits, making it ideal for recovery studies. Mice received receive a single ET-1 injection (400 μmol, 1 μl; Calbiochem) into the sensory-motor cortex (+0.6 mm anterior, −2.25 mm lateral and 1.0 mm ventral, relative to bregma) or 3× ET-1 injections (400 pmol, 1 μl/injection, +0.6 AP, −2.25 ML, −1.0 DV; +1.6 AP, −2.25 ML, −1.0 DV; and −0.4 AP, −2.25 ML, −1.0 DV from bregma) (triple-ET-1) in the sensory-motor cortex. Experiments were performed in adult (8-12 week old) male and female cohorts of reporter mice (including R26R-YFP (Jackson Labs: B6.129X1-Gt(ROSA)26Sortm1(EYFP)Cos/J) and R26R-tdTomato (Jackson Labs: Gt(ROSA)26Sor^{tm9(CAG-tdTomato)Hze}) Cre-conditional transgenic mice and C57/B16 mice. Briefly, mice were anaesthetized using isoflourane and placed onto a stereotaxic apparatus. The scalp was incised and a small hole was drilled into the skull at the injection locations (above). ET-1 dissolved in sterile PBS was injected using a bevel tip 26-gauge Hamilton syringe at a rate of 0.1 ul/min. The needle was left in place for 10 minutes after completion of the ET-1 injection then slowly withdrawn. Body temperature was maintained at 37° C. using a heating pad and animals recovered under a heat lamp. Ketoprofen (5.0 mg/kg; s.c.) was administered for post-surgery analgesia.

AAV injections. AAV2/5-GFAP-Cre or AAV2/5-GFAP-Ascl1-t2a-Cre or AAV2/5-GFAP-Ascl1-SA6-t2a-Cre (in Cre-conditional transgenic mice) or AAV2/5-Flex::GFP+AAV2/5-GFAP-Ascl1-Cre or AAV2/5-Flex::GFP+AAV2/5-GFAP-Ascl1-SA6-Cre or empty vector (AAV2/5-Flex::GFP+AAV2/5-GFAP-Cre) in C57/B16 mice were injected into the ipsilesional cortex 7 days post-stroke or sham injury. 1.0×10¹²/ml in a 1 μL total volume at 0.1 μl/min using a 5 μl Hamilton syringe with 26-gauge needle at each the following coordinates: [+0.6 AP, +2.25 ML, −1.0 DV]; [+1.6 AP, +2.25 ML, −1.0 DV]; and [−0.4 AP, +2.25 ML, −1.0 DV] mm from bregma, representing regions encompassing the anterior-to-posterior extent of the stroke lesion. The needle was left in place for 10 minutes after each AAV injection to prevent backflow and then slowly withdrawn. Body temperature was maintained at 37° C. using a heating pad, and mice recovered under a heat lamp. Ketoprofen (5.0 mg/kg; s.c.) is administered for post-surgery analgesia. Groups included stroke+AAV (Cre only, Ascl1, Ascl1-SA6) and Sham (uninjured mice)+AAV (Cre), depending on the experiment. All mice were tested on a battery of behaviour tasks prior to stroke (baseline) and on post-stroke day 3 or 4 (PSD3/4) (to ensure motor impairments were observed following stroke). Stroke injured mice that show a functional impairment on were used for behavioural assessments in order to confirm that the therapeutic intervention was assessing behavioural recovery. Mice underwent behaviour testing for up to 4 months post-stroke, depending on the task as detailed below.

AAV constructs. All AAV constructs were generated and packaged by VectorBuilder™ Inc.

Tissue Processing and Analyses. Mice were anesthetized with an overdose of Avertin (250 mg/kg; i.p.), and perfused transcardially with saline followed by 4% paraformaldehyde (PFA). Brains were removed, post-fixed for 4 hours with 4% PFA then placed in 30% sucrose to cryopreserve before cryosectioning (20 μm) and mounting on superfrost slides. Sections were blocked with 10% normal goat serum (NGS) and 0.3% triton in PBS and labeled with primary antibodies (as per the table below) in PBS overnight at 4° C., followed by incubation with secondary antibodies and DAPI in PBS for 1 h at room temperature. Sections were analyzed for overlapping expression of markers in 5-9 images from ≥3 coronal sections per mouse viewed at 20× magnification, located within the region of +1.6 and −1.0 AP from bregma.

Behaviour assessments. A battery of behavioural/functional tests were utilized that are well-established in the mouse models used in this study. These include: (1) BIOSEP Grip Strength Test which provides a readout of the maximal peak force of paw grip; (2) horizontal ladder task and (3) foot fault task which are used to evaluate skilled coordination; and (4) digital gait analysis using the CatWalk™ to measure differences in various parameters during spontaneous gait.

Foot Fault Analysis: Mice were placed onto a metal grid (1 cm spacing) that was suspended 12 inches above a table surface. Animals were allowed to walk around the grid for 3 mins and were recorded from below. The number of steps and paw slips made with the contralesional forepaw and hindpaw were analyzed (and considered separately), and the percent slips was calculated as #slips/#steps. The test was performed prior to stroke (baseline), at post-stroke day 4 (PSD4; after stroke and before reprogramming) and PSD28 (21 days post-AAV). The following inclusion criteria were applied: (i) demonstrated deficit following stroke (defined as 30% more slips compared to baseline performance) and (ii) >50 steps taken during the test.

Gait Analysis: Gait analysis was performed using the automated Noldus CatWalk XT system (Noldus, The Netherlands). Mice were allowed to traverse a glass walkway illuminated with a light, and a number of gait parameters were measured. A minimum of 3 successful trials was required wherein the mouse took a direct path and speed did not vary more than 60% during the passage. Footprints were recorded using a high-speed video camera and analyzed using CatWalk XT 8.1 software across a range of variables surrounding paw placement and limb coordination while walking. Parameters that were significantly different between groups at baseline, or were not significantly impaired following stroke, were excluded from analysis. Gait analyses was performed on PSD28 and parameters compared between treatment groups.

Ladder Task: The horizontal ladder test consisted of a raised ladder with unevenly spaced rungs with a goal box at the end of the apparatus. Mice were acclimated in the goal box for 2 minutes and then prompted to across the ladder 3 consecutive times per trial day. Mice were recorded during the trials and the videos were examined for the number of contralateral forepaw steps and slips to calculate percent slippage of the injured forepaw. Mice were tested prior to stroke (baseline) and at PSD3/4 (prior to AAV) and at PSD 28. Mice that did not demonstrate an increase in % slippage at PSD3/4 were excluded from further analyses.

Grip Strength: A grip strength meter (Bioseb™) was used to assess forelimb neuromuscular function. Mice were held by the tail and allowed to grab the metal pull bar with their forepaws. The mouse was gently pulled away in a horizontal plane along the axis of the force sensor until the bar is released and the value of the sensor is recorded. Mice perform the grip test 3 times in one trial and the average value is recorded for the trial days (baseline, PSD3/4, PSD14 and PSD28/29). Mice that received triple ET-1 stroke were assessed in the task on PSD28, PSD59, PSD89, and PSD120 to examine long term deficits and recovery. Mice that had >30% change in grip strength (force exerted in grams) relative to baseline on PSD3/4, were included in the analyses.

Data acquisition and statistical analyses: All experiments were conducted with an experiments blind to the injury/treatment condition. Statistical analyses were conducted using Prism™ (v.9, GraphPad™). One-factor analysis of variance (ANOVAs) was used to compare 3 or more groups. Two-way repeated measures ANOVA was used for behavioural data analysis. Differences were considered significant at p<0.05. Values are presented as means±SEM.

TABLE 2
Summary of reagents used for immunohistochemistry

| | Dilution | Company | Catalogue Number |
|---|---|---|---|
| Primary Antibodies | | | |
| Ch anti GFP | 1:200 | Aves | GFP-1020 |
| Rb anti NeuN | 1:200 | Millipore | ABN78 |
| Secondary Antibodies | | | |
| Anti rb Alexa 568 ™ | 1:400 | Life Tech | A11036 |
| Anti ch Alexa 488 ™ | 1:400 | Life Tech | A11039 |
| Nuclear Stains | | | |
| DAPI | 50 ng/μl | Life Tech | D3571 |

### Results

Mutating the Ascl1 serine phosphoacceptor sites leads to enhanced astrocyte to neuron conversion in the injured cortex.

Previous studies have shown that the proneural gene Ascl1 (wild type) is able to convert astrocytes to neurons in the cerebral cortex of control (uninjured) adult mice following intracranial injections into the sensory-motor cortex. Further, the mutant form of Ascl1 (engineered with six serine (S) nucleotides adjacent to prolines being replaced with alanines (A), termed Ascl1-SA6), which is designed to be insensitive to the inhibitory cues in the microenvironment, is more efficient at the conversion in the uninjured cortex. The Ascl1 and Ascl1-SA6 were cloned into an AAV2/5 vector and under the control of the GFAP promoter to target astrocytes and also carried a t2A-Cre cassette, which served as the control vector in the absence of Ascl1 or Ascl1-SA6. The first question asked was whether Ascl1-SA6 was more efficacious at astrocyte to neuron reprogramming in the stroke injured cortical milieu.

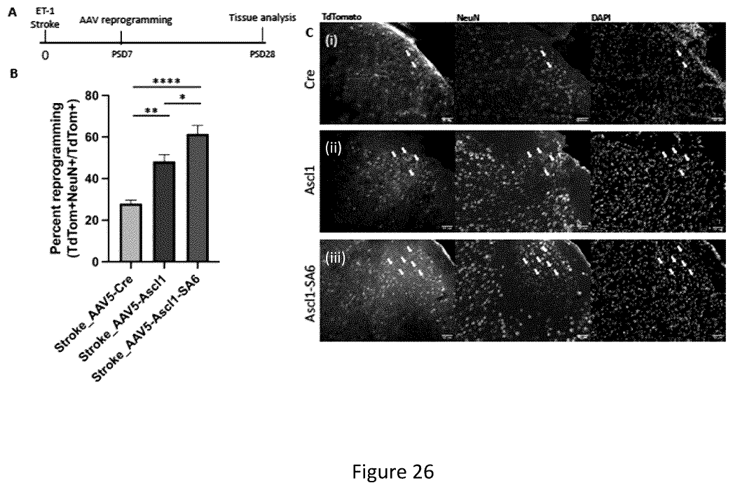

To ask this question, AAV vectors (AAV-Ascl1, AAV-Ascl1SA6 or AAV-Cre) were injected intracranially into adult cre-reporter mice (Rosa-YFP or Rosa-TdTom) that received a sensory-motor cortical stroke, to permit fate tracking of the AAV-transduced astrocytes. Mice received an ET-1 injection to induce a focal, ischemic stroke on day 0 and AAV injections into the lesioned cortex on day 7, which represents the subacute phase post-stroke. The Et-1 stroke results in neuronal cell death and glial scar formation with surrounding the lesion site (Shen X-Y et al, 2021; Livingston et al., 2020). At post-stroke day 28 (PSD28), three weeks after AAV delivery, the brains were removed, sectioned and stained for mature neurons (NeuN+). The total numbers of tdTomato+ (AAV transduced GFAP expressing cells) and tdTomato+/NeuN+(reprogrammed neurons) is shown in FIG. 26. As predicted, Ascl1 resulted in a significant increase in the percentage reprogrammed neurons compared to Cre controls (48.32±3.21 vs. 28.01±1.71% TdTomato+NeuN+/TdTomato+ cells, Ascl1 vs. Cre, respectively) and the neuronal conversion was significantly increased in Ascl1-SA6 mice (61.52±4.03% TdTomato+NeuN+/TdTomato+ cells). Hence, the mutated Ascl1-SA6 gene leads to enhanced neuronal reprogramming compared to native Ascl1.

### Overexpression of Ascl1 and Ascl1SA6 Lead to Improved Functional Outcomes Following Stroke Injury

Mice that receive an ET-1 sensory-motor cortical stroke have functional impairments that can be measured using a number of behavioural tasks. Behavioural tasks were employed that assessed motor function including (1) skilled walking (foot fault and horizontal ladder task); (2) gait analysis (fine motor coordination when walking) and (3) grip strength to assess neuromuscular strength.

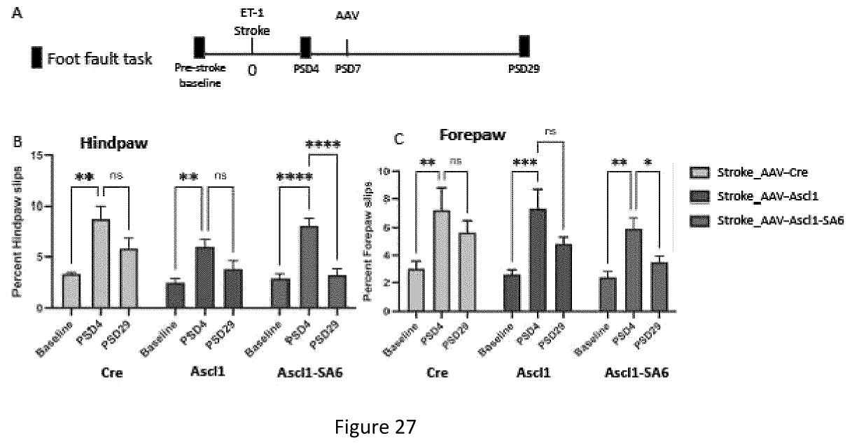

The foot fault task was performed in mice that received a single ET-1 stroke and AAV injections (AAV-Ascl1, AAV-Ascl1-SA6 or AAV-Cre controls) on day 7 post-stroke. Contralesional steps and slips (i.e., on the impaired side of the body that is opposite to the side of the stroke) were recorded for the forepaw and hindpaw. As shown in FIG. 27, all mice that received stroke were significantly impaired at PSD3/4, with increased % slippage of the hindpaw (A) and forepaw (B). By PSD29, mice that received AAV-Ascl1-SA6 were significantly improved and performed similar to baseline. These data reveal that 21 days post-reprogramming is sufficient for Ascl1-SA6 overexpression, but not Ascl1 overexpression, to improve motor outcomes following injury.

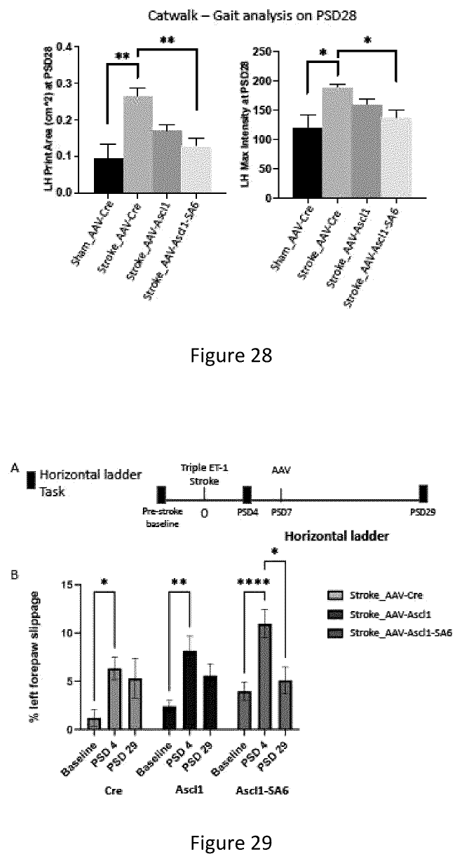

Gait analysis was also performed, which is a sensitive assay to detect subtle motor deficits. In support of the foot fault data demonstrating impaired motor function on PSD28, stroke injured mice that received AAV-Cre were significantly impaired in contralesional (left) hindpaw print area and weight distribution (max intensity) compared to sham mice (uninjured mice that received AAV-Cre) on PSD28 (FIG. 28). Notably, similar to what was observed in the foot fault task, stroke injured mice that received AAV-Ascl1 and AAV-Ascl1-SA6 had improved gait performance, but only the AAV-Ascl1-SA6 treated mice performed significantly better compared to stroke injured controls at PSD28.

### Improved Functional Outcomes are Observed in a More Severe Model of ET-1 Stroke Following Ascl1-SA6 Treatment.

Induction of stroke using a single injection of ET-1 into the motor cortex is a highly reproducible model of stroke that leads to motor impairments. To determine whether behavioural improvement would also be observed in a more severe model of motor cortex stroke hence a triple injection of endothelin-1 (ET-1) was performed^{32,35}, followed by AAV-treatment and behavioural assessment.

Cohorts of C57BL/6 mice received stroke induced with triple ET-1 injections into the motor cortex. On day 7 post-stroke, Ascl1 (AAV5-Flex::GFP+AAV5-GFAP-Ascl1-Cre), or Ascl1-SA6 (AAV5-Flex::GFP+AAV5-GFAP-Ascl1-SA6-Cre) or empty vector (AAV5-Flex::GFP+AAV5-GFAP-Cre) was delivered to the ET-1 stroke injection sites. We performed the horizontal ladder task to examine functional outcomes in the different treatment groups. As shown in FIG. 29, mice that received stroke and Ascl1-SA6 treatment were significantly improved (less forepaw slippage) at PSD29 and not impaired compared to their own baseline performance. Hence, in a more severe model of cortical stroke, Ascl1-SA6 treatment improves functional outcomes.

### Grip Strength is Improved Following Ectopic Expression of Ascl1 and Ascl1-SA6.

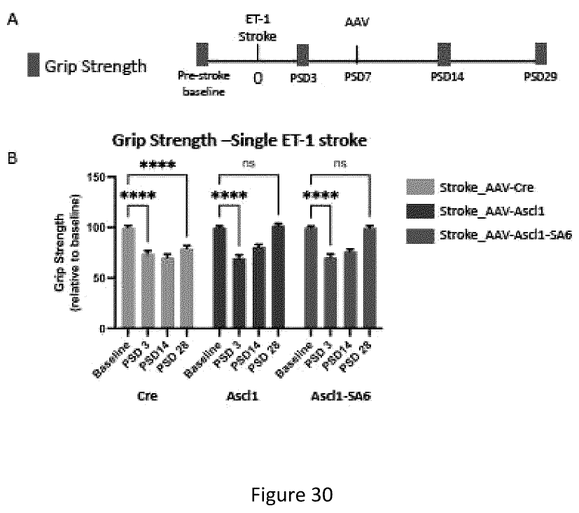

A grip strength analysis was performed in cohorts of mice that received either a single ET-1 stroke or a triple ET-1 stroke. Mice that received a single ET-1 stroke were significantly impaired on PSD3 and AAV-Cre (control) treated mice were still impaired on PSD28. However, AAV-Ascl1 and AAV-Ascl1-SA6 treated mice improved back to baseline levels by PSD28, revealing that the ectopic expression of the native or mutated Ascl1 gene was sufficient to promote functional recovery in a less severe model of stroke (FIG. 30).

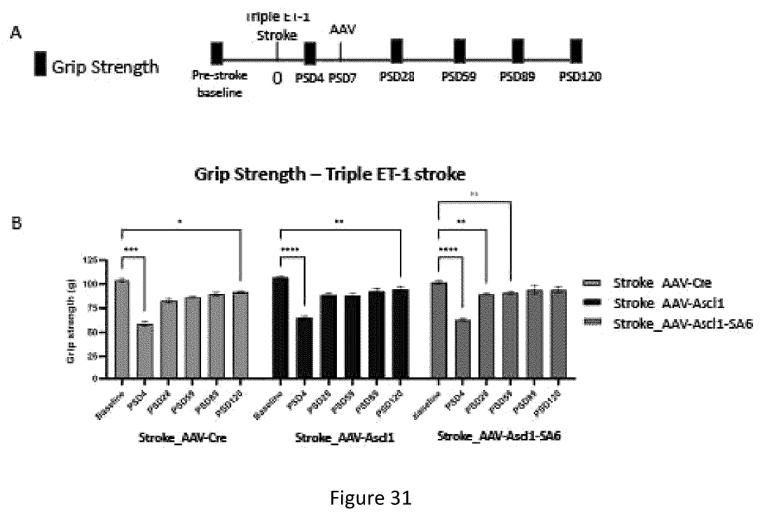

To determine whether the wild type and mutated Ascl1 genes were equally efficient at promoting recovery in a more severe model of stroke (triple ET-1) grip strength was examined for an extended period (up to 4 months) to capture any potential difference in the rate of recovery. As shown in FIG. 31, AAV-Cre (control) treated mice remained significantly impaired relative to their baseline performance out to PSD120 following triple ET-1 stroke. Similar to single ET-1 stroke injured mice, ectopic expression of either AAV-Ascl1 and AAV-Ascl1-SA6 was sufficient to recover performance in the grip strength task to baseline, which occurred by PSD120 in AAV-Ascl1 treated mice. Interestingly, the AAV-Ascl1-SA6 treated mice were recovered to baseline performance by PSD59, indicating that the mutated Ascl1 gene is more efficient at improving functional outcomes than the wild-type Ascl1.

### Conclusion

Taken together, the findings in this Example confirm that use of the modified version of Ascl1, Ascl1-SA6, was more efficient than Ascl1 at inducing astrocyte to neuron conversion following stroke. This increased efficiency was correlated with improved functional outcomes in the skilled motor tasks (foot fault and ladder task); gait and grip strength. Ectopic expression of Ascl1-SA6 was more efficient than Ascl1 at improving functional outcomes in mice that received single ET-1 or triple ET-1 lesions. Hence, Ascl1-SA6 mediated neuronal lineage conversion of cortical astrocytes is a useful strategy to treat the injured brain.

### REFERENCES

1. Global, regional, and national burden of neurological disorders, 1990-2016: a systematic analysis for the Global Burden of Disease Study 2016. Lancet Neurol. 18: 459-480, PM:30879893.
2. Anderson C S, Carter K N, Brownlee W J, Hackett M L, Broad J B, Bonita R (2004) Very long-term outcome after stroke in Auckland, New Zealand. Stroke 35: 1920-1924, PM:15178818.
3. Feigin V L, Forouzanfar M H, Krishnamurthi R, Mensah G A, Connor M, Bennett D A, Moran A E, Sacco R L, Anderson L, Truelsen T, O'Donnell M, Venketasubramanian N, Barker-Collo S, Lawes C M, Wang W, Shinohara Y, Witt E, Ezzati M, Naghavi M, Murray C (2014) Global and regional burden of stroke during 1990-2010: findings from the Global Burden of Disease Study 2010. Lancet 383: 245-254, PM:24449944.
4. Feigin V L et al., (2020) The global burden of neurological disorders: translating evidence into policy. Lancet Neurol., 19:255-265.
5. Andersen, K. K., Olsen, T. S., Dehlendorff, C., and Kammersgaard, L. P. (2009). Hemorrhagic and Ischemic Strokes Compared: Stroke Severity, Mortality, and Risk Factors. Stroke 40, 2068-2072.
6. Mozaffarian, D., Benjamin, E. J., Go, A. S., Arnett, D. K., Blaha, M. J., Cushman, M., de Ferranti, S., Despres, J.-P., Fullerton, H. J., Howard, V. J., et al. (2015). Heart Disease and Stroke Statistics—2015 Update: A Report From the American Heart Association. Circulation 131, e29-e322
7. Iadecola, C., and Anrather, J. (2011). The immunology of stroke: from mechanisms to translation. Nat. Med. 17, 796-808.
8. Rayasam, A., Hsu, M., Hernández, G., Kijak, J., Lindstedt, A., Gerhart, C., Sandor, M., and Fabry, Z. (2017). Contrasting roles of immune cells in tissue injury and repair in stroke: The dark and bright side of immunity in the brain. Neurochem. Int. 107, 104-116.
9. Huang, L., Wu, Z.-B., ZhuGe, Q., Zheng, W., Shao, B., Wang, B., Sun, F., and Jin, K. (2014). Glial Scar Formation Occurs in the Human Brain after Ischemic Stroke. Int. J. Med. Sci. 11, 344-348.
10. Burda, J., and Sofroniew, M. (2014). Reactive Gliosis and the Multicellular Response to CNS Damage and Disease. Neuron 81, 229-248.
11. Silver, J. (2016). The glial scar is more than just astrocytes. Exp. Neurol. 286, 147-149.
12. Sofroniew, M. V. (2009). Molecular dissection of reactive astrogliosis and glial scar formation. Trends Neurosci. 32, 638-647.
13. Diamandis, T. and Borlongan, C. V. (2015) One, Two, Three StepsToward Cell Therapy for Stroke, Stroke 46(2), 588-591
14. Saver, J. L., Fonarow, G. C., Smith, E. E., Reeves, M. J., Navalkele, D., Grotta, J. C., Grau-Sepulveda, M. V., Hernandez, A. F., Peterson, E. D., and Schwamm, L. (2015). Abstract 50: Shape of the TPA Time—Benefit Curve: Insights From The National U S Get With The Guidelines—Stroke Population. Stroke 46, A50-A50.
15. Shen X-Y et al., (2021) Activation and Role of Astrocytes in Ischemic Stroke. Frontiers in Cellular Neuroscience., 15: DOI=10.3389/fncel.2021.755955
16. Carmichael, S. T. (2005). Rodent models of focal stroke: size, mechanism, and purpose. NeuroRx 2, 396-409.
17. Fisher, M., Feuerstein, G., Howells, D. W., Hurn, P. D., Kent, T. A., Savitz, S. I., Lo, E. H., and for the STAIR Group (2009). Update of the Stroke Therapy Academic Industry Roundtable Preclinical Recommendations. Stroke 40, 2244-2250.
18. Fluri, F., Schuhmann, M., and Kleinschnitz, C. (2015). Animal models of ischemic stroke and their application in clinical research. Drug Des. Devel. Ther. 3445.
19. Jolkkonen, J., and Kwakkel, G. (2016). Translational Hurdles in Stroke Recovery Studies. Transl. Stroke Res. 7, 331-342.
20. Savitz, S. I., Chopp, M., Deans, R., Carmichael, S. T., Phinney, D., Wechsler, L., The STEPS Participants, and The STEPS Writing Committee: Sean I. Savitz, Larry Wechsler, Robert Deans, Michael Chopp, Tom Carmichael, and Donald Phinney. Contributors: Jaraslow Aronowski, Martin Bednar, Johannes Boltze, Cesar Borlongan, Tom Carmichael, Thomas Chase, Michael Chopp, D (2011a). Stem Cell Therapy as an Emerging Paradigm for Stroke (STEPS) II. Stroke 42, 825-829.
21. Savitz, S. I., Chopp, M., Deans, R., Carmichael, S. T., Phinney, D., and Wechsler, L. (2011b). Stem cell therapy as an emerging paradigm for stroke (STEPS) II. Stroke 42, 825-829.
22. Sommer, C. J. (2017). Ischemic stroke: experimental models and reality. Acta Neuropathol. (Berl.) 133, 245-261.
23. Winstein, C. J., Stein, J., Arena, R., Bates, B., Cherney, L. R., Cramer, S. C., Deruyter, F., Eng, J. J., Fisher, B., Harvey, R. L., et al. (2016). Guidelines for Adult Stroke Rehabilitation and Recovery: A Guideline for Healthcare Professionals From the American Heart Association/American Stroke Association. Stroke 47, e98-e169.
24. Napoli, E., and Borlongan, C. V. (2016). Recent Advances in Stem Cell-Based Therapeutics for Stroke. Transl. Stroke Res. 7, 452-457.
25. Vonderwalde I, Azimi A, Rolvink G, et al., 2019 Transplantation of directly reprogrammed human neural precursor cells following stroke promotes synaptogenesis
26. Chen, L., Zhang, G., Gu, Y., and Guo, X. (2016). Meta-Analysis and Systematic Review of Neural Stem Cells therapy for experimental ischemia stroke in preclinical studies. Sci. Rep. 6.
27. Wang, Y., Ji, X., Leak, R. K., Chen, F., and Cao, G. (2017). Stem cell therapies in age-related neurodegenerative diseases and stroke. Ageing Res. Rev. 34, 39-50.
28. Janowski, M., Wagner, D.-C., and Boltze, J. (2015). Stem Cell-Based Tissue Replacement After Stroke: Factual Necessity or Notorious Fiction? Stroke 46, 2354-2363.
29. Guo, Z., Zhang, L., Wu, Z., Chen, Y., Wang, F., and Chen, G. (2014). In vivo direct reprogramming of reactive glial cells into functional neurons after brain injury and in an Alzheimer's disease model. Cell Stem Cell 14, 188-202.
30. Faiz, M., Sachewsky, N., Gascon, S., Bang, K. W., Morshead, C. M., and Nagy, A. (2015). Adult Neural Stem Cells from the Subventricular Zone Give Rise to Reactive Astrocytes in the Cortex after Stroke. Cell Stem Cell 17, 624-634.
31. Livingston J, Lee T, Enbar T, Daniele E, Phillips C, Krassikova A, Enbar T, Kortebi I, Bang K W A, Donville B, Ibragimov O, Sachewsky N, Morshead C M, Faiz M. Direct reprogramming of astrocytes to neurons leads to functional recovery after stroke. BioRx, doi: https://doi.org/10.1101/2020.02.02.929091
32. Chen, Y. C., Ma, N. X., Pei, Z. F., Wu, Z., Do-Monte, F. H., Keefe, S., Yellin, E., Chen, M. S., Yin, J. C., Lee, G., Minier-Toribio, A., Hu, Y., Bai, Y. T., Lee, K., Quirk, G. J., and Chen, G. (2020). A NeuroD1 AAV-Based Gene Therapy for Functional Brain Repair after Ischemic Injury through In Vivo Astrocyte-to-Neuron Conversion. Mol Ther. 28(1):217-234.
33. Ge, L. J., Yang, F. H., Li, W., Wang, T., Lin, Y., Feng, J., Chen, N. H., Jiang, M., Wang, J. H., Hu, X. T., and Chen, G. (2020). In vivo Neuroregeneration to Treat Ischemic Stroke Through NeuroD1 AAV-Based Gene Therapy in Adult Non-human Primates. Front Cell Dev Biol 8, 590008.
34. Rivetti di Val Cervo, P. et al. (2017) Induction of functional dopamine neurons from human astrocytes in vitro and mouse astrocytes in a Parkinson's disease model. Nature biotechnology, 35, 444-452.
35. Roome R B, et al., (2014) A reproducible Endothelin-1 model of forelimb motor cortex stroke in the mouse. J Neurosci Methods. 2014 Aug. 15; 233:34-44. doi: 10.1016/j.jneumeth.2014.05.014.

### Example 4: Cerebral Organoids (COs) Culture

### Methods

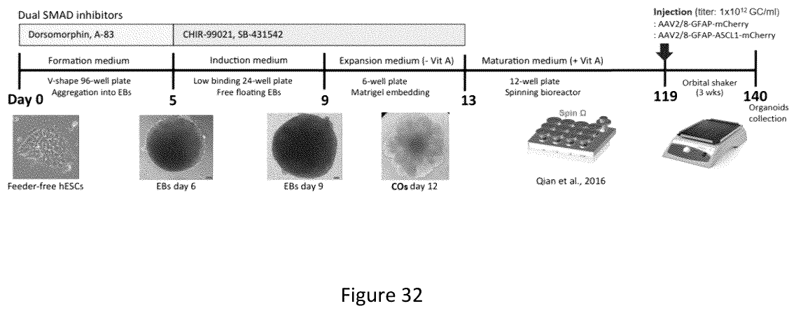

The overall workflow for this study is depicted in FIG. 32.

Feeder-free H1 human embryonic stem cells (hESCs, WiCell) were cultured on Matrigel in TeSR™-E8™ kit for hESC/hiPSC maintenance (StemCell Tech; #05990). hESCs were used to generate COs using media included in the STEMdiff Cerebral Organoid Kit (StemCell Tech; #08570) and STEMdiff Cerebral Organoid Maturation Kit (StemCellTech; #08571), with some modifications. Briefly, hESCs were plated in 96-well V-shaped bottom ultra-low attachment plates at 12,000 cells/well in embryoid body (EB) formation medium with the presence of 50 μM Y-27632 (ROCK inhibitor; StemCell Tech; #72302) until day 2. Dual SMAD inhibitors (2 μM Dorsomorphin; StemCellTech; #72102, and 2 μM A83-01; StemCell Tech; #72022) were added from seeding day until day 5. Newly formed EBs were transferred to low binding 24-well plates containing induction medium. On day 9, EBs with optically translucent edges were embedded in matrigel (Corning; #354277) and deposited into 6-well ultra-low adherent plate with StemCell Tech expansion medium. From day 5 to day 13, media was supplemented with 1 μM CHIR-99021 (StemCell Tech; #72052) and 1 μM SB-431542 (StemCellTech; #72232) to support formation of well-defined, polarized neuroepithelia-like structures. On day 13, embedded EBs exhibiting expanded neuroepithelia as budding surfaces were transferred to a 12-well spinning bioreactor (Spin Omega) containing maturation medium in a 37° C. incubator. COs were fed with maturation medium twice a week. CO's generated by the 2014 Lancaster methodology (Lancaster M A, Knoblich J A. Generation of cerebral organoids from human pluripotent stem cells. Nat Protoc. 2014; 9:2329-2340. doi: 10.1038/nprot.2014.158) were similarly employed in these studies.

Injection of COs with Adeno-Associated Virus (AAV)

On day 90 or 119, individual COs were injected with either AAV2/8-GFAP-mCherry or AAV2/8-GFAP-ASCL1-mCherry at 1×10¹² GC/ml in the maturation medium and transferred to the orbital shaker. Media were changed 48 h after injection and COs were cultured for 3 weeks before the collection.

### Cos Collection and Analysis

COs were washed with PBS once and fixed in 4% paraformaldehyde (PFA) overnight on the rocker at 4° C. On the next day, COs were transferred into 30% sucrose overnight and snap frozen in OCT for cryosectioning. For immunohistochemistry, samples (10 am thickness) were washed with PBS containing 0.1% TritonX™-100 to remove excess OCT and performed antigen retrieval in citrate buffer. Blocking was done with PBS containing 0.1% TritonX-100 and 10% horse serum for 1 h. Primary antibodies (GFAP; Novus biologicals; NB100-53809, mCherry; Millipore; M11217, Doublecortin; Abcam; ab77450) diluted in blocking solution were incubated overnight, and followed by secondary antibodies for 1 h before nuclear staining and mounting.

### Results

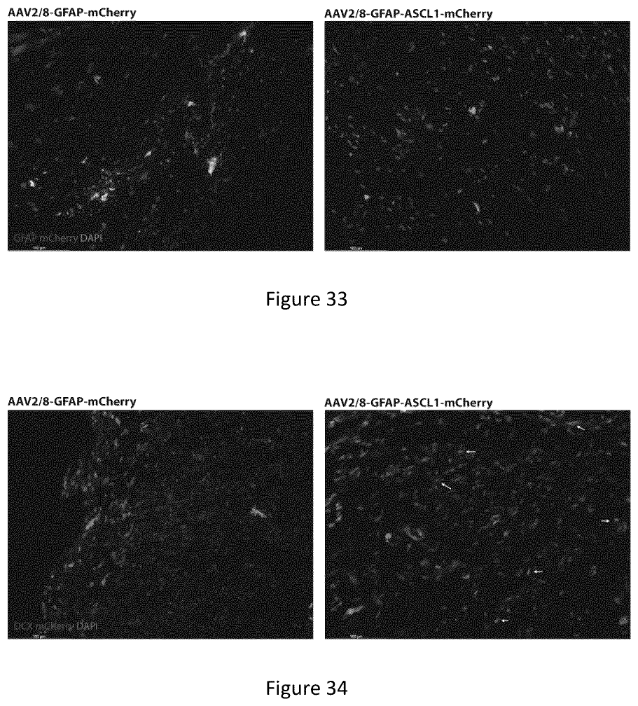

As illustrated in FIG. 33, in COs injected with AAV2/8-GFAP-mcherry (left panel), GFAP expression (astrocyte marker) was co-localized with mCherry expressed under GFAP promoter. On the other hand, the expression of GFAP was lost in COs injected with AAV2/8-GFAP-ASCL1-mCherry (right panel) and the morphology of cells expressing mCherry was changed from astrocyte-like structure into neural-like structure.

As illustrated in FIG. 34, in COs injected with AAV2/8-GFAP-mcherry (left panel), doublecortin (DCX; early neuron marker) expression was not co-localized with mCherry expressed under GFAP promoter. However, the expression of DCX was co-localized with mCherry (bright arrows) in COs injected with AAV2/8-GFAP-ASCL1-mCherry (right panel).

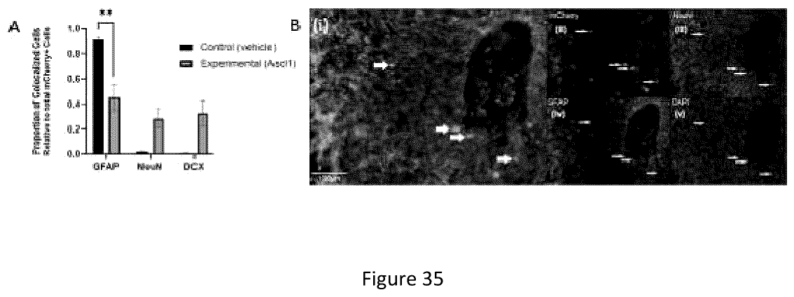

As shown in FIG. 35, in human CO's (90 days) injected with AAV2/8-GFAP-Ascl1-mCherry or AAV2/8-mCherry showed a decreased proportion of astrocytes (GFAP) with a concomitant increase in neuronal markers (NeuN, mature neurons; DCX, immature neurons at 21 days following transduction.

These results demonstrate successful reprogramming of astrocytes to neurons following expression of a mouse bHLH transcription factor in human COs. It should be appreciated based on the herein demonstrated effects of the mutant bHLH transcription factors, it is expected that the reprogramming of astrocytes to neurons in human COs can be improved with the use of a mutant bHLH transcription factor.

### Example 5: Ascl1 Phospho-Site Mutations Enhance Neuronal Conversion of Adult Cortical Astrocytes In Vivo

Neurological diseases are most often associated with the loss or dysfunction of specific neuronal populations. Once lost, neurons are not replaced, except in rare circumstances and in restricted brain niches [1; 2]. The lack of a regenerative response, combined with a paucity of neurotherapeutics, has prompted exploration of various neuronal replacement strategies, including exogenous cell transplants and the stimulation of endogenous neural stem cells. However, these approaches have yet to result in sufficient neuronal integration for long-term functional recovery [3; 4]. Moreover, introducing exogenous human cells, especially fetal stem or progenitor cells, raises ethical concerns, and may be confounded by immune rejection, tumorigenicity, and supply constraints. Identifying an endogenous neuronal repair strategy in which new neurons functionally integrate into existing neural circuitry would be transformative as it would provide new therapeutic strategies to treat neurodegenerative disease.

We have begun to exploit the potential of direct neuronal reprogramming for endogenous neuronal replacement [5; 6]. This feat exploits decades of research into the roles of lineage-specifying basic-helix-loop-helix (bHLH) transcription factors (TF) in driving subtype-specific neurogenesis in the embryonic brain [1; 6]. The proneural bHLH TFs, including Neurog2, Ascl1 and Neurod4, and downstream bHLH genes, such as Neurod1, have emerged as critical architects of neurogenesis in the embryonic brain [7] and are now being exploited to drive neuronal conversion of heterologous cell types [5; 6]. During development, proneural bHLH TFs act at the top of transcriptional cascades, turning on other TFs, such as Neurod1, which function at later developmental stages to control neuronal differentiation. However, bHLH TFs are not active in all cellular contexts and can be inhibited by environmental signals. For example, in the embryonic cortex, Neurog2 is only sufficient (by gain-of-function; [8]) and necessary (by loss-of-function; [9; 10; 11]) to specify a glutamatergic neuronal fate between embryonic day (E) 11.5 to E14.5, despite continued expression at later stages during the neurogenic period, which ends at E17. Similarly, Ascl1, which specifies a GABAergic interneuron fate in the embryonic ventral telencephalon [12], can only induce ectopic GABAergic genes in dorsal telencephalic progenitors at early (E12.5) and not late (E14.5) embryonic stages [9; 10; 11].

The cell context-dependent activities of the proneural genes extends to neuronal reprogramming where there is growing consensus that the conversion of somatic cells to an induced neuron (iNeuron) fate is more efficient when the starter cell is more similar in identity (i.e., neural lineage). Thus, to efficiently convert distantly related fibroblasts to iNeurons, Ascl1 is combined with other TFs, as in the initial “BAM” combination (Brn2/Pou3f2, Ascl1, Myt1l) [13; 14]. In this context, Ascl1 plays a crucial role as a pioneer TF, opening chromatin associated with a specific trivalent signature (H3K4me1, H3K27ac, H3K9me3), which is then accessed by Brn2 and other neurogenic TFs [14]. Other studies have reported that Ascl1 can convert fibroblasts to iNeurons directly, but the maturation of these iNeurons is limited [15]. Similarly, Ascl1 can trigger human pericytes to transdifferentiate into iNeurons, but only when co-expressed with Sox2, which facilitates the transiting of cells through a neural stem/progenitor cell-like stage (i.e., conversion is not direct) [16; 17]. The ability of Ascl1 to induce neural progenitor cells to differentiate into neurons is in keeping with its developmental role [7], and has been recapitulated using progenitor cell lines [18] or pluripotent stem cells in vitro, with Ascl1 acting as a pioneer TF [15; 19; 20].

Astrocytes are common target cells for neuronal conversion as their activated state in neurodegenerative diseases and in injuries such as stroke contributes to disease pathology [5; 6]. Ascl1 can convert cortical astrocytes to iNeurons in vitro, either when misexpressed alone [21; 22; 23; 24] or together with other TFs, for instance, to make dopaminergic iNeurons [25]. Spinal cord astrocytes can also be reprogrammed to iNeurons, but interestingly, a distinct V2 interneuron like-identity is achieved, rather than a cortical phenotype [24]. While there are reports that Ascl1 can convert adult midbrain astrocytes to iNeurons in vivo [26], most studies suggest Ascl1 has low conversion efficacy in the adult cortex and hippocampus in vivo [27; 28]. Thus, understanding how proneural genes such as Ascl1 are regulated (i.e., inhibited), especially in vivo, is important for their efficient use in regenerative medicine. Several approaches have been taken to enhance the neuronal conversion efficacy of Ascl1 and Neurog2. For instance, expressing Ascl1 together with other TFs, as recently shown with a CRISPR-based approach, can enhance neuronal conversion, with resultant iNeurons having therapeutic benefits in a Parkinson's disease model [29]. Similarly, Neurog2 can be combined with other signals, such as Bcl2, to become a potent lineage converter in vivo [30]. The knockdown of REST, a transcriptional repressor of neurogenic genes, also enhances neuronal lineage conversion [31; 32]. Finally, in another ground-breaking study, CRISPR-activation of mitochondrial genes enriched in neurons enhanced Neurog2 and Ascl1 reprogramming efficacy [33]. Identifying and targeting the regulatory events that block bHLH TF activity is thus proving a fruitful strategy to improve neuronal reprogramming.

To address the challenge of lower neuronal conversion efficiency in vivo compared to in vitro [6], the importance of phosphorylation was explored as a critical post-translational modification of Ascl1. It is now well accepted that when neurogenic bHLH TFs are expressed outside of their normal cellular context [8; 34], they are subject to phosphorylation-dependent inhibition that limits their neurogenic activity. Indeed, bHLH TF function is inhibited via phosphorylation by proline-directed serine threonine kinases (e.g. GSK3, ERK1/2, Cdks), which act in a “rheostat-like fashion”; the more serine-proline (SP) or threonine-proline (TP) sites phosphorylated, the less TF binding to DNA and transactivate their target genes to promote neuronal fate specification and differentiation [8; 34; 35; 36]. To keep bHLH TFs active, the present inventors [8; 34] and others [35; 36; 37; 38; 39] have mutated serines (S) and threonines (T) in proline (P)-directed phospho-sites to alanines (A) (i.e., SP/TP to SA/TA mutations). These mutations prevent phosphorylation by inhibitory proline-directed kinases and increase the neurogenic potential of bHLH TFs in the embryonic mouse and frog nervous systems [8; 34; 35; 36; 37; 38; 39; 40].

The goal of this Example was to determine whether a mutated version of Ascl1, termed Ascl1-SA6, is more efficient at inducing neuronal conversion of cortical astrocytes in the adult brain in vivo. We initiated this study using AAV and GFAP promoters [41], a combination that has since been shown to be less astrocyte-specific than initially reported [42]. Nevertheless, by directly comparing Ascl1 to Ascl1-SA6 in all of our studies, we demonstrate that Ascl1-SA6 has a superior capacity to induce neuronal marker expression, and to repress astrocytic genes in the adult cerebral cortex. The enhanced capacity of Ascl1-SA6 to induce neuronal gene expression is in keeping with embryonic studies conducted previously.

### Materials and Methods

Animals and genotyping. Animal procedures were approved by the Sunnybrook Research Institute (21-757) in compliance with the Guidelines of the Canadian Council of Animal Care. In all experiments, we used male C57BL/6 wild-type mice, Rosa-ZsGreen (JAX #007906) and Rosa-tdtomato (JAX #007914) transgenic mice [43], maintained on a C57BL/6 background, and obtained from Jackson Laboratory. Mice were housed under 12 hour light/12 hour dark cycles with free access to food and water. PCR primers and conditions for genotyping were conducted using Jackson Laboratory protocols: Rosa-ZsGreen: wild-type forward: 5′-CTG GCT TCT GAG GAC CG-3′ (SED ID NO:28); wild-type reverse: 5′-AAT CTG TGG GAA GTC TTG TCC-3′ (SEQ ID NO:29); mutant forward: 5′-ACC AGA AGT GGC ACC TGA C-3′ (SEQ ID NO:30); mutant reverse: 5′-CAA ATT TTG TAA TCC AGA GGT TGA-3′ (SEQ ID NO:31). Rosa-tdtomato: mutant reverse: 5′-GGC ATT AAA GCA GCG TAT CC-3′ (SEQ ID NO:32); mutant forward: 5′-CTG TTC CTG TAC GGC ATG G-3′ (SEQ ID NO:33). PCR cycles were as follows: 94° C. 2 min, 10× (94° C. 20 sec, 65° C. 15 sec*−0.5c per cycle decrease, 68° C. 10 sec), 28× (94° C. 15 sec min, 60° C. 15 sec, 72° C. 10 sec), 72° C. 2 min.

AAV cloning and packaging. AAV2/8-GFAPlong-mCherry and AAV2/8-GFAPlong-Ascl1-mCherry were a gift from Leping Cheng and include a 2.2 kb GFAP promoter [26]. The Ascl1 was replaced with Ascl1-SA6 in AAV2/8-GFAPlong-Ascl1-mCherry. In Ascl1-SA6, serines in all six SP sites was mutated to alanines (as in [34]). AAV5-GFAPshort-iCre was previously described [44] and includes a 681 bp (gfaABC(1)D modified GFAP promoter [41]. Cloning of Ascl1-t2a-iCre and Ascl1-SA6-t2a-iCre into AAV5-GFAPshort and replacement of the GFAPshort promoter with the GFAPlong promoter in the AAVs from Leping Cheng [26] was outsourced to Genscript. After cloning, all AAVs were packaged by VectorBuilder Inc. either with AAV capsid 8 or capsid 5. For optogenetic experiments, AAV5-EF1a-double floxed-hChR2(H134R)-EYFP-WPRE-HGFpA (catalog #51502-AAV5) was purchased from Addgene (20298).

Intracranial injection of AAVs. Intracranial injections, 16-week-old male C57BL/6 mice were performed as described in Example 1, using either buprenorphine (0.1 mg/kg; Vetergesic, 02342510] or Tramadol-HCL (20 mg/kg; Chiron, RxN704598) as an analgesic, along with baytril (2.5 mg/kg; Bayer, 02249243), and saline (0.5 ml, Braun, L8001).

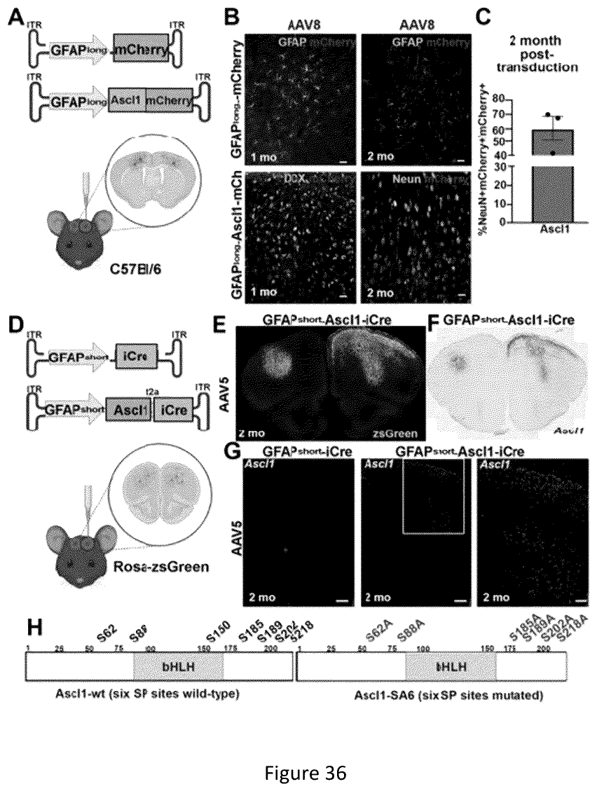

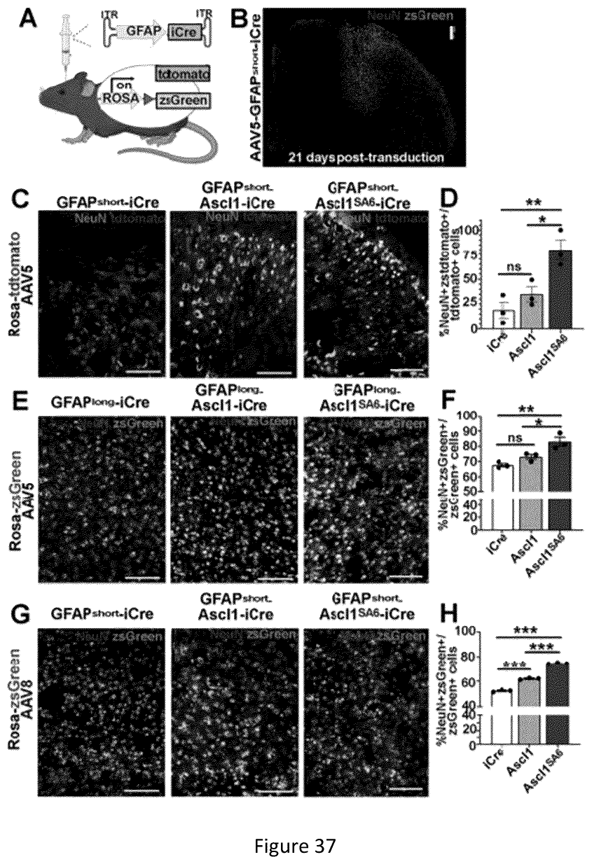

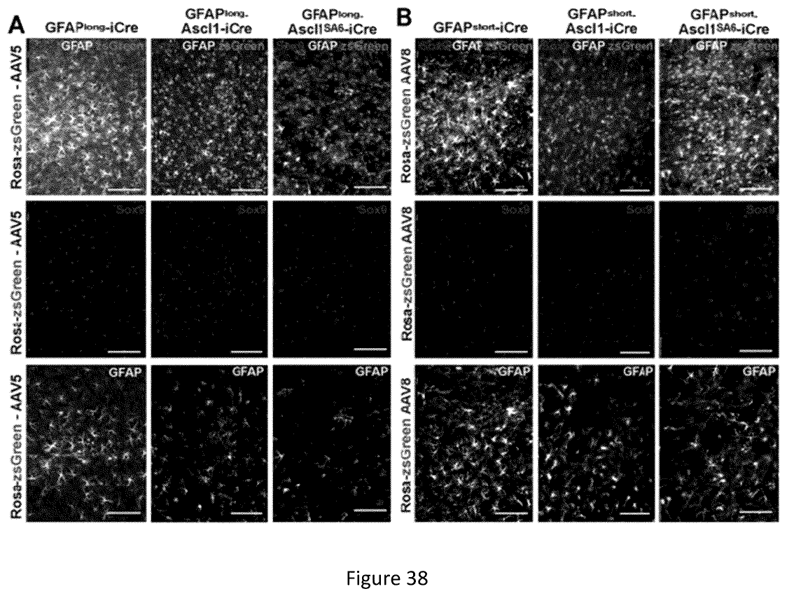

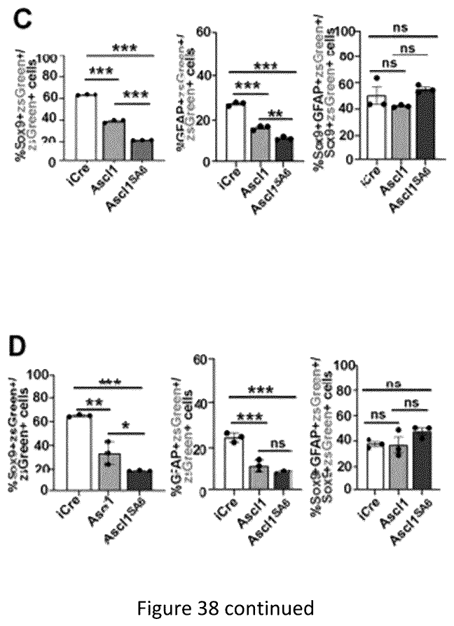

For mCherry AAV injections in FIG. 36B, a total of 1.0×10⁸ GC in a 1 μL total volume at coordinates (AP: +1.1, L/M: ±1.2 DV: −2) was injected. For iCre AAV injections in Rosa-tdtomato mice (FIG. 37C), 1×10¹² GC/ml in a 1 μL total volume (or 1×10⁹ GC total) was injected with coordinates (AP: 2.2 LM: 0.6 DV:1.0). In all other experiments performed in Rosa-zsGreen animals with iCre AAVs (FIG. 37E,G, FIGS. 38, 39 and 40), 4.8×10¹² GC/ml in a 1 μL total volume (or 4.8×10⁹ GC total) was injected with the following coordinates (AP: +2.15, L/M: ±1.7, DV: −1.7).

Optogenetics and electrophysiology. AAV-injected mice were anesthetized using 2% isoflurane and a 4 mm craniotomy was performed (from bregma: AP 1.7 mm, ML+-2.15). After the removal of the dura, a silicone-based polydimethylsiloxane (PDMS) window was placed over a thin layer of 1% agarose (in PBS) covering the cortical tissue. The mice were transferred to an FVMPE-RS multiphoton microscope (Olympus) and placed under a 25×/1.05 NA objective lens (Olympus) while a tungsten electrode (0.255 mm Ø, A-M System) was inserted at a 30° angle, through the PDMS window, reaching a depth of 100 μm into the cortex. An Insight Ti:Sapphire laser (SpectraPhysics) tuned to 900 nm was used to excite the Zs-green fluorescence, whose emission was then collected by a PMT aligned with a band-pass filter (485-540 nm). A second channel (575-630 nm) was also recorded simultaneously to better visualize the position of the Tungsten electrode's tip into the tissue. For simultaneous focused photostimulation (PS) of ChR2, a raster scanned visible wave-length laser (458 nm) with a separate galvanometer was used. The PS was presented over a circular area of 250 μm in diameter where Zs-green-positive cells were present. The PS was repeated ten times over the same area with 10 sec intervals (PS off). The PS was delivered over the circular area at 4 Hz with a power of 4 mW/mm2, and lasted for a total of 3 s. The low-impedance tungsten electrode was used to acquire voltage changes in the LFP band (1-300 Hz), recorded in current clamp mode by the Axon multiclamp 700B amplifier (Molecular devices). The analog signal was amplified 40 times (40 mV/mV) and digitized by the data acquisition system Digidata 1440A™ (Molecular devices). A two-phase decay model was used to describe the repolarization phase of the LFP signal and the slower component was reported as the decay constant.

Tissue processing and sectioning. Mice were anesthetized with ketamine (75 mg/kg, Narketan, 0237499) and xylazine (10 mg/kg, Rompun, 02169592) prior to perfusion. Intracardial perfusion was performed with approximately 20× blood volume using a peristaltic pump at a flow rate of 10 ml/min with ice cold saline (0.9% NaCl, Braun, L8001), followed by 4% paraformaldehyde (PFA, Electron Microscopy Sciences, 19208) in phosphate buffer saline (PBS, Wisent, 311-011-CL) for five minutes. Brains were collected and post-fixed overnight in 4% PFA in PBS, cryoprotected at 4° C. in 20% sucrose (Sigma, 84097)/1×PBS overnight. Coronal brain sections were cut at 10-30 μm on a Leica CM3050™ cryostat (Leica Microsystems Canada Inc., Richmond Hill, ON, Canada) and collected on Fisherbrand™ Superfrost™ Plus Microscope Slides (Thermo Fisher Scientific, 12-550-15).

Immunostaining. Slides were washed in 0.3% Triton X-100 in PBS, then blocked for 1 hour at room temperature in 10% horse serum (HS, Wisent, 065-150) and 0.1% Triton X-100 (Sigma, T8787) in PBS (PBST). Primary antibodies were diluted in blocking solution as follows: rabbit anti-NeuN (1:500, Abcam #ab177487), goat anti-GFAP (1:500, Novus #100-53809), rabbit anti-Sox9 (1:500, Millipore #AB5535), and rabbit anti-Dcx (1/500, Abcam #ab77450). Slides were washed three times for 10 min each in 0.1% Triton-X-100 in PBS, and incubated with species-specific secondary antibodies conjugated to Alexa568 (1:500; Invitrogen Molecular Probes™, ThermoFisher Scientific, Markham, ON, Canada), Alexa488 (1:500; Molecular Probes) for 1 hour in room temperature. Slides were washed three times in PBS and counterstained with 4′,6-diamidino-2-phenylindole (DAPI, Invitrogen, D1306). Finally, the slides were washed three times in PBS and mounted in Aqua-polymount (Polysciences Inc., 18606-20).

RNA in situ hybridization. Colorimetric RNA-in situ hybridization (ISH) was performed using a digoxygenin-labeled Ascl1 riboprobe as previously described [45]. Fluorescent RNA-ISH was performed using an RNAscope® Multiplex Fluorescent Detection Kit v2 (ACD #323110) and followed the manufacturer's instructions. Briefly, brain sections were post-fixed (4% PFA/1×PBS) for 15 min at 4° C., and then, at room-temperature, dehydrated in 50%, 70% and 100% ethanol (Commercial Alcohols, P016EAAN) for 5 min each, and incubated in H₂O₂ solution for 10 min. Sections were then incubated in 1× target retrieval solution for 5 min at 95° C., washed in dH₂O, and then incubated in Protease Plus™ (ACD, 322331) for 15 min at 40° C. before washing in washing buffer. A labeled RNA probe was used for Ascl1 (Mm-Asc1 #313291) and the negative and positive control probes provided were also used. Sections were incubated with the probes for 2 hrs at 40° C. Amplification and staining steps were completed following the manufacturer's instructions, using an Opal™ 570 (1:1500, Akoya #FP1488001KT) fluorophore.

Imaging, quantification and statistics. All images were taken using a Leica DMi8 Inverted Microscope (Leica Microsystems CMS, 11889113) with the following exceptions. Images in FIG. 36E were acquired using a Zeiss Z1 Observer/Yokogawa spinning disk (Carl Zeiss) microscope. Tiled images encompassing the entire motor cortex were acquired using 30 m z-stacks with a 1 m step-size at with a 20× objective. In FIG. 36F, whole section images were scanned with the Zeiss AxioScan Z1 unit (Carl Zeiss Canada) using a Plan-Apochromat 10× objective and acquired with a Hamamatsu CCD camera. Quantification of immunostained cells was performed on three brains per condition and a minimum of three sections per brain. Comparisons were made using a One-Way ANOVA and Tukey multiple comparisons. Significance was defined as p-values less than 0.05 and denoted as follows: ns—not significant, <0.05*, <0.01**, <0.001***.

### Results

### Aav-GFAP-mCherry Expression in Cortical Astrocytes is not Maintained Long-Term In Vivo

The goal of this Example was to determine whether Ascl1-SA6 is more efficient than Ascl1 at inducing neuronal marker expression when misexpressed in adult cortical astrocytes in vivo. To address this question, first AAV8 carrying a GFAPlong promoter (2.2 kb, as in [41]) was used to express mCherry (control) or Ascl1-mCherry in cortical astrocytes (FIG. 36A). Notably, this viral delivery system was reported to successfully convert adult midbrain astrocytes to iNeurons in vivo [26]. A total of 1∩genome copies (GC) of AAV8-GFAPlong-mCherry or AAV8-GFAPlong-Ascl1-mCherry was injected into each hemisphere of the cerebral cortex using stereotactic surgery (FIG. 36A, as in [46]). After one- and two months, animals were harvested from each AAV injection group. At one-month post-transduction, the expected co-incident expression of mCherry and GFAP in astrocytes was observed in AAV8-GFAPlong-mCherry-transduced brains (FIG. 36B). However, when the timeline of analysis was extended to 2 months, mCherry expression was no longer detected in any of the control AAV8-GFAPlong-mCherry-transduced brains (FIG. 36B). In contrast, by 1-month post-transduction, many mCherry+ cells transduced with AAV8-GFAPlong-Ascl1-mCherry expressed Dcx, an early neuroblast marker, and after 2 months, most mCherry+ cells co-expressed NeuN, a late neuronal marker (FIG. 36B,C). Nevertheless, given that the control reporter expression was lost, it was not possible to compare the induction efficiency of neuronal marker expression in mCherry control and Ascl1-mCherry experimental groups, prompting a change in the tracing system employed.

### AAV-GFAP-iCre can be Used for Long-Term Tracing of the Fate of Transduced Cortical Astrocytes In Vivo

To circumvent the issues observed with mCherry reporter expression long-term, a Cre-based, permanent lineage tracing system was used, specifically an AAV5-GFAPshort-iCre vector previously used in a neuronal reprogramming study to misexpress Neurod1 [44], replacing Neurod1 with Ascl1 to create AAV5-GFAPshort-Ascl1-t2a-iCre (FIG. 36D). Notably, the GFAPshort promoter is a 681 bp (gfaABC(1)D modified promoter that shows similar astrocyte-specificity as GFAPlong, but drives two-fold higher levels of gene expression [41].

Packaged AAVs (4.8×10⁹ GC total in a 1 μL total volume) were stereotactically injected into the cortex of Rosa-zsGreen mice, using the same coordinates as in FIG. 36B. Robust zsGreen expression was observed 2 months after transduction of AAV5-GFAPshort-iCre control virus (not shown) or AAV5-GFAPshort-Ascl1-t2a-iCre (FIG. 36E). It was further confirmed that Ascl1 transcripts were detected in the Ascl1-t2a-iCre transduced brains, even after 2 months, using RNA in situ hybridization with a digoxygenin-labeled Ascl1 riboprobe (FIG. 36F). A final confirmation was performed using RNAscope™ which definitively showed that while Ascl1 transcripts were not detected in the adult cortex transduced with iCre control vectors, robust Ascl1 expression was detected in the Ascl1-t2a-iCre transduced brains two months post-transduction (FIG. 36G). The iCre system thus can be used to express Ascl1 and to trigger Cre-dependent reporter expression, which in turn can be used to trace the fate of transduced cells in the adult cerebral cortex

### Mutating Serine Phospho-Acceptor Sites in Ascl1 Augments Neuronal Lineage Conversion in the Adult Cortex

The main goal of this study was to determine whether serine-to-alanine mutations in the six SP sites in Ascl1 (FIG. 36H) would enhance neuronal conversion efficacy. Notably, it has been previously demonstrated that Ascl1-SA6 was more efficient at neuronal conversion when introduced into E12.5 cortical progenitors [34], but the question remained, would this modified bHLH transcription factor more effectively convert adult cortical astrocytes to iNeurons. To address this question, AAV5 and AAV8 capsids, both of which have been reported to transduce cortical astrocytes [47], GFAPlong and GFAPshort promoters [41], and Rosa-zsGreen and Rosa-tdtomato reporter mice, two of the brightest fluorescent reporters [43] were compared (FIG. 37). Each comparative group had a set of three genetic cargos: iCre alone (control), Ascl1-t2a-iCre, or Ascl1-SA6-t2a-iCre. The two main comparisons were AAV8 versus AAV5 with the short GFAP promoter, and GFAPshort versus GFAPlong in AAV5. As above, all packaged AAVs were injected using stereotactic surgery into the cerebral cortex of either Rosa-zsGreen or Rosa-tdtomato mice.

First the ability of AAV5-GFAPshort constructs to induce NeuN expression when injected into the cortex of Rosa-tdtomato mice were compared (FIG. 37C). At 21 days post-transduction, similar numbers of iCre and Ascl1 transduced cells expressed NeuN expression, with only Ascl1-SA6 able to increase the number of NeuN expressing cells above iCre “baseline” levels (FIG. 37D). Next how AAV5-GFAPlong constructs act when introduced into the cortex of Rosa-zsGreen mice was examined (FIG. 37E). At 21 days post-transduction, ˜70% of the iCre transduced control cells expressed NeuN (FIG. 37F), suggesting that this long promoter is not as restricted to astrocytes as the GFAPshort promoter. Nevertheless, when Ascl1-SA6 was expressed from the GFAPlong promoter, more transduced cells expressed NeuN compared to Ascl1 or iCre (FIG. 37F). Finally, the GFAPshort promoter was tested the AAV8 capsid (FIG. 37G). With this system, it also was found that ˜50% of iCre control cells expressed NeuN, but both Ascl1, and more strikingly, Ascl1-SA6 could induce an increase in the number of NeuN expressing cells at 21 days post-transduction (FIG. 37H).

From these studies, it was concluded that Ascl1-SA6 transduced cells more frequently express NeuN compared to Ascl1 transduced cells when delivered to the adult cerebral cortex using GFAP promoter elements. In addition, this study supports previous studies using transgenic mice that suggested that the GFAPshort promoter is more specific to cortical astrocytes than the GFAPlong promoter [41]. Finally, the AAV5 capsid labels fewer cortical neurons than AAV8 when combined with GFAP promoter elements, and may be better suited for neuronal reprogramming in vivo.

### Ascl1-SA6 and Ascl1 Inhibit Astrocytic Marker Expression

True lineage conversion requires that the targeted cells, in this case astrocytes, must not only turn on neuronal markers, but also extinguish the expression of glial markers. Indeed, in the embryonic cortex, it has been shown that Ascl1-SA6 is more efficient at turning on neuronal gene expression, and less efficient at transactivating the Sox9 glial promoter compared to Ascl1 [34]. Here it was thus asked whether in the adult cortex, Ascl1-SA6 could more efficiently downregulate Sox9 expression. In this set of experiments, two systems were compared: the AAV5 vector with the GFAPlong promoter and the AAV8 vector with the GFAPshort promoter. As expected, ˜60% of cells transduced with AAV5-GFAP-long-iCre (FIG. 38A,C) or AAV8-GFAPshort-iCre (FIG. 38B,D) expressed Sox9 after 21 days, confirming their astrocytic identity. Interestingly, the ratio of iCre control cells that co-expressed GFAP was lower, ˜30%, for both AAV5-GFAP-long-iCre (FIG. 38A,C) or AAV8-GFAPshort-iCre (FIG. 38B,D). One possibility is that astrocytes that initially expressed GFAP at the time of transduction turned off their GFAP expression within the 21 days before analysis. Nevertheless, regardless of the discrepancy, it was concluded that over half of the iCre control cells are indeed astrocytes.

The next comparison was glial marker expression after transduction of AAV5-GFAP-long-Ascl1-SA6-iCre (FIG. 38A,C) or AAV8-GFAPshort-Ascl1-SA6-iCre (FIG. 38B,D). As expected, both Ascl1 and Ascl1-SA6 transduced cells less frequently expressed Sox9 and GFAP compared to iCre control cells after 21 days post-transduction (FIG. 38C,D). Notably, this drop in marker expression did not alter the ratio of cells that co-expressed Sox9 and GFAP, so both populations of cells were equally affected (FIG. 38C,D). Taken together, this data supports the contention that Ascl1 and Ascl1-SA6 both suppress an astrocytic fate in the adult cortex, and further suggests that Ascl1-SA6 is more efficient at glial repression, as demonstrated in the embryonic cortex [34].

### Ascl1 and Ascl1-SA6 Transduced Cells go Through a Dcx+ Neuroblast Stage

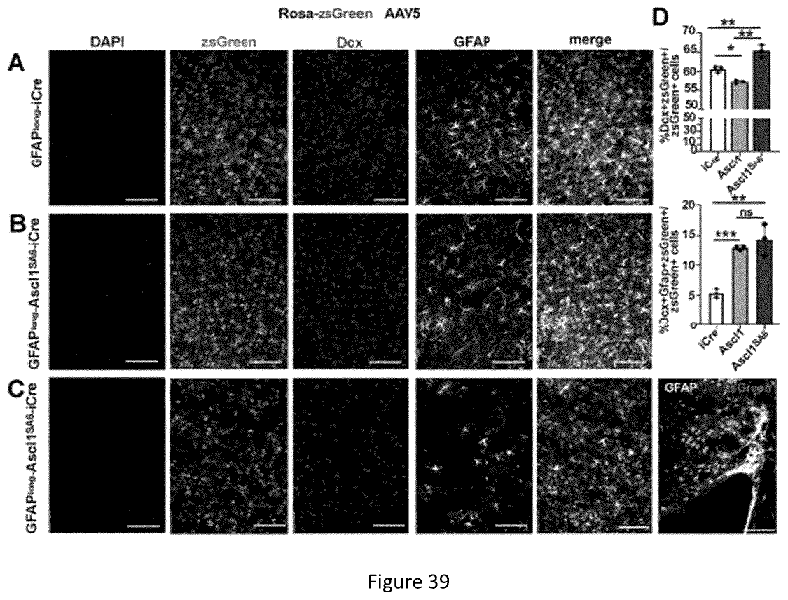

Another requirement to show lineage conversion is to demonstrate that transduced astrocytes go through an immature Dcx+ neuroblast stage. Thus conversion efficiency using the AAV5-GFAP-long constructs was evaluated by examining Dcx expression at 21 days post-transduction of the adult cerebral cortex (FIG. 39). After 21 days, there was a small but significant increase in the number of Ascl1-SA6 transduced cells that expressed Dcx above iCre and Ascl1 levels (FIG. 39A-D). However, as reporter expression was observed in both parenchymal astrocytes and stem cells in the subependymal zone with the present injection strategy (FIG. 39C), GFAP and Dcx co-expression was used to identify astrocytes in a “transitory” neuroblast stage. Quantification of Dcx+GFAP+zsGreen+ cells, revealed that both Ascl1 and Ascl1-SA6 transduced cells were more likely to acquire a transitory neuroblast-like state than iCre transduced cortical astrocytes (FIG. 39D). Thus, Ascl1 and Ascl1-SA6 transduced cells have an equivalent capacity to initiate Dcx expression, but only Ascl1-SA6 can sustain high Dcx levels (FIG. 39D), and ultimately turn on NeuN, a mature neuronal marker (FIG. 37).

Ascl1-SA6 Induces Electrophysiological Properties of iNeurons in Targeted Astrocytes

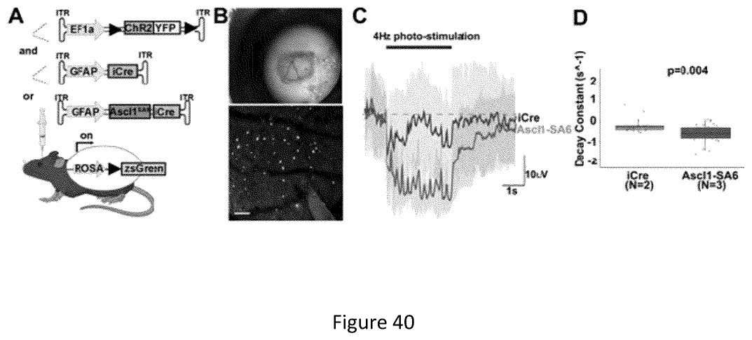

To promote functional recovery in pathological conditions, iNeurons must integrate into existing neural circuits by making synaptic connections with endogenous neurons and sending axons to appropriate neuronal targets. To test neural network integration of iNeurons in vivo, AAVs carrying GFAP-iCre or GFAP-Ascl1-SA6 were co-transduced with FLEX-ChR2-(H134R)-YFP, a Cre-dependent optogenetic actuator that offers a sensitive way to photoactivate neurons and elicit large evoked potentials (FIG. 40A). After 36 days, a cranial window was made and intracortical electrophysiological recordings were performed to assess local field potentials, a measure of aggregate neuronal activity, in response to ChR2 photoactivation (20 Hz, 10 ms pulse length, 5 s total) (FIG. 40B). In a representative trace, and quantified for several sites, Ascl1-SA6 iNeurons transduced cortices exhibited a faster decay of evoked potentials to baseline than did iCre transduced cortices (FIG. 40C,D), a neuronal feature. This data thus supports the contention that Ascl1-SA6 successfully converts transduced astrocytes into functional iNeurons.

### Discussion

This Example provides a detailed comparison of the capacity of Ascl1 and Ascl1-SA6 to induce neuronal markers and suppress glial markers when expressed in adult cortical astrocytes in vivo. It was found that with each combination of AAV capsids, GFAP promoters and Rosa-reporter lines tested, a higher proportion of Ascl1-SA6 transduced cells consistently expressed NeuN, a mature neuronal marker, compared to cells transduced with Ascl1 or iCre controls. In contrast, an equivalent number of Ascl1 and Ascl1-SA6 transduced cells had the signature of a transitory GFAP+Dcx+ neuroblast stage, suggesting that while Ascl1 may induce some features of immature neurons, it is less efficient than Ascl1-SA6 at inducing a complete differentiation cascade. In addition, both Ascl1 and Ascl1-SA6 could suppress the expression of astrocytic markers (Sox9 and GFAP), although Ascl1-SA6 was again superior in this regard. In addition, it was recently demonstrated that ASCL1-SA5 (note that the human ASCL1 gene has 5 SP sites) can induce a glioblastoma stem cell line to undergo terminal differentiation and exit the cell cycle more effectively than native ASCL1, leading to growth suppression of this tumor cell line [39]. Taken together with the work in the current Example, there is now cumulative support for the enhanced pro-neurogenic and anti-astrocytic capacity of bHLH mutants, such as Ascl1-SA6, versus Ascl1.

### REFERENCES

1. S. Grade, and M. Gotz, Neuronal replacement therapy: previous achievements and challenges ahead. NPJ Regen Med 2 (2017) 29.
2. R. A. Barker, M. Gotz, and M. Parmar, New approaches for brain repair-from rescue to reprogramming. Nature 557 (2018) 329-334.
3. K. V. Adams, and C. M. Morshead, Neural stem cell heterogeneity in the mammalian forebrain. Prog Neurobiol 170 (2018) 2-36.
4. R. M. Ruddy, and C. M. Morshead, Home sweet home: the neural stem cell niche throughout development and after injury. Cell Tissue Res 371 (2018) 125-141.
5. R. Bocchi, G. Masserdotti, and M. Götz, Direct neuronal reprogramming: Fast forward from new concepts toward therapeutic approaches. Neuron (2021).
6. L. Vasan, E. Park, L. A. David, T. Fleming, and C. Schuurmans, Direct Neuronal Reprogramming: Bridging the Gap Between Basic Science and Clinical Application. Front Cell Dev Biol 9 (2021) 681087.
7. A. M. Oproescu, S. Han, and C. Schuurmans, New Insights Into the Intricacies of Proneural Gene Regulation in the Embryonic and Adult Cerebral Cortex. Front Mol Neurosci 14 (2021) 642016.
8. S. Li, P. Mattar, D. Zinyk, K. Singh, C. P. Chaturvedi, C. Kovach, R. Dixit, D. M. Kurrasch, Y. C. Ma, J. A. Chan, V. Wallace, F. J. Dilworth, M. Brand, and C. Schuurmans, GSK3 temporally regulates neurogenin 2 proneural activity in the neocortex. J Neurosci 32 (2012) 7791-805.
9. O. Britz, P. Mattar, L. Nguyen, L. M. Langevin, C. Zimmer, S. Alam, F. Guillemot, and C. Schuurmans, A role for proneural genes in the maturation of cortical progenitor cells. Cereb Cortex 16 Suppl 1 (2006) i138-51.
10. C. Schuurmans, O. Armant, M. Nieto, J. M. Stenman, O. Britz, N. Klenin, C. Brown, L. M. Langevin, J. Seibt, H. Tang, J. M. Cunningham, R. Dyck, C. Walsh, K. Campbell, F. Polleux, and F. Guillemot, Sequential phases of cortical specification involve Neurogenin-dependent and -independent pathways. Embo J 23 (2004) 2892-902.
11. C. Fode, Q. Ma, S. Casarosa, S. L. Ang, D. J. Anderson, and F. Guillemot, A role for neural determination genes in specifying the dorsoventral identity of telencephalic neurons. Genes Dev 14 (2000) 67-80.
12. S. Casarosa, C. Fode, and F. Guillemot, Mash1 regulates neurogenesis in the ventral telencephalon. Development 126 (1999) 525-34.
13. T. Vierbuchen, A. Ostermeier, Z. P. Pang, Y. Kokubu, T. C. Sudhof, and M. Wernig, Direct conversion of fibroblasts to functional neurons by defined factors. Nature 463 (2010) 1035-41.
14. O. L. Wapinski, T. Vierbuchen, K. Qu, Q. Y. Lee, S. Chanda, D. R. Fuentes, P. G. Giresi, Y. H. Ng, S. Marro, N. F. Neff, D. Drechsel, B. Martynoga, D. S. Castro, A. E. Webb, T. C. Sudhof, A. Brunet, F. Guillemot, H. Y. Chang, and M. Wernig, Hierarchical mechanisms for direct reprogramming of fibroblasts to neurons. Cell 155 (2013) 621-35.
15. S. Chanda, C. E. Ang, J. Davila, C. Pak, M. Mall, Q. Y. Lee, H. Ahlenius, S. W. Jung, T. C. Sudhof, and M. Wernig, Generation of induced neuronal cells by the single reprogramming factor ASCL1. Stem Cell Reports 3 (2014) 282-96.
16. M. Karow, R. Sanchez, C. Schichor, G. Masserdotti, F. Ortega, C. Heinrich, S. Gascon, M. A. Khan, D. C. Lie, A. Dellavalle, G. Cossu, R. Goldbrunner, M. Gotz, and B. Berninger, Reprogramming of pericyte-derived cells of the adult human brain into induced neuronal cells. Cell Stem Cell 11 (2012) 471-6.
17. M. Karow, J. G. Camp, S. Falk, T. Gerber, A. Pataskar, M. Gac-Santel, J. Kageyama, A. Brazovskaja, A. Garding, W. Fan, T. Riedemann, A. Casamassa, A. Smiyakin, C. Schichor, M. Gotz, V. K. Tiwari, B. Treutlein, and B. Berninger, Direct pericyte-to-neuron reprogramming via unfolding of a neural stem cell-like program. Nat Neurosci 21 (2018) 932-940.
18. A. Raposo, F. F. Vasconcelos, D. Drechsel, C. Marie, C. Johnston, D. Dolle, A. Bithell, S. Gillotin, D. L. C. van den Berg, L. Ettwiller, P. Flicek, G. E. Crawford, C. M. Parras, B. Berninger, N.J. Buckley, F. Guillemot, and D. S. Castro, Ascl1 Coordinately Regulates Gene Expression and the Chromatin Landscape during Neurogenesis. Cell Rep 10 (2015) 1544-1556.
19. B. Aydin, A. Kakumanu, M. Rossillo, M. Moreno-Estelles, G. Garipler, N. Ringstad, N. Flames, S. Mahony, and E. O. Mazzoni, Proneural factors Ascl1 and Neurog2 contribute to neuronal subtype identities by establishing distinct chromatin landscapes. Nat Neurosci 22 (2019) 897-908.
20. N. Yang, S. Chanda, S. Marro, Y. H. Ng, J. A. Janas, D. Haag, C. E. Ang, Y. Tang, Q. Flores, M. Mall, O. Wapinski, M. Li, H. Ahlenius, J. L. Rubenstein, H. Y. Chang, A. A. Buylla, T. C. Sudhof, and M. Wernig, Generation of pure GABAergic neurons by transcription factor programming. Nat Methods 14 (2017) 621-628.
21. B. Berninger, F. Guillemot, and M. Gotz, Directing neurotransmitter identity of neurones derived from expanded adult neural stem cells. Eur J Neurosci 25 (2007) 2581-90.
22. C. Heinrich, R. Blum, S. Gascon, G. Masserdotti, P. Tripathi, R. Sanchez, S. Tiedt, T. Schroeder, M. Gotz, and B. Berninger, Directing astroglia from the cerebral cortex into subtype specific functional neurons. PLoS Biol 8 (2010) e1000373.
23. C. Heinrich, M. Gotz, and B. Berninger, Reprogramming of postnatal astroglia of the mouse neocortex into functional, synapse-forming neurons. Methods Mol Biol 814 (2012) 485-98.
24. J. Kempf, K. Knelles, B. A. Hersbach, D. Petrik, T. Riedemann, V. Bednarova, A. Janjic, T. Simon-Ebert, W. Enard, P. Smialowski, M. Gotz, and G. Masserdotti, Heterogeneity of neurons reprogrammed from spinal cord astrocytes by the proneural factors Ascl1 and Neurogenin2. Cell Rep 36 (2021) 109409.
25. P. Rivetti di Val Cervo, R. A. Romanov, G. Spigolon, D. Masini, E. Martin-Montanez, E. M. Toledo, G. La Manno, M. Feyder, C. Pifl, Y. H. Ng, S. P. Sanchez, S. Linnarsson, M. Wernig, T. Harkany, G. Fisone, and E. Arenas, Induction of functional dopamine neurons from human astrocytes in vitro and mouse astrocytes in a Parkinson's disease model. Nat Biotechnol 35 (2017) 444-452.
26. Y. Liu, Q. Miao, J. Yuan, S. Han, P. Zhang, S. Li, Z. Rao, W. Zhao, Q. Ye, J. Geng, X. Zhang, and L. Cheng, Ascl1 Converts Dorsal Midbrain Astrocytes into Functional Neurons In Vivo. J Neurosci 35 (2015) 9336-55.
27. S. Jessberger, N. Toni, G. D. Clemenson, Jr., J. Ray, and F. H. Gage, Directed differentiation of hippocampal stem/progenitor cells in the adult brain. Nat Neurosci 11 (2008) 888-93.
28. A. Grande, K. Sumiyoshi, A. Lopez-Juarez, J. Howard, B. Sakthivel, B. Aronow, K. Campbell, and M. Nakafuku, Environmental impact on direct neuronal reprogramming in vivo in the adult brain. Nat Commun 4 (2013) 2373.
29. J. Giehrl-Schwab, F. Giesert, B. Rauser, C. L. Lao, S. Hembach, S. Lefort, I. L. Ibarra, C. Koupourtidou, M. D. Luecken, D. J. Truong, J. Fischer-Sternjak, G. Masserdotti, N. Prakash, J. Ninkovic, S. M. Holter, D. M. Vogt Weisenhorn, F. J. Theis, M. Gotz, and W. Wurst, Parkinson's disease motor symptoms rescue by CRISPRa-reprogramming astrocytes into GABAergic neurons. EMBO Mol Med (2022) e14797.
30. S. Gascon, E. Murenu, G. Masserdotti, F. Ortega, G. L. Russo, D. Petrik, A. Deshpande, C. Heinrich, M. Karow, S. P. Robertson, T. Schroeder, J. Beckers, M. Irmler, C. Berndt, J. P. Angeli, M. Conrad, B. Berninger, and M. Gotz, Identification and Successful Negotiation of a Metabolic Checkpoint in Direct Neuronal Reprogramming. Cell Stem Cell 18 (2016) 396-409.
31. G. Masserdotti, S. Gillotin, B. Sutor, D. Drechsel, M. Irmler, H. F. Jorgensen, S. Sass, F. J. Theis, J. Beckers, B. Berninger, F. Guillemot, and M. Gotz, Transcriptional Mechanisms of Proneural Factors and REST in Regulating Neuronal Reprogramming of Astrocytes. Cell Stem Cell 17 (2015) 74-88.
32. J. Drouin-Ouellet, S. Lau, P. L. Brattas, D. Rylander Ottosson, K. Pircs, D. A. Grassi, L. M. Collins, R. Vuono, A. Andersson Sjoland, G. Westergren-Thorsson, C. Graff, L. Minthon, H. Toresson, R. A. Barker, J. Jakobsson, and M. Parmar, REST suppression mediates neural conversion of adult human fibroblasts via microRNA-dependent and -independent pathways. EMBO Mol Med 9 (2017) 1117-1131.
33. G. L. Russo, G. Sonsalla, P. Natarajan, C. T. Breunig, G. Bulli, J. Merl-Pham, S. Schmitt, J. Giehrl-Schwab, F. Giesert, M. Jastroch, H. Zischka, W. Wurst, S. H. Stricker, S. M. Hauck, G. Masserdotti, and M. Gotz, CRISPR-Mediated Induction of Neuron-Enriched Mitochondrial Proteins Boosts Direct Glia-to-Neuron Conversion. Cell Stem Cell 28 (2021) 524-534 e7.
34. S. Li, P. Mattar, R. Dixit, S. O. Lawn, G. Wilkinson, C. Kinch, D. Eisenstat, D. M. Kurrasch, J. A. Chan, and C. Schuurmans, RAS/ERK signaling controls proneural genetic programs in cortical development and gliomagenesis. J Neurosci 34 (2014) 2169-90.
35. F. Ali, C. Hindley, G. McDowell, R. Deibler, A. Jones, M. Kirschner, F. Guillemot, and A. Philpott, Cell cycle-regulated multi-site phosphorylation of Neurogenin 2 coordinates cell cycling with differentiation during neurogenesis. Development 138 (2011) 4267-77.
36. C. Hindley, F. Ali, G. McDowell, K. Cheng, A. Jones, F. Guillemot, and A. Philpott, Post-translational modification of Ngn2 differentially affects transcription of distinct targets to regulate the balance between progenitor maintenance and differentiation. Development 139 (2012) 1718-23.
37. F. R. Ali, D. Marcos, I. Chernukhin, L. M. Woods, L. M. Parkinson, L. A. Wylie, T. D. Papkovskaia, J. D. Davies, J. S. Carroll, and A. Philpott, Dephosphorylation of the Proneural Transcription Factor ASCL1 Re-Engages a Latent Post-Mitotic Differentiation Program in Neuroblastoma. Mol Cancer Res 18 (2020) 1759-1766.
38. R. Azzarelli, L. J. Hardwick, and A. Philpott, Emergence of neuronal diversity from patterning of telencephalic progenitors. Wiley Interdiscip Rev Dev Biol 4 (2015) 197-214.
39. R. Azzarelli, A. McNally, C. Dell'Amico, M. Onorati, B. Simons, and A. Philpott, ASCL1 phosphorylation and ID2 upregulation are roadblocks to glioblastoma stem cell differentiation. Sci Rep 12 (2022) 2341.
40. E. A. Marcus, C. Kintner, and W. Harris, The role of GSK3beta in regulating neuronal differentiation in Xenopus laevis. Mol Cell Neurosci 12 (1998) 269-80.
41. Y. Lee, A. Messing, M. Su, and M. Brenner, GFAP promoter elements required for region-specific and astrocyte-specific expression. Glia 56 (2008) 481-93.
42. L. L. Wang, C. Serrano, X. Zhong, S. Ma, Y. Zou, and C. L. Zhang, Revisiting astrocyte to neuron conversion with lineage tracing in vivo. Cell 184 (2021) 5465-5481 e16.
43. L. Madisen, T. A. Zwingman, S. M. Sunkin, S. W. Oh, H. A. Zariwala, H. Gu, L. L. Ng, R. D. Palmiter, M. J. Hawrylycz, A. R. Jones, E. S. Lein, and H. Zeng, A robust and high-throughput Cre reporting and characterization system for the whole mouse brain. Nat Neurosci 13 (2010) 133-40.
44. J. Livingston, T. Lee, E. Daniele, C. Phillips, A. Krassikova, T. Enbar, I. Kortebi, K. W. A. Bang, B. Donville, O. Ibragimov, N. Sachewsky, C. M. Morshead, and M. Faiz, Direct reprogramming of astrocytes to neurons leads to functional recovery after stroke. bioRxiv (2020) 2020.02.02.929091.
45. Y. Touahri, L. Adnani, P. Mattar, K. Markham, N. Klenin, and C. Schuurmans, Non-isotopic RNA In Situ Hybridization on Embryonic Sections. Curr Protoc Neurosci 70 (2015) 1 22 1-1 22 25.
46. C. S. Khademullah, A. J. Aqrabawi, K. M. Place, Z. Dargaei, X. Liang, J. C. Pressey, S. Bedard, J. W. Yang, D. Garand, I. Keramidis, A. Gasecka, D. Cote, Y. De Koninck, J. Keith, L. Zinman, J. Robertson, J. C. Kim, and M. A. Woodin, Cortical interneuron-mediated inhibition delays the onset of amyotrophic lateral sclerosis. Brain 143 (2020) 800-810.
47. D. F. Aschauer, S. Kreuz, and S. Rumpel, Analysis of transduction efficiency, tropism and axonal transport of AAV serotypes 1, 2, 5, 6, 8 and 9 in the mouse brain. PLoS One 8 (2013) e76310.
48. Y. Rao, S. Du, B. Yang, Y. Wang, Y. Li, R. Li, T. Zhou, X. Du, Y. He, Y. Wang, X. Zhou, T. F. Yuan, Y. Mao, and B. Peng, NeuroD1 induces microglial apoptosis and cannot induce microglia-to-neuron cross-lineage reprogramming. Neuron 109 (2021) 4094-4108.e5.
49. Y. Sun, M. Nadal-Vicens, S. Misono, M. Z. Lin, A. Zubiaga, X. Hua, G. Fan, and M. E. Greenberg, Neurogenin promotes neurogenesis and inhibits glial differentiation by independent mechanisms. Cell 104 (2001) 365-76.
50. C. Kovach, R. Dixit, S. Li, P. Mattar, G. Wilkinson, G. E. Elsen, D. M. Kurrasch, R. F. Hevner, and C. Schuurmans, Neurog2 simultaneously activates and represses alternative gene expression programs in the developing neocortex. Cereb Cortex 23 (2013) 1884-900.
51. S. Han, S. Okawa, G. A. Wilkinson, H. Ghazale, L. Adnani, R. Dixit, L. Tavares, I. Faisal, M. J. Brooks, V. Cortay, D. Zinyk, A. Sivitilli, S. Li, F. Malik, Y. Ilnytskyy, V. E. Angarica, J. Gao, V. Chinchalongporn, A. M. Oproescu, L. Vasan, Y. Touahri, L. A. David, E. Raharjo, J. W. Kim, W. Wu, W. Rahmani, J. A. Chan, I. Kovalchuk, L. Attisano, D. Kurrasch, C. Dehay, A. Swaroop, D. S. Castro, J. Biernaskie, A. Del Sol, and C. Schuurmans, Proneural genes define ground-state rules to regulate neurogenic patterning and cortical folding. Neuron 109 (2021) 2847-2863 eli.
52. T. Matsuda, T. Irie, S. Katsurabayashi, Y. Hayashi, T. Nagai, N. Hamazaki, A. M. D. Adefuin, F. Miura, T. Ito, H. Kimura, K. Shirahige, T. Takeda, K. Iwasaki, T. Imamura, and K. Nakashima, Pioneer Factor NeuroD1 Rearranges Transcriptional and Epigenetic Profiles to Execute Microglia-Neuron Conversion. Neuron 101 (2019) 472-485 e7.
53. T. Matsuda, and K. Nakashima, Clarifying the ability of NeuroD1 to convert mouse microglia into neurons. Neuron 109 (2021) 3912-3913.
54. D. Leib, Y. H. Chen, A. M. Monteys, and B. L. Davidson, Limited astrocyte-to-neuron conversion in the mouse brain using NeuroD1 overexpression. Mol Ther 30 (2022) 982-986.
55. C. M. Parras, C. Schuurmans, R. Scardigli, J. Kim, D. J. Anderson, and F. Guillemot, Divergent functions of the proneural genes Mash1 and Ngn2 in the specification of neuronal subtype identity. Genes Dev 16 (2002) 324-38.
56. N. Sharif, F. Calzolari, and B. Berninger, Direct In Vitro Reprogramming of Astrocytes into Induced Neurons. Methods Mol Biol 2352 (2021) 13-29.
57. H. Qian, X. Kang, J. Hu, D. Zhang, Z. Liang, F. Meng, X. Zhang, Y. Xue, R. Maimon, S. F. Dowdy, N. K. Devaraj, Z. Zhou, W. C. Mobley, D. W. Cleveland, and X. D. Fu, Reversing a model of Parkinson's disease with in situ converted nigral neurons. Nature 582 (2020) 550-556.
58. T. Irie, T. Matsuda, Y. Hayashi, A. Kamiya, J.-i. Kira, and K. Nakashima, Direct neuronal conversion of microglia/macrophages reinstates neurological function after stroke. bioRxiv (2021) 2021.09.26.461831.

### Example 6: Spatial Transcriptomic Analysis of In Vivo Astrocyte-to-Neuron Lineage Conversion in a Mouse Model of Amyotrophic Lateral Sclerosis

Direct neuronal reprogramming refers to the conversion of mature somatic cells to an induced (i) Neuron without going through pluripotency^{1,2}, and was first documented even prior to the initial demonstration of reprogramming to a pluripotent state³. Since this original discovery, basic-helix-loop-helix (bHLH) transcription factors (TFs) encoded by the proneural genes Neurog2 and Ascl1 and the neuronal differentiation gene Neurod1 have become the TFs of choice to convert somatic cells, including brain glia (e.g., astrocytes, microglia) to iNeurons². These bHLH TFs were selected based on their roles in driving embryonic neurogenesis and neuronal subtype specification⁴. For example, in the embryonic forebrain, Ascl1 is necessary and sufficient to specify a GABAergic, inhibitory neuronal identity, whereas Neurog2 specifies a glutamatergic excitatory neuronal fate in a transcriptional cascade that includes Neurod1⁵⁻⁹. While several groups have used these bHLH TFs in neuronal reprogramming studies in vitro¹⁰⁻¹⁷, the efficiency of iNeuron conversion remains low when applied to endogenous cells in the brain in vivo². One mode of inhibitory control is phosphorylation by proline-directed serine-threonine (S/T) kinases, including Cdks, Erk and Gsk3, which phosphorylate and inhibit bHLH TFs in the embryonic mouse brain, during primary Xenopus neurogenesis and in cancer cell lines¹⁸⁻²⁵.

Using this knowledge, the serine-to-alanine (SA) phosphosite mutations were used in Ascl1 (Ascl1SA6) and showed that Ascl1SA6 is more efficient than Ascl1 at inducing neuronal marker expression when expressed in astrocytes in adult brain astrocytes in vivo (Example 5), consistent with an enhanced capacity to trigger neuronal lineage conversion. I In the present Example, spatial transcriptomics were used as a reporter agnostic method to comprehensively map the impact of neuronal reprogramming TFs on gene expression at a tissue-wide scale.

Ultimately, the goal of neuronal reprogramming is to replace lost neurons in neurodegenerative disease models, with astrocytes often the target cell of choice. While yet to be fully characterized, neuronal reprogramming efficiency may differ in different brain regions and in a healthy versus neurodegenerative environment¹, especially given the high degree of heterogeneity of astrocytes depending on anatomical location^{28,29} and disease state³⁰. In this Example, reprogramming TFs were introduced into a mouse model of amyotrophic lateral sclerosis (ALS). ALS is associated with the degeneration of upper motor neurons (MNs) in deep layers V and VI of the neocortex, which project to subcortical targets including the striatum (corticostriatal pathway) and spinal cord (corticospinal pathway). Subsequently, lower MNs in the brainstem and spinal cord, which innervate skeletal muscles and other muscle types (cardiac, visceral), also degenerate^{31,32}. Ultimately the loss of lower MNs causes muscle denervation, muscle atrophy, and ultimately death within 3-5 years post diagnosis³². Most ALS diagnoses are sporadic, with only 5-10% of cases inherited³³. Of the familial cases, several mutations have been identified in SOD1, which encodes for Cu—Zn superoxide dismutase 1³⁴. A G93A missense mutation in SOD1 results in a glycine to alanine substitution; this mutation has been introduced into mice to generate hSOD1^{G93A} transgenic mice, which are used for disease modeling³⁵. A transgenic mouse line carrying a human (h) SOD1^{G93A} mutant transgene recapitulates ALS motor deficits³⁶, with upper motor neurons in the cortex degenerating before lower motor neurons in the spinal cord³⁷.

Astrocytes are a major contributor to neuronal cell death in an ALS animal model, as they can induce pathology when transplanted into a healthy host brain³⁸. To prevent toxicity associated with ALS astrocytes, and to potentially replace lost upper motor neurons, the present Example examined whether adeno-associated viruses (AAV) containing a glial fibrillary acidic protein (GFAP) promoter to drive the expression of neurogenic TFs (Ascl1, Ascl1^{SA6}) could be used to induce astrocyte-to-iNeuron conversion in the SOD1^{G93A} motor cortex. In Example 5, above, it was shown that Ascl1^{SA6} was more efficient than Ascl1 at inducing neuronal marker expression in the motor cortex of wild-type mice in vivo²⁶. Here, to determine whether Ascl1 and Ascl1^{SA6} induce neuronal lineage conversion in vivo, the 10× Genomics Visium spatial transcriptomics (ST) platform was used to investigate gene expression changes in a spatially-defined manner. Brain regions were delineated histologically, clustered transcriptionally, and annotated using a Molecular Brain Atlas³⁹. Unexpectedly, the control iCre vector induced an inflammatory gene signature, a phenotype that was dampened when the same AAVs were used to express either Ascl1 or Ascl1^{SA6} Instead, Ascl1 and Ascl1^{SA6} downregulated a subset of astrocytic genes (Nr4a1, Thra, Npas4, Clu) and activated a set of neurogenic genes, including those upregulated during in vitro neuronal conversion (e.g., Zbtb18, Id2, Id3)^{10,23} By building a gene regulatory network (GRN) based on a prior in vitro neuronal conversion dataset in which human ASCL1 or ASCL1^{SA5} were expressed in a glioblastoma cell line²³, ZBTB18 was identified as an important hub TF, the in silico knock-out (KO) of which disrupted the GRN. Taken together, these data support the idea that bHLH TFs activate, at least in part, neurogenic GRNs in motor cortex astrocytes in vivo, demonstrating that this approach can serve as a therapy for neurodegenerative diseases, such as, but not limited to ALS.

### Materials and Methods

Animals. All animal procedures were approved by the Sunnybrook Research Institute Animal Care Committee (ACC Protocol #22-757) in agreement with the Guidelines of the Canadian Council of Animal Care (CCAC). Animals were housed on a 12-hour light-dark cycle with ad libitum access to food and water. Rosa-zsGreen; hSOD1^{G93A} double heterozygous mice were generated by breeding Rosa-zsGreen females (B6.Cg-Gt(ROSA)26Sor^{tm6(CAG-ZsGreen1)Hze}/J; JAX® stock #: 007906) with hSOD1^{G93A} males (B6; Cg-Tg(SOD1-G93A)1Gur/J; JAX® stock #: 004435), which were both maintained on a C57B6/J genetic background. Double transgenic animals were identified by PCR-genotyping using Jackson Laboratory protocols. hSOD1^{G93A} transgene and internal positive control primers (transgene forward, 5′-CAT CAG CCC TAA TCC ATC TGA-3′ (SEQ ID NO:26); reverse, 5′-CGC GAC TAA CAA TCA AAG TGA-3′ (SEQ ID NO:27); internal positive control forward, 5′-CTA GGC CAC AGA ATT GAA AGA TCT-3′ (SEQ ID NO:34); internal positive control reverse, 5′-GTA GGT GGA AAT TCT AGC ATC C-3′ (SEQ ID NO:35)). The hSOD1^{G93A} transgene band is 236 bp and the locus of insertion wild-type band is 324 bp. Rosa-ZsGreen: wild-type forward: 5′-CTG GCT TCT GAG GAC CG-3′ (SEQ ID NO:28); wild-type reverse: 5′-AAT CTG TGG GAA GTC TTG TCC-3′ (SEQ ID NO:29); mutant forward: 5′-ACC AGA AGT GGC ACC TGA C-3′ (SEQ ID NO:30); mutant reverse: 5′-CAA ATT TTG TAA TCC AGA GGT TGA-3′ (SEQ ID NO:31).

AAV Delivery. The AAV5-GFAP-iCre plasmid was a gift from Dr. Maryam Faiz¹⁰⁸ AAV5-GFAP-iCre (2.31×10¹³ GC/ml), AAV5-GFAP-Ascl1-t2a-iCre (5.35×10¹³ GC/ml) and AAV5-GFAP-Ascl1^{SA6}-t2a-iCre (12.30×10¹³ GC/ml) were packaged by VectorBuilder Inc. (Chicago, IL). Each virus was diluted to deliver a total of 4.8×10⁹ GC in a 1 μL volume, delivered at 0.1 μl/min over 10 mins with a 5 μl Hamilton syringe and 30-gauge needle (Hamilton, 7803-07). The primary motor cortex of 16-week-old hSOD1^{G93A}; Rosa-zsGreen mice was targeted using a stereotaxic instrument (AP: +2.15, L/M: 1.7, DV: −1.7). Animals were anesthetized with isofluorane (2%, 1 L/min; Fresenius Kabi, CP0406V2) and administered buprenorphine (0.1 mg/kg; Vetergesic, 02342510), baytril (2.5 mg/kg; Bayer, 02249243), and saline (0.5 ml, Braun, L8001) subcutaneously. Mice were sacrificed after 28 dpi.

10× Visium spatial transcriptomic section and library preparation. Mice were anesthetized with ketamin (75 mg/kg) and xylazine (10 mg/kg) prior to dissection. Fresh brains were collected and placed in an isopentane (2-methylbutane, Sigma) bath in a benchtop liquid nitrogen container for five minutes. Frozen brains were transferred into prechilled 50 ml falcon tubes and stored at −80° C. Fresh frozen tissues were cryosectioned using a Leica CM3050 cryostat (Leica Microsystems Canada Inc., Richmond Hill, ON, Canada) at −20° C. and 10 μm coronal section was immediately placed within the framed capture areas on 10× Visium spatial slides. Slides were transferred on dry ice to the Princess Margaret Genomics Centre for further processing and spatial transcriptomic (ST) library production using Visium Spatial Tissue Optimization Reagents Kit (10× Genomics). Briefly, slides were dehydrated in −20° C. prechilled MeOH for 30 min, dried in the Thermocycler with the lid open for 1 min at 37° C. and then stained with hematoxylin and eosin. Tissue sections were exposed to 70 μl of permeabilization enzyme for 18 min, which was selected as the optimal time using the tissue optimization slide (10× Genomics), releasing the poly-adenylated mRNA that could then be captured by poly-dT primers containing Illumina TruSeq Read™, spatial barcode and UMI on the Visium slides. First and second strand cDNA synthesis and amplification were performed in the Visium slide cassette in a Thermal cycler using a Visium Spatial Gene Expression Reagent Kit (10× Genomics) as per the manufacturer's protocol. Dual Indexed, Illumina compatible libraries were created by Sample Index PCR as per the manufacturer's instructions using the Dual Index Kit TT Set A (10× Genomics) and E3, E4, E5 and E6 indexing primers. 3′ end sequencing was performed in the Princess Margaret Genomics Facility using an Illumina NovaSeq 6000™ system.

Processing of ST data. Initial data analysis was performed using 10× Genomics open software, including Space Ranger™ (version 1.3.1) and the Loupe Browser 4.0.™ bcl2fastq version 2.20, to which zsGreen and iCre sequences were added, was used to generate the FASTQ files and Space Ranger version 1.3.1 to align sequencing reads to a custom mouse genome (refdata-gex-mm10-2020-A_zsgreen_iCre). This pipeline generates a matrix of unique molecular identifier (UMI) counts for each feature-spot and performs filtering, such that only genes with 10 UMIs or more are considered. Space Ranger uses a two-component Gaussian Mixture Model to identify enriched genes that have higher mean reads per UMI. The Loupe Browser was also used to create a Spatial Map that shows the tissue image with color-coded sequencing spots that correspond to unique cellular clusters. It was also used to create a 2D T-distributed Stochastic Neighbor Embedding (tSNE) plots. tSNE projection to identify unique cellular clusters within the corresponding color-coded regions of the tissue section. Finally, the expression patterns of individual selected genes of interest were visualized using the Loupe Browser.

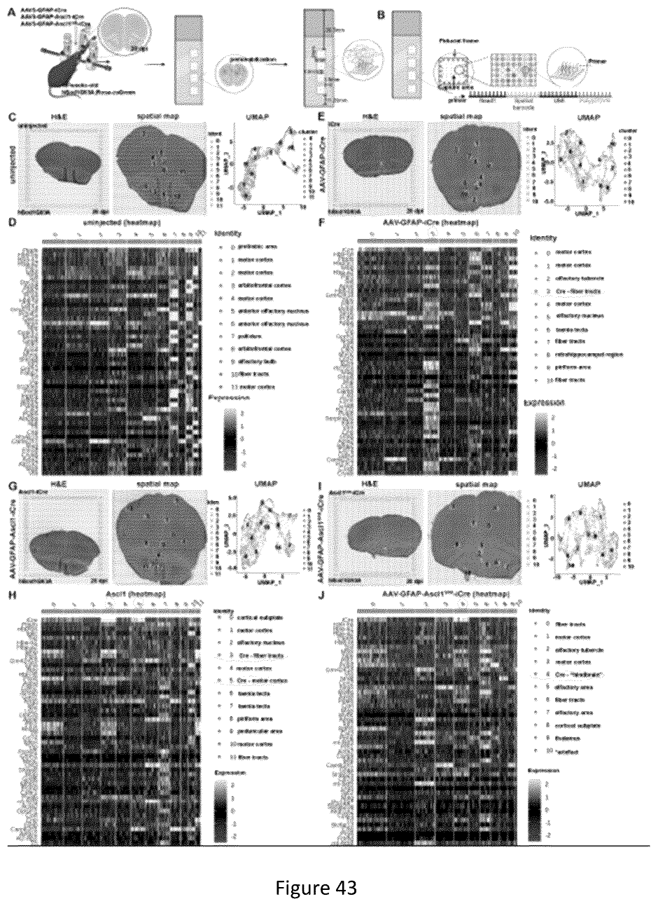

Differentially Expressed Genes (DEGs). To perform additional Features Selection and clustering analyses, a Seurat v.3.2.3 R package tool for single cell genomics¹⁰⁹ was used. Seurat was used to select highly variable genes in the dataset (2000 genes by default). These highly variable genes were then used in down-stream clustering analysis, resulting in a list of the 2000 top variable genes between all of the clusters in each brain sample. A RunUMAP function was used to generate UMAP plots with assigned clusters correlating to positions in the spatial map. Two types of differential gene expression were conducted: First, differentially expressed genes (DEGs) were examined between iCre positive cells and iCre negative cells in each of the brains by performing pair-wise comparisons, allowing identification a list of genes that are highly expressed in the iCre cluster, and not in the rest. The top 50 variable gene in each brain are shown in FIG. 43D,F,H,J. These tables consist of all 3000 genes included into the integrated data set. The integrated data set was created with SelectintegrationFeatures from Seurat. The function integrates the data set based on N most variable features, which was 3000 in the present case. Therefore, these data sets have no p-value cutoff. Secondly, DEGs restricted to iCre positive were also examined between the samples (Ascl1 vs control; Ascl1^{SA6} vs control; Ascl1 vs Ascl1^{SA6}). The p-values were calculated from individual pairwise comparisons between iCre positive cell pools.

For differential gene expression, pathway clustering was obtained using pathfindR Bioconductor package¹¹⁰. GO categories were plotted as treemaps, which reflect semantic similarities. The treemaps were prepared using the rrvgo Bioconductor package.

Cluster Annotation. Molecular annotation of the tissues represented in individual clusters was inferred using a Brain Palette™ of 266 unique genes expressed in different brain regions³⁹. Average gene expression of all spots in each assigned cluster was used to compare to regional identities using a 3D ST Viewer at github and atlas visualization from the Brain Palette study³⁹. Enrichment of GO terms in each cluster was performed by querying GO.BP and GO.CC (2021 version) with an enrichR R package, examined only differentially up-regulated genes with an adjusted p-value cutoff of 0.05¹¹¹.

Gene regulation network (GRN) inference. GRN inference was performed as described in (Okawa et al., 2015)¹¹² for iCRE-CT, Ascl1 and Ascl1^{SA6}. PKNs were assembled from MetaCore (GeneGo Inc. (15871913)), TRRUST (29087512) and RegNetwork (26424082). The Pearson correlation coefficient for each pair of TF and gene was computed over respective spatial transcriptomics sample. The intersection between the PKNs and Pearson correlation coefficient above 90 percentile was maintained. Cytoscape v.3 was used for the visualization of GRNs (14597658), in which differentially up-regulated and down-regulated genes were indicated as red or orange and blue or purple nodes, respectively, whereas non-differentially expressed genes were indicated as white nodes.

In silico ZBTB18 knockdown simulation. The differentially expressed TFs after 24 hours of ASCL1 overexpression in comparison to the 0 hour control in the neuroblastoma cell line SH-SY5Y was obtained using the dataset from (35366798). Genes were binned into two bins by the intensity and the t-test was applied to each bin using the limma R package (16646809). The p-value was corrected for multiple testing by the Benjamini-Hochberg method with an adjusted p-value cut-off of 0.05. Genes with the mean absolute fold change <2 were also discarded. The GRN among the differentially expressed TFs was reconstructed as described above, but using the scRNA-seq data of developing human cortex (26406371) for computing the Pearson correlation coefficient for each pair of TF and gene. In addition, the ZBTB18 binding target genes were obtained from (30392794) and added to the GRN. In silico perturbation was performed by constantly repressing the expression of ZBTB18. The GRN simulation was carried out by the Boolean network formalism using the majority voting logic rule. The Boolean initial expression states 1 and 0 were assigned to differentially up- and down-regulated TFs, respectively. A synchronous update was repeated 1000 times and the final GRN state was recorded. Cytoscape v.3 was used for the visualization of the GRN.

Tissue processing for immunostaining. For immunostaining, mice were anesthetized with ketamine (75 mg/kg) and xylazine (10 mg/kg) prior to perfusion. Intracardially perfusion was performed with approximately 20× of their blood volume using a peristaltic pump at a flow rate of 10 ml/min with ice cold saline (0.9% NaCl), followed by 4% paraformaldehyde (PFA) in phosphate buffer saline (PBS) for approximately five minutes. Brains were collected and post-fixed overnight in 4% PFA in PBS, transferred to a 20% sucrose/1×PBS at 4° C. overnight for cryoprotection, embedded in O.C.T. compound (Tissue-Tek® O.C.T. Compound, Sakura® Finetek, PA, USA) and stored at −80° C. Brains were frozen at −26° C. inside of a Leica CM3050 cryostat (Leica Microsystems Canada Inc., Richmond Hill, Ontario, Canada) and cut coronally at 30 μm. Sections were collected on Fisherbrand™ Superfrost™ Plus Microscope Slides (Thermo Fisher Scientific, Ottawa, ON, Canada).

Immunostaining. Slides were washed in PBS, then incubated for 1 hour at room temperature in 10% horse serum (HS), 0.075% bovine serum albumin (BSA), and 0.3% Triton-X-100 in PBS. Blocking was followed by 72 hours incubation at 4° C. in an antibody solution (10% HS, 0.75% BSA, and 0.1% Triton-X-100 in PBS) using the following primary antibodies: goat anti-GFAP (1:500, #NB100-53809, Novus), rat anti-Ctip2 (1:300, #AB18465, Abcam), rat anti-Iba1 (1:500, #019-19741, FUJIFILM Wako Pure Chemical Corporation). Slides were washed three times for 10 min each in 0.1% Triton-X-100 in PBS and incubated with species-specific secondary antibodies conjugated to Alexa568™ (1:500; Invitrogen Molecular Probes™, ThermoFisher Scientific), Alexa488™ (1:500; Molecular Probes), or Alexa647™ (1:500; Molecular Probes) at 4° C. overnight. Slides were washed three times in PBS and counterstained with 4,6-Diamidino-2-pheylindole (DAPI) (Sigma Aldrich Canada, Mississauga, ON, Canada). Finally, the slides were washed three times and mounted in Aqua-polymount (Polysciences Inc., PA, USA).

Quantification of immunopositive cells. GFAP, Ctip2 and Iba1 expressing cells were quantified using FIJI/ImageJ⁹³. In brief, cortical regions of interest were delineated using the polygon tool. For Ctip2, immunofluorescent signal was thresholded using and positive cells were quantified using the “analyze particles” command. For GFAP, immunofluorescent signal was thresholded using the adjust threshold “Triangle” algorithm and measured using the “Measure” command. For each mouse, mean intensity and percent area were evaluated as a weighted average collected across multiple sections. Microglia were analyzed in FIJI/ImageJ using the macros provided with the MORPHIOUS analysis tool⁴¹. Using this macro, Iba1 mean intensity and percent area were evaluated using an Autolocal threshold (Method: Phansalkar, parameter 1: 0, parameter 2: 0. MORPHIOUS provides functionality to quantify skeleton metrics (e.g., number of branches, branch length) and cell soma metrics (e.g., soma size). Skeleton metrics were normalized to the number of microglia somas. The nearest neighbor distance (NND), which represents the Euclidean distance for a microglial soma to its nearest neighboring microglial soma, was also evaluated. For each mouse, metrics were evaluated as a weighted average collected across multiple sections.

Imaging and statistics. Quantitative analyses were performed on matched coronal motor cortex sections at Bregma 1.34, 1.85, 2.15, which were identified using the Mouse Brain. All other images were acquired using a Zeiss Z1 Observer/Yokogawa spinning disk (Carl Zeiss) microscope. Tiled images encompassing the entire motor cortex were acquired using 30 m z-stacks with a 1 m step-size at with a 20× objective. All analysis was conducted using images at 20× magnification. Adobe Photoshop was used to make figures and a license to BioRender.com was used to prepare schematics. GraphPad Prism 9.2.0 Software was used to prepare graphs and conduct statistical tests. Mean values and error bars representing standard error of the mean (s.e.m.) are plotted. Quantification of immunostained cells was performed on three brains per condition and a minimum of three sections per brain. Comparisons were made using a One-Way ANOVA and Tukey multiple comparisons. Significance was defined as p-values less than 0.05 and denoted as follows: ns not significant, <0.05*, <0.01**, <0.001***.

### Results

Assessing Neuronal Degeneration and Glial Activation in hSOD1^{G93a} Motor Cortices

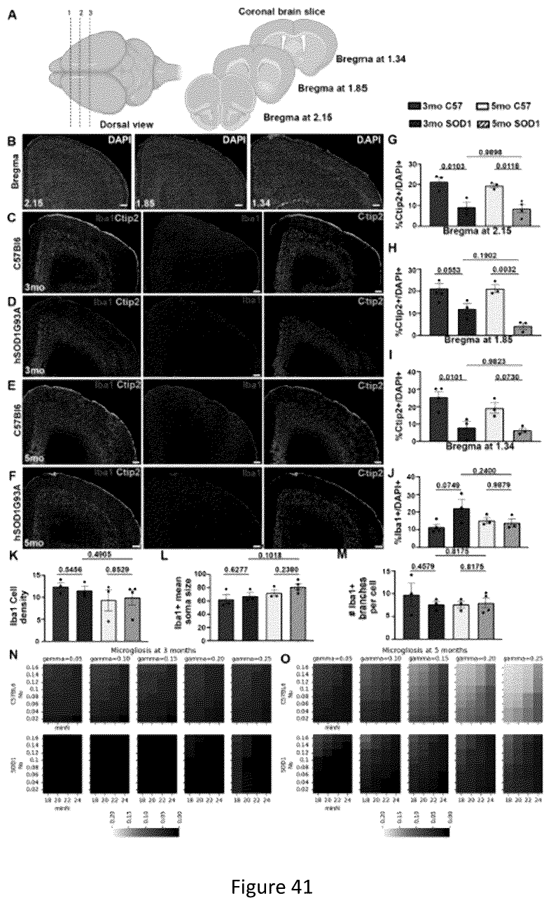

In response to a recent debate created by two high profile 2021 publications that questioned whether brain glia (astrocytes, microglia) can be converted to neurons in vivo^{16,27}, the Example set out to spatially map the transcriptional impact of neuronal reprogramming TFs on adult brain astrocytes in vivo. It was reasoned that neuronal reprogramming might be most efficient in a state of neurodegeneration when there is a need to replace lost neurons. Neuronal reprogramming vectors were therefore delivered to the motor cortex of hSOD1^{G93A} mice, a model of ALS in which deep-layer pyramidal neurons in the motor cortex have been reported to degenerate by one-month of age³⁷. To confirm neuronal loss, Ctip2 was used to immunolabel projection neurons in cortical layers V and VI⁴⁰. Compared to C57 control brains, fewer Ctip2⁺ neurons were detected in the hSOD1^{G93A} motor cortex at three anterioposterior positions (Bregma 1.34, 1.85, 2.15) at both 3- and 5-months of age (FIG. 41A-I).

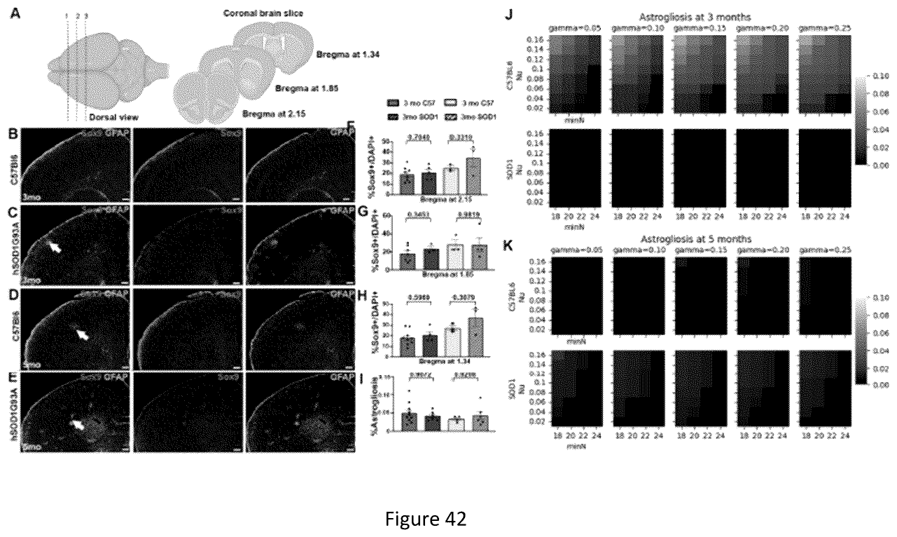

As neuronal degeneration is often associated with glial activation, MORPHIOUS, a custom machine learning software package, was used to identify and quantify activated microglia and astrocytes⁴¹. Using this method, the number of Iba1⁺ microglia, their density, soma size and branch number were similar between C57 and hSOD1^{G93A} mice at 3-month and 5-months of age (FIG. 41J). Indeed, by using MORPHIOUS-based tuning to optimally identify activated cells, more “activation” was apparent in the baseline control dataset (C57B/6) compared to hSOD1^{G93A} mice, indicating that there is not an increase in activated microglia clusters in hSOD1^{G93A} cortices (FIG. 41N,O). A previous report similarly did not find an increase in microglial number in hSOD1^{G93A} mice⁴², although they did find morphological alterations that were not detected using MORPHIOUS⁴¹. Reactive gliosis was also assessed, but no sign of a proliferative response was evident as similar numbers of Sox9′ astrocytes were detected in C57 control and hSOD1^{G93A} motor cortices at both 3- and 5-months of age (FIG. 42A-H). Moreover, an examination of GFAP staining using MORPHIOUS-based tuning to optimally identify activated cells similarly revealed more “activation” in baseline control mice (C57Bl/6) compared to hSOD1^{G93A} mice, suggesting there is not a substantial increase in the clustering of activated astrocytes in hSOD1^{G93A} cortices (FIG. 42I-K). Thus, despite the degeneration of Ctip2⁺ pyramidal neurons in hSOD1^{G93A} motor cortices at 3- and 5-months of age, glial reactivity above baseline levels was not detected.

Targeting hSOD1^{G93a} Motor Cortices with Neuronal Reprogramming Vectors

To assess the transcriptional impact of expressing reprogramming TFs in astrocytes in vivo, adeno-associated viruses (AAVs) carrying a short human GFAP promoter was used to drive the expression of native Ascl1 or mutated Ascl1^{SA6}, both tethered to a t2a-iCre cassette, which was also in the control vector. Notably, in Example 5, using these constructs, it was demonstrated that Ascl1^{SA6}, and to a lesser extent Ascl1, can increase neuronal marker expression when expressed in motor cortex astrocytes in Rosa-zsGreen reporter mice, which do not undergo neuronal degeneration. In this Example, AAVs were instead injected in 4-month-old hSOD1^{G93A}; Rosa-zsGreen mice, at a stage when neurodegeneration had already ensued, and behavioural symptoms had just initiated⁴² (FIG. 43A). Ascl1^{SA6} carries six serine-to-alanine mutations that enhance the capacity of this bHLH TF to convert embryonic cortical neural progenitor cells to neurons¹⁹ and to induce neuronal marker expression and reduce glial marker expression in adult brain astrocytes in vivo²⁶ and in glioblastoma²³ and neuroblastoma²⁵ cells in vitro. Ascl1 and Ascl1^{SA6} were linked to a t2a-iCre sequence so that transduced cells could be detected in Rosa-zsGreen reporter mice. Sequences were cloned and packaged in AAV5 to generate the following three gene delivery vectors: AAV5 GFAP-iCre (hereafter, iCre control), AAV5-GFAP-Ascl1-t2a-iCre (hereafter, Ascl) and AAV5-GFAP-Ascl1^{SA6}_t2a-iCre (hereafter, Ascl1SA6).

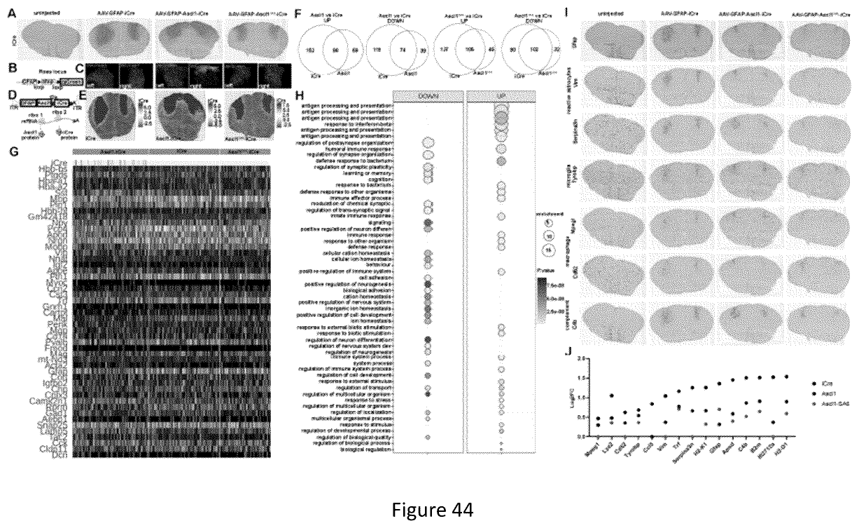

Each of the three AAVs were delivered using stereotaxic coordinates to the motor cortex (AP: +2.15, L/M: ±1.7, DV: −1.7) of 4-month-old hSOD1^{G93A}; Rosa-zsGreen mice. Brains were harvested 28 days later, at 5-months of age, and fresh frozen brains were sectioned and examined for zsGreen expression (FIG. 43A). When maximal zsGreen expression was detected in the motor cortex, a single section from each brain (uninjected, iCre, Ascl1, Ascl1^{SA6}) was collected on one of the four capture windows in a Visium spatial transcriptomic (ST) slide (FIG. 43A). Slides were permeabilized to release poly-adenylated mRNA, which was captured by the barcoded oligo-dT primers in the capture windows (FIG. 43B). Hematoxylin and eosin (H&E) staining confirmed that sections collected from each of the four brains were within the motor cortex, albeit at slightly different rostrocaudal positions (FIG. 43C,E,G,I). At this level of resolution, sites of AAV injection were not readily visible in the H&E-stained tissue, but as zsGreen expression was evident (see FIG. 44C), viral transduction was successful.

### Computational Analysis and Quality Check of Spatial Transcriptomic Data

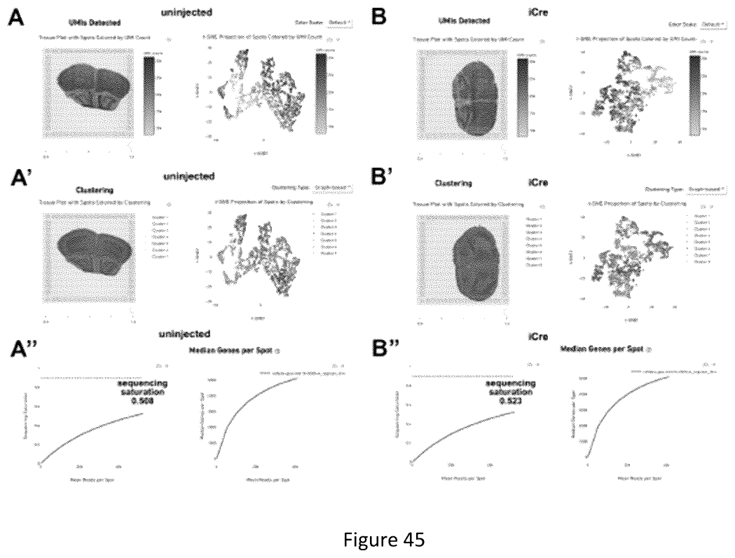

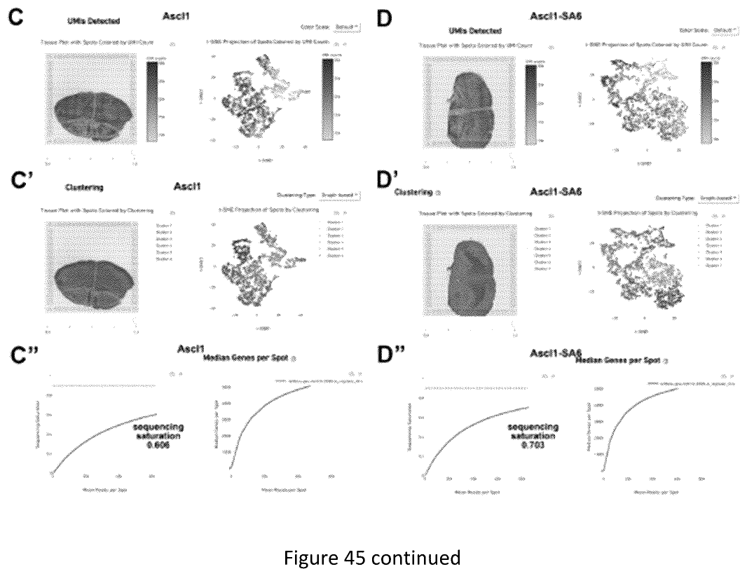

ST libraries were generated and sequenced, revealing similar levels of raw sequence read saturation from the uninjected (0.508), control iCre (0.523), Ascl1 (0.606), and Ascl1^{SA6} (0.703) injected brains (FIG. 45A″-D″). Sequenced reads were filtered, annotated and normalized, leading to the identification of 19,925, 20,200, 20,485 and 20,241 expressed genes across 2,079, 2,348, 2,334 and 2,285 individual capture locations (spots) in uninjected, iCre, Ascl1, and Ascl1^{SA6} injected brains, respectively (FIG. 45). Empty spots not covered by tissue were excluded from analysis. A preliminary analysis of data quality was performed using automated 10× Visium clustering algorithms, creating spatial graph-based maps and 2D T-distributed Stochastic Neighbor Embedding (tSNE) projections (FIG. 45A-D,A′-D′). Unique molecular identifier (UMI) counts were high in all cell clusters (10,000-30,000 range/spot), confirming the high depth of sequencing (FIG. 45A-D). At this level of resolution, 7-8 transcriptionally unique cell clusters were identified and mapped with a color code onto corresponding spatial positions in each of the sequenced brains. In the uninjected brain, 7 distinct cellular clusters were detected based on the similarity of their transcriptomes, (FIG. 45A′). In contrast, in iCre-injected brains, 8 transcriptionally distinct cell clusters were detected, with the additional “new” cluster having a spatial location that matched the site of AAV injection in the motor cortex (annotated as cluster 7; FIG. 45B′). Surprisingly, additional clusters matching the site of injection were not similarly observed in the Ascl1 (6 clusters; FIG. 45C′) and Ascl1^{SA6} (7 clusters; FIG. 45D′) injected brains, suggesting that the transcriptomes of the cells in these sequenced ‘spots’ were more similar to endogenous brain tissue than the iCre transduced cells.

### Assigning Cluster Identities and Defining the Spatial Distribution of AAV Transduction

To better distinguish cell clusters in the ST data, Seurat was used to select highly variable genes for downstream clustering analysis. For this purpose, iCre and zsGreen were added to the reference mouse genome (refdata-gex-mm10-2020-A_zsgreen_iCre) so that cell clusters associated with AAV transduction could be assigned. Using the 2000 top variable genes between all clusters, high dimensional uniform manifold approximation and projection (UMAP) plots were generated, identifying 10-11 unique cellular clusters for each brain (FIG. 43C,E,G,I). With the increased resolution and stratification of the cell populations in the UMAP projections, clusters of cells corresponding to the iCre-transduced areas were now evident in the three transduced brains (FIG. 43F,H,J). Regional identities were assigned to each cluster using the Allen mouse brain atlas to identify anatomical positions (www.brain-map.org).

As expected, high iCre-expressing clusters were positioned within the motor cortex in the iCre (cluster 3, FIG. 43F), Ascl1 (Cluster 3, and partly cluster 5; FIG. 43H) and Ascl1^{SA6} (cluster 4; FIG. 43J) transduced brains. Indeed, iCre was at the top of the 50 most variable genes across the tissue, as displayed in heatmaps for each of the transduced brains (FIG. 43F,H,J). Surprisingly, zsGreen transcripts were not detected, even though the mRNA sequence was added to the reference genome, and zsGreen epifluorescence was seen in each of the three AAV-injected brains (see FIG. 44C). Therefore, iCre expression was relied on exclusively to identify the cell clusters transduced with the AAV vectors.

As an independent measure of the veracity of the molecular data, differentially expressed genes (DEGs) were compared within each given cluster (as compared to all other clusters) to a brain palette of 266 genes defined in a molecular brain atlas³⁹. As expected from the anatomical assignments, most clusters in all four brain sections were molecularly identified as “isocortex”, “olfactory areas” or “fiber tracts”. A few mis-identified hindbrain clusters and unidentified (NA) assignments were obtained, but in general, transcriptional programs corresponded to the anatomical brain regions of our collected tissue. Notably, for high iCre expressing clusters in the iCre control and Ascl1-transduced brains, the molecular assignment was “fiber tract”, while the iCre cluster in the Ascl1^{SA6} transduced brain was assigned a “hindbrain identity”, either reflecting a mis-assignment, or possibly reflecting the unique nature of the transcriptional program initiated by this TF (FIG. 43D,F,H,I).

Taken together, these data confirm that the collected ST data is of high quality and well represent the transcriptomes of brain cells in situ.

Distinct Transcriptional Responses to iCre, Ascl1 and Ascl1^{SA6} Transduction in Vivo

In the iCre control-transduced brains, unsupervised clustering identified a total of 10 clusters, with cluster 3 enriched in iCre expression mapping spatially onto the targeted injection site in the motor cortex (FIG. 43E,F). Strikingly, many of the top 50 variably expressed genes across the iCre transduced brain were associated with an inflammatory and/or neurodegenerative response (e.g., Apod, Trf, B2m, Ifi2712a, Ccl5, C4b, Serpina3n, Gfap, Vim, C1qb, H2-K1, H2-D1) (FIG. 43F), as described further below. Comparatively, the control uninjected brain had 11 cellular clusters, one more than in the iCre injected brain, reflecting the slightly more rostral positioning of this brain section (FIG. 43C). Indeed, the top-most variably expressed genes in the uninjected brain were primarily in olfactory regions of the spatial map (clusters 5-7,9,10; FIG. 43D), which were less prevalent in the iCre-transduced brain (FIG. 43F). Despite being in a state of neurodegeneration, in the non-injected hSOD1^{G93A} motor cortex, only two of the inflammatory genes observed in the iCre-transduced brains were among the top 50 variable genes; Apolipoprotein d (Apod), a lipid carrier upregulated in the brain of many neurodegenerative diseases^{43,44}, and Transferrin (Trf), involved in iron uptake, and upregulated in microglia in response to pro-inflammatory signals⁴⁵. Thus, the AAV control vector induced an inflammatory transcriptional response that was above and beyond any degenerative signals seen in uninjected 5 mo-old hSOD1^{G93A} motor cortices (FIG. 43D).

Next the spatial transcriptomes of the Ascl1- and Ascl1^{SA6}-transduced brains were analyzed, which were stratified into 11 and 10 cell clusters, respectively (FIG. 43G,I). In the Ascl1-transduced brain, sequenced spots enriched in iCre expression fell within two clusters (clusters 3 and 5), straddling two transcriptionally distinct regions, both of which spatially mapped onto different domains of the motor cortex (FIG. 43G). In the Ascl1^{SA6} transduced brain, high iCre expression fell within a single cluster 4 (FIG. 43I). Regardless of the spatial differences of the two injection sites, a striking lack of inflammatory gene expression was observed among the top 50 variably expressed genes after Ascl1 or Ascl1^{SA6} transduction (FIG. 43H,J). Indeed, only Apod and Trf, also elevated in the uninjected control hSOD1^{G93A} brain, as well as Gfap, were among the variable gene sets associated with Ascl1 or Asc1^{SA6} transduction (FIG. 43H,J). Instead, several neuronal-specific genes, such as Mbp and Pvalb, were among the top 50 variably expressed genes that were enriched in the iCre expressing cell clusters in Ascl1 and Ascl1^{SA6} transduced brains (FIG. 43H,J).

Taken together, these data show that AAVs expressing Ascl1 or Ascl1^{SA6} elicit a distinct transcriptional response in the target tissue compared to the iCre control vector.

Identification of Differentially Expressed Genes within the AAV-Transduced Cell Clusters

To perform a broader comparison of DEGs in the AAV-transduced spots, the next step in the study focused on cell clusters enriched in iCre expression in the control iCre, Ascl1 and Ascl1^{SA6} transduced brains. Spatial mapping of iCre expression confirmed that iCre was indeed expressed in the motor cortex of the brains transduced with the three vectors (FIG. 44A). Furthermore, iCre transcripts were translated into functional protein as zsGreen was expressed in the three injected brains, indicating that the Rosa-zsGreen locus was recombined (FIG. 44B,C). Notably, for downstream computational analyses, iCre mRNA was used as a surrogate measure of Ascl1 transcripts as the two genes are transcribed from a single, bicistronic message separated by a t2a peptide sequence that promotes ribosome skipping and effective translation of two proteins⁴⁶ (FIG. 44D). As 3′end sequencing was performed in this study, only iCre (the 3′ gene) was detected in the sequencing data set.

Next, gene expression was normalized and scaled to isolate the DEGs that were specifically associated with the iCre transduced cells (FIG. 44E). A series of Venn diagrams were used to compare DEGs that were up- or down-regulated in iCre⁺ cells in the three transduced brains (FIG. 44F). When comparing up-regulated DEGs in Ascl1 or Ascl1^{SA6} versus iCre transduced brains, there were 2-3-fold more DEGs uniquely upregulated in the iCre transduced control brains (137-152 genes) compared to the uniquely upregulated DEGs in Ascl1 (69 genes) or Ascl1^{SA6} (32 genes) transduced brains (FIG. 44F). A similar bias was observed when comparing downregulated DEGs in each of the injected conditions; 90-118 genes were downregulated in the control iCre-transduced brain relative to a much lower number of DEGs after Ascl1 (39 genes) or Ascl1^{SA6} (32 genes) transduction (FIG. 44F). Regardless of these differences in the numbers of DEGs, in each condition, the transduced iCre⁺ cells clearly expressed unique sets of genes relative to the surrounding tissue.

iCre Control Transduced Cells Express Elevated Levels of Inflammatory Markers

To characterize the impact of AAV transduction in vivo in more detail, for each sample, a heatmap was generated of the top 50 DEGs that are highly expressed in the iCre clusters, and not in the rest of the tissue (FIG. 44G). The molecular signature associated with the transduction of the iCre control vector was first assessed by performing gene ontology (GO) analysis of the DEGs (FIG. 44H). Semantic similarity based treemap plots were used to image the GO terms, which allow hierarchical structures to be visualized, with the size of the space proportional to the GO score. Strikingly, top GO categories associated with control iCre transduction included several immune system-specific terms, including “defense response to other organism”, “positive regulation of immune system”, “immune system process”, “antigen processing and presentation of peptide antigen”, “macrophage activation” and “microglial cell activation” (FIG. 44H). In addition, another branch of upregulated GO terms were associated with neuronal development, function and maturation, such as “regulation of trans-synaptic signaling”, “modulation of chemical synaptic transmission”, “regulation of neurogenesis”, “axon development” and “synapse pruning” (FIG. 44H).

To assess this inflammatory response further, the expression of signatory genes was mapped onto the spatial maps (FIG. 44I). Markers of reactive astrocytes were examined, including Gfap and Vimentin⁴⁷, both of which were clearly upregulated in the vicinity of the iCre-transduced patches of all three injected brains (FIG. 44I). A similar enriched expression in the injection sites was observed for Lyz2⁴⁸ and Tyrobp⁴⁹, markers of activated microglia, Mpeg1⁵⁰ and Cd52⁵¹, macrophage markers, and C4b, a complement factor⁵². In all cases, enriched gene expression was not seen in the uninjected control brain and was most notable in the bilateral injection sites for iCre, and to a lesser extent for Asc1l and Ascl1^{SA6} (FIG. 44I). The increase in relative expression of inflammatory genes in the iCre transduced brain compared to the Ascl1 and Ascl1^{SA6} injections was confirmed by plotting Log 2 fold-changes (FC) in gene expression comparing the iCre transduced cluster to other sequenced spot in the brains (FIG. 44J).

Taken together, these data indicate that there is a striking pro-inflammatory immune response in the iCre transduced cells, along with a deregulation of neuronal genes that may disrupt neuronal function.

Ascl1- and Ascl1^{SA6}-Transduced Cells Express Genes Associated with Neuronal-Specific Gene Ontology Categories

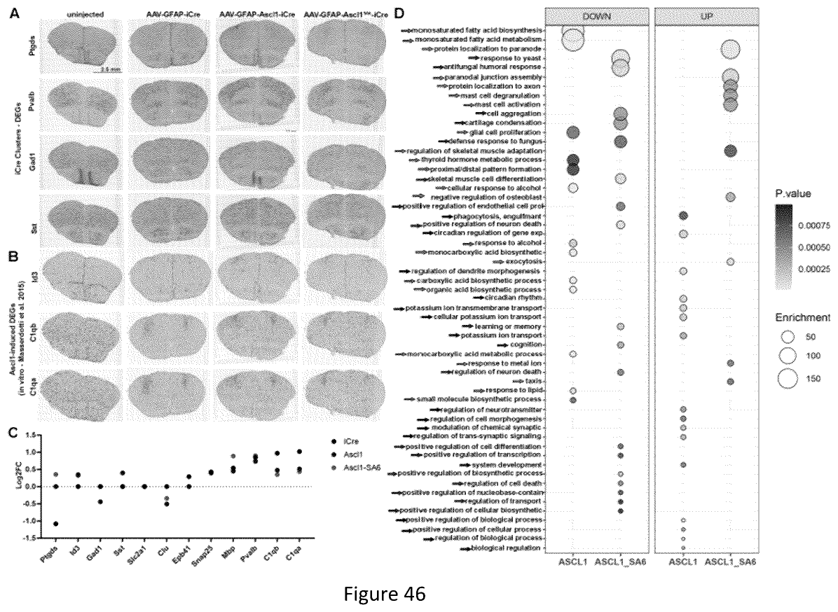

Next spatial maps of gene expression were generated for a subset of the top 50 DEGs that were expressed at elevated levels in the Ascl1- and Ascl16-transduced spots (i.e., iCre⁺) compared to the rest of the tissue (FIG. 44G). One of the most variably expressed genes was prostaglandin D2 synthase (Ptgds), which synthesizes PGD2, a neuroprotective prostaglandin that is upregulated in astrocytes and oligodendrocytes in dymyelinating diseases as a stress reaction⁵³. In addition, GABAergic neuronal markers, such as Pvalb, Gad1 and Sst, which are induced by Ascl1 expression in postnatal astroglia in vitro¹³, were among the top 50 DEGs (FIG. 44G). However, while spatial mapping of Ptgds, Pvalb1, Gad1 and Sst revealed unique enriched expression patterns for each gene, expression differences in the injection sites compared to the rest of the tissue were not readily discernable in each of the uninjected, iCre, Ascl1 and Asc1^{SA6} injected brains, likely due to the relatively high expression of each of these genes in cortical tissue in general (FIG. 46A). Indeed, these spatial maps do not reflect actual gene expression, but rather the enrichment of gene expression in one part of the tissue versus all other sites. To provide a quantitative measure of gene expression, we thus plotted log 2FC in gene expression, revealing that Ptgds, Sst and Mbp were all expressed at elevated levels in Ascl1- or Ascl1^{SA6}-transduced brains compared to control iCre injected brains.

Next, the expression of a handful of DEG identified in a prior in vitro neuronal reprogramming experiment targeting early postnatal astrocytes¹⁰ were similarly spatially mapped. While most of the neuronally-expressed genes were not noticeably altered in the AAV-transduced brains (data not shown), a handful of genes were identified that showed enriched expression in the site of AAV transduction, including Id3, which encodes an HLH TF that lacks a basic domain and binds to and prevents the activity of bHLH factors (FIG. 46B). Notably, Id3 maintains the regulatory T-cell pool⁵⁴, but it is also a critical regulator of neurogenesis⁵⁵. In addition, two components of the complement cascade, C1qb and C1qa, were upregulated both during in vitro neuronal reprogramming, and in the injection site in all three brains (FIG. 46B). To provide a quantitative measure of gene expression, we plotted log 2FC in gene expression, revealing that Id3, was expressed at elevated levels in Ascl1- or Ascl1^{SA6}-transduced brains compared to control iCre injected brains, whereas C1qb and C1qa levels were expressed at comparatively higher levels in the iCre-transduced brains (FIG. 46C). These findings were consistent with the elevated inflammatory response associated with the control iCre vector.

Finally, to better understand the transcriptional impact of the reprogramming TFs, GO analysis of the DEGs in the Ascl1 and Ascl1^{SA6} transduced brains was performed. Upregulated GO terms in the Ascl1-transduced brains included several neuronal terms, including “potassium ion transport”, “regulation of dendrite morphogenesis” and “modulation of chemical synaptic transmission” (FIG. 46D). Strikingly, a different set of GO terms were enriched in Ascl1^{SA6} transduced brains, suggesting that these two TFs have distinct effects. Included were neuronal categories, such as “protein localization to axon and junctional assembly”, along with general GO terms, such as “negative regulation of cell differentiation” (FIG. 46F).

Also of interest were the GO terms that were downregulated upon Ascl1 transduction, which included “glial cell proliferation”, consistent with a potential loss of glial identity. In addition, “metabolic process” was a downregulated GO term associated with Ascl1 expression (FIG. 46E). This finding is of potential interest given that metabolic gene expression changes during neural progenitor cell differentiation⁵⁶, and furthermore, several metabolic genes are differentially expressed in neuroendocrine tumors that are classified as ASCL1^{low} and ASCL1^{High 57}. Finally, in the Ascl1^{SA6} transduced brains, a different set of genes were downregulated, belonging to GO categories including “positive regulation of cell death”, suggesting Ascl1^{SA6} transduced cells may have an enhanced survival phenotype, and “cell differentiation” (FIG. 46G).

Taken together, GO analyses are consistent with Ascl1 and Ascl1^{SA6} inducing changes in differentiation pathways, and astrocytic and neuronal specific gene expression, prompting a more detailed analysis of the induced transcriptomes.

Inflammatory Signature of the Gene Regulatory Network Associated with Control AAV Transduction

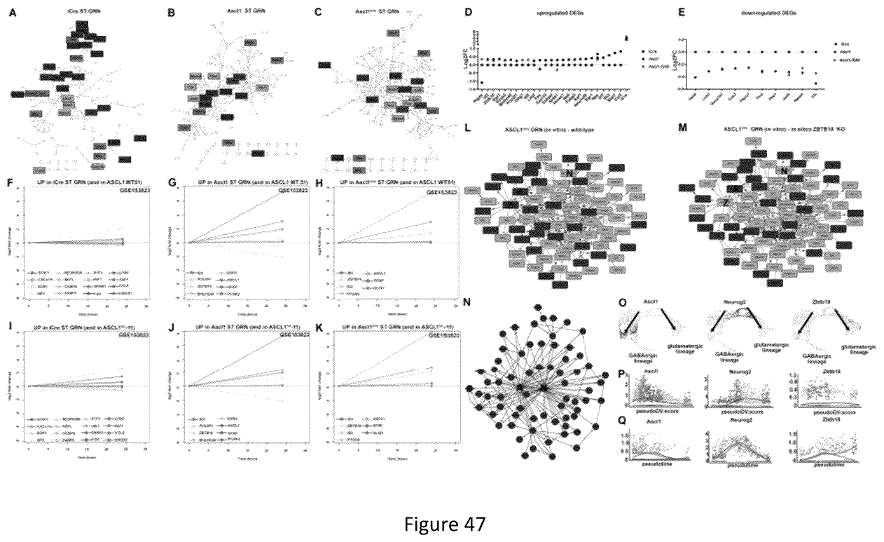

Gene regulatory networks (GRNs) provide a systems level view of how genes interact to confer cell fates and/or states, and can be used to identify critical TF hubs. Using the list of expressed genes within each iCre⁺ cell cluster, we inferred GRNs for control, Ascl1 and Ascl1^{SA6} transduced cells. The three GRNs had distinct topologies and each included genes that were significantly up- or down-regulated compared to each other (adjusted p-value <=0.05). Genes induced by iCre and one or both of Ascl1 or Ascl1^{SA6} are likely related to AAV delivery rather than the specific gene cargo. Commonly upregulated genes included Gfap, an inflammatory astrocytic marker, Nfic, a TF required for Gfap expression⁵⁸, Satb2 and Tbr1, layer-specific neuronal markers⁵⁹, Mbp, a marker of myelination that is expressed at reduced levels in neurodegenerative diseases⁶⁰, and Ctsb, a lysosomal protease expressed in microglia and astrocytes that is associated with neuroinflammation⁶¹ (FIG. 47A-C). However, as shown in FIG. 2J, log 2FC for inflammatory genes such as Gfap were highest in the iCre-transduced cells. Similarly some DEGs were specifically downregulated by iCre and at least one of the Ascl1 or Ascl1^{SA6} vectors, including Hopx⁶², Id4⁶³, Bhlhe22⁶⁴ and Meis2⁶⁵ (FIG. 47A-C).

Taken together, these data further support the idea that AAV transduction alone can alter gene expression independent of cargo. However, as the goal was to identify cargo- (i.e., bHLH TF)-specific effects, this study instead focused on genes that were uniquely upregulated by iCre or Ascl1 and/or Ascl1^{SA6}.

In the iCre GRN, several genes were uniquely upregulated, including pro-inflammatory genes such as Stat1, a cytokine regulated TF⁶⁶, Irf7, a TF regulator that forms a positive feedback loop with type 1 interferons⁶⁶, Cxcl1, a neutrophil attracting cytokine⁶⁷, Ccl5, a pro-inflammatory chemokine⁶⁸, Nr4a1, a nuclear receptor upregulated in activated microglia following ischemic insult⁶⁹, Egr1, an oxidative stress responsive TF⁷⁰, Fabp5, which regulates blood-brain-barrier permeability⁷¹, Litaf, a TF regulator of the inflammatory response⁷², and Cepbd, a TF that increases Sod1 expression and oxidative stress in astrocytes⁷³ (FIG. 47A). Interestingly, Neurod6 was also upregulated by iCre transduction, a bHLH gene that protects against oxidative stress by conferring cellular tolerance⁷⁴, which may be a response to the pro-inflammatory state induced by AAV transduction (FIG. 47A). These differences in gene expression relative to Ascl1 and Ascl1^{SA6} transduction were confirmed by plotting log 2FCs (FIG. 47D). Conversely, a set of DEGs was specifically down regulated by iCre 7 including the neural stem and progenitor cell (NSPC) markers Lhx2⁷⁵, Hes5⁷⁶, and Cux2⁷⁷, and Ptprz1, a susceptibility gene for schizophrenia^{25,78} 7 (FIG. 47A), also validated in log 2FC plots (FIG. 47D).

Taken together, these data are consistent with the pro-inflammatory GRN associated with transduction of the control iCre vector, and suggest other possible detrimental effects on neuronal function, consistent with prior reports of AAV transduction in vivo altering dendritic patterning⁷⁹ and hippocampal neurogenesis⁸⁰.

Gene Regulatory Networks Associated with Ascl1 and Ascl1^{SA6} Transduction Include Upregulated Neuronal and Downregulated Glial Genes

Ascl1 and Ascl1^{SA6} GRNs differed substantively from the iCre GRN, but also displayed some similarities to each other, including enriched expression of Ascl1 itself, along with Zbtb18 and Id2 (FIG. 47B,C). Zbtb18 is a BTB/POZ-domain, zinc-finger transcriptional repressor, the mutation of which is associated with abnormal neocortical development, including microcephaly or macrocephaly, intellectual disability and epilepsy⁸¹. Notably, Zbtb18 regulates the expression of Id family TFs (Id1-4)⁶³, and Id3 expression was also elevated in the Ascl1^{SA6} GRN (FIG. 47C). These differences in gene expression relative to iCre transduction were confirmed by plotting log 2FCs (FIG. 47D).

Thereafter, the two GRNs differed, with genes elevated in the Ascl1 GRN including Egr3, which plays a role in neurite outgrowth⁸², Pcsk2, a prohormone convertase required for neuropeptide biosynthesis⁸³, Bhlhe40, a bHLH TF, the loss of which leads to cortical hyperexcitability⁸⁴, and Pou4f1, required to maintain mature postmitotic neurons⁸⁵ (FIG. 4B,D). In contrast, Ascl1^{SA6} additionally increased expression of Ptgds, a neuroprotective factor³, and Olig1, a bHLH TF that promotes oligodendrocyte differentiation⁸⁶, as confirmed in log 2FC plots (FIG. 4C,D). Thus, both Ascl1 and Ascl1^{SA6} have GRNs that share some common TF nodes (Zbtb18, Id3), but also differ in several respects.

Next gene expression that was downregulated Ascl1 or Ascl1^{SA6} GRNs (FIGS. 4B,C) was examined. Included in both datasets were genes expressed in neurons and/or glia, including Npas4, a neuroprotective bHLH-PAS domain TF, the loss of which activates cortical microglia and astrocytes⁸⁷, Thra, a thyroid hormone nuclear receptor, which when knocked-out promotes precocious neurogenesis⁸⁸, Junb, an immediate early gene, the expression of which is associated with short-term neuronal activity⁸⁹, and Clu, or clusterin, also known as ApoJ, a gene expressed in astrocytes and in presynaptic terminals, the overexpression of which promotes excitatory neurotransmission and reduces AD risk⁹⁰. Pbx1, a terminal selector gene⁹¹ was also downregulated in the Ascl1 GRN (FIG. 47B,E), while Fos, also an immediate early gene⁸⁹, and Nr4a1, associated with activated microglia, were additionally downregulated in the Ascl1^{SA6} GRN (FIG. 47C,E). While the significance of these changes might not be immediately apparent, the overlap in the transcriptional response between Ascl1 and Ascl1^{SA6} provides support for these TFs inducing transcriptional changes in vivo that are different than those induced by the control vector alone.

Taken together, this data demonstrates that there are both common and distinct features of the GRNs activated by Ascl1 and Ascl1^{SA6} when mis-expressed in the motor cortex of hSOD1^{G93A} mice.

Astrocytes Transduced with Ascl1 and Asc1^{SA6} In Vivo Activate a Subset of in Vitro Target Genes

To better understand the significance of the changes in gene expression associated with the expression of Ascl1 and Ascl1^{SA6} in brain astrocytes in vivo, the DEGs from the present ST analysis were compared to previously identified DEGs from two in vitro reprogramming studies. Comparisons were made to a study in which a 4-hydroxy-tamoxifen inducible Ascl1^{ERT2} retroviral construct was transduced into proliferating astrocytes isolated from postnatal day (P) 6-7 cortices in vitro, followed by RNA-seq analyses at 4-, 24- and 48-hr post-transduction¹⁰. In study two, human ASCL1 and mutant ASCL1^{SA6} (human gene has only 5 SP sites) were overexpressed in a neuroblastoma cell line (SH-SY5Y) using a doxycycline-inducible lentiviral system, with gene expression assessed only at 24-hr post-transduction²⁵. While neither system exactly mimicked the adult astrocytes target in the present study, in both instances, neuronal differentiation was triggered.

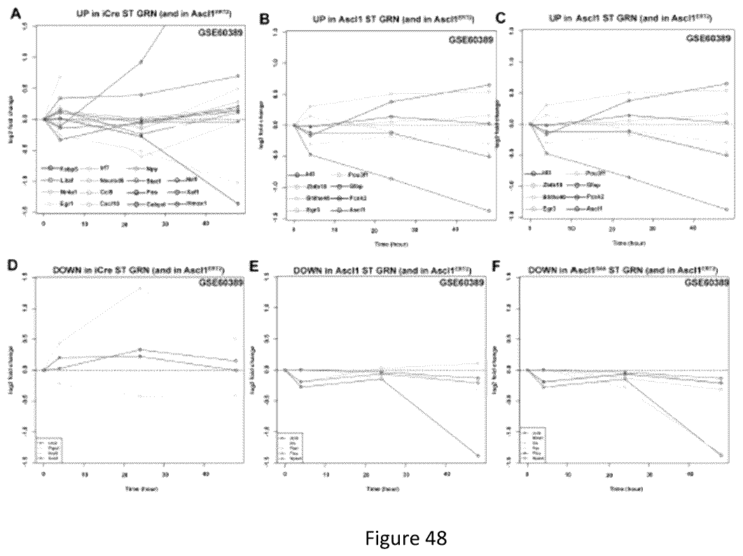

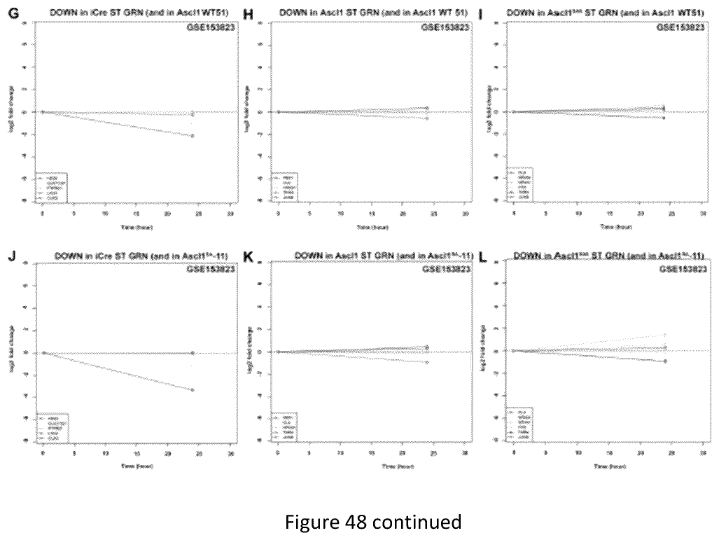

Strikingly, several of the pro-inflammatory genes induced by AAV5-GFAP-iCre transduction in the motor cortex of ALS mice in vivo (this study) were similarly transiently upregulated by in vitro retroviral transduction of Ascl1, including Irf7, Hmox1, Cxcl1, Ccl5, Stat1, Litaf, Xaf1 and Egr1 at the 4-hr time point (FIG. 48A). However, this response was transient, with only Hmox1 expression persisting at 24 hr and 48 hr post-transduction, and Nefl also increasing in expression at these later time points (FIG. 48A). NEFL encodes a neurofilament light chain protein that forms neuronal intermediate filaments, and loss-of-function mutations are associated with neurodegeneration in Charcot-Marie-Tooth disease⁹². The upregulation of Nefl may thus be a neuroprotective response to viral transduction in the brain (our study), and in vitro in brain astrocytes cultured in a dish¹⁰. Hemoxygenase 1 (Hmox1) similarly offers neuroprotection and is normally downregulated in ALS⁹³. A smaller number of these genes were also induced by ASCL1 (EGR1, NR4A1, LITAF, NEFL) and ASCL1^{SA6} (EGR1, NR4A1, LITAF) at 24 hr post-transduction of neuroblastoma cells²⁵ (FIG. 47D,G).

A subset of genes was also downregulated by control AAVs in our ST analysis in vivo and the retroviral and lentiviral vectors used in in vitro neuronal reprogramming studies. Included were Ptprz1, which was downregulated by Ascl1^{ERT2} expression in postnatal astrocytes¹⁰. Ptprz1 encodes a multifunctional receptor protein tyrosine phosphatase that is expressed in neurons and glia and is a susceptibility gene for schizophrenia⁷⁸. Ptprz1 may also participate in the immune response as it serves as a receptor for Interleukin-34 (IL-34)⁹⁴, a pro-inflammatory cytokine⁹⁵. In addition, the neurogenic gene Cux2, as well as Gucy1b3, a guanylyl cyclase subunit not yet studied in the brain (FIG. 47G,J).

Thus, control viral transductions have shared transcriptional profiles, whether transduced in vitro or in vivo, and irrespective of the viral vector source.

### Zbtb18 is an Essential Node TF in Ascl1 GRNs

Next the DEGs identified in the Ascl1 and Ascl1′6 GRNs in this study in vivo were compared to the in vitro neuronal reprogramming studies. Strikingly, the expression of Ascl1^{ERT2} in postnatal astrocytes, or ASCL1 and ASCL1^{SA6} in neuroblastoma cells, all upregulated the expression of Zbtb18 and Id3 (FIG. 47G,J, FIG. 48B,C). In addition, Egr3 and Pcsk2 were upregulated by Ascl1^{ERT2} in P5 astrocytes (FIG. 48B,C) and by ASCL1 and ASCL1^{SA6} in neuroblastoma cells (FIG. 47G,H,J,K). In contrast, Id3 expression was induced only by Ascl1^{SA6} in the present study in vivo, and by ASCL1^{SA6} in neuroblastoma cells in vitro (FIG. 47H,K). Zbtb18 and Id2 are the two genes that are commonly upregulated by Ascl1 and Ascl1^{SA} mutants in both studies, but given the link between ZBTB18 mutations and cortical malformations⁸¹, the present study further focused on this TF.

To further investigate the potential significance of ZBTB18 in driving neuronal conversion downstream of ASCL1, a cortical GRN was inferred using scRNA-seq data from embryonic human cortex⁹⁶, and genes that were either up- (dark grey boxes) or down-regulated (light grey boxes) 24 hr after ASCL1^{SA5}-overexpression in glioblastoma cells²³ were mapped onto this GRN (FIG. 47L). With this network analysis, ZBTB18 was identified as a transcriptional target of ASCL1, while NEUROG2 expression was repressed (FIG. 47L), in keeping with the cross-repressive interactions between these two bHLH TFs⁴. To assess the significance of ZBTB18 expression, an in silica knock-out (KO) analysis was performed, which revealed that of the 82 TF nodes, 22 TFs (or 26.8% of all TFs) were projected to have altered expression, including ID3 and EGR3, also induced by Ascl1 overexpression in the present study (FIG. 47M).

To provide further support for the presence of Ascl1, Neurog2 and Zbtb18 within the same cortical GRN, a GRN previously produced from embryonic day (E) 12.5, Neurog2 and Ascl1 expressing cortical neural progenitor cells (NPCs)⁹⁷ was re-analyzed, which revealed that Zbtb18 was indeed part of this transcriptional network (FIG. 47N). Additional insights into the relationship between Ascl1, Neurog2 and Zbtb18 function were acquired using a searchable scRNA-seq dataset (https://apps.institutimagine.org/mouse_pallium/) generated from E12.5 telencephalic tissue that allowed an assessment of lineage and differentiation trajectories⁹⁸. SPRING plots generated from E12.5 telencephalic scRNAseq data, which stratified sequenced cells into dorsal glutamatergic and ventral GABAergic lineage trajectories⁹⁸, revealed that, as expected, Ascl1 and Neurog2 were expressed on opposite sides, in GABAergic and glutamatergic lineages, respectively (FIG. 47O). Notably, Zbtb18 was expressed at higher levels in the glutamatergic lineage, although there were some positive cells in GABAergic cells as well⁹⁸. The main source of transcript variability in this data set corresponds to dorsoventral (DV) position, which can be represented graphically on a pseudo-DV plot (FIG. 47P). As expected, Ascl1 and Neurog2 had ventral and dorsal biases, respectively, while Zbtb18 was evenly expressed across the D/V axis (FIG. 47P). Finally, with this data set pseudotime comparisons were performed of Ascl1, Neurog2 and Zbtb18 in the dorsal (dark grey) and ventral (light grey) lineages (FIG. 47Q). In these plots, genes expressed in progenitor cells peak in the center of the plot, while differentiation genes peak at the end of the plots. From this analysis, Ascl1 and Neurog2 plots are more consistent with their identified roles as neural progenitor cell genes, while Zbtb18 expression increased as cells differentiated over time (FIG. 47Q). These studies are consistent with prior reports that Zbtb18 is essential for neuronal migration and morphological maturation during cortical development⁹⁹.

Taken together, these studies support the importance of Zbtb18 as a critical neuronal differentiation factor acting downstream of Ascl1 during neuronal reprogramming (present study) and downstream of likely both Neurog2 and Ascl1 in the developing neocortex.

### Discussion

The goal of this Example was to provide evidence for neuronal lineage conversion in vivo using a ST approach. Specifically, a 10× Visium platform was used to examine the global effect of expressing Ascl1 and Ascl1^{SA6} TFs in astrocytes in the hSOD1^{G93A} motor cortex, at a time point when deep layer neuronal degeneration has initiated. The delivery paradigm was based on AAV5 viral vectors carrying an astrocyte-specific GFAP promoter, along with a t2a-iCre sequence to allow visualization of viral transduction using a Rosa-zsGreen reporter. This approach was previously used to show that compared to Ascl1, Ascl1^{SA6} could more effectively induce neuronal marker expression (and downregulate glial markers) when expressed in astrocytes in the adult motor cortex (Example 5). However, in that study it was difficult to make a direct claim of neuronal lineage conversion, due to the cis effects of neurogenic TF sequences on the GFAP promoter²⁷. With the ST approach used herein, evidence is provided that the transcriptome induced by Ascl1 and Ascl1^{SA6} in vivo share common transcriptional targets with reprogramming studies conducted in vitro^{10,25}. Furthermore, evidence is provided that a neurogenic program centered on a novel TF, Zbtb18, is activated by Ascl1 and Ascl1^{SA6} in vivo and in vitro.

ZBTB18 (aka RP58) is of interest as mutations in this gene are associated with abnormal neocortical development, including microcephaly or macrocephaly, intellectual disability and epilepsy⁸¹. Moreover, the knockdown of Zbtb18 in the embryonic cortex not only disrupts neuronal migration, but also the morphological maturation of newborn neurons, placing this TF as an essential neuronal differentiation gene⁹⁹. The pseudotime analysis in this study similarly supports a role for Zbtb18 as a neuronal differentiation gene, but suggests that this TF may operate in both dorsal and ventral lineages. Interestingly, Zbtb18 has been reported to inhibit the expression of all Id TFs⁶³. In contrast, it was found that Id2 and Id3 were upregulated by Ascl1 and Ascl1^{SA6}, along with Zbtb18, suggesting this inhibitory relationship is not the same in all cellular contexts.

Strikingly, in GBM, Ascl1 phosphorylation on SP sites and the upregulation of ID2 expression were the two identified roadblocks to neuronal differentiation²³. In this in vivo study, both Ascl1 and Acsl1^{SA6} induced the expression of Id TFs when expressed in brain astrocytes, similar to prior in vitro studies¹⁰. At first glance, this induction might be considered inhibitory, as Id transcription factors block type II bHLH TFs such as Ascl1, either by forming non-functional heterodimers or by sopping up type I E-protein cofactors. However, Id2 is induced by kainic acid injuries in the hippocampus that induce neuronal sprouting, which can be phenocopied when Id2 is mis-expressed in the adult brain, Id2 is also capable of driving sprouting of mossy fibers in the adult hippocampus¹⁰⁰. In this study, Id2 activated JAK/STAT and interferon signaling¹⁰⁰. At the transcriptional level, Id2 induces the expression of downstream TF that include Bhlhe40 and Egr1¹⁰⁰, and interestingly, both TFs are induced when Ascl1 is upregulated in adult astrocytes in this study. In a complementary study, Id2 was also shown to be necessary for Ascl1-driven neurogenesis in the embryonic chick spinal cord¹⁰¹. Interestingly, the reliance on Id2 was stronger for ventral spinal cord progenitors that rely on Ascl1 than the dorsal spinal cord progenitors that rely on Neurog2¹⁰¹.

Unexpectedly, transcriptional profiling revealed that the iCre control vector had the greatest effect on global gene expression, initiating a transcriptional pro-inflammatory response that included markers of reactive astrocytes, activated microglia and macrophage, and the complement system. Notably, in iCre clusters associated with Ascl1 or Ascl1^{SA6} overexpression, there was no observation of the same elevated expression of immune response genes triggered by the control vector. The inflammatory signature of the iCre transduced cells was unexpected, especially given how far AAVs have moved into the clinic, with 11 FDA approvals¹⁰². One of the promising outcomes of the present study is that the inflammatory effects of the AAV carrying Ascl1 or Ascl1^{SA6} were much reduced compared to the iCre control vector, suggesting, without limitation, that these TFs may be able to stymie the inflammatory response.

In summary, the present Example provides spatially resolved transcriptomic data that supports the use of bHLH transcription factors, particularly the mutant bHLH transcription factors described herein, promote neuronal lineage conversion when expressed in brain astrocytes in vivo. The present data further identifies Zbtb18 as being useful as a new neuronal conversion factor, optionally together with a mutant bHLH transcription factor as described herein.

Accordingly, the present application further provides a ZBTB18 transcription factor or a nucleic acid encoding the ZBTB18 transcription factor for use in neuronal transformation of glial cells in a subject. In some embodiments, the ZBTB18 transcription factor has an amino acid sequence that is at least 80%, 85%, 90%, 95%, or 97% identical to to one of SEQ ID NOs:36, 38, 40, 42, 44, 46, 48, 50, 52 or 54.

The amino acid sequences of SEQ ID NOs: 36, 38, 40, 42, 44, 46, and 48 correspond with the sequence of mouse ZBTB18 variants transcription factor 1-7, respectively. These sequences were retrieved from the National Library of Medicine, National Center for Biotechnology Information (NCBI) database and correspond with NCBI Reference Sequences for the corresponding mRNA transcripts: NM_00102330, NM_013915, NM_001355098, NM_001355099, NM_001355100, NM_001355101 and NM_001355102, respectively. The mRNA sequences are listed in the present application as SEQ ID NOs: 37, 39, 41, 43, 45, 47, and 49, respectively.

The amino acid sequences of SEQ ID NOs:50, 52 or 54 correspond with the sequences of the human ZBTB18 transcription factor variants 1, 2 and 3, respectively. These sequences were retrieved from the National Library of Medicine, NCBI database and correspond with NCBI Reference Sequences for the corresponding mRNA transcripts: NM_2055768, NM_006352, and NM_001278196, respectively. The mRNA sequences are listed in the present application as SEQ ID NOs: 51, 53 and 55, respectively.

There is high conservation between ZBTB18 transcription factor gene in human, mouse and other species (including rat, dog, chicken and chimpanzee).

Also provided herein are pharmaceutical compositions comprising a ZBTB18 transcription factor having an amino acid sequence that is at least 80%, 85%, 90%, 95%, or 97% identical to one of SEQ ID NOs:36, 38, 40, 42, 44, 46, 48, 50, 52 or 54, or a nucleic acid encoding the the ZBTB18 transcription factor having an amino acid sequence that is at least 80%, 85%, 90%, 95%, or 97% identical to one of SEQ ID NOs:36, 38, 40, 42, 44, 46, 48, 50, 52 or 54, which may be within a viral or non-viral vector.

The present application further provides vectors comprising a nucleic acid sequence encoding a ZBTB18 transcription factor having an amino acid sequence that is at least 80%, 85%, 90%, 95%, or 97% identical to one of SEQ ID NOs:36, 38, 40, 42, 44, 46, 48, 50, 52 or 54. Suitable vectors are described in detail above, in relation to the mutant bHLH transcription factor expressing vectors. The nucleic acid sequence is optionally a sequence that at least 80%, 85%, 90%, 95%, or 97% identical to one of SEQ ID NOs: 37, 39, 41, 43, 45, 47,49, 51, 53 or 55.

Also provided are methods of use for neuronal transformation of glial cells in a subject, by administration of a ZBTB18 transcription factor, an encoding nucleic acid, a vector or pharmaceutical composition, as defined above.

### REFERENCES

1 Bocchi, R., Masserdotti, G. & Götz, M. Direct neuronal reprogramming: Fast forward from new concepts toward therapeutic approaches. Neuron, doi:10.1016/j.neuron.2021.11.023 (2021).
2 Vasan, L., Park, E., David, L. A., Fleming, T. & Schuurmans, C. Direct Neuronal Reprogramming: Bridging the Gap Between Basic Science and Clinical Application. Front Cell Dev Biol 9, 681087, doi:10.3389/fcell.2021.681087 (2021).
3 Heins, N. et al. Glial cells generate neurons: the role of the transcription factor Pax6. Nat Neurosci 5, 308-315, doi:10.1038/nn828 (2002).
4 Oproescu, A. M., Han, S. & Schuurmans, C. New Insights Into the Intricacies of Proneural Gene Regulation in the Embryonic and Adult Cerebral Cortex. Front Mol Neurosci 14, 642016, doi:10.3389/fnmol.2021.642016 (2021).
5 Casarosa, S., Fode, C. & Guillemot, F. Mash1 regulates neurogenesis in the ventral telencephalon. Development 126, 525-534 (1999).
6 Fode, C. et al. A role for neural determination genes in specifying the dorsoventral identity of telencephalic neurons. Genes Dev 14, 67-80 (2000).
7 Schuurmans, C. et al. Sequential phases of cortical specification involve Neurogenin-dependent and -independent pathways. Embo J 23, 2892-2902 (2004).
8 Parras, C. M. et al. Divergent functions of the proneural genes Mash1 and Ngn2 in the specification of neuronal subtype identity. Genes Dev 16, 324-338, doi:10.1101/gad.940902 (2002).
9 Kovach, C. et al. Neurog2 simultaneously activates and represses alternative gene expression programs in the developing neocortex. Cereb Cortex 23, 1884-1900, doi:10.1093/cercor/bhs176 (2013).
10 Masserdotti, G. et al. Transcriptional Mechanisms of Proneural Factors and REST in Regulating Neuronal Reprogramming of Astrocytes. Cell Stem Cell 17,74-88, doi:10.1016/j.stem.2015.05.014 (2015).
11 Gascon, S. et al. Identification and Successful Negotiation of a Metabolic Checkpoint in Direct Neuronal Reprogramming. Cell Stem Cell 18, 396-409, doi:10.1016/j.stem.2015.12.003 (2016).
12 Berninger, B., Guillemot, F. & Gotz, M. Directing neurotransmitter identity of neurones derived from expanded adult neural stem cells. Eur J Neurosci 25, 2581-2590, doi:EJN5509 [pii]10.1111/j.1460-9568.2007.05509.x (2007).
13 Heinrich, C. et al. Directing astroglia from the cerebral cortex into subtype specific functional neurons. PLoS Biol 8, e1000373, doi:10.1371/journal.pbio.1000373 (2010).
14 Heinrich, C., Gotz, M. & Berninger, B. Reprogramming of postnatal astroglia of the mouse neocortex into functional, synapse-forming neurons. Methods Mol Biol 814, 485-498, doi:10.1007/978-1-61779-452-0_32 (2012).
15 Chouchane, M. et al. Lineage Reprogramming of Astroglial Cells from Different Origins into Distinct Neuronal Subtypes. Stem Cell Reports 9, 162-176, doi:10.1016/j.stemcr.2017.05.009 (2017).
16 Rao, Y. et al. NeuroD1 induces microglial apoptosis and cannot induce microglia-to-neuron cross-lineage reprogramming. Neuron 109, 4094-4108.e4095, doi:10.1016/j.neuron.2021.11.008 (2021).
17 Russo, G. L. et al. CRISPR-Mediated Induction of Neuron-Enriched Mitochondrial Proteins Boosts Direct Glia-to-Neuron Conversion. Cell Stem Cell 28, 584, doi:10.1016/j.stem.2020.11.017 (2021).
18 Ali, F. R. et al. Dephosphorylation of the Proneural Transcription Factor ASCL1 Re-Engages a Latent Post-Mitotic Differentiation Program in Neuroblastoma. Mol Cancer Res 18, 1759-1766, doi:10.1158/1541-7786.MCR-20-0693 (2020).
19 Li, S. et al. RAS/ERK signaling controls proneural genetic programs in cortical development and gliomagenesis. J Neurosci 34, 2169-2190, doi:10.1523/JNEUROSCI.4077-13.2014 (2014).
20 Li, S. et al. GSK3 temporally regulates neurogenin 2 proneural activity in the neocortex. J Neurosci 32, 7791-7805, doi:10.1523/JNEUROSCI.1309-12.2012 (2012).
21 Ali, F. et al. Cell cycle-regulated multi-site phosphorylation of Neurogenin 2 coordinates cell cycling with differentiation during neurogenesis. Development 138, 4267-4277, doi:10.1242/dev.067900 (2011).
22 Azzarelli, R., Hardwick, L. J. & Philpott, A. Emergence of neuronal diversity from patterning of telencephalic progenitors. Wiley Interdiscip Rev Dev Biol 4, 197-214, doi:10.1002/wdev.174 (2015).
23 Azzarelli, R. et al. ASCL1 phosphorylation and ID2 upregulation are roadblocks to glioblastoma stem cell differentiation. Sci Rep 12, 2341, doi:10.1038/s41598-022-06248-x (2022).
24 Hindley, C. et al. Post-translational modification of Ngn2 differentially affects transcription of distinct targets to regulate the balance between progenitor maintenance and differentiation. Development 139, 1718-1723, doi:10.1242/dev.077552 (2012).
25 Woods, L. M. et al. Elevated ASCL activity creates de novo regulatory elements associated with neuronal differentiation. BMC Genomics 23, 255, doi:10.1186/s12864-022-08495-8 (2022).
26 Example 5, above
27 Wang, L. L. et al. Revisiting astrocyte to neuron conversion with lineage tracing in vivo. Cell 184, 5465-5481 e5416, doi:10.1016/j.cell.2021.09.005 (2021).
28 Bayraktar, O. A. et al. Astrocyte layers in the mammalian cerebral cortex revealed by a single-cell in situ transcriptomic map. Nat Neurosci 23, 500-509, doi:10.1038/s41593-020-0602-1 (2020).
29 Lanjakornsiripan, D. et al. Layer-specific morphological and molecular differences in neocortical astrocytes and their dependence on neuronal layers. Nat Commun 9, 1623, doi:10.1038/s41467-018-03940-3 (2018).
30 Matias, I., Morgado, J. & Gomes, F. C. A. Astrocyte Heterogeneity: Impact to Brain Aging and Disease. Front Aging Neurosci 11, 59, doi:10.3389/fnagi.2019.00059 (2019).
31 de Carvalho, M. & Swash, M. Lower motor neuron dysfunction in ALS. Clin Neurophysiol 127, 2670-2681, doi:10.1016/j.clinph.2016.03.024 (2016).
32 Turner, M. R. et al. Controversies and priorities in amyotrophic lateral sclerosis. Lancet Neurol 12, 310-322, doi:10.1016/S1474-4422(13)70036-X (2013).
33 Pasinelli, P. & Brown, R. H. Molecular biology of amyotrophic lateral sclerosis: insights from genetics. Nat Rev Neurosci 7, 710-723, doi:10.1038/nrn1971 (2006).
34 Rosen, D. R. Mutations in Cu/Zn superoxide dismutase gene are associated with familial amyotrophic lateral sclerosis. Nature 364, 362, doi:10.1038/364362c0 (1993).
35 Gurney, M. E. et al. Motor neuron degeneration in mice that express a human Cu,Zn superoxide dismutase mutation. Science 264, 1772-1775, doi:10.1126/science.8209258 (1994).
36 Van Den Bosch, L., Van Damme, P., Bogaert, E. & Robberecht, W. The role of excitotoxicity in the pathogenesis of amyotrophic lateral sclerosis. Biochim Biophys Acta 1762, 1068-1082, doi:10.1016/j.bbadis.2006.05.002 (2006).
37 Ozdinler, P. H. et al. Corticospinal motor neurons and related subcerebral projection neurons undergo early and specific neurodegeneration in hSOD1G(9)(3)A transgenic ALS mice. J Neurosci 31, 4166-4177, doi:10.1523/JNEUROSCI.4184-10.2011 (2011).
38 Qian, K. et al. Sporadic ALS Astrocytes Induce Neuronal Degeneration In Vivo. Stem Cell Reports 8, 843-855, doi:10.1016/j.stemcr.2017.03.003 (2017).
39 Ortiz, C. et al. Molecular atlas of the adult mouse brain. Sci Adv 6, eabb3446, doi:10.1126/sciadv.abb3446 (2020).
40 Arlotta, P. et al. Neuronal subtype-specific genes that control corticospinal motor neuron development in vivo. Neuron 45, 207-221, doi:10.1016/j.neuron.2004.12.036 (2005).
41 Silburt, J., Lipsman, N. & Aubert, I. Disrupting the blood-brain barrier with focused ultrasound: Perspectives on inflammation and regeneration. Proc Natl Acad Sci USA 114, E6735-E6736, doi:10.1073/pnas.1710761114 (2017).
42 Hatzipetros, T. et al. A Quick Phenotypic Neurological Scoring System for Evaluating Disease Progression in the SOD1-G93A Mouse Model of ALS. J Vis Exp, doi:10.3791/53257 (2015).
43 Terrisse, L. et al. Increased levels of apolipoprotein D in cerebrospinal fluid and hippocampus of Alzheimer's patients. J Neurochem 71, 1643-1650, doi:10.1046/j.1471-4159.1998.71041643.x (1998).
44 Muffat, J., Walker, D. W. & Benzer, S. Human ApoD, an apolipoprotein up-regulated in neurodegenerative diseases, extends lifespan and increases stress resistance in Drosophila. Proc Natl Acad Sci USA 105, 7088-7093, doi:10.1073/pnas.0800896105 (2008).
45 McCarthy, R. C. et al. Inflammation-induced iron transport and metabolism by brain microglia. J Biol Chem 293, 7853-7863, doi:10.1074/jbc.RA118.001949 (2018).
46 Trichas, G., Begbie, J. & Srinivas, S. Use of the viral 2A peptide for bicistronic expression in transgenic mice. BMC Biol 6, 40, doi:10.1186/1741-7007-6-40 (2008).
47 Clarke, L. E. et al. Normal aging induces A1-like astrocyte reactivity. Proc Natl Acad Sci USA 115, E1896-E1905, doi:10.1073/pnas.1800165115 (2018).
48 DePaula-Silva, A. B. et al. Differential transcriptional profiles identify microglial- and macrophage-specific gene markers expressed during virus-induced neuroinflammation. J Neuroinflammation 16, 152, doi:10.1186/s12974-019-1545-x (2019).
49 Kiialainen, A., Hovanes, K., Paloneva, J., Kopra, O. & Peltonen, L. Dap12 and Trem2, molecules involved in innate immunity and neurodegeneration, are co-expressed in the CNS. Neurobiol Dis 18, 314-322, doi:10.1016/j.nbd.2004.09.007 (2005).
50 Bayly-Jones, C., Pang, S. S., Spicer, B. A., Whisstock, J. C. & Dunstone, M. A. Ancient but Not Forgotten: New Insights Into MPEG1, a Macrophage Perforin-Like Immune Effector. Front Immunol 11, 581906, doi:10.3389/fimmu.2020.581906 (2020).
51 Turner, M. J. et al. Reduction of inflammation and preservation of neurological function by anti-CD52 therapy in murine experimental autoimmune encephalomyelitis. J Neuroimmunol 285,4-12, doi:10.1016/j.jneuroim.2015.05.018 (2015).
52 Schartz, N. D. & Tenner, A. J. The good, the bad, and the opportunities of the complement system in neurodegenerative disease. J Neuroinflammation 17, 354, doi:10.1186/s12974-020-02024-8 (2020).
53 Kagitani-Shimono, K. et al. Lipocalin-type prostaglandin D synthase (beta-trace) is upregulated in the alphaB-crystallin-positive oligodendrocytes and astrocytes in the chronic multiple sclerosis. Neuropathol Appl Neurobiol 32, 64-73, doi:10.1111/j.1365-2990.2005.00690.x (2006).
54 Miyazaki, M. et al. Id2 and Id3 maintain the regulatory T cell pool to suppress inflammatory disease. Nat Immunol 15, 767-776, doi:10.1038/ni.2928 (2014).
55 Tzeng, S. F. Inhibitors of DNA binding in neural cell proliferation and differentiation. Neurochem Res 28,45-52, doi:10.1023/a:1021691911011 (2003).
56 Hanna J, D. L., Fleming T, Screaton R A, Schuurmans C. Beyond genetics: the role of metabolism in photoreceptor survival, development and repair. Frontiers in Cell and Developmental Biology. in press (2022).
57 Cargill, K. R., Hasken, W. L., Gay, C. M. & Byers, L. A. Alternative Energy: Breaking Down the Diverse Metabolic Features of Lung Cancers. Front Oncol 11, 757323, doi:10.3389/fonc.2021.757323 (2021).
58 Wilczynska, K. M. et al. Nuclear factor I isoforms regulate gene expression during the differentiation of human neural progenitors to astrocytes. Stem Cells 27, 1173-1181, doi:10.1002/stem.35 (2009).
59 Dennis, D. J. et al. Neurog2 and Ascl1 together regulate a postmitotic derepression circuit to govern laminar fate specification in the murine neocortex. Proc Natl Acad Sci USA 114, E4934-E4943, doi:10.1073/pnas.1701495114 (2017).
60 Hoch-Kraft, P., Trotter, J. & Gonsior, C. Missing in Action: Dysfunctional RNA Metabolism in Oligodendroglial Cells as a Contributor to Neurodegenerative Diseases? Neurochem Res 45, 566-579, doi:10.1007/s11064-019-02763-γ (2020).
61 Ni, J. et al. Increased expression and altered subcellular distribution of cathepsin B in microglia induce cognitive impairment through oxidative stress and inflammatory response in mice. Aging Cell 18, e12856, doi:10.1111/acel.12856 (2019).
62 Penisson, M., Ladewig, J., Belvindrah, R. & Francis, F. Genes and Mechanisms Involved in the Generation and Amplification of Basal Radial Glial Cells. Front Cell Neurosci 13, 381, doi:10.3389/fncel.2019.00381 (2019).
63 Okado, H. Regulation of brain development and brain function by the transcriptional repressor RP58. Brain Res 1705, 15-23, doi:10.1016/j.brainres.2018.02.042 (2019).
64 Srinivasan, K. et al. A network of genetic repression and derepression specifies projection fates in the developing neocortex. Proc Natl Acad Sci USA 109, 19071-19078, doi:10.1073/pnas.1216793109 (2012).
65 Manuel, M. N., Mi, D., Mason, J. O. & Price, D. J. Regulation of cerebral cortical neurogenesis by the Pax6 transcription factor. Front Cell Neurosci 9, 70, doi:10.3389/fncel.2015.00070 (2015).
66 Mogensen, T. H. IRF and STAT Transcription Factors—From Basic Biology to Roles in Infection, Protective Immunity, and Primary Immunodeficiencies. Front Immunol 9, 3047, doi:10.3389/fimmu.2018.03047 (2018).
67 Zhang, Z. J., Jiang, B. C. & Gao, Y. J. Chemokines in neuron-glial cell interaction and pathogenesis of neuropathic pain. Cellular and molecular life sciences: CMLS 74, 3275-3291, doi:10.1007/s00018-017-2513-1 (2017).
68 Marques, R. E., Guabiraba, R., Russo, R. C. & Teixeira, M. M. Targeting CCL5 in inflammation. Expert Opin Ther Targets 17,1439-1460, doi:10.1517/14728222.2013.837886 (2013).
69 Zhang, Y. J., Song, J. R. & Zhao, M. J. NR4A1 regulates cerebral ischemia-induced brain injury by regulating neuroinflammation through interaction with NF-kappaB/p65. Biochemical and biophysical research communications 518, 59-65, doi:10.1016/j.bbrc.2019.08.008 (2019).
70 Yu, Q. et al. Early activation of Egr-1 promotes neuroinflammation and dopaminergic neurodegeneration in an experimental model of Parkinson's disease. Exp Neurol 302, 145-154, doi:10.1016/j.expneurol.2018.01.009 (2018).
71 Paskevicius, T. et al. The Fabp5/calnexin complex is a prerequisite for sensitization of mice to experimental autoimmune encephalomyelitis. FASEB journal: official publication of the Federation of American Societies for Experimental Biology 34, 16662-16675, doi:10.1096/fj.202001539RR (2020).
72 Tang, X. et al. Novel transcriptional regulation of VEGF in inflammatory processes. J Cell Mol Med17, 386-397, doi:10.1111/jcmm.12020 (2013).
73 Wang, S. M. et al. Astrocytic CCAAT/Enhancer-binding protein delta contributes to reactive oxygen species formation in neuroinflammation. Redox Biol 16, 104-112, doi:10.1016/j.redox.2018.02.011 (2018).
74 Tutukova, S., Tarabykin, V. & Hernandez-Miranda, L. R. The Role of Neurod Genes in Brain Development, Function, and Disease. Front Mol Neurosci 14, 662774, doi:10.3389/fnmol.2021.662774 (2021).
75 Kinare, V. et al. An evolutionarily conserved Lhx2-Ldb1 interaction regulates the acquisition of hippocampal cell fate and regional identity. Development 147, doi:10.1242/dev.187856 (2020).
76 Ohtsuka, T. & Kageyama, R. Regulation of temporal properties of neural stem cells and transition timing of neurogenesis and gliogenesis during mammalian neocortical development. Semin Cell Dev Biol 95,4-11, doi:10.1016/j.semcdb.2019.01.007 (2019).
77 Weiss, L. A. & Nieto, M. The crux of Cux genes in neuronal function and plasticity. Brain Res 1705, 32-42, doi:10.1016/j.brainres.2018.02.044 (2019).
78 Buxbaum, J. D. et al. Molecular dissection of NRG1-ERBB4 signaling implicates PTPRZ1 as a potential schizophrenia susceptibility gene. Mol Psychiatry 13, 162-172, doi:10.1038/sj.mp.4001991(2008).
79 Suriano, C. M. et al. Adeno-associated virus (AAV) reduces cortical dendritic complexity in a TLR9-dependent manner. bioRxiv, 2021.2009.2028.462148, doi:10.1101/2021.09.28.462148 (2021).
80 Johnston, S. et al. AAV ablates neurogenesis in the adult murine hippocampus. Elife 10, doi:10.7554/eLife.59291 (2021).
81 Heng, J. I., Viti, L., Pugh, K., Marshall, O. J. & Agostino, M. Understanding the impact of ZBTB18 missense variation on transcription factor function in neurodevelopment and disease. J Neurochem 161, 219-235, doi:10.1111/jnc.15572 (2022).
82 Nie, F. et al. Schizophrenia risk candidate EGR3 is a novel transcriptional regulator of RELN and regulates neurite outgrowth via the Reelin signal pathway in vitro. J Neurochem 157, 1745-1758, doi:10.1111/jnc.15225 (2021).
83 Chang, S. Y. et al. Hippocampal changes in mice lacking an active prohormone convertase 2. Hippocampus 30, 715-723, doi:10.1002/hipo.23195 (2020).
84 Hamilton, K. A. et al. Mice lacking the transcriptional regulator Bhlhe40 have enhanced neuronal excitability and impaired synaptic plasticity in the hippocampus. PLoS One 13, e0196223, doi:10.1371/journal.pone.0196223 (2018).
85 Serrano-Saiz, E., Leyva-Diaz, E., De La Cruz, E. & Hobert, O. BRN3-type POU Homeobox Genes Maintain the Identity of Mature Postmitotic Neurons in Nematodes and Mice. Curr Biol 28, 2813-2823 e2812, doi:10.1016/j.cub.2018.06.045 (2018).
86 Li, H. & Richardson, W. D. Evolution of the CNS myelin gene regulatory program. Brain Res 1641, 111-121, doi:10.1016/j.brainres.2015.10.013 (2016).
87 Choy, F. C., Klaric, T. S., Leong, W. K., Koblar, S. A. & Lewis, M. D. Reduction of the neuroprotective transcription factor Npas4 results in increased neuronal necrosis, inflammation and brain lesion size following ischaemia. J Cereb Blood Flow Metab 36, 1449-1463, doi:10.1177/0271678X15606146 (2016).
88 Krieger, T. G. et al. Mutations in thyroid hormone receptor alpha1 cause premature neurogenesis and progenitor cell depletion in human cortical development. Proc Nat Acad Sci USA 116, 22754-22763, doi:10.1073/pnas.1908762116 (2019).
89 Tischmeyer, W. & Grimm, R. Activation of immediate early genes and memory formation. Cellular and molecular life sciences: CMLS 55, 564-574, doi:10.1007/s000180050315 (1999).
90 Chen, F. et al. Clusterin secreted from astrocyte promotes excitatory synaptic transmission and ameliorates Alzheimer's disease neuropathology. Mol Neurodegener 16, 5, doi:10.1186/s13024-021-00426-7 (2021).
91 Remesal, L. et al. PBX1 acts as terminal selector for olfactory bulb dopaminergic neurons. Development 147, doi:10.1242/dev.186841 (2020).
92 Sainio, M. T. et al. Absence of NEFL in patient-specific neurons in early-onset Charcot-Marie-Tooth neuropathy. Neural Genet 4, e244, doi:10.1212/NXG.0000000000000244 (2018).
93 Minj, E., Yadav, R. K. & Mehan, S. Targeting Abnormal Nrf2/HO-1 Signaling in Amyotrophic Lateral Sclerosis: Current Insights on Drug Targets and Influences on Neurological Disorders. Curr Mol Med 21, 630-644, doi:10.2174/1566524021666210111104920 (2021).
94 Nandi, S. et al. Receptor-type protein-tyrosine phosphatase zeta is a functional receptor for interleukin-34. J Biol Chem 288, 21972-21986, doi:10.1074/jbc.M112.442731 (2013).
95 Munoz-Garcia, J. et al. The twin cytokines interleukin-34 and CSF-1: masterful conductors of macrophage homeostasis. Theranostics 11, 1568-1593, doi:10.7150/thno.50683 (2021).
96 Pollen, A. A. et al. Low-coverage single-cell mRNA sequencing reveals cellular heterogeneity and activated signaling pathways in developing cerebral cortex. Nat Biotechnol 32, 1053-1058, doi:10.1038/nbt.2967 (2014).
97 Han, S. et al. Proneural genes define ground-state rules to regulate neurogenic patterning and cortical folding. Neuron 109, 2847-2863 e2811, doi:10.1016/j.neuron.2021.07.007 (2021).
98 Moreau, M. X., Saillour, Y., Cwetsch, A. W., Pierani, A. & Causeret, F. Single-cell transcriptomics of the early developing mouse cerebral cortex disentangle the spatial and temporal components of neuronal fate acquisition. Development 148, doi:10.1242/dev.197962 (2021).
99 Clement, O. et al. Rp58 and p27(kip1) coordinate cell cycle exit and neuronal migration within the embryonic mouse cerebral cortex. Neural Dev 12, 8, doi:10.1186/s13064-017-0084-3 (2017).
100 Luo, W. et al. Recurrent rewiring of the adult hippocampal mossy fiber system by a single transcriptional regulator, d2. Proc Natl Acad Sci USA 118, doi:10.1073/pnas.2108239118 (2021).
101 Le Dreau, G. et al. E proteins sharpen neurogenesis by modulating proneural bHLH transcription factors' activity in an E-box-dependent manner. Elife 7, doi:10.7554/eLife.37267 (2018).
102 Gene therapy at the crossroads. Nat Biotechnol 40, 621, doi:10.1038/s41587-022-01346-7 (2022).
103 Ferla, R. et al. Low incidence of hepatocellular carcinoma in mice and cats treated with systemic adeno-associated viral vectors. Mol Ther Methods Clin Dev 20, 247-257, doi:10.1016/j.omtm.2020.11.015 (2021).
104 Irie, T. et al. Direct neuronal conversion of microglia/macrophages reinstates neurological function after stroke. bioRxiv, 2021.2009.2026.461831, doi:10.1101/2021.09.26.461831(2021).
105 Matsuda, T. et al. Pioneer Factor NeuroD1 Rearranges Transcriptional and Epigenetic Profiles to Execute Microglia-Neuron Conversion. Neuron 101, 472-485 e477, doi:10.1016/j.neuron.2018.12.010 (2019).
106 Matsuda, T. & Nakashima, K. Clarifying the ability of NeuroD1 to convert mouse microglia into neurons. Neuron 109, 3912-3913, doi:10.1016/j.neuron.2021.11.012 (2021).
107 Sun, Y. et al. Neurogenin promotes neurogenesis and inhibits glial differentiation by independent mechanisms. Cell 104, 365-376, doi:10.1016/s0092-8674(01)00224-0 (2001).
108 Livingston, J. et al. Direct reprogramming of astrocytes to neurons leads to functional recovery after stroke. bioRxiv, 2020.2002.2002.929091, doi:10.1101/2020.02.02.929091 (2020).
109 Butler, A., Hoffman, P., Smibert, P., Papalexi, E. & Satija, R. Integrating single-cell transcriptomic data across different conditions, technologies, and species. Nat Biotechnol 36,411-420, doi:10.1038/nbt.4096 (2018).
110 Ulgen, E., Ozisik, O. & Sezerman, O. U. pathfindR: An R Package for Comprehensive Identification of Enriched Pathways in Omics Data Through Active Subnetworks. Front Genet 10, 858, doi:10.3389/fgene.2019.00858 (2019).
111 Kuleshov, M. V. et al. Enrichr: a comprehensive gene set enrichment analysis web server 2016 update. Nucleic Acids Res 44, W90-97, doi:10.1093/nar/gkw377 (2016).
112 Okawa, S., Angarica, V., Lemischka, I. et al. A differential network analysis approach for lineage specifier prediction in stem cell subpopulations. npj Syst Biol Appl 1, 15012 (2015).

### Example 7: Neuronal Lineage Conversion Targeting Motor Cortex Astrocytes Delays Motor Symptom Progression in an ALS Mouse Model

Amyotrophic lateral sclerosis (ALS) is associated with the death of upper motor neurons in deep layers of the motor cortex as well as their innervation targets, lower motor neurons in the brainstem and spinal cord. Spinal cord motor neurons innervate skeletal, visceral and cardiac muscles, and their loss leads to muscle denervation and atrophy, which ultimately causes death due to respiratory muscle failure¹. The dying back hypothesis suggests that ALS pathology initiates in lower motor neurons in the spinal cord, which leads to progressive neurodegeneration of upper motor neurons in the deep layers of the motor cortex^{2,3}. More recently a dying forward model has been proposed, which suggests instead that glutamate excitotoxicity begins in the motor cortex and spreads to the spinal cord to trigger lower motor neuron death⁴⁻⁶. Indeed, in patients with ALS, transcranial magnetic stimulation (TMS) reveals that cortical hyperexcitability occurs prior to clinical motor symptoms⁷. Similarly, in hSOD1^{G93A} transgenic animals, dendritic regression, spine loss and hyperexcitability are observed in the motor cortex before motor symptom onset⁸. Notably, UMN death may initiate disease progression. Indeed, knockdown of mutant SOD1 in the motor cortex of SOD1^{G93A} rats prior to symptom onset delays disease progression, increases survival, and delays LMN and neuromuscular junction degeneration 9. AAV-mediated retrograde labelling of neocortical neurons in hSOD1^{G93A} transgenics also revealed upper MN degeneration and a loss of layer II/III apical dendrites at P60¹⁰. Finally, there is also evidence for a loss of cholinergic neurons in the basal forebrain of hSOD1^{G93A} mice, but it is not clear whether this occurs before or after upper MN degeneration¹¹.

The dying-forward model also proposed that pathology in the motor cortex may not only precede spinal cord disease, but also drive the disease forward. As a corollary to this model, it has been suggested that by preventing the death of upper motor neurons, disease progression could be stalled. Consistent with this finding, knockdown of mutant SOD1 in the motor cortex of hSOD1^{G93A} rats prior to symptom onset delays disease progression, increases survival, and delays lower motor neuron and neuromuscular junction degeneration 9. In addition, NPCs transplanted into the motor cortex of SOD1^{G93A} rats differentiate into astrocytes that secrete glial cell-derived neurotrophic factor (GDNF), delaying both UMN and LMN death¹². Finally, chemogenetic stimulation of cortical Pvalb⁺ GABAergic neurons, which are hypoactive in hSOD1^{G93A} mice, reduces excitotoxic death of motor neurons and preserves motor function¹³.

There is growing support for the idea that neurodegeneration in ALS is a non cell autonomous consequence of a pathogenic change in astrocytes^{14,15}. In normal homeostatic conditions, astrocytes provide nutrients and neuroprotective molecules to sustain neuronal health¹⁶. However, in a pathological pro-inflammatory state, astrocytes undergo a reactive transformation and begin to produce neurotoxic molecules that trigger neurodegeneration¹⁷. Neuroinflammation is triggered initially by pro-inflammatory microglia, which secrete cytokines that activate A1 astrocytes^{14,15,18}. Conversely, A2 astrocytes are activated by anti-inflammatory cytokines and in response, secrete neurotrophic factors to support neuronal survival^{17,19}. Interestingly, human induced pluripotent stem cells (hiPSCs) derived from patients with ALS differentiate into astrocytes that spontaneously become reactive and induce complement component 3 (C3) gene expression even in the absence of a proinflammatory cue²⁰. Finally, astrocytes induce neuronal cell death in an ALS animal model when transplanted into a healthy host brain¹⁶, highlighting the importance of these cells for therapeutic intervention.

To prevent toxicity associated with ALS astrocytes, and to potentially replace lost upper motor neurons, the present Example took a neuronal lineage conversion approach, using the proneural transcription factor (TF) Ascl1 to reprogram motor cortex astrocytes to induced neurons (iNeurons)²¹. Specifically, the mutated version of Ascl1 in which six serines adjacent to prolines (SP sites) were converted to alanines, generating Ascl1^{SA6} was used, which is less subject to inhibitory controls in the environment. The conversion of astrocytes to iNeurons can promote neural repair by reducing astrocyte toxicity and cortical hyperexcitability and/or by replacing lost neurons. Here it is shown that Ascl1^{SA6} induced neuronal conversion in the motor cortex of hSOD1^{G93A} mice has beneficial effects on lifespan and motor function.

### Materials and Methods

Animal models. All experiments used age and gender matched mice. Homozygous zsGreen mice were generated by breeding zsGreen (Jackson laboratory:B6.Cg-Gt(ROSA)26Sor^{tm6(CAG-ZsGreen1)Hze}/J) males and females. hSOD1^{G93A} (Jackson laboratory: B6; Cg-Tg(SOD1-G93A)1Gur/J) male and Female litter mice were maintained on C57Bl/6J genetic background. The models were bred and verified as described above in Example 6.

AAV Intracranial injections. AAV5-GFAP-iCre plasmid was gift from Dr. Faiz's lab. AAV5-GFAP-iCre and AAV5-GFAP-Ascl1/SA6-t2a-iCre were packaged by VectorBuilder Inc. AAV5-CAG-Flex-EGFP (catalog #51502-AAV5) was purchased from Addgene. Mice were anaesthetized using isoflurane (2%, 1 L/min; Fresenius Kabi, CP0406V2) and injected subcutaneously with an analgesic, either buprenorphine (0.1 mg/kg; Vetergesic, 023425101 or Tramadol-HCL (20 mg/kg; Chiron, RxN704598), along with baytril (2.5 mg/kg; Bayer, 02249243), and saline (0.5 ml, Braun, L8001). A burr hole was drilled through the skull over the cortex and a stereotaxic instrument was used to identify bregma and lambda for injection at layer V region (AP: +2.15, L/M: ±1.7, DV: −1.7). To label specific astrocytes and converted neuronal population, Cre driver AAV with Cre dependent AAV were combined. 4.8×10⁹ GC in a 1 uL total volume of AAV5-GFAP-iCre and AAV5-CAG-Flex-EGFP or AAV5-GFAP-Ascl1/SA6-t2a-iCre with AAV5-CAG-Flex-EGFP were injected into 16 weeks old hSOD1^{G93A} transgenic ALS mice. Mice were scarified after 21 dpi.

AAV5-GFAP-iCre and AAV5-GFAP-Ascl1/SA6-t2a-iCre viral vectors were bilaterally injected into the motor cortex layer V region (AP: +2.15, L/M: ±1.7, DV: −1.7) at a volume 1 μl over 10 min using 5 μl Hamilton syringe with 30-gauge needle. Surgeries were performed at week 16 C57Bl/6J mice and scarified after 21 dpi to count neuronal conversion rate. The same injection protocols were performed into 16 weeks old zsGreen mice for control and 16 weeks old zsGreen/hSOD1^{G93A} transgenic ALS mice. Also, 19 weeks old zsGreen/hSOD1^{G93A} transgenic ALS mice were tested. All these mice were monitored for their disease progression by checking their body weight, NS, motor behavior test (Rotarod, grip strength, and gait analysis or catwalk) until the experimental endpoint. When mice were not able to right themselves within 30 sec after being placed on either side, they were sacrificed.

Tissue Collection, Processing, and Sectioning. Mice were anesthetized with ketamine (75 mg/kg, Narketan, 0237499) and xylazine (10 mg/kg, Rompun, 02169592) prior to perfusion. Intracardially perfusion was performed with approximately 20× of their blood volume using a peristaltic pump at a flow rate of 10 ml/min with ice cold saline (0.9% NaCl, Braun, L8001), followed by 4% paraformaldehyde (PFA, Electron Microscopy Sciences, 19208) in phosphate buffer saline (PBS, Wisent, 311-011-CL) for approximately five minutes. Brains were collected and post-fixed overnight in 4% PFA in PBS, then transfer to a 20% sucrose for cryoprotection. Brains were frozen at −26° C. inside of a Leica CM3050 cryostat (Leica Microsystems Canada Inc., Richmond Hill, Ontario, Canada) and cut in 30 μm coronal sectioning by collecting on Fisherbrand™ Superfrost™ Plus Microscope Slides (Thermo Fisher Scientific, 12-550-15).

Body weight and Neuroscore (NS). Body weight was measured before intracranial injections and continued to measure their bodyweight every week until their end-point. NS (0-4) for SOD1 was assigned weekly by observing the mouse under the following four conditions: a) suspended by its tail, b) walking, c) examining how long it takes for the mouse to right itself when placed on its side. NS 0 was a pre-symptomatic stage. NS 1 started ALS phenotype. A symptomatic stage was when mice walk normally but their hindlimbs were collapsed or partially collapsed towards the midline. NS 2 was the onset of paresis. Animals can walk but their toes curl downwards while walking. At NS 3, mice can walk but they did not use their hindlimbs to make a forward motion. NS 4 was the humane end-point, when animals cannot right themselves in 10 sec.

Motor Coordination Analysis. Motor coordination, strength, and balance were assessed in rotarod apparatus. Mice started train on the rotarod one week before recording the data. Mice were placed onto a cylinder and rotation started with 2 rpm. Within 180 sec the rotation speed increased up to 18 rpm over a min. With the occurrence of motor neuron symptoms animals get weaker and fall onto the sensing platforms. The time of drop is sensed by magnetic switches and shown on the display at the end of the experiment. The average of three runs from each week on the rotarod were assessed and the group performance was compared.

Grip Strength. Grip strength meter (Bioseb™) was used to study neuromuscular function. Mice were held by the tail over a 100×80 mm at an angle of 20° grid holder. Once mice were firmly grasping the grid, they were pulled along the axis of the force sensor until they release the grid. Grip strength tests were done in three rounds of one trial with 5 min in home cages between the rounds. All grip strength tests were normalized to body weight.

Gait Analysis. Gait abnormalities were assessed by analyzing the footprint pattern of mice as they walked along a 50 cm long and 10 cm wide runway. Forelimbs were painted with a red dye and hindlimbs were painted with a blue dye. Footprint patterns were analyzed for three step parameters: 1) stride length, to measure the average distance of forward movement between each stride; 2) sway distance, to measure the average distance between left and right hind footprints; 3) stance length, to measure the average distance from left or right front footprint to right or left hind footprint.

CatWalk XT®. In the current study the CatWalk® (Noldus Information Technology, Wageningen, The Netherlands) version 11 was used for gait assessment. The CatWalk® gait analysis system used to capture gait parameters uses illuminated footprints. Unlike painting paws of mouse, the ALS mouse is positioned at the beginning of the glass floor walkway and freely walk toward the home cage voluntarily. In most cases the maximum six runs are conducted and three runs which is a maximum speed variation of 60% were selected for analysis. The distance between successive placements of the same left and right paws are analyzed to capture gait parameters every week until mice are unable to continue the tests.

### Results

### In Vivo Delivery of AAV Neuronal Reprogramming Vectors Preserves Motor Function of ALS Mice

The reprogramming of astrocytes to neurons is readily induced by proneural TFs, such as Ascl1, in vitro, but in vivo neuronal conversion remains inefficient. We previously showed that we can increase the capacity of Ascl1 to induce neuronal marker expression in motor cortex astrocytes by using a mutated Ascl1^{SA6} that is more efficient at inducing neuronal conversion compared to native Ascl1. Adeno-associated virus (AAV) 5 carrying a glial fibrillary acid promoter (GFAP) was used to express Ascl1^{A}6 in motor neuron astrocytes. A (v2a)-iCre cassette was included so that transduced cells could be detected using a Rosa-zsGreen reporter. The following two constructs were used in this study: a control AAV expressing iCre alone (AAV5-GFAP-iCre, hereafter iCre) and the test vector expressing Ascl1^{SA6} (AAV5-GFAP-Asc1^{SA6}-t2a-iCre, hereafter Ascl1^{SA6}). The two AAVs were injected into the motor cortex (AP: +2.15, L/M: ±1.7, DV: −1.7) in four cohorts of mice each; male and female Rosa-zsGreen (control) and male and female hSOD1^{G93A}; Rosa-zsGreen (ALS model) mice.

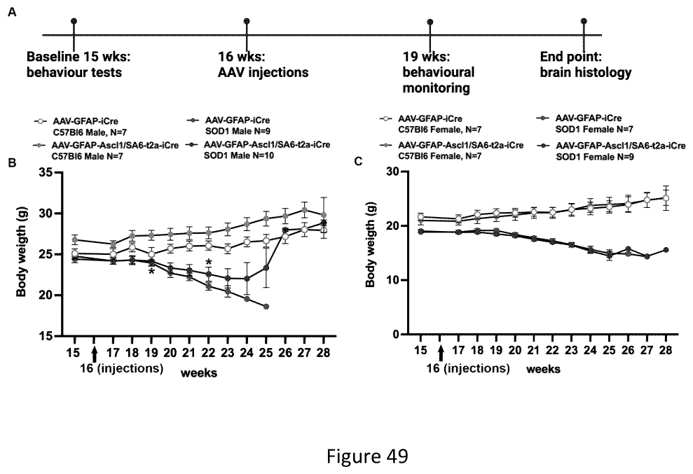

As ALS is a wasting disease, weekly measures of body weight and motor behaviour were used to assess the impact of expressing Ascl1^{SA6}, a neurogenic TF, in motor cortex astrocytes. Baseline measurements were made at 15-weeks of age for each test in the four animal cohorts, one week prior to AAV injection at 16-weeks of age, with 28 weeks set as the experimental endpoint. Control male and female animals gradually gained weight throughout the duration of the experiment (FIG. 49). In contrast, most hSod1^{G93A} male and female mice began to lose weight from 20 weeks of age, a trend that continued to the ˜24-week time point when animals reached their humane endpoint and were sacrificed (FIG. 1). Strikingly, following Ascl1^{SA6} injection, hSod1^{G93A} mice were better able to sustain their body weight over time vs. controls. Indeed, comparisons of iCre and Ascl1^{SA6} injected males revealed a significant difference at 19 and 22 weeks, indicative of a better retention of body weight (two-way ANOVA, p=0.0470 and p=0.0370, respectively). However, females did not improve their body weight, a difference in therapeutic response that is not well understood in the field (FIG. 49). Finally, in the wild-type Rosa-zsGreen mouse cohort, Ascl1^{SA6} injected animals also had larger body weights than the control mice at each week of measurement (FIG. 49). However, baseline measures were also lower for the iCre mice at the starting point, and the percentage of body weight change did not significantly differ.

Taken together, this data supports the capacity of Ascl1^{SA6} induced neuronal conversion to have a positive impact on animal health.

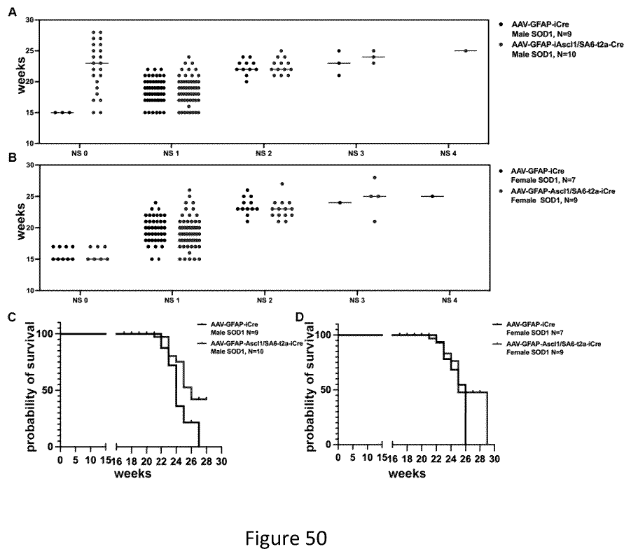

Animals were monitored for changes in overall motor function using a Neuroscore (NS) scale that assigns values ranging from NS 0 (asymptomatic) to NS 4, when animals could no longer right themselves and were humanely sacrificed (FIG. 50). NS was assessed weekly by observing the mouse suspended by its tail, walking, and examining how long it takes for the mouse to right itself when placed on its side. All wild-type male and female mice remained at NS 0 throughout the study, contrasting to hSOD1^{G93A} mice, most of which reached NS 4 before the experimental endpoint of 28 weeks. Based on the NS score, male hSOD1^{G93A} mice retained motor function longer than females, as they typically remained at NS 1 for 37 days while female hSOD1^{G93A} mice stayed at NS 1 for 28 days. However, once symptom onset was observed, animals of both sexes deteriorated rapidly, with male and female mice spending only 5 days at NS 2 and less than 5 days at NS 3, before proceeding to NS 4 and humane sacrifice²². Importantly, within the Ascl1^{SA6} injected cohorts, two male mice survived and stayed at NS 0 until the 28-week experimental endpoint (FIG. 50A) and one female mouse stayed at NS 1 until 26 weeks (FIG. 50B). Thus, Ascl1^{SA6} improves survival times for male hSOD1^{G93A} mice when expressed in motor cortex astrocytes at 16 weeks of age.

Next it was asked whether Ascl1^{SA6}-induced neuronal reprogramming of cortical astrocytes improved survival times. Strikingly, while only 10% of iCre treated hSOD1^{G93A} male mice survived to the experimental endpoint of 28 weeks, 40% treated with Ascl1^{SA6} survived to 28 weeks (FIG. 50C) (median survival 24 days for iCre and 26 days for Ascl1^{SA6}, log-rank test, p=0.031). In contrast, while one Ascl1^{SA6} treated hSOD1^{G93A} female mice survived until 28 weeks of age, with improved motor performance, median survival was similar for iCre (26 weeks) and Ascl1^{SA6} (25 weeks) treated hSOD1^{G93A} female mice (log-rank test, p=0.465) (FIG. 50D).

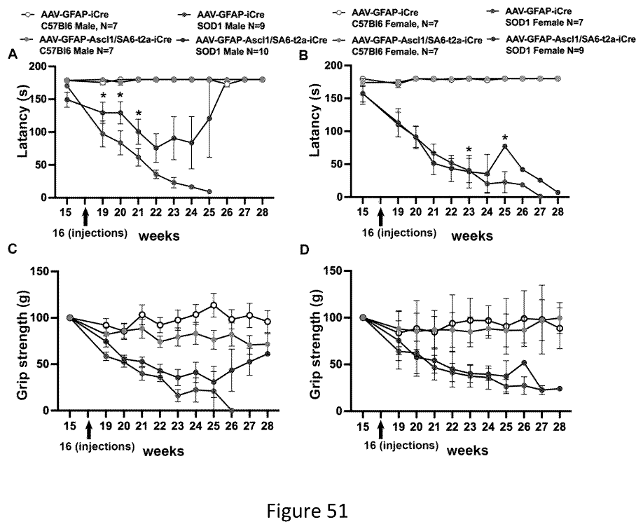

The rotarod test was then used to measure motor coordination, balance, and motor learning. At baseline (15 weeks) and from 17 weeks post-injection to 28 weeks (experimental endpoint), mice were placed on a cylinder that was rotated for 180 sec at 18 rpm. While control mice remained on the cylinder throughout the 180 sec, hSOD1^{G93A} mice lost motor capacity over time, and showed an age-dependent decrease in latency to fall compared to control mice. hSOD1^{G93A} male mice injected with Ascl1^{SA6} had an increased ability to stay on the rotarod compared to control mice overtime and showed a significant difference at 19, 20, and 21 compared to iCre injected mice (FIG. 51A) (Šídák's multiple comparisons test, p=0.0471, p=0.0379, and p=0.0329 respectively). Similarly, female mice treated with Ascl1 were also able to stay on the rotarod longer than the control iCre-treated animals and 23 and 25 weeks of age (FIG. 51B) (Šídák's multiple comparisons test, p=0.0359 and p<0.0001, respectively).

Grip strength assesses neuromuscular function by measuring maximal peak force exerted when gripping a wire holder which was normalized to body weight. hSOD1^{G93A} male mice injected with Ascl1^{SA6} displayed an increase in grip strength compared to control mice as they aged, but these measures did not reach statistical significance (FIG. 51C). Female mice also showed no significant differences in grip strength between iCre and Ascl1^{SA6} injected mice (FIG. 51D).

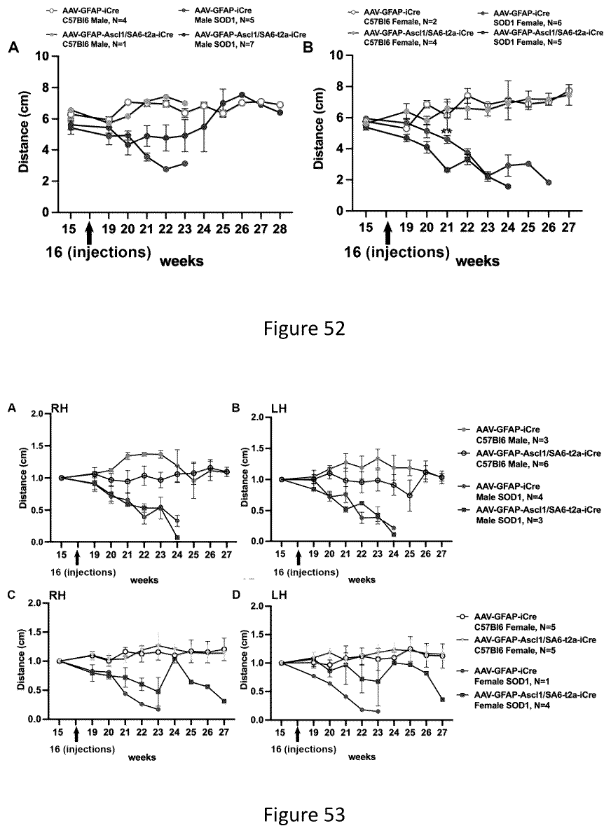

A critical outcome of motor deficits are changes in gait, which were assessed by analyzing footprint patterns as mice walked along a 50 cm long and 10 cm wide runway. hSOD1^{G93A} male mice injected with Ascl1^{SA6} displayed a clear trend towards longer stride lengths compared to iCre treated mice, but statistical significance was not reached, largely because control animals died earlier and so control animals were not available for comparisons (FIG. 52A). Conversely, hSOD1^{G93A} female mice injected with iCre had an increased stride compared to Ascl1^{SA6} treated animals at 15 and 21 weeks (FIG. 52B) (Šídák's multiple comparisons test, p=0.0319 and p=0.0057, respectively). These results suggest that Ascl1^{SA6} expression in motor cortex might be detrimental in hSOD1^{G93A} female mice at 16 weeks of age. However, no detrimental effects were observed in wild-type male (FIG. 52A) or female (FIG. 52B) wild-type mice, which displayed similar stride lengths whether they received iCre or Ascl1^{SA6} treatment.

Catwalk is a gait analysis system that can be used to assess a larger number of gait abnormalities in an automated manner. Analyses are ongoing, and N numbers are still low, but with the first set of analyses, control and hSOD1^{G93A} male mice injected with iCre and Ascl1^{SA6} at 16 weeks did not show significant differences in right (FIG. 53A) and left (FIG. 53B) hindlimb stride length distances. Similarly, control and hSOD1^{G93A} female mice injected with iCre and Ascl1^{SA6} at 16 weeks did not show significant differences in right (FIG. 53C) and left (FIG. 53D) hindlimb stride length distances.

Behavior Tests after Treatment with iCre (Control) and Ascl1^{SA6} at 19 Weeks

In the first series of motor behaviour assessments, iCre and Ascl1^{SA6} were administered at 16 weeks of age, which is at the start of symptom onset. The more relevant question for therapeutic purposes is whether Ascl1^{SA6} can improve motor function when delivered at a later stage during disease course. Indeed, patients with ALS experience a delay average of 10-16 months from the initial symptom onset to formal diagnosis²³, meaning that therapeutic intervention will only be possible once disease has progressed such that neurological symptoms are clearly evident. Thus, the question is whether administration of Ascl1^{SA6} into hSOD1^{G93A} mice at 19 weeks would be able to improve or prevent the progression of motor symptoms. Baseline assessments were performed at 18 weeks-of-age in hSOD1^{G93A} mice and weight measurements and behavioural tests were performed weekly after injection of iCre and Ascl1^{SA6} into the motor cortex.

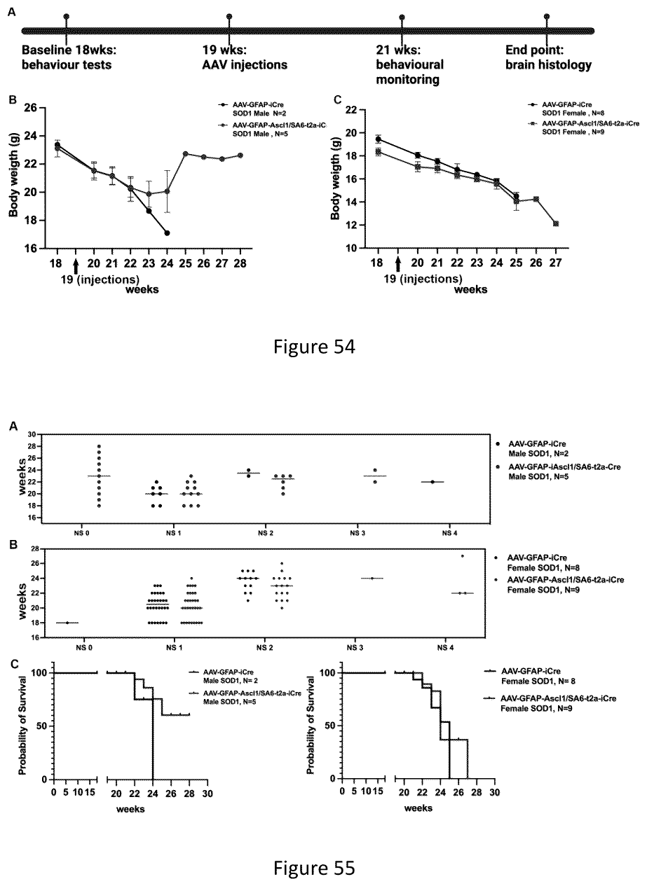

As shown with 16-week injections, hSOD1^{G93A} male (FIG. 54A) and female (FIG. 54B) mice injected at 19 weeks continuously lost body weight over time with disease progression. Ascl1^{SA6} treated male mice displayed a trend towards increasing their body weight overtime compared to iCre controls (FIG. 54A). Indeed, Ascl1^{SA6} injected male mice showed 60% survival probability until 28 weeks compared to 0% for iCre injected male mice. However, statistical tests could not be performed as iCre control animal numbers were still too low (N=2; FIG. 6A). In contrast, while injection of either iCre or Ascl1SA into female hSOD1^{G93A} mice did not alter body weight, Ascl1^{SA6} injected female mice showed 35% survival probability until 26 weeks compared to 0% for iCre injected female mice (FIG. 54B).

To assess the impact of 19-week injections on motor function, the neuroscore assay was again applied. In hSOD1^{G93A} male and female mice, motor symptoms gradually increased in severity, correlating with neuroscore increases from 18-week baseline measurements to the disease or experimental endpoint (FIG. 55). In both Ascl1^{SA6} and iCre treated animals, hSOD1^{G93A} male mice stayed at NS 1 until 23 weeks while female mice stayed at NS1 until 24 weeks. One Ascl1^{SA6} treated male mice stayed at NS 0 until sacrificed, while others stayed longer at NS 2 (FIG. 55A). In addition, one female hSOD1^{G93A} mouse stayed at NS 2 until 26 weeks (FIG. 55B). These results provide some evidence of functional benefits from Ascl1SA6 delivery on neuroscore progression in hSOD1^{G93A} mice.

The impact of iCre and Ascl1^{SA6} on survival of hSOD1^{G93A} mice when delivered at 19 weeks was also assessed. Statistical analysis of lifespan of iCre and Ascl1^{SA6} treated male mice did not reveal significant differences (FIG. 55C). Notably, 60% of Ascl1^{SA6} treated male mice survived until 24 weeks and an additional male mouse survived without symptoms until sacrificed at 28, surpassing the normal period of survival for hSOD1^{G93A} mice (FIG. 55C). In the female cohort of hSOD1^{G93A} mice injected mice at 19 weeks, one animal injected with Ascl1^{SA6} survived until 27 weeks, but overall, median survival was 25 weeks for iCre and 24 weeks for Ascl1^{SA6} and no statistical differences in survival time were noted (log-rank test, p=0.3503) (FIG. 55C).

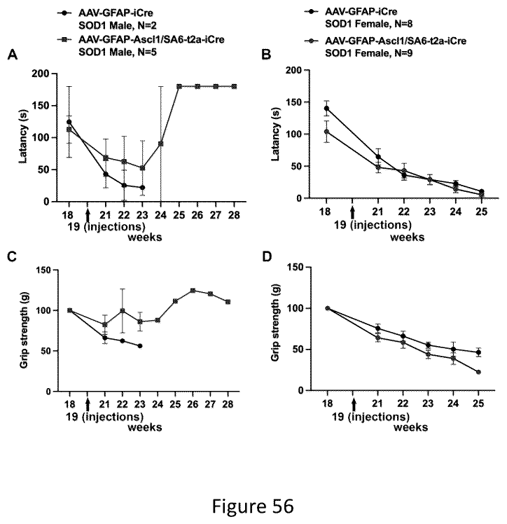

Next examined was latency to fall on the rotarod as a more sensitive measure of motor function. Treatment with Ascl1^{SA6} at 19 weeks in hSOD1^{G93A} male mice showed a trend towards longer time spent on the rotarod compared to the iCre group (FIG. 56A). In contrast, female hSOD1^{G93A} mice treated with iCre or Ascl1^{SA6} at 19 weeks spent very similar amounts of time on the rotarod, suggesting a lack of motor differences in the two groups (FIG. 56B).

Grip strength was also measured as an assessment of neuromuscular function, normalized to body weight. hSOD1^{G93A} male mice injected with Ascl1^{SA6} at 19 weeks trended towards an increase in grip strength compared to control mice as they aged, but these measures did not reach statistical significance due to the small sample size of the control group (FIG. 56C). Female mice also showed no significant differences in grip strength between 19 week iCre and Ascl1^{SA6} treated hSOD1^{G93A} mice (FIG. 56D).

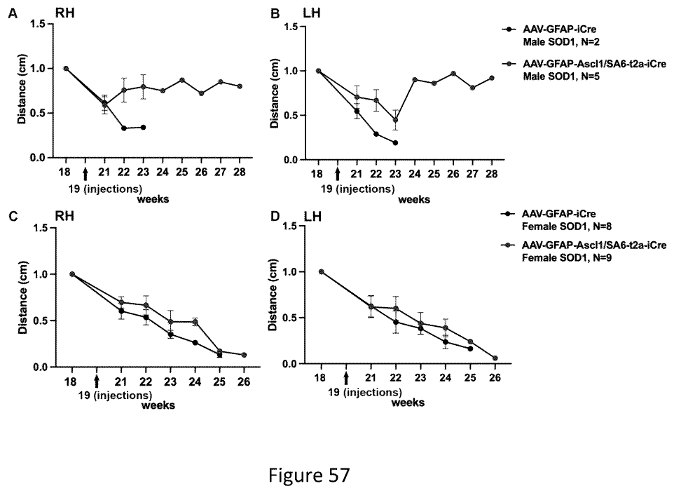

Finally, gait was analyzed using the Catwalk system. In hSOD1^{G93A} male mice injected with Ascl1^{SA6} at 19 weeks, there was a trend towards increased right and left hindlimb stride lengths compared to iCre controls, but statistical significance was not reached due to small control group size (FIG. 57A,B). Similarly, there was a smaller trend towards increased right and left hindlimb stride lengths in hSOD1^{G93A} female mice injected with Ascl1^{SA6} versus iCre at 19 weeks, but statistical significance was not achieved (FIG. 57C,D).

### Discussion

In view of the increasing evidence showing that UMN in the cortex degenerate ALS, it is crucial for replacing lost UMN to minimize reactive astrocytes and excitotoxic motor neuron death²⁴ to delay disease progression. In this study, a neuronal lineage conversion approach was used by injecting AAV5 carrying a short hGFAP promoter²⁵ in the motor cortex to drive the expression of mutated Ascl1 (Ascl1SA6). Converting serines to alanines in SP sites of Ascl1 will prevent inhibitory phosphorylation and increase neurogenic potential^{26,27} to regenerate enough functional GABAergic neuron. In addition, a recent study found that female and male ALS patients showed different level of neutrophils which correlated to disease progression²⁸.

The data in this Example show that neuronal lineage conversion of motor cortex astrocytes can delay disease progression in hSOD1^{G93A} transgenic mice, a mouse model of ALS. To promote astrocyte to neuron conversion, a GFAP was used to express a mutated version of the proneural gene Ascl1 (Ascl1^{SA6}) in motor cortex astrocytes. Gene delivery was achieved using adeno-associated virus 5 (AAV5), which was injected into the motor cortex in 16-week-old mice, at the onset of motor symptoms, and at 19-weeks, when motor symptoms were already quite severe. Male mice receiving Ascl1^{SA6} were better able to maintain their body weight and retain motor function, as revealed by a better (i.e., lower) neuroscore, and increased latency to fall on a rotarod. Taken together, these data demonstrate that neuronal lineage conversion targeted to motor cortex astrocytes can delay the onset of motor symptoms and increase lifespan in a mouse model of ALS.

### REFERENCES

1 Turner, M. R. et al. Controversies and priorities in amyotrophic lateral sclerosis. Lancet Neurol 12, 310-322 (2013).
2 Dadon-Nachum, M., Melamed, E. & Offen, D. The “dying-back” phenomenon of motor neurons in ALS. J Mol Neurosci 43,470-477 (2011).
3 Fischer, L. R. et al. Amyotrophic lateral sclerosis is a distal axonopathy: evidence in mice and man. Exp Neurol 185, 232-240 (2004).
4 Eisen, A. et al. Cortical influences drive amyotrophic lateral sclerosis. J Neurol Neurosurg Psychiatry 88, 917-924 (2017).
5 Eisen, A. The Dying Forward Hypothesis of ALS: Tracing Its History. Brain Sci 11 (2021).
6 Talbot, K. Amyotrophic Lateral Sclerosis: network vulnerability and monosynaptic connections. J Neurol Neurosurg Psychiatry 91, 906 (2020).
7 Vucic, S., Nicholson, G. A. & Kiernan, M. C. Cortical hyperexcitability may precede the onset of familial amyotrophic lateral sclerosis. Brain 131, 1540-1550 (2008).
8 Ozdinler, P. H. et al. Corticospinal motor neurons and related subcerebral projection neurons undergo early and specific neurodegeneration in hSOD1G(9)(3)A transgenic ALS mice. J Neurosci 31, 4166-4177 (2011).
9 Thomsen, G. M. et al. Delayed disease onset and extended survival in the SOD1G93A rat model of amyotrophic lateral sclerosis after suppression of mutant SOD1 in the motor cortex. J Neurosci 34, 15587-15600 (2014).
10 Jara, J. H., Villa, S. R., Khan, N. A., Bohn, M. C. & Ozdinler, P. H. AAV2 mediated retrograde transduction of corticospinal motor neurons reveals initial and selective apical dendrite degeneration in ALS. Neurobiol Dis 47, 174-183 (2012).
11 Crochemore, C., Pena-Altamira, E., Virgili, M., Monti, B. & Contestabile, A. Disease-related regressive alterations of forebrain cholinergic system in SOD1 mutant transgenic mice. Neurochem Int 46, 357-368 (2005).
12 Thomsen, G. M. et al. Transplantation of Neural Progenitor Cells Expressing Glial Cell Line-Derived Neurotrophic Factor into the Motor Cortex as a Strategy to Treat Amyotrophic Lateral Sclerosis. Stem Cells (2018).
13 Khademullah, C. S. et al. Cortical interneuron-mediated inhibition delays the onset of amyotrophic lateral sclerosis. Brain 143, 800-810 (2020).
14 Nagai, M. et al. Astrocytes expressing ALS-linked mutated SOD1 release factors selectively toxic to motor neurons. Nat Neurosci 10, 615-622 (2007).
15 Di Giorgio, F. P., Carrasco, M. A., Siao, M. C., Maniatis, T. & Eggan, K. Non-cell autonomous effect of glia on motor neurons in an embryonic stem cell-based ALS model. Nat Neurosci 10, 608-614 (2007).
16 Qian, K. et al. Sporadic ALS Astrocytes Induce Neuronal Degeneration In Vivo. Stem Cell Reports 8, 843-855 (2017).
17 Kwon, H. S. & Koh, S. H. Neuroinflammation in neurodegenerative disorders: the roles of microglia and astrocytes. Transl Neurodegener 9, 42 (2020).
18 Liddelow, S. A. et al. Neurotoxic reactive astrocytes are induced by activated microglia. Nature 541, 481-487 (2017).
19 Guttenplan, K. A. et al. Knockout of reactive astrocyte activating factors slows disease progression in an ALS mouse model. Nat Commun 11, 3753 (2020).
20 Taha, D. M. et al. Astrocytes display cell autonomous and diverse early reactive states in familial amyotrophic lateral sclerosis. Brain (2022).
21 Masserdotti, G., Gascon, S. & Gotz, M. Direct neuronal reprogramming: learning from and for development. Development 143, 2494-2510 (2016).
22 Hatzipetros, T. et al. A Quick Phenotypic Neurological Scoring System for Evaluating Disease Progression in the SOD1-G93A Mouse Model of ALS. J Vis Exp (2015).
23 Richards, D., Morren, J. A. & Pioro, E. P. in Amyotrophic Lateral Sclerosis (ed T. Araki) (Exon Publications Copyright: The Authors., 2021).
24 Van Den Bosch, L., Van Damme, P., Bogaert, E. & Robberecht, W. The role of excitotoxicity in the pathogenesis of amyotrophic lateral sclerosis. Biochim Biophys Acta 1762, 1068-1082 (2006).
25 Lee, Y., Messing, A., Su, M. & Brenner, M. GFAP promoter elements required for region-specific and astrocyte-specific expression. Glia 56, 481-493 (2008). https://doi.org:10.1002/glia.20622
26 Li, S. et al. RAS/ERK signaling controls proneural genetic programs in cortical development and gliomagenesis. J Neurosci 34, 2169-2190 (2014).
27 Li, S. et al. GSK3 Temporally Regulates Neurogenin 2 Proneural Activity in the Neocortex. Journal of Neuroscience 32, 7791-7805 (2012).
28 Murdock, B. J., Goutman, S. A., Boss, J., Kim, S. & Feldman, E. L. Amyotrophic Lateral Sclerosis Survival Associates With Neutrophils in a Sex-specific Manner. Neurol Neuroimmunol Neuroinflamm 8 (2021).
29 Wang, L. L. et al. Revisiting astrocyte to neuron conversion with lineage tracing in vivo. Cell 184, 5465-5481.e5416 (2021).
30 Fischer, K. B., Collins, H. K. & Callaway, E. M. Sources of off-target expression from recombinase-dependent AAV vectors and mitigation with cross-over insensitive ATG-out vectors. Proc Natl Acad Sci USA 116, 27001-27010 (2019).
31 Basso, M. et al. Mutant copper-zinc superoxide dismutase (SOD1) induces protein secretion pathway alterations and exosome release in astrocytes: implications for disease spreading and motor neuron pathology in amyotrophic lateral sclerosis. J Biol Chem 288, 15699-15711 (2013).
32 Frühbeis, C., Fröhlich, D., Kuo, W. P. & Krämer-Albers, E. M. Extracellular vesicles as mediators of neuron-glia communication. Front Cell Neurosci 7, 182 (2013).
33 Wang, L.-L., Garcia, C. S., Zhong, X., Ma, S. & Zhang, C.-L. (2020).
34 Gong Chen, Wen Li, Zongqin Xiang, Liang Xu, Minhui Liu, Qingsong Wang, Wenliang Lei, bioRxiv 2020.09.02.279273. DOI: 10.1101/2020.09.02.279273
35 Hol, E. M. et al. Neuronal expression of GFAP in patients with Alzheimer pathology and identification of novel GFAP splice forms. Mol Psychiatry 8, 786-796 (2003).
36 Wang, L. L. et al. The p53 Pathway Controls SOX2-Mediated Reprogramming in the Adult Mouse Spinal Cord. Cell Rep 17, 891-903 (2016).
37 Gascon, S. et al. Identification and Successful Negotiation of a Metabolic Checkpoint in Direct Neuronal Reprogramming. Cell Stem Cell 18, 396-409 (2016).
38 Hachem, L. D., Mothe, A. J. & Tator, C. H. Effect of BDNF and Other Potential Survival Factors in Models of In Vitro Oxidative Stress on Adult Spinal Cord-Derived Neural Stem/Progenitor Cells. Biores Open Access 4, 146-159 (2015).
39 Singh, A., Kukreti, R., Saso, L. & Kukreti, S. Oxidative Stress: A Key Modulator in Neurodegenerative Diseases. Molecules 24 (2019).
40 Arlotta, P. et al. Neuronal subtype-specific genes that control corticospinal motor neuron development in vivo. Neuron 45, 207-221 (2005).
41 Molyneaux, B. J., Arlotta, P., Menezes, J. R. & Macklis, J. D. Neuronal subtype specification in the cerebral cortex. Nat Rev Neurosci 8, 427-437 (2007).
42 Fogarty, M. J., Noakes, P. G. & Bellingham, M. C. Motor cortex layer V pyramidal neurons exhibit dendritic regression, spine loss, and increased synaptic excitation in the presymptomatic hSOD1(G93A) mouse model of amyotrophic lateral sclerosis. J Neurosci 35, 643-647 (2015).
43 Alkaslasi, M. R. et al. Poor Corticospinal Motor Neuron Health Is Associated with Increased Symptom Severity in the Acute Phase Following Repetitive Mild TBI and Predicts Early ALS Onset in Genetically Predisposed Rodents. Brain Sci 11(2021).
44 Khademullah, C. S. et al. Cortical interneuron-mediated inhibition delays the onset of amyotrophic lateral sclerosis. Brain 143, 800-810 (2020).
45 Liu, Y. et al. Ascl1 Converts Dorsal Midbrain Astrocytes into Functional Neurons In Vivo. J Neurosci 35, 9336-9355 (2015).
46 Chouchane, M. et al. Lineage Reprogramming of Astroglial Cells from Different Origins into Distinct Neuronal Subtypes. Stem Cell Reports 9, 162-176 (2017).
47 Hogg, M. C., Halang, L., Woods, I., Coughlan, K. S. & Prehn, J. H. M. Riluzole does not improve lifespan or motor function in three ALS mouse models. Amyotroph Lateral Scler Frontotemporal Degener 19,438-445 (2018).

### Example 8: Ascl1-SA7 Repression of Sox9 Expression in Transduced Cortical Cells

This Example was performed to determine what, if any, difference in direct lineage reprogramming results from transduction with an Ascl1-SA7 mutant in comparison to transduction with the Ascl1-SA6 mutant. Both were expected to provide an advantage over the Ascl1 transcription factor due to the incorporation of phosphoacceptor site mutations allowing the mutant bHLH transcription factor Ascl1 to maintain activity even in cellular contexts in which it may not normally be active.

The cortex of Rosa-zsGreen mice were transduced with AAV8-GFAPshort-iCre, AAV8-GFAPshort-Ascl1-t2a-iCre, AAV8-GFAPshort-Ascl1-SA6-t2a-iCre and AAV8-GFAPshort-Ascl1-SA7-t2a-iCre, where the packaged AAVs were injected using stereotactic surgery into the cerebral cortex of Rosa-zsGreen mice, as described in the Examples above. At 21 days post-transduction, the mice were sacrificed and cortical tissue analyzed, as also described above.

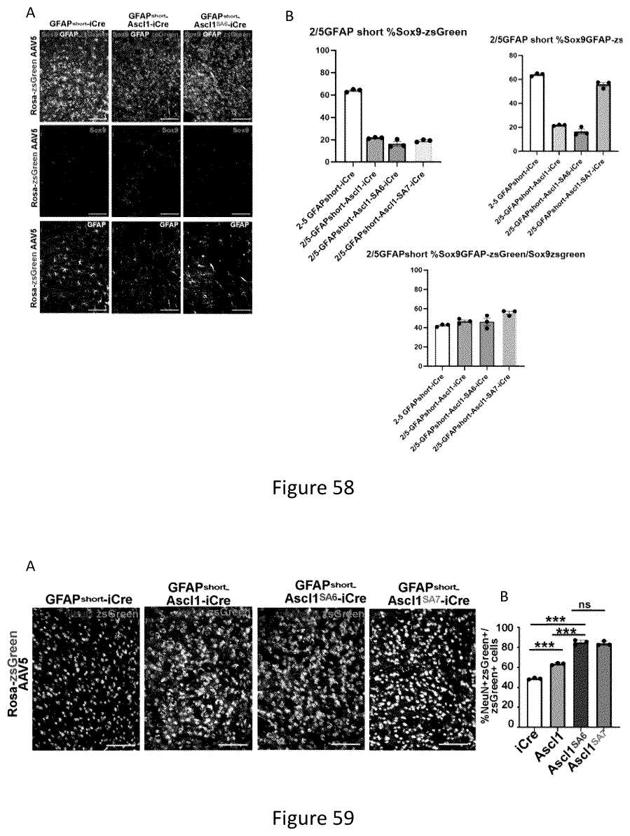

The data provided in FIGS. 58 and 59, confirms that Ascl1-SA7 is equally as efficient as Ascl1-SA6 at direct lineage reprogramming of reactive astrocytes to neurons.

All publications, patents and patent applications mentioned in this Specification are indicative of the level of skill of those skilled in the art to which this invention pertains and are herein incorporated by reference to the same extent as if each individual publication, patent, or patent applications was specifically and individually indicated to be incorporated by reference.

The invention being thus described, it will be obvious that the same may be varied in many ways. Such variations are not to be regarded as a departure from the spirit and scope of the invention, and all such modifications as would be obvious to one skilled in the art are intended to be included within the scope of the following claims.

1. A mutant basic-helix-loop-helix (bHLH) transcription factor, wherein the mutant bHLH transcription factor comprises a mutation of one or more phosphoacceptor sites for proline-directed serine-threonine kinases found in the corresponding wild-type bHLH transcription factor, and wherein the mutant bHLH transcription factor exhibits reduced inhibition of function from phosphorylation by proline-directed serine-threonine kinases than the corresponding wild-type bHLH transcription factor.

2. The mutant bHLH transcription factor according to claim 1, which comprises a mutation of two or more, or three or more, or four or more, phosphoacceptor sites for proline-directed serine-threonine kinases found in the corresponding wild-type bHLH transcription factor.

3. The mutant bHLH transcription factor according to claim 2, which comprises a mutation of all of the phosphoacceptor sites for proline-directed serine-threonine kinases found in the corresponding wild-type bHLH transcription factor.

4. The mutant bHLH transcription factor according to claim 1, wherein each mutation of a phosphoacceptor site for proline-directed serine-threonine kinases comprises substitution of a serine or threonine within the phosphoacceptor site with another amino acid selected from the group consisting of alanine, glycine, valine, leucine, isoleucine, D-serine, D-threonine, D-alanine, D-glycine, D-valine, D-leucine and D-isoleucine.

5. The mutant bHLH transcription factor according to claim 1, wherein the mutant bHLH transcription factor is a mutant of a proneural bHLH transcription factor selected from the group consisting of Neurog2, Ascl1, and Neurod4.

6. The mutant bHLH transcription factor according to claim 5, which is:
a) at least 80%, 85%, 90%, 95%, or 97% identical to SEQ ID NO:1 or SEQ ID NO:2;
b) at least 75%, 80%, 85%, 90%, 95%, or 97% identical to SEQ ID NO:13 or SEQ ID NO:14; or
c) at least 70%, 75%, 80%, 85%, 90%, 95%, or 97% identical to SEQ ID NO:19 or SEQ ID NO:20.

7. The mutant bHLH transcription factor according to claim 6, wherein the mutant bHLH transcription factor is human Ascl1-SA5, mouse Ascl1-SA6, human Ascl1-SA6, mouse Ascl1-SA7, human Neurod4-SA4TA6, Neurod4-SA3TA4, human Neurod4-SA5TA6, mouse Neurod4-SA4TA4, human Neurog2-SA8TA1, mouse Neurog2-SA9TA1, human Neurog2-SA8TA2, mouse Neurog2-SA9TA2 or mouse Neurog2-SA9.

8. The mutant bHLH transcription factor according to claim 1, wherein the mutant bHLH transcription factor is a mutant of a Neurod1 neuronal differentiation transcription factor.

9. The mutant bHLH transcription factor according to claim 8, which is at least 80%, 85%, 90%, 95%, or 97% identical to SEQ ID NO:7 or SEQ ID NO:8.

10. The mutant bHLH transcription factor according to claim 9, wherein the mutant bHLH transcription factor is human Neurod1-SA6, mouse Neurod1-SA6, Neurod1-SA7, or mouse Neurod1-SA7.

11. The mutant bHLH transcription factor according to claim 10, which additionally comprises a mutation in a conserved protein kinase A (PKA) site in the helix-loop-helix domain of the corresponding wild-type bHLH transcription factor.

12. The mutant bHLH transcription factor according to claim 11, wherein the mutation of the conserved PKA site comprises substitution of a serine or threonine within the phosphoacceptor site with another amino acid selected from the group consisting of alanine, glycine, valine, leucine, isoleucine, D-serine, D-threonine, D-alanine, D-glycine, D-valine, D-leucine and D-isoleucine.

13. A nucleic acid encoding the mutant bHLH according to claim 1.

14. The nucleic acid according to claim 13, wherein the mutant bHLH transcription factor coding sequence is operably linked to a glial cell-selective promoter.

15. The nucleic acid according to claim 14, wherein the glial cell-selective promoter is a glial fibrillary acidic protein (GFAP) promoter.

16. A vector comprising the nucleic acid according to claim 13, wherein the vector is a non-viral vector or a viral vector.

17. The vector according to claim 16, which is an adeno-associated viral (AAV) vector, retroviral vector or lentiviral vector.

18. The vector according to claim 17, wherein the AAV vector is an AAV2/5 vector, an AAV2/8 vector, or an AAV 9 vector.

19. The vector according to claim 16, wherein nucleic acid encoding the mutant bHLH transcription factor is operably linked to a glial cell-selective promoter.

20. The vector according to claim 16, wherein the vector additionally comprises a nucleic acid sequence encoding a second transcription factor.

21. (canceled)

22. (canceled)

23. A pharmaceutical composition comprising the mutant bHLH transcription factor according to claim 1 or a nucleic acid encoding the mutant bHLH transcription factor, wherein the nucleic acid is optionally comprised in a vector and a pharmaceutically acceptable carrier or excipient.

24. The pharmaceutical composition of claim 23, wherein the composition is formulated for direct intracranial administration, intracranial injection, intracerebroventricular injection, intracisternal injection, intrathecal injection, intravenous injection, intraperitoneal injection, intraarterial, delivery via nanoparticles, and/or administration using focused ultrasound.

25. (canceled)

26. (canceled)

27. (canceled)

28. A method for neuronal transformation of glial cells in a subject, said method comprising administering to the subject the mutant bHLH transcription factor according to claim 1 or a nucleic acid encoding the mutant bHLH transcription factor, wherein the nucleic acid is optionally comprised in a vector.

29. A method for preventing or treating a neurodegenerative disease or disorder or for treating of a CNS or brain injury in a subject, said method comprising administering to the subject the mutant bHLH transcription factor according to or a nucleic acid encoding the mutant bHLH transcription factor, wherein the nucleic acid is optionally comprised in a vector.

30. (canceled)

31. (canceled)

32. A method for neuronal transformation of glial cells in a subject, said method comprising administering to the subject a ZBTB18 transcription factor or a nucleic acid encoding the ZBTB18 transcription factor.

33. A vector comprising a nucleic acid encoding a ZBTB18 transcription factor comprising an amino acid sequence that is at least 80%, 85%, 90%, 95%, or 97% identical to one of SEQ ID NOs:36, 38, 40, 42, 44, 46, 48, 50, 52 or 54, wherein the vector is a non-viral vector or a viral vector.


SEQUENCE LISTING (55 sequences)

SEQ ID NO: 1 - protein, 237 aa, Homo sapiens
MESSAKMESGGAGQQPQPQPQQPFLPPAACFFATAAAAAAAAAAAAAQSAQQQQQQQQQQQQAPQLRPAADGQPSGGGHKSAPKQVKRQRSSSPELMRCKRRLNFSGFGYSLPQQQPAAVARRNERERNRVKLVNLGFATLREHVPNGAANKKMSSKVETLRSAVEYIRALQQLLDEHDAVSAAFQAGVLSPTISPNYSNDLNSMAGSPVSSYSSDEGSYDPLSPEEQELLDFTNWF

SEQ ID NO: 2 - protein, 231 aa, Mus musculus
MESSGKMESGAGQQPQPPQPFLPPAACFFATAAAAAAAAAAAAQSAQQQQPQAPPQQAPQLSPVADSQPSGGGHKSAAKQVKRQRSSSPELMRCKRRLNFSGFGYSLPQQQPAAVARRNERERNRVKLVNLGFATLREHVPNGAANKKMSKVETLRSAVEYIRALQQLLDEHDAVSAAFQAGVLSPTISPNYSNDLNSMAGSPVSSYSSDEGSYDPLSPEEQELLDFTNWF

SEQ ID NO: 3 - protein, 231 aa, Mus musculus
MESSGKMESGAGQQPQPPQPFLPPAACFFATAAAAAAAAAAAAQSAQQQQPQAPPQQAPQLAPVADSQPSGGGHKSAAKQVKRQRSSAPELMRCKRRLNFSGFGYSLPQQQPAAVARRNERERNRVKLVNLGFATLREHVPNGAANKKMSKVETLRSAVEYIRALQQLLDEHDAVSAAFQAGVLAPTIAPNYSNDLNSMAGAPVSSYSSDEGSYDPLAPEEQELLDFTNWF

SEQ ID NO: 4 - DNA, 696 bp, Mus musculus
atggagagctctggcaagatggagagtggagccggccagcagccgcagcccccgcagcccttcctgcctcccgcagcctgcttctttgcgaccgcggcggcggcggcagcggcggcggccgcggcagctcagagcgcgcagcagcaacagccgcaggcgccgccgcagcaggcgccgcagctggccccggtggccgacagccagccctcagggggcggtcacaagtcagcggccaagcaggtcaagcgccagcgctcgtccgctccggaactgatgcgctgcaaacgccggctcaacttcagcggcttcggctacagcctgccacagcagcagccggccgccgtggcgcgccgcaacgagcgcgagcgcaaccgggtcaagttggtcaacctgggctttgccaccctccgggagcatgtccccaacggcgcggccaacaagaagatggccaaggtggagacgctgcgctcggcggtcgagtacatccgcgcgctgcagcagctgctggacgagcacgacgcggtgagcgctgcctttcaggcgggcgtcctggcgcccaccatcgcccccaactactccaacgacttgaactctatggcgggtgctccggtctcgtcctactcctccgacgagggatcctacgaccctcttgccccagaggaacaagagctgctggactttaccaactggttctga

SEQ ID NO: 5 - protein, 231 aa, Mus musculus
MESSGKMESGAGQQPQPPQPFLPPAACFFATAAAAAAAAAAAAQSAQQQQPQAPPQQAPQLAPVADSQPSGGGHKSAAKQVKRQRSSAPELMRCKRRLNFSGFGYSLPQQQPAAVARRNERERNRVKLVNLGFATLREHVPNGAANKKMAKVETLRSAVEYIRALQQLLDEHDAVSAAFQAGVLAPTIAPNYSNDLNSMAGAPVSSYSSDEGSYDPLAPEEQELLDFTNWF

SEQ ID NO: 6 - DNA, 696 bp, Mus musculus; identical to SEQ ID NO: 4

SEQ ID NO: 7 - protein, 356 aa, Homo sapiens
MTKSYSESGLMGEPQPQGPPSWTDECLSSQDEEHEADKKEDDLETMNAEEDSLRNGGEEEDEDEDLEEEEEEEEEDDDQKPKRRGPKKKKMTKARLERFKLRRMKANARERNRMHGLNAALDNLRKVVPCYSKTQKLSKIETLRLAKNYIWALSEILRSGKSPDLVSFVQTLCKGLSQPTTNLVAGCLQLNPRTFLPEQNQDMPPHLPTASASFPVHPYSYQSPGLPSPPYGTMDSSHVFHVKPPPHAYSAALEPFFESPLTDCTSPSFDGPLSPPLSINGNFSFKHEPSAEFEKNYAFTMHYPAATLAGAQSHGSIFSGTAAPRCEIPIDNIMSFDSHSHHERVMSAQLNAIFHD

SEQ ID NO: 8 - protein, 357 aa, Mus musculus
MTKSYSESGLMGEPQPQGPPSWTDECLSSQDEEHEADKKEDELEAMNAEEDSLRNGGEEEEEDEDLEEEEEEEEEEEDQKPKRRGPKKKKMTKARLERFKLRRMKANARERNRMHGLNAALDNLRKVVPCYSKTQKLSKIETLRLAKNYIWALSEILRSGKSPDLVSFVQTLCKGLSQPTTNLVAGCLQLNPRTFLPEQNPDMPPHLPTASASFPVHPYSYQSPGLPSPPYGTMDSSHVFHVKPPPHAYSAALEPFFESPLTDCTSPSFDGPLSPPLSINGNFSFKHEPSAEFEKNYAFTMHYPAATLAGPQSHGSIFSSGAAAPRCEIPIDNIMSFDSHSHHERVMSAQLNAIFHD

SEQ ID NO: 9 - protein, 357 aa, Mus musculus
MTKSYSESGLMGEPQPQGPPSWTDECLSSQDEEHEADKKEDELEAMNAEEDSLRNGGEEEEEDEDLEEEEEEEEEEEDQKPKRRGPKKKKMTKARLERFKLRRMKANARERNRMHGLNAALDNLRKVVPCYSKTQKLSKIETLRLAKNYIWALSEILRSGKAPDLVSFVQTLCKGLSQPTTNLVAGCLQLNPRTFLPEQNPDMPPHLPTASASFPVHPYSYQAPGLPAPPYGTMDSSHVFHVKPPPHAYSAALEPFFEAPLTDCTAPSFDGPLAPPLSINGNFSFKHEPSAEFEKNYAFTMHYPAATLAGPQSHGSIFSSGAAAPRCEIPIDNIMSFDSHSHHERVMSAQLNAIFHD

SEQ ID NO: 10 - DNA, 1074 bp, Mus musculus
atgaccaaatcatacagcgagagcgggctgatgggcgagcctcagccccaaggtcccccaagctggacagatgagtgtctcagttctcaggacgaggaacacgaggcagacaagaaagaggacgagcttgaagccatgaatgcagaggaggactctctgagaaacgggggagaggaggaggaggaagatgaggatctagaggaagaggaggaagaagaagaggaggaggaggatcaaaagcccaagagacggggtcccaaaaagaaaaagatgaccaaggcgcgcctagaacgttttaaattaaggcgcatgaaggccaacgcccgcgagcggaaccgcatgcacgggctgaacgcggcgctggacaacctgcgcaaggtggtaccttgctactccaagacccagaaactgtctaaaatagagacactgcgcttggccaagaactacatctgggctctgtcagagatcctgcgctcaggcaaagcccctgatctggtctccttcgtacagacgctctgcaaaggtttgtcccagcccactaccaatttggtcgccggctgcctgcagctcaaccctcggactttcttgcctgagcagaacccggacatgcccccgcatctgccaaccgccagcgcttccttcccggtgcatccctactcctaccaggcccctggactgcccgccccgccctacggcaccatggacagctcccacgtcttccacgtcaagccgccgccacacgcctacagcgcagctctggagcccttctttgaagcccccctaactgactgcaccgccccttcctttgacggacccctcgccccgccgctcagcatcaatggcaacttctctttcaaacacgaaccatccgccgagtttgaaaaaaattatgcctttaccatgcactaccctgcagcgacgctggcagggccccaaagccacggatcaatcttctcttccggtgccgctgcccctcgctgcgagatccccatagacaacattatgtctttcgatagccattcgcatcatgagcgagtcatgagtgcccagcttaatgccatctttcacgattag

SEQ ID NO: 11 - protein, 357 aa, Mus musculus
MTKSYSESGLMGEPQPQGPPSWTDECLSSQDEEHEADKKEDELEAMNAEEDSLRNGGEEEEEDEDLEEEEEEEEEEEDQKPKRRGPKKKKMTKARLERFKLRRMKANARERNRMHGLNAALDNLRKVVPCYSKTQKLAKIETLRLAKNYIWALSEILRSGKAPDLVSFVQTLCKGLSQPTTNLVAGCLQLNPRTFLPEQNPDMPPHLPTASASFPVHPYSYQAPGLPAPPYGTMDSSHVFHVKPPPHAYSAALEPFFEAPLTDCTAPSFDGPLAPPLSINGNFSFKHEPSAEFEKNYAFTMHYPAATLAGPQSHGSIFSSGAAAPRCEIPIDNIMSFDSHSHHERVMSAQLNAIFHD

SEQ ID NO: 12 - DNA, 1074 bp, Mus musculus
atgaccaaatcatacagcgagagcgggctgatgggcgagcctcagccccaaggtcccccaagctggacagatgagtgtctcagttctcaggacgaggaacacgaggcagacaagaaagaggacgagcttgaagccatgaatgcagaggaggactctctgagaaacgggggagaggaggaggaggaagatgaggatctagaggaagaggaggaagaagaagaggaggaggaggatcaaaagcccaagagacggggtcccaaaaagaaaaagatgaccaaggcgcgcctagaacgttttaaattaaggcgcatgaaggccaacgcccgcgagcggaaccgcatgcacgggctgaacgcggcgctggacaacctgcgcaaggtggtaccttgctactccaagacccagaaactggctaaaatagagacactgcgcttggccaagaactacatctgggctctgtcagagatcctgcgctcaggcaaagcccctgatctggtctccttcgtacagacgctctgcaaaggtttgtcccagcccactaccaatttggtcgccggctgcctgcagctcaaccctcggactttcttgcctgagcagaacccggacatgcccccgcatctgccaaccgccagcgcttccttcccggtgcatccctactcctaccaggcccctggactgcccgccccgccctacggcaccatggacagctcccacgtcttccacgtcaagccgccgccacacgcctacagcgcagctctggagcccttctttgaagcccccctaactgactgcaccgccccttcctttgacggacccctcgccccgccgctcagcatcaatggcaacttctctttcaaacacgaaccatccgccgagtttgaaaaaaattatgcctttaccatgcactaccctgcagcgacgctggcagggccccaaagccacggatcaatcttctcttccggtgccgctgcccctcgctgcgagatccccatagacaacattatgtctttcgatagccattcgcatcatgagcgagtcatgagtgcccagcttaatgccatctttcacgattag

SEQ ID NO: 13 - protein, 331 aa, Homo sapiens
MSKTFVKSKEMGELVNTPSWMDKGLGSQNEVKEEESRPGTYGMLSSLTEEHDSIEEEEEEEEDGEKPKRRGPKKKKMTKARLERFRARRVKANARERTRMHGLNDALDNLRRVMPCYSKTQKLSKIETLRLARNYIWALSEVLETGQTPEGKGFVEMLCKGLSQPTSNLVAGCLQLGPQSVLLEKHEDKSPICDSAISVHNFNYQSPGLPSPPYGHMETHLLHLKPQVFKSLGESSFGSHLPDCSTPPYEGPLTPPLSISGNFSLKQDGSPDLEKSYSFMPHYPSSSLSSGHVHSTPFQAGTPRYDVPIDMSYDSYPHHGIGTQLNTVFTE

SEQ ID NO: 14 - protein, 330 aa, Mus musculus
MAKMYMKSKDMVELVNTQSWMDKGLSSQNEMKEQERRPGSYGMLGTLTEEHDSIEEDEEEEEDGDKPKRRGPKKKKMTKARLERFRARRVKANARERTRMHGLNDALDNLRRVMPCYSKTQKLSKIETLRLARNYIWALSEVLETGQTLEGKGFVEMLCKGLSQPTSNLVAGCLQLGPQSTLLEKHEEKSSICDSTISVHSFNYQSPGLPSPPYGHMETHSLHLKPQPFKSLGDSFGSHPPDCSTPPYEGPLTPPLSISGNFSLKQDGSPDLEKSYNFMPHYTSASLSSGHVHSTPFQTGTPRYDVPVDLSYDSYSHHSIGTQLNTIFSD

SEQ ID NO: 15 - protein, 330 aa, Mus musculus
MAKMYMKSKDMVELVNTQSWMDKGLSSQNEMKEQERRPGSYGMLGTLTEEHDSIEEDEEEEEDGDKPKRRGPKKKKMTKARLERFRARRVKANARERTRMHGLNDALDNLRRVMPCYSKTQKLSKIETLRLARNYIWALSEVLETGQTLEGKGFVEMLCKGLSQPTSNLVAGCLQLGPQSTLLEKHEEKSSICDSTISVHSFNYQAPGLPAPPYGHMETHSLHLKPQPFKSLGDSFGSHPPDCSAPPYEGPLAPPLSISGNFSLKQDGAPDLEKSYNFMPHYTSASLSSGHVHSAPFQTGAPRYDVPVDLSYDSYSHHSIGTQLNTIFSD

SEQ ID NO: 16 - DNA, 993 bp, Mus musculus
atggcaaaaatgtatatgaaatccaaggacatggtggagctggtcaacacacaatcctggatggacaaaggtctgagctctcaaaatgagatgaaggagcaagagagaagaccgggctcttatggaatgctcggaaccttaactgaagagcatgacagtattgaggaggatgaagaagaggaagaagatggagataaacctaaaagaagaggtcccaagaaaaagaagatgactaaagctcgccttgaaagattcagggctcgaagagtcaaggccaatgctagagaacggacccggatgcatggcctgaatgatgccttggataatcttaggagagtcatgccatgttactctaaaactcaaaagctttccaagatagagactcttcgactggcaaggaactacatctgggccttgtctgaagtcctggagactggtcagacacttgaagggaagggatttgtagagatgctatgtaaaggtctctctcaacccacaagcaacctggttgctggatgcctccaactggggcctcaatctaccctcctggagaagcatgaggaaaaatcttcaatttgtgactctactatctctgtccacagcttcaactatcaggctccagggctccccgcccctccttatggccatatggaaacacattctctccatctcaagcctcaaccatttaagagtttgggtgactcttttgggagccatccacctgactgcagtgcccccccttatgagggtccactcgcaccacccctgagcattagtggcaacttctccttaaagcaagacggcgcccctgatttggaaaaatcctacaatttcatgccacattatacctctgcaagtctaagttcagggcatgtgcattcagctccctttcagactggcgctccccgctatgatgttcctgtagacctgagctatgattcctactcccaccatagcattggaactcagctcaatacgatcttctctgattag

SEQ ID NO: 17 - protein, 330 aa, Mus musculus
MAKMYMKSKDMVELVNTQSWMDKGLSSQNEMKEQERRPGSYGMLGTLTEEHDSIEEDEEEEEDGDKPKRRGPKKKKMTKARLERFRARRVKANARERTRMHGLNDALDNLRRVMPCYSKTQKLAKIETLRLARNYIWALSEVLETGQTLEGKGFVEMLCKGLSQPTSNLVAGCLQLGPQSTLLEKHEEKSSICDSTISVHSFNYQAPGLPAPPYGHMETHSLHLKPQPFKSLGDSFGSHPPDCSAPPYEGPLAPPLSISGNFSLKQDGAPDLEKSYNFMPHYTSASLSSGHVHSAPFQTGAPRYDVPVDLSYDSYSHHSIGTQLNTIFSD

SEQ ID NO: 18 - DNA, 993 bp, Mus musculus
atggcaaaaatgtatatgaaatccaaggacatggtggagctggtcaacacacaatcctggatggacaaaggtctgagctctcaaaatgagatgaaggagcaagagagaagaccgggctcttatggaatgctcggaaccttaactgaagagcatgacagtattgaggaggatgaagaagaggaagaagatggagataaacctaaaagaagaggtcccaagaaaaagaagatgactaaagctcgccttgaaagattcagggctcgaagagtcaaggccaatgctagagaacggacccggatgcatggcctgaatgatgccttggataatcttaggagagtcatgccatgttactctaaaactcaaaagcttgccaagatagagactcttcgactggcaaggaactacatctgggccttgtctgaagtcctggagactggtcagacacttgaagggaagggatttgtagagatgctatgtaaaggtctctctcaacccacaagcaacctggttgctggatgcctccaactggggcctcaatctaccctcctggagaagcatgaggaaaaatcttcaatttgtgactctactatctctgtccacagcttcaactatcaggctccagggctccccgcccctccttatggccatatggaaacacattctctccatctcaagcctcaaccatttaagagtttgggtgactcttttgggagccatccacctgactgcagtgcccccccttatgagggtccactcgcaccacccctgagcattagtggcaacttctccttaaagcaagacggcgcccctgatttggaaaaatcctacaatttcatgccacattatacctctgcaagtctaagttcagggcatgtgcattcagctccctttcagactggcgctccccgctatgatgttcctgtagacctgagctatgattcctactcccaccatagcattggaactcagctcaatacgatcttctctgattag

SEQ ID NO: 19 - protein, 272 aa, Homo sapiens
MFVKSETLELKEEEDVLVLLGSASPALAALTPLSSSADEEEEEEPGASGGARRQRGAEAGQGARGGVAAGAEGCRPARLLGLVHDCKRRPSRARAVSRGAKTAETVQRIKKTRRLKANNRERNRMHNLNAALDALREVLPTFPEDAKLTKIETLRFAHNYIWALTETLRLADHCGGGGGGLPGALFSEAVLLSPGGASAALSSSGDSPSPASTWSCTNSPAPSSSVSSNSTSPYSCTLSPASPAGSDMDYWQPPPPDKHRYAPHLPIARDCI

SEQ ID NO: 20 - protein, 263 aa, Mus musculus
MFVKSETLELKEEEEVLMLLGSASPASATLTPMSSSADEEEDEELRRPGSARGQRGAEAGQGVQGSPASGAGGCRPGRLLGLMHECKRRPSRSRAVSRGAKTAETVQRIKKTRRLKANNRERNRMHNLNAALDALREVLPTFPEDAKLTKIETLRFAHNYIWALTETLRLADHCAGAGGLQGALFTEAVLLSPGAALGASGDSPSPPSSWSCTNSPASSSNSTSPYSCTLSPASPGSDVDYWQPPPPEKHRYAPHLPLARDCI

SEQ ID NO: 21 - protein, 263 aa, Mus musculus
MFVKSETLELKEEEEVLMLLGSAAPASATLAPMSSSADEEEDEELRRPGSARGQRGAEAGQGVQGAPASGAGGCRPGRLLGLMHECKRRPSRSRAVSRGAKTAETVQRIKKTRRLKANNRERNRMHNLNAALDALREVLPTFPEDAKLTKIETLRFAHNYIWALTETLRLADHCAGAGGLQGALFTEAVLLAPGAALGASGDAPAPPSSWSCTNAPASSSNSTAPYSCTLAPAAPGSDVDYWQPPPPEKHRYAPHLPLARDCI

SEQ ID NO: 22 - DNA, 792 bp, Mus musculus
atgttcgtcaaatctgagactctggagttgaaggaggaagaggaggtactgatgctgctgggctcggctgccccggcctcggcgaccctggccccgatgtcctccagcgcggacgaggaggaggacgaggagctgcgccggccgggctccgcgcgtgggcagcgtggagcggaagccgggcagggggtgcagggcgctccggcgtcgggtgccgggggttgccggccagggcggctgctgggcctgatgcacgagtgcaagcgtcgcccgtcgcgctcacgggccgtctcccgaggtgccaagacggcggagacggtgcagcgcatcaagaagacccgcaggctcaaggccaacaaccgcgagcgcaaccgcatgcacaacctaaacgccgcgctggacgcgctgcgcgaggtgctgcccaccttccccgaggatgccaagctcacgaagatcgagacgctgcgcttcgcccacaattacatctgggcgctcaccgagactctgcgcctggcggaccactgcgccggcgccggtggcctccagggggcgctcttcacggaggcggtgctcctggccccgggagctgcgctcggcgccagcggggacgcccctgctccaccttcctcctggagctgcaccaacgccccggcgtcatcctccaactccacggccccatacagctgcactttagcgcccgctgcccccgggtcagacgtggactactggcagcccccacctccggagaagcatcgttatgcgcctcacctgcccctcgccagggactgtatctag

SEQ ID NO: 23 - protein, 263 aa, Mus musculus
MFVKSETLELKEEEEVLMLLGSAAPASATLAPMSSSADEEEDEELRRPGSARGQRGAEAGQGVQGAPASGAGGCRPGRLLGLMHECKRRPSRSRAVSRGAKTAETVQRIKKTRRLKANNRERNRMHNLNAALDALREVLPTFPEDAKLAKIETLRFAHNYIWALTETLRLADHCAGAGGLQGALFTEAVLLAPGAALGASGDAPAPPSSWSCTNAPASSSNSTAPYSCTLAPAAPGSDVDYWQPPPPEKHRYAPHLPLARDCI

SEQ ID NO: 24 - DNA, 792 bp, Mus musculus
atgttcgtcaaatctgagactctggagttgaaggaggaagaggaggtactgatgctgctgggctcggctgccccggcctcggcgaccctggccccgatgtcctccagcgcggacgaggaggaggacgaggagctgcgccggccgggctccgcgcgtgggcagcgtggagcggaagccgggcagggggtgcagggcgctccggcgtcgggtgccgggggttgccggccagggcggctgctgggcctgatgcacgagtgcaagcgtcgcccgtcgcgctcacgggccgtctcccgaggtgccaagacggcggagacggtgcagcgcatcaagaagacccgcaggctcaaggccaacaaccgcgagcgcaaccgcatgcacaacctaaacgccgcgctggacgcgctgcgcgaggtgctgcccaccttccccgaggatgccaagctcgcgaagatcgagacgctgcgcttcgcccacaattacatctgggcgctcaccgagactctgcgcctggcggaccactgcgccggcgccggtggcctccagggggcgctcttcacggaggcggtgctcctggccccgggagctgcgctcggcgccagcggggacgcccctgctccaccttcctcctggagctgcaccaacgccccggcgtcatcctccaactccacggccccatacagctgcactttagcgcccgctgcccccgggtcagacgtggactactggcagcccccacctccggagaagcatcgttatgcgcctcacctgcccctcgccagggactgtatctag

SEQ ID NO: 25 - protein, 263 aa, Mus musculus
MFVKSETLELKEEEEVLMLLGSAAPASATLTPMSSSADEEEDEELRRPGSARGQRGAEAGQGVQGAPASGAGGCRPGRLLGLMHECKRRPSRSRAVSRGAKTAETVQRIKKTRRLKANNRERNRMHNLNAALDALREVLPTFPEDAKLTKIETLRFAHNYIWALTETLRLADHCAGAGGLQGALFTEAVLLAPGAALGASGDAPAPPSSWSCTNAPASSSNSTAPYSCTLAPAAPGSDVDYWQPPPPEKHRYAPHLPLARDCI

SEQ ID NO: 26 - DNA, 21 bp, Artificial Sequence
  Primer
catcagccctaatccatctga

SEQ ID NO: 27 - DNA, 21 bp, Artificial Sequence
  Primer
cgcgactaacaatcaaagtga

SEQ ID NO: 28 - DNA, 17 bp, Artificial Sequence
  Primer
ctggcttctgaggaccg

SEQ ID NO: 29 - DNA, 21 bp, Artificial Sequence
  Primer
aatctgtgggaagtcttgtcc

SEQ ID NO: 30 - DNA, 19 bp, Artificial Sequence
  Primer
accagaagtggcacctgac

SEQ ID NO: 31 - DNA, 24 bp, Artificial Sequence
  Primer
caaattttgtaatccagaggttga

SEQ ID NO: 32 - DNA, 20 bp, Artificial Sequence
  Primer
ggcattaaagcagcgtatcc

SEQ ID NO: 33 - DNA, 19 bp, Artificial Sequence
  Primer
ctgttcctgtacggcatgg

SEQ ID NO: 34 - DNA, 24 bp, Artificial Sequence
  Primer
ctaggccacagaattgaaagatct

SEQ ID NO: 35 - DNA, 22 bp, Artificial Sequence
  Primer
gtaggtggaaattctagcatcc

SEQ ID NO: 36 - protein, 531 aa, Mus musculus
MCPKGYEDSMEFPDHSRHLLQCLSEQRHQGFLCDCTVLVGDAQFRAHRAVLASCSMYFHLFYKDQLDKRDIVHLNSDIVTAPAFALLLEFMYEGKLQFKDLPIEDVLAAASYLHMYDIVKVCKKKLKEKATTEADSTKKEEDASSCSDKVESLSDGSSHMAGDLPSDEDEGEDDKLNILPSKRDLAAEPGNMWMRLPSDSAGIPQAGGEAEPHATAAGKTVASPCSSTESLSQRSVTSVRDSADVDCVLDLSVKSSLSGVENLNSSYFSSQDVLRSNLVQVKVEKEASCDESDVGTNDYDMEHSTVKESVSTNNRVQYEPAHLAPLREDSVLRELDREDKASDDEMMTPESERVQVEGGMENSLLPYVSNILSPAGQIFMCPLCNKVFPSPHILQIHLSTHFREQDGIRSKPAADVNVPTCSLCGKTFSCMYTLKRHERTHSGEKPYTCTQCGKSFQYSHNLSRHAVVHTREKPHACKWCERRFTQSGDLYRHIRKFHCELVNSLSVKSEALSLPTVRDWTLEDSSQELWK

SEQ ID NO: 37 - DNA, 3848 bp, Mus musculus
agtgtgtgcggatctgtggtggagaaggtatctcattcctctccaacatcatctccactttgcatctgtctctcttagtctgctttttttccaccgatgtaacagacctgcagccagcaagactccgagggaaggacttaatcaagtttatgtgtcctaaaggttatgaagacagtatggagtttccagaccacagcagacatttgctgcagtgtctgagcgagcagaggcaccagggctttctttgtgactgcactgttctggtgggagatgcccagttccgggcacaccgagctgttctggcttcatgcagcatgtatttccacctcttttacaaggaccagctggacaaaagagacattgttcatctgaacagcgacattgtgacagcccccgctttcgctctcctgcttgaattcatgtacgaagggaaactccagttcaaagacttgcccattgaggacgtgctagcagctgccagttatctccacatgtatgacattgtcaaagtctgcaaaaagaagctgaaagagaaagctaccacagaggcggacagcaccaaaaaagaagaagatgcttcaagttgttcggataaagtcgagagcctctcagacggcagcagccacatggcaggtgacctgcccagtgatgaagatgaaggcgaagatgacaaattgaacattctacccagcaaaagggacttggcagctgagcctgggaacatgtggatgcgattgccctcagactcagcaggcatcccccaggctggcggagaggccgagccacacgccacagcagctggaaaaacagtagccagcccctgcagctcaacagagtctttgtcccagaggtctgtcacctccgtgagggattcggcagatgttgactgtgtgctggacctgtctgtcaagtccagcctttcaggagttgaaaatctgaacagctcttatttctcttcacaggacgtgctgagaagcaacctggtgcaggtgaaggtggagaaagaggcgtcctgtgatgagagtgatgttggcactaatgactatgacatggaacatagcactgtgaaagagagtgtgagcactaacaacagggtacagtatgagccagcccacctggctcctctgagggaggactcggtcttaagggaactggatcgggaggacaaagccagcgatgatgagatgatgaccccagagagcgagcgggtccaggtggaagggggcatggagaacagtctgcttccctacgtctccaatatcctgagccccgcaggccagatcttcatgtgccccctgtgcaacaaagtcttccccagcccccacatcctgcagatccacctgagcacgcacttccgagagcaggatggcatccgtagcaagcctgccgccgatgtcaatgtgcccacgtgctccctgtgtgggaagactttctcctgcatgtacaccctcaagcgccacgagaggactcactcgggggagaagccctacacgtgcacccaatgcggcaagagtttccagtactcgcacaacctgagccgtcatgccgtggtgcacacccgcgagaagccccatgcctgcaagtggtgcgagcgcaggttcacgcagtccggagacctgtacagacacatccgcaagttccactgtgagttggtgaactccttgtcggtcaaaagtgaagcgctgagcttgcccactgtcagagactggaccttagaagatagctctcaagaactttggaaataattttatatatatatatatataaataatatatatatatatatatacatatatatacatagatctctatatagttgtggtacggtctaaaagcagtcttgtttcctggaactgaaaagttgggatcttaacttgtttttgcactttagaataatagcatgagaatctcactaatttagcattctgataaaagaaactagagcaagtcacaagtagagagctgtctttcttttgagggttaggccaagttagccaataaaaccttctcaacaaatggttctgtcacaaacgctttcacatggcttacttttgtacacactgcattgtcttgagcgctttccctgtgctccacactgttttgttttgttttgttttcagagtagaaatcccctccagtttattagcctctttatatgtctcaaattgcatgaatattttctggctgttggaaacctgaatgcttttagacccaaatggaaaatttctgaaatgctggattatctatttttaaacaagcagttgacttaaaactttgtgtggcaacttctggttttctgatggtccccagtgagagaaatctgacagtacactgggtcactgggactgtctctgtgaaggtttgcttgagatgaaaaaggatattgcagcttcagcagtgttgaactgtgtgtttaaaatgtgaattactgttcttgtatactgtaattgattacacgggctggggggggtgtcaaagaacttgacaggttgtgttgatgctcttagttgagtcttgaaaagtaaatattaacgctacagaaatgcatgagtttcaatatattttttgtctttgtttgcattgtataactttaacgagtgagtttaaaattatttaatttccttagaaaaatagcaccatttgggaaaaaaactggtgttacgaagaacataaatgcactgtctttatttttatttttattttatataatttaaattgacttttcccacgtctttaagtttgaaactgttaagctgaataaaaacttaagctgcaaattgataacttcgctacattacaaggaacatataaatgtttacaaacggcttaaagaattgcatgtgcagtgtgcattcataccacacttgtaattgcacagaacccatgccagctcagagtttaggtgtacacacgtacccagccgagtgtttttagaataactactgcacaaattgacaataggtcataggtcattctctctctccctcctttttgcttcccctttctttttctcccctatttcctttccttccccctaccccctaactcatgaaaagattctatggactgaaaaagccccaggctgaaaggactgaaccgccttgattgacatggggaggggggttagtagactgtgtatcggcagcagaggctgcagcccgatgtgtggttctgatggccagcgcggggcagaagcgttttgcacactgtttgttttcctgtcttgcacttacaaataaggtctatgggagtagcatgggaaacagtggttgtttttccctttttcttttttaattgcttttgtttaaaatttgatcgccttaactactgtaaacatagcctatttttgtgcttaagatactgaatggaaaactccattgtgtgttgctggactgttttggaaatatttggtcaaatgtatgttaatttggctgtaatggcatttaaagcaaacaaacaaacaaacaaacaaagctgtgaaaatggccttggagcattatctttagttacttgaagagtttctagtttttttttttttaatacagtttatgttaaaataatctttattaatctagaggagacaatcaatgtctgtggaagaacggacttttttggattttcttttttaatggtcattgtgagtgattgctctttcttttacttagtttcacattcttcctttgttctaaatcttagactgacatctagctttgacaatcatagtatgttttatttcctgagggggaataacttataatgctgtttagttttgtactattggtgtgttggtgaatttttaaactgtgtgctaactgcaataaattatatgaactgagaaaaaaaaaaaa

SEQ ID NO: 38 - protein, 522 aa, Mus musculus
MEFPDHSRHLLQCLSEQRHQGFLCDCTVLVGDAQFRAHRAVLASCSMYFHLFYKDQLDKRDIVHLNSDIVTAPAFALLLEFMYEGKLQFKDLPIEDVLAAASYLHMYDIVKVCKKKLKEKATTEADSTKKEEDASSCSDKVESLSDGSSHMAGDLPSDEDEGEDDKLNILPSKRDLAAEPGNMWMRLPSDSAGIPQAGGEAEPHATAAGKTVASPCSSTESLSQRSVTSVRDSADVDCVLDLSVKSSLSGVENLNSSYFSSQDVLRSNLVQVKVEKEASCDESDVGTNDYDMEHSTVKESVSTNNRVQYEPAHLAPLREDSVLRELDREDKASDDEMMTPESERVQVEGGMENSLLPYVSNILSPAGQIFMCPLCNKVFPSPHILQIHLSTHFREQDGIRSKPAADVNVPTCSLCGKTFSCMYTLKRHERTHSGEKPYTCTQCGKSFQYSHNLSRHAVVHTREKPHACKWCERRFTQSGDLYRHIRKFHCELVNSLSVKSEALSLPTVRDWTLEDSSQELWK

SEQ ID NO: 39 - DNA, 4954 bp, Mus musculus
tcagaaggtatcggggttaggatgaaagtttcactgtgtctcagcatagtgtggaggagagaagtagtttctgaatagtagcttcatatccatccaagatgcacttgcagaggtagatgttggcgagctctgcctttggtcccaggatggggaagtgggctccgctgatcttaagagagttttagccactacagatgaattttgactgatctttgccttcccttctttcaccctctcccttgccaaaaaacatgggaaagatgcataattggaagaatagaaggtactgggaaaacaaaatagatcaggttattattgaaacaaagtattttctgttggatacctgaggcagagccagaaaaaaaagttttatttattttattttttctaggaaggctttaaaaaatatttaacttacaaagtttttgcacctcattttacagtggtaaaagtttgattaattttaatatgaagttccttttgccctatagcagatcaaagttcagatgaaaatgttcagagttaacagactctctggttgctaatgttgttgaggtagaggatctacaaagaaacaggaccccccccccattcatttttaaaatgtttggatttgttcatagtaaagatgatctttcccatctgtcggtacagttctgctcctctgtgtgtgtagtacctgcagatcttaccgcaccaacatctgttcttatacacagggcttttgttgttgtaacactctgactttaaaagtaagttgtttgctgatttctgcactaattatgcaatttcattttttttccccttcatttcagccaaatgtgtgtgttcagtctgtgctttctaagtagttgggtatcagacgtgttacaaagaatggctggacatgtcttcttgactcttccagaatttgtgtgggactttgcctgatattatgagttctttccctttgaagttaaagctctcttttaggttttgtaggatgtcatttgatgagttgttggttaaatttctggaagaggtctgagttcttgttctaagtcataagctaaaggaagctttaaacttgaccaggagttatgagtggggtgagcaaagccttggtgattgcatttaggttgaatgtgtggcttgggagtccttggggtgtgagcctgatttaaaattcagacatgtaactctccaccactaaggcggatattttttaaagtggcgactagagtctaaattcctgactaggctctaacgagaaactcctctttccccaggttatgaagacagtatggagtttccagaccacagcagacatttgctgcagtgtctgagcgagcagaggcaccagggctttctttgtgactgcactgttctggtgggagatgcccagttccgggcacaccgagctgttctggcttcatgcagcatgtatttccacctcttttacaaggaccagctggacaaaagagacattgttcatctgaacagcgacattgtgacagcccccgctttcgctctcctgcttgaattcatgtacgaagggaaactccagttcaaagacttgcccattgaggacgtgctagcagctgccagttatctccacatgtatgacattgtcaaagtctgcaaaaagaagctgaaagagaaagctaccacagaggcggacagcaccaaaaaagaagaagatgcttcaagttgttcggataaagtcgagagcctctcagacggcagcagccacatggcaggtgacctgcccagtgatgaagatgaaggcgaagatgacaaattgaacattctacccagcaaaagggacttggcagctgagcctgggaacatgtggatgcgattgccctcagactcagcaggcatcccccaggctggcggagaggccgagccacacgccacagcagctggaaaaacagtagccagcccctgcagctcaacagagtctttgtcccagaggtctgtcacctccgtgagggattcggcagatgttgactgtgtgctggacctgtctgtcaagtccagcctttcaggagttgaaaatctgaacagctcttatttctcttcacaggacgtgctgagaagcaacctggtgcaggtgaaggtggagaaagaggcgtcctgtgatgagagtgatgttggcactaatgactatgacatggaacatagcactgtgaaagagagtgtgagcactaacaacagggtacagtatgagccagcccacctggctcctctgagggaggactcggtcttaagggaactggatcgggaggacaaagccagcgatgatgagatgatgaccccagagagcgagcgggtccaggtggaagggggcatggagaacagtctgcttccctacgtctccaatatcctgagccccgcaggccagatcttcatgtgccccctgtgcaacaaagtcttccccagcccccacatcctgcagatccacctgagcacgcacttccgagagcaggatggcatccgtagcaagcctgccgccgatgtcaatgtgcccacgtgctccctgtgtgggaagactttctcctgcatgtacaccctcaagcgccacgagaggactcactcgggggagaagccctacacgtgcacccaatgcggcaagagtttccagtactcgcacaacctgagccgtcatgccgtggtgcacacccgcgagaagccccatgcctgcaagtggtgcgagcgcaggttcacgcagtccggagacctgtacagacacatccgcaagttccactgtgagttggtgaactccttgtcggtcaaaagtgaagcgctgagcttgcccactgtcagagactggaccttagaagatagctctcaagaactttggaaataattttatatatatatatatataaataatatatatatatatatatacatatatatacatagatctctatatagttgtggtacggtctaaaagcagtcttgtttcctggaactgaaaagttgggatcttaacttgtttttgcactttagaataatagcatgagaatctcactaatttagcattctgataaaagaaactagagcaagtcacaagtagagagctgtctttcttttgagggttaggccaagttagccaataaaaccttctcaacaaatggttctgtcacaaacgctttcacatggcttacttttgtacacactgcattgtcttgagcgctttccctgtgctccacactgttttgttttgttttgttttcagagtagaaatcccctccagtttattagcctctttatatgtctcaaattgcatgaatattttctggctgttggaaacctgaatgcttttagacccaaatggaaaatttctgaaatgctggattatctatttttaaacaagcagttgacttaaaactttgtgtggcaacttctggttttctgatggtccccagtgagagaaatctgacagtacactgggtcactgggactgtctctgtgaaggtttgcttgagatgaaaaaggatattgcagcttcagcagtgttgaactgtgtgtttaaaatgtgaattactgttcttgtatactgtaattgattacacgggctggggggggtgtcaaagaacttgacaggttgtgttgatgctcttagttgagtcttgaaaagtaaatattaacgctacagaaatgcatgagtttcaatatattttttgtctttgtttgcattgtataactttaacgagtgagtttaaaattatttaatttccttagaaaaatagcaccatttgggaaaaaaactggtgttacgaagaacataaatgcactgtctttatttttatttttattttatataatttaaattgacttttcccacgtctttaagtttgaaactgttaagctgaataaaaacttaagctgcaaattgataacttcgctacattacaaggaacatataaatgtttacaaacggcttaaagaattgcatgtgcagtgtgcattcataccacacttgtaattgcacagaacccatgccagctcagagtttaggtgtacacacgtacccagccgagtgtttttagaataactactgcacaaattgacaataggtcataggtcattctctctctccctcctttttgcttcccctttctttttctcccctatttcctttccttccccctaccccctaactcatgaaaagattctatggactgaaaaagccccaggctgaaaggactgaaccgccttgattgacatggggaggggggttagtagactgtgtatcggcagcagaggctgcagcccgatgtgtggttctgatggccagcgcggggcagaagcgttttgcacactgtttgttttcctgtcttgcacttacaaataaggtctatgggagtagcatgggaaacagtggttgtttttccctttttcttttttaattgcttttgtttaaaatttgatcgccttaactactgtaaacatagcctatttttgtgcttaagatactgaatggaaaactccattgtgtgttgctggactgttttggaaatatttggtcaaatgtatgttaatttggctgtaatggcatttaaagcaaacaaacaaacaaacaaacaaagctgtgaaaatggccttggagcattatctttagttacttgaagagtttctagtttttttttttttaatacagtttatgttaaaataatctttattaatctagaggagacaatcaatgtctgtggaagaacggacttttttggattttcttttttaatggtcattgtgagtgattgctctttcttttacttagtttcacattcttcctttgttctaaatcttagactgacatctagctttgacaatcatagtatgttttatttcctgagggggaataacttataatgctgtttagttttgtactattggtgtgttggtgaatttttaaactgtgtgctaactgcaataaattatatgaactgagaaaaaaaaaaaa

SEQ ID NO: 40 - protein, 530 aa, Mus musculus
CPKGYEDSMEFPDHSRHLLQCLSEQRHQGFLCDCTVLVGDAQFRAHRAVLASCSMYFHLFYKDQLDKRDIVHLNSDIVTAPAFALLLEFMYEGKLQFKDLPIEDVLAAASYLHMYDIVKVCKKKLKEKATTEADSTKKEEDASSCSDKVESLSDGSSHMAGDLPSDEDEGEDDKLNILPSKRDLAAEPGNMWMRLPSDSAGIPQAGGEAEPHATAAGKTVASPCSSTESLSQRSVTSVRDSADVDCVLDLSVKSSLSGVENLNSSYFSSQDVLRSNLVQVKVEKEASCDESDVGTNDYDMEHSTVKESVSTNNRVQYEPAHLAPLREDSVLRELDREDKASDDEMMTPESERVQVEGGMENSLLPYVSNILSPAGQIFMCPLCNKVFPSPHILQIHLSTHFREQDGIRSKPAADVNVPTCSLCGKTFSCMYTLKRHERTHSGEKPYTCTQCGKSFQYSHNLSRHAVVHTREKPHACKWCERRFTQSGDLYRHIRKFHCELVNSLSVKSEALSLPTVRDWTLEDSSQELWK

SEQ ID NO: 41 - DNA, 4123 bp, Mus musculus
aggctgcggccgccgctgggctgctgccatggggagccgttgaaagagtggggccctgtggctcggccgcgcgcctcaggctgccagcgcgggcggggggcggcgggggcgcgcggggcccgggccggggccggcgggcgggcgggggccggcggaggacgcccacaagtccagcggcggcggcggcggcagaggaggaggaggaggaggacgcggcggggacgcggcgactcgggccgctccgtaacctccgagggtccgcggtagcggtggacgggtcgatccgtggagtggatgtcttcgggccattgcggtgcttgggactcattaaactgtcactcacccccaccaccaccccactggtctgctttttttccaccgatgtaacagacctgcagccagcaagactccgagggaaggacttaatcaagtttatgtgtcctaaaggttatgaagacagtatggagtttccagaccacagcagacatttgctgcagtgtctgagcgagcagaggcaccagggctttctttgtgactgcactgttctggtgggagatgcccagttccgggcacaccgagctgttctggcttcatgcagcatgtatttccacctcttttacaaggaccagctggacaaaagagacattgttcatctgaacagcgacattgtgacagcccccgctttcgctctcctgcttgaattcatgtacgaagggaaactccagttcaaagacttgcccattgaggacgtgctagcagctgccagttatctccacatgtatgacattgtcaaagtctgcaaaaagaagctgaaagagaaagctaccacagaggcggacagcaccaaaaaagaagaagatgcttcaagttgttcggataaagtcgagagcctctcagacggcagcagccacatggcaggtgacctgcccagtgatgaagatgaaggcgaagatgacaaattgaacattctacccagcaaaagggacttggcagctgagcctgggaacatgtggatgcgattgccctcagactcagcaggcatcccccaggctggcggagaggccgagccacacgccacagcagctggaaaaacagtagccagcccctgcagctcaacagagtctttgtcccagaggtctgtcacctccgtgagggattcggcagatgttgactgtgtgctggacctgtctgtcaagtccagcctttcaggagttgaaaatctgaacagctcttatttctcttcacaggacgtgctgagaagcaacctggtgcaggtgaaggtggagaaagaggcgtcctgtgatgagagtgatgttggcactaatgactatgacatggaacatagcactgtgaaagagagtgtgagcactaacaacagggtacagtatgagccagcccacctggctcctctgagggaggactcggtcttaagggaactggatcgggaggacaaagccagcgatgatgagatgatgaccccagagagcgagcgggtccaggtggaagggggcatggagaacagtctgcttccctacgtctccaatatcctgagccccgcaggccagatcttcatgtgccccctgtgcaacaaagtcttccccagcccccacatcctgcagatccacctgagcacgcacttccgagagcaggatggcatccgtagcaagcctgccgccgatgtcaatgtgcccacgtgctccctgtgtgggaagactttctcctgcatgtacaccctcaagcgccacgagaggactcactcgggggagaagccctacacgtgcacccaatgcggcaagagtttccagtactcgcacaacctgagccgtcatgccgtggtgcacacccgcgagaagccccatgcctgcaagtggtgcgagcgcaggttcacgcagtccggagacctgtacagacacatccgcaagttccactgtgagttggtgaactccttgtcggtcaaaagtgaagcgctgagcttgcccactgtcagagactggaccttagaagatagctctcaagaactttggaaataattttatatatatatatatataaataatatatatatatatatatacatatatatacatagatctctatatagttgtggtacggtctaaaagcagtcttgtttcctggaactgaaaagttgggatcttaacttgtttttgcactttagaataatagcatgagaatctcactaatttagcattctgataaaagaaactagagcaagtcacaagtagagagctgtctttcttttgagggttaggccaagttagccaataaaaccttctcaacaaatggttctgtcacaaacgctttcacatggcttacttttgtacacactgcattgtcttgagcgctttccctgtgctccacactgttttgttttgttttgttttcagagtagaaatcccctccagtttattagcctctttatatgtctcaaattgcatgaatattttctggctgttggaaacctgaatgcttttagacccaaatggaaaatttctgaaatgctggattatctatttttaaacaagcagttgacttaaaactttgtgtggcaacttctggttttctgatggtccccagtgagagaaatctgacagtacactgggtcactgggactgtctctgtgaaggtttgcttgagatgaaaaaggatattgcagcttcagcagtgttgaactgtgtgtttaaaatgtgaattactgttcttgtatactgtaattgattacacgggctggggggggtgtcaaagaacttgacaggttgtgttgatgctcttagttgagtcttgaaaagtaaatattaacgctacagaaatgcatgagtttcaatatattttttgtctttgtttgcattgtataactttaacgagtgagtttaaaattatttaatttccttagaaaaatagcaccatttgggaaaaaaactggtgttacgaagaacataaatgcactgtctttatttttatttttattttatataatttaaattgacttttcccacgtctttaagtttgaaactgttaagctgaataaaaacttaagctgcaaattgataacttcgctacattacaaggaacatataaatgtttacaaacggcttaaagaattgcatgtgcagtgtgcattcataccacacttgtaattgcacagaacccatgccagctcagagtttaggtgtacacacgtacccagccgagtgtttttagaataactactgcacaaattgacaataggtcataggtcattctctctctccctcctttttgcttcccctttctttttctcccctatttcctttccttccccctaccccctaactcatgaaaagattctatggactgaaaaagccccaggctgaaaggactgaaccgccttgattgacatggggaggggggttagtagactgtgtatcggcagcagaggctgcagcccgatgtgtggttctgatggccagcgcggggcagaagcgttttgcacactgtttgttttcctgtcttgcacttacaaataaggtctatgggagtagcatgggaaacagtggttgtttttccctttttcttttttaattgcttttgtttaaaatttgatcgccttaactactgtaaacatagcctatttttgtgcttaagatactgaatggaaaactccattgtgtgttgctggactgttttggaaatatttggtcaaatgtatgttaatttggctgtaatggcatttaaagcaaacaaacaaacaaacaaacaaagctgtgaaaatggccttggagcattatctttagttacttgaagagtttctagtttttttttttttaatacagtttatgttaaaataatctttattaatctagaggagacaatcaatgtctgtggaagaacggacttttttggattttcttttttaatggtcattgtgagtgattgctctttcttttacttagtttcacattcttcctttgttctaaatcttagactgacatctagctttgacaatcatagtatgttttatttcctgagggggaataacttataatgctgtttagttttgtactattggtgtgttggtgaatttttaaactgtgtgctaactgcaataaattatatgaactgagaa

SEQ ID NO: 42 - protein, 521 aa, Mus musculus
EFPDHSRHLLQCLSEQRHQGFLCDCTVLVGDAQFRAHRAVLASCSMYFHLFYKDQLDKRDIVHLNSDIVTAPAFALLLEFMYEGKLQFKDLPIEDVLAAASYLHMYDIVKVCKKKLKEKATTEADSTKKEEDASSCSDKVESLSDGSSHMAGDLPSDEDEGEDDKLNILPSKRDLAAEPGNMWMRLPSDSAGIPQAGGEAEPHATAAGKTVASPCSSTESLSQRSVTSVRDSADVDCVLDLSVKSSLSGVENLNSSYFSSQDVLRSNLVQVKVEKEASCDESDVGTNDYDMEHSTVKESVSTNNRVQYEPAHLAPLREDSVLRELDREDKASDDEMMTPESERVQVEGGMENSLLPYVSNILSPAGQIFMCPLCNKVFPSPHILQIHLSTHFREQDGIRSKPAADVNVPTCSLCGKTFSCMYTLKRHERTHSGEKPYTCTQCGKSFQYSHNLSRHAVVHTREKPHACKWCERRFTQSGDLYRHIRKFHCELVNSLSVKSEALSLPTVRDWTLEDSSQELWK

SEQ ID NO: 43 - DNA, 3937 bp, Mus musculus
aggctgcggccgccgctgggctgctgccatggggagccgttgaaagagtggggccctgtggctcggccgcgcgcctcaggctgccagcgcgggcggggggcggcgggggcgcgcggggcccgggccggggccggcgggcgggcgggggccggcggaggacgcccacaagtccagcggcggcggcggcggcagaggaggaggaggaggaggacgcggcggggacgcggcgactcgggccgctccgtgttatgaagacagtatggagtttccagaccacagcagacatttgctgcagtgtctgagcgagcagaggcaccagggctttctttgtgactgcactgttctggtgggagatgcccagttccgggcacaccgagctgttctggcttcatgcagcatgtatttccacctcttttacaaggaccagctggacaaaagagacattgttcatctgaacagcgacattgtgacagcccccgctttcgctctcctgcttgaattcatgtacgaagggaaactccagttcaaagacttgcccattgaggacgtgctagcagctgccagttatctccacatgtatgacattgtcaaagtctgcaaaaagaagctgaaagagaaagctaccacagaggcggacagcaccaaaaaagaagaagatgcttcaagttgttcggataaagtcgagagcctctcagacggcagcagccacatggcaggtgacctgcccagtgatgaagatgaaggcgaagatgacaaattgaacattctacccagcaaaagggacttggcagctgagcctgggaacatgtggatgcgattgccctcagactcagcaggcatcccccaggctggcggagaggccgagccacacgccacagcagctggaaaaacagtagccagcccctgcagctcaacagagtctttgtcccagaggtctgtcacctccgtgagggattcggcagatgttgactgtgtgctggacctgtctgtcaagtccagcctttcaggagttgaaaatctgaacagctcttatttctcttcacaggacgtgctgagaagcaacctggtgcaggtgaaggtggagaaagaggcgtcctgtgatgagagtgatgttggcactaatgactatgacatggaacatagcactgtgaaagagagtgtgagcactaacaacagggtacagtatgagccagcccacctggctcctctgagggaggactcggtcttaagggaactggatcgggaggacaaagccagcgatgatgagatgatgaccccagagagcgagcgggtccaggtggaagggggcatggagaacagtctgcttccctacgtctccaatatcctgagccccgcaggccagatcttcatgtgccccctgtgcaacaaagtcttccccagcccccacatcctgcagatccacctgagcacgcacttccgagagcaggatggcatccgtagcaagcctgccgccgatgtcaatgtgcccacgtgctccctgtgtgggaagactttctcctgcatgtacaccctcaagcgccacgagaggactcactcgggggagaagccctacacgtgcacccaatgcggcaagagtttccagtactcgcacaacctgagccgtcatgccgtggtgcacacccgcgagaagccccatgcctgcaagtggtgcgagcgcaggttcacgcagtccggagacctgtacagacacatccgcaagttccactgtgagttggtgaactccttgtcggtcaaaagtgaagcgctgagcttgcccactgtcagagactggaccttagaagatagctctcaagaactttggaaataattttatatatatatatatataaataatatatatatatatatatacatatatatacatagatctctatatagttgtggtacggtctaaaagcagtcttgtttcctggaactgaaaagttgggatcttaacttgtttttgcactttagaataatagcatgagaatctcactaatttagcattctgataaaagaaactagagcaagtcacaagtagagagctgtctttcttttgagggttaggccaagttagccaataaaaccttctcaacaaatggttctgtcacaaacgctttcacatggcttacttttgtacacactgcattgtcttgagcgctttccctgtgctccacactgttttgttttgttttgttttcagagtagaaatcccctccagtttattagcctctttatatgtctcaaattgcatgaatattttctggctgttggaaacctgaatgcttttagacccaaatggaaaatttctgaaatgctggattatctatttttaaacaagcagttgacttaaaactttgtgtggcaacttctggttttctgatggtccccagtgagagaaatctgacagtacactgggtcactgggactgtctctgtgaaggtttgcttgagatgaaaaaggatattgcagcttcagcagtgttgaactgtgtgtttaaaatgtgaattactgttcttgtatactgtaattgattacacgggctggggggggtgtcaaagaacttgacaggttgtgttgatgctcttagttgagtcttgaaaagtaaatattaacgctacagaaatgcatgagtttcaatatattttttgtctttgtttgcattgtataactttaacgagtgagtttaaaattatttaatttccttagaaaaatagcaccatttgggaaaaaaactggtgttacgaagaacataaatgcactgtctttatttttatttttattttatataatttaaattgacttttcccacgtctttaagtttgaaactgttaagctgaataaaaacttaagctgcaaattgataacttcgctacattacaaggaacatataaatgtttacaaacggcttaaagaattgcatgtgcagtgtgcattcataccacacttgtaattgcacagaacccatgccagctcagagtttaggtgtacacacgtacccagccgagtgtttttagaataactactgcacaaattgacaataggtcataggtcattctctctctccctcctttttgcttcccctttctttttctcccctatttcctttccttccccctaccccctaactcatgaaaagattctatggactgaaaaagccccaggctgaaaggactgaaccgccttgattgacatggggaggggggttagtagactgtgtatcggcagcagaggctgcagcccgatgtgtggttctgatggccagcgcggggcagaagcgttttgcacactgtttgttttcctgtcttgcacttacaaataaggtctatgggagtagcatgggaaacagtggttgtttttccctttttcttttttaattgcttttgtttaaaatttgatcgccttaactactgtaaacatagcctatttttgtgcttaagatactgaatggaaaactccattgtgtgttgctggactgttttggaaatatttggtcaaatgtatgttaatttggctgtaatggcatttaaagcaaacaaacaaacaaacaaacaaagctgtgaaaatggccttggagcattatctttagttacttgaagagtttctagtttttttttttttaatacagtttatgttaaaataatctttattaatctagaggagacaatcaatgtctgtggaagaacggacttttttggattttcttttttaatggtcattgtgagtgattgctctttcttttacttagtttcacattcttcctttgttctaaatcttagactgacatctagctttgacaatcatagtatgttttatttcctgagggggaataacttataatgctgtttagttttgtactattggtgtgttggtgaatttttaaactgtgtgctaactgcaataaattatatgaactgagaaaaaaaaaaaaaaaaaa

SEQ ID NO: 44 - protein, 521 aa, Mus musculus; identical to SEQ ID NO: 42

SEQ ID NO: 45 - DNA, 4039 bp, Mus musculus
aggctgcggccgccgctgggctgctgccatggggagccgttgaaagagtggggccctgtggctcggccgcgcgcctcaggctgccagcgcgggcggggggcggcgggggcgcgcggggcccgggccggggccggcgggcgggcgggggccggcggaggacgcccacaagtccagcggcggcggcggcggcagaggaggaggaggaggaggacgcggcggggacgcggcgactcgggccgctccgtaacctccgagggtccgcggtagcggtggacgggtcgatccgtggagtggatgtcttcgggccattgcggtgcttgggactcattaaactgtcactcacccccaccaccaccccactgggttatgaagacagtatggagtttccagaccacagcagacatttgctgcagtgtctgagcgagcagaggcaccagggctttctttgtgactgcactgttctggtgggagatgcccagttccgggcacaccgagctgttctggcttcatgcagcatgtatttccacctcttttacaaggaccagctggacaaaagagacattgttcatctgaacagcgacattgtgacagcccccgctttcgctctcctgcttgaattcatgtacgaagggaaactccagttcaaagacttgcccattgaggacgtgctagcagctgccagttatctccacatgtatgacattgtcaaagtctgcaaaaagaagctgaaagagaaagctaccacagaggcggacagcaccaaaaaagaagaagatgcttcaagttgttcggataaagtcgagagcctctcagacggcagcagccacatggcaggtgacctgcccagtgatgaagatgaaggcgaagatgacaaattgaacattctacccagcaaaagggacttggcagctgagcctgggaacatgtggatgcgattgccctcagactcagcaggcatcccccaggctggcggagaggccgagccacacgccacagcagctggaaaaacagtagccagcccctgcagctcaacagagtctttgtcccagaggtctgtcacctccgtgagggattcggcagatgttgactgtgtgctggacctgtctgtcaagtccagcctttcaggagttgaaaatctgaacagctcttatttctcttcacaggacgtgctgagaagcaacctggtgcaggtgaaggtggagaaagaggcgtcctgtgatgagagtgatgttggcactaatgactatgacatggaacatagcactgtgaaagagagtgtgagcactaacaacagggtacagtatgagccagcccacctggctcctctgagggaggactcggtcttaagggaactggatcgggaggacaaagccagcgatgatgagatgatgaccccagagagcgagcgggtccaggtggaagggggcatggagaacagtctgcttccctacgtctccaatatcctgagccccgcaggccagatcttcatgtgccccctgtgcaacaaagtcttccccagcccccacatcctgcagatccacctgagcacgcacttccgagagcaggatggcatccgtagcaagcctgccgccgatgtcaatgtgcccacgtgctccctgtgtgggaagactttctcctgcatgtacaccctcaagcgccacgagaggactcactcgggggagaagccctacacgtgcacccaatgcggcaagagtttccagtactcgcacaacctgagccgtcatgccgtggtgcacacccgcgagaagccccatgcctgcaagtggtgcgagcgcaggttcacgcagtccggagacctgtacagacacatccgcaagttccactgtgagttggtgaactccttgtcggtcaaaagtgaagcgctgagcttgcccactgtcagagactggaccttagaagatagctctcaagaactttggaaataattttatatatatatatatataaataatatatatatatatatatacatatatatacatagatctctatatagttgtggtacggtctaaaagcagtcttgtttcctggaactgaaaagttgggatcttaacttgtttttgcactttagaataatagcatgagaatctcactaatttagcattctgataaaagaaactagagcaagtcacaagtagagagctgtctttcttttgagggttaggccaagttagccaataaaaccttctcaacaaatggttctgtcacaaacgctttcacatggcttacttttgtacacactgcattgtcttgagcgctttccctgtgctccacactgttttgttttgttttgttttcagagtagaaatcccctccagtttattagcctctttatatgtctcaaattgcatgaatattttctggctgttggaaacctgaatgcttttagacccaaatggaaaatttctgaaatgctggattatctatttttaaacaagcagttgacttaaaactttgtgtggcaacttctggttttctgatggtccccagtgagagaaatctgacagtacactgggtcactgggactgtctctgtgaaggtttgcttgagatgaaaaaggatattgcagcttcagcagtgttgaactgtgtgtttaaaatgtgaattactgttcttgtatactgtaattgattacacgggctggggggggtgtcaaagaacttgacaggttgtgttgatgctcttagttgagtcttgaaaagtaaatattaacgctacagaaatgcatgagtttcaatatattttttgtctttgtttgcattgtataactttaacgagtgagtttaaaattatttaatttccttagaaaaatagcaccatttgggaaaaaaactggtgttacgaagaacataaatgcactgtctttatttttatttttattttatataatttaaattgacttttcccacgtctttaagtttgaaactgttaagctgaataaaaacttaagctgcaaattgataacttcgctacattacaaggaacatataaatgtttacaaacggcttaaagaattgcatgtgcagtgtgcattcataccacacttgtaattgcacagaacccatgccagctcagagtttaggtgtacacacgtacccagccgagtgtttttagaataactactgcacaaattgacaataggtcataggtcattctctctctccctcctttttgcttcccctttctttttctcccctatttcctttccttccccctaccccctaactcatgaaaagattctatggactgaaaaagccccaggctgaaaggactgaaccgccttgattgacatggggaggggggttagtagactgtgtatcggcagcagaggctgcagcccgatgtgtggttctgatggccagcgcggggcagaagcgttttgcacactgtttgttttcctgtcttgcacttacaaataaggtctatgggagtagcatgggaaacagtggttgtttttccctttttcttttttaattgcttttgtttaaaatttgatcgccttaactactgtaaacatagcctatttttgtgcttaagatactgaatggaaaactccattgtgtgttgctggactgttttggaaatatttggtcaaatgtatgttaatttggctgtaatggcatttaaagcaaacaaacaaacaaacaaacaaagctgtgaaaatggccttggagcattatctttagttacttgaagagtttctagtttttttttttttaatacagtttatgttaaaataatctttattaatctagaggagacaatcaatgtctgtggaagaacggacttttttggattttcttttttaatggtcattgtgagtgattgctctttcttttacttagtttcacattcttcctttgttctaaatcttagactgacatctagctttgacaatcatagtatgttttatttcctgagggggaataacttataatgctgtttagttttgtactattggtgtgttggtgaatttttaaactgtgtgctaactgcaataaattatatgaactgagaa

SEQ ID NO: 46 - protein, 521 aa, Mus musculus; identical to SEQ ID NO: 42

SEQ ID NO: 47 - DNA, 4147 bp, Mus musculus
aggctgcggccgccgctgggctgctgccatggggagccgttgaaagagtggggccctgtggctcggccgcgcgcctcaggctgccagcgcgggcggggggcggcgggggcgcgcggggcccgggccggggccggcgggcgggcgggggccggcggaggacgcccacaagtccagcggcggcggcggcggcagaggaggaggaggaggaggacgcggcggggacgcggcgactcgggccgctccgtaacctccgagggtccgcggtagcggtggacgggtcgatccgtggagtggatgtcttcgggccattgcggtgcttgggactcattaaactgtcactcacccccaccaccaccccactggtatttcagaagtgcagtttgagtttgttgatgctgaaagtggaaatttgcactaacggtggcagtgtgaactattttcctgagaggtagatccagataaattgcttaggttatgaagacagtatggagtttccagaccacagcagacatttgctgcagtgtctgagcgagcagaggcaccagggctttctttgtgactgcactgttctggtgggagatgcccagttccgggcacaccgagctgttctggcttcatgcagcatgtatttccacctcttttacaaggaccagctggacaaaagagacattgttcatctgaacagcgacattgtgacagcccccgctttcgctctcctgcttgaattcatgtacgaagggaaactccagttcaaagacttgcccattgaggacgtgctagcagctgccagttatctccacatgtatgacattgtcaaagtctgcaaaaagaagctgaaagagaaagctaccacagaggcggacagcaccaaaaaagaagaagatgcttcaagttgttcggataaagtcgagagcctctcagacggcagcagccacatggcaggtgacctgcccagtgatgaagatgaaggcgaagatgacaaattgaacattctacccagcaaaagggacttggcagctgagcctgggaacatgtggatgcgattgccctcagactcagcaggcatcccccaggctggcggagaggccgagccacacgccacagcagctggaaaaacagtagccagcccctgcagctcaacagagtctttgtcccagaggtctgtcacctccgtgagggattcggcagatgttgactgtgtgctggacctgtctgtcaagtccagcctttcaggagttgaaaatctgaacagctcttatttctcttcacaggacgtgctgagaagcaacctggtgcaggtgaaggtggagaaagaggcgtcctgtgatgagagtgatgttggcactaatgactatgacatggaacatagcactgtgaaagagagtgtgagcactaacaacagggtacagtatgagccagcccacctggctcctctgagggaggactcggtcttaagggaactggatcgggaggacaaagccagcgatgatgagatgatgaccccagagagcgagcgggtccaggtggaagggggcatggagaacagtctgcttccctacgtctccaatatcctgagccccgcaggccagatcttcatgtgccccctgtgcaacaaagtcttccccagcccccacatcctgcagatccacctgagcacgcacttccgagagcaggatggcatccgtagcaagcctgccgccgatgtcaatgtgcccacgtgctccctgtgtgggaagactttctcctgcatgtacaccctcaagcgccacgagaggactcactcgggggagaagccctacacgtgcacccaatgcggcaagagtttccagtactcgcacaacctgagccgtcatgccgtggtgcacacccgcgagaagccccatgcctgcaagtggtgcgagcgcaggttcacgcagtccggagacctgtacagacacatccgcaagttccactgtgagttggtgaactccttgtcggtcaaaagtgaagcgctgagcttgcccactgtcagagactggaccttagaagatagctctcaagaactttggaaataattttatatatatatatatataaataatatatatatatatatatacatatatatacatagatctctatatagttgtggtacggtctaaaagcagtcttgtttcctggaactgaaaagttgggatcttaacttgtttttgcactttagaataatagcatgagaatctcactaatttagcattctgataaaagaaactagagcaagtcacaagtagagagctgtctttcttttgagggttaggccaagttagccaataaaaccttctcaacaaatggttctgtcacaaacgctttcacatggcttacttttgtacacactgcattgtcttgagcgctttccctgtgctccacactgttttgttttgttttgttttcagagtagaaatcccctccagtttattagcctctttatatgtctcaaattgcatgaatattttctggctgttggaaacctgaatgcttttagacccaaatggaaaatttctgaaatgctggattatctatttttaaacaagcagttgacttaaaactttgtgtggcaacttctggttttctgatggtccccagtgagagaaatctgacagtacactgggtcactgggactgtctctgtgaaggtttgcttgagatgaaaaaggatattgcagcttcagcagtgttgaactgtgtgtttaaaatgtgaattactgttcttgtatactgtaattgattacacgggctggggggggtgtcaaagaacttgacaggttgtgttgatgctcttagttgagtcttgaaaagtaaatattaacgctacagaaatgcatgagtttcaatatattttttgtctttgtttgcattgtataactttaacgagtgagtttaaaattatttaatttccttagaaaaatagcaccatttgggaaaaaaactggtgttacgaagaacataaatgcactgtctttatttttatttttattttatataatttaaattgacttttcccacgtctttaagtttgaaactgttaagctgaataaaaacttaagctgcaaattgataacttcgctacattacaaggaacatataaatgtttacaaacggcttaaagaattgcatgtgcagtgtgcattcataccacacttgtaattgcacagaacccatgccagctcagagtttaggtgtacacacgtacccagccgagtgtttttagaataactactgcacaaattgacaataggtcataggtcattctctctctccctcctttttgcttcccctttctttttctcccctatttcctttccttccccctaccccctaactcatgaaaagattctatggactgaaaaagccccaggctgaaaggactgaaccgccttgattgacatggggaggggggttagtagactgtgtatcggcagcagaggctgcagcccgatgtgtggttctgatggccagcgcggggcagaagcgttttgcacactgtttgttttcctgtcttgcacttacaaataaggtctatgggagtagcatgggaaacagtggttgtttttccctttttcttttttaattgcttttgtttaaaatttgatcgccttaactactgtaaacatagcctatttttgtgcttaagatactgaatggaaaactccattgtgtgttgctggactgttttggaaatatttggtcaaatgtatgttaatttggctgtaatggcatttaaagcaaacaaacaaacaaacaaacaaagctgtgaaaatggccttggagcattatctttagttacttgaagagtttctagtttttttttttttaatacagtttatgttaaaataatctttattaatctagaggagacaatcaatgtctgtggaagaacggacttttttggattttcttttttaatggtcattgtgagtgattgctctttcttttacttagtttcacattcttcctttgttctaaatcttagactgacatctagctttgacaatcatagtatgttttatttcctgagggggaataacttataatgctgtttagttttgtactattggtgtgttggtgaatttttaaactgtgtgctaactgcaataaattatatgaactgagaa

SEQ ID NO: 48 - protein, 522 aa, Mus musculus; identical to SEQ ID NO: 38

SEQ ID NO: 49 - DNA, 3810 bp, Mus musculus
atgaagtgatggtcttactggattgaaagtcttgggcttgcttggtttacgattttatttcttctatttgagcaagtaaataactttaatacacaaaaatatttcaagttttcagatggttatgaagacagtatggagtttccagaccacagcagacatttgctgcagtgtctgagcgagcagaggcaccagggctttctttgtgactgcactgttctggtgggagatgcccagttccgggcacaccgagctgttctggcttcatgcagcatgtatttccacctcttttacaaggaccagctggacaaaagagacattgttcatctgaacagcgacattgtgacagcccccgctttcgctctcctgcttgaattcatgtacgaagggaaactccagttcaaagacttgcccattgaggacgtgctagcagctgccagttatctccacatgtatgacattgtcaaagtctgcaaaaagaagctgaaagagaaagctaccacagaggcggacagcaccaaaaaagaagaagatgcttcaagttgttcggataaagtcgagagcctctcagacggcagcagccacatggcaggtgacctgcccagtgatgaagatgaaggcgaagatgacaaattgaacattctacccagcaaaagggacttggcagctgagcctgggaacatgtggatgcgattgccctcagactcagcaggcatcccccaggctggcggagaggccgagccacacgccacagcagctggaaaaacagtagccagcccctgcagctcaacagagtctttgtcccagaggtctgtcacctccgtgagggattcggcagatgttgactgtgtgctggacctgtctgtcaagtccagcctttcaggagttgaaaatctgaacagctcttatttctcttcacaggacgtgctgagaagcaacctggtgcaggtgaaggtggagaaagaggcgtcctgtgatgagagtgatgttggcactaatgactatgacatggaacatagcactgtgaaagagagtgtgagcactaacaacagggtacagtatgagccagcccacctggctcctctgagggaggactcggtcttaagggaactggatcgggaggacaaagccagcgatgatgagatgatgaccccagagagcgagcgggtccaggtggaagggggcatggagaacagtctgcttccctacgtctccaatatcctgagccccgcaggccagatcttcatgtgccccctgtgcaacaaagtcttccccagcccccacatcctgcagatccacctgagcacgcacttccgagagcaggatggcatccgtagcaagcctgccgccgatgtcaatgtgcccacgtgctccctgtgtgggaagactttctcctgcatgtacaccctcaagcgccacgagaggactcactcgggggagaagccctacacgtgcacccaatgcggcaagagtttccagtactcgcacaacctgagccgtcatgccgtggtgcacacccgcgagaagccccatgcctgcaagtggtgcgagcgcaggttcacgcagtccggagacctgtacagacacatccgcaagttccactgtgagttggtgaactccttgtcggtcaaaagtgaagcgctgagcttgcccactgtcagagactggaccttagaagatagctctcaagaactttggaaataattttatatatatatatatataaataatatatatatatatatatacatatatatacatagatctctatatagttgtggtacggtctaaaagcagtcttgtttcctggaactgaaaagttgggatcttaacttgtttttgcactttagaataatagcatgagaatctcactaatttagcattctgataaaagaaactagagcaagtcacaagtagagagctgtctttcttttgagggttaggccaagttagccaataaaaccttctcaacaaatggttctgtcacaaacgctttcacatggcttacttttgtacacactgcattgtcttgagcgctttccctgtgctccacactgttttgttttgttttgttttcagagtagaaatcccctccagtttattagcctctttatatgtctcaaattgcatgaatattttctggctgttggaaacctgaatgcttttagacccaaatggaaaatttctgaaatgctggattatctatttttaaacaagcagttgacttaaaactttgtgtggcaacttctggttttctgatggtccccagtgagagaaatctgacagtacactgggtcactgggactgtctctgtgaaggtttgcttgagatgaaaaaggatattgcagcttcagcagtgttgaactgtgtgtttaaaatgtgaattactgttcttgtatactgtaattgattacacgggctggggggggtgtcaaagaacttgacaggttgtgttgatgctcttagttgagtcttgaaaagtaaatattaacgctacagaaatgcatgagtttcaatatattttttgtctttgtttgcattgtataactttaacgagtgagtttaaaattatttaatttccttagaaaaatagcaccatttgggaaaaaaactggtgttacgaagaacataaatgcactgtctttatttttatttttattttatataatttaaattgacttttcccacgtctttaagtttgaaactgttaagctgaataaaaacttaagctgcaaattgataacttcgctacattacaaggaacatataaatgtttacaaacggcttaaagaattgcatgtgcagtgtgcattcataccacacttgtaattgcacagaacccatgccagctcagagtttaggtgtacacacgtacccagccgagtgtttttagaataactactgcacaaattgacaataggtcataggtcattctctctctccctcctttttgcttcccctttctttttctcccctatttcctttccttccccctaccccctaactcatgaaaagattctatggactgaaaaagccccaggctgaaaggactgaaccgccttgattgacatggggaggggggttagtagactgtgtatcggcagcagaggctgcagcccgatgtgtggttctgatggccagcgcggggcagaagcgttttgcacactgtttgttttcctgtcttgcacttacaaataaggtctatgggagtagcatgggaaacagtggttgtttttccctttttcttttttaattgcttttgtttaaaatttgatcgccttaactactgtaaacatagcctatttttgtgcttaagatactgaatggaaaactccattgtgtgttgctggactgttttggaaatatttggtcaaatgtatgttaatttggctgtaatggcatttaaagcaaacaaacaaacaaacaaacaaagctgtgaaaatggccttggagcattatctttagttacttgaagagtttctagtttttttttttttaatacagtttatgttaaaataatctttattaatctagaggagacaatcaatgtctgtggaagaacggacttttttggattttcttttttaatggtcattgtgagtgattgctctttcttttacttagtttcacattcttcctttgttctaaatcttagactgacatctagctttgacaatcatagtatgttttatttcctgagggggaataacttataatgctgtttagttttgtactattggtgtgttggtgaatttttaaactgtgtgctaactgcaataaattatatgaactgagaaaaaaaaaaaaaaaaaa

SEQ ID NO: 50 - protein, 531 aa, Homo sapiens
MCPKGYEDSMEFPDHSRHLLQCLSEQRHQGFLCDCTVLVGDAQFRAHRAVLASCSMYFHLFYKDQLDKRDIVHLNSDIVTAPAFALLLEFMYEGKLQFKDLPIEDVLAAASYLHMYDIVKVCKKKLKEKATTEADSTKKEEDASSCSDKVESLSDGSSHIAGDLPSDEDEGEDEKLNILPSKRDLAAEPGNMWMRLPSDSAGIPQAGGEAEPHATAAGKTVASPCSSTESLSQRSVTSVRDSADVDCVLDLSVKSSLSGVENLNSSYFSSQDVLRSNLVQVKVEKEASCDESDVGTNDYDMEHSTVKESVSTNNRVQYEPAHLAPLREDSVLRELDREDKASDDEMMTPESERVQVEGGMESSLLPYVSNILSPAGQIFMCPLCNKVFPSPHILQIHLSTHFREQDGIRSKPAADVNVPTCSLCGKTFSCMYTLKRHERTHSGEKPYTCTQCGKSFQYSHNLSRHAVVHTREKPHACKWCERRFTQSGDLYRHIRKFHCELVNSLSVKSEALSLPTVRDWTLEDSSQELWK

SEQ ID NO: 51 - DNA, 3851 bp, Homo sapiens
agtgtgtgcggatctgtggtggagaaggtatctcattcctctctaacatcatctccactttgcatctgtctctcttagtctgctttttttccaccgatgtaacagacctggagccagcaggactcagaggaaaggacttaatcaggtttatgtgtcctaaaggttatgaagacagtatggagtttccagaccatagtagacatttgctacagtgtctgagcgagcagagacaccagggttttctttgtgactgcactgttctggtgggagatgcccagttccgagcgcaccgagctgtactggcttcatgcagcatgtatttccacctcttttacaaggaccagctggacaaaagagacattgttcatctgaacagcgacattgttacagcccccgctttcgctctcctgcttgaattcatgtatgaagggaaactccagttcaaagacttgcccattgaagacgtgctagcagctgccagttatctccacatgtatgacattgtcaaagtctgcaaaaagaagctgaaagagaaagccaccacggaggcagacagcaccaaaaaggaagaagatgcttcaagttgttcggacaaagtcgagagtctctccgatggcagcagccacatagcaggcgatttgcccagtgatgaagatgaaggagaagatgaaaaattgaacatcctgcccagcaaaagggacttggcggccgagcctgggaacatgtggatgcgattgccctcagactcagcaggcatcccccaggctggcggagaggcagagccacacgccacagcagctggaaaaacagtagccagcccctgcagctcaacagagtctttgtcccagaggtctgtcacctccgtgagggattcggcagatgttgactgtgtgctggacctgtctgtcaagtccagcctttcaggagttgaaaatctgaacagctcttatttctcttcacaggacgtgctgagaagcaacctggtgcaggtgaaggtggagaaagaggcttcctgtgatgagagtgatgttggcactaatgactatgacatggaacatagcactgtgaaagaaagtgtgagcactaataacagggtacagtatgagccggcccatctggctcccctgagggaggactcggtcttgagggagctggaccgggaggacaaagccagtgatgatgagatgatgaccccagagagcgagcgtgtccaggtggagggaggcatggagagcagtctgctcccctacgtctccaacatcctgagccccgcgggccagatcttcatgtgccccctgtgcaacaaggtcttccccagcccccacatcctgcagatccacctgagcacgcacttccgcgagcaggacggcatccgcagcaagcccgccgccgatgtcaacgtgcccacgtgctcgctgtgtgggaagactttctcttgcatgtacaccctcaagcgccacgagaggactcactcgggggagaagccctacacatgcacccagtgcggcaagagcttccagtactcgcacaacctgagccgccatgccgtggtgcacacccgcgagaagccgcacgcctgcaagtggtgcgagcgcaggttcacgcagtccggggacctgtacagacacattcgcaagttccactgtgagttggtgaactccttgtcggtcaaaagcgaagcactgagcttgcctactgtcagagactggaccttagaagatagctctcaagaactttggaaataattttatatatatataaataatatatatatatatacatatatataaatagatctctatatagttgtggtacggtctaaaagcagtcttgtttcctggaaataaaaagttgggatattaacttgtttttgcactttagaatagcatgagaatctcactaatttagcattctgataaaagaaactttagagcaagtcagaatagagaggtgtttttcctttgaggggataggggaagtaagccaataagaaccttttaaacaaatcgtcctgtcacaaaatgctttcatatggcttaattttgtcaacactgcattgtcttttgagctcttttttcccccccaacaaagtttttttgttttttgtttttttttttaagtagaaattccctccagttttattagcctctttatatgtctcaaattgcatgaattttttctggctgttggaaacctgaatgcttttagacccaaatggaaaatttctgaaatgctggattatctatttttaaacaagcagttgacttaaaactttctgtggcaacttctggttttctgacagttcccagtgagagaaatgctgaaagtacactgggatcactgggacactgtcttatgaaggtttgcttgggatgaaaaaggatattgcagcttcagcagtgttgaactgtgtgtttaaaaatgtgaattactgttattgtatactgtaattgattacatgggctgggggggtgtcaaagaacttgacaggttgtgttgatgctcttagttgagtcttgaaaagtaaatattaacgctacagaaatgcatgagtttcaatatattttttgtctttgtttgcattgtataactttaacgagtgagtttaaaattatttaatttccttagaaaaataacaccatttggaaaaaaaaactggtgttatgaagaacgtaaatgcactgtttttatttttattttatataatttaaattgactttcccactgtctttaagttgaaactgttaagctgaataaaaacttaagctgcaaattgataacttcgctacataacaaggaaaatataaatgtttacaaacagcttaaagatttgcatgtgcagtgtgcatttataacaaacttctaattgcacaaaacccatgccagctcagagtttaggtgtacacatttacccagttgagcgttcttagaataactactgcacaagttgacaataggtcgttctctctttttttttgtttgctccctttttctttttctccccttcctccttaccctccctcccttactctccccccccaccaccaccctccacccccaactcatgaaaagattctatggactgaaaaagccccaggctgaaaggactggactgccttgattgacatggggaagggggttagtagactatgtggattgcggcagcagaggctgcagcctaacgtgtggttttaatgaccagcacgcaaggcaaaagcattttgcacagtgtttgttttcctgtcttgcacttacaaataaggtctatgggagtagcatggaaaacgtttgctgtttttcccttttttttttaattgcttttgtttaaaatttgatcgccttaactactgtaaacatagcctatttttgtgcttaagatactgaatggaaaactccattgtgtgttgctggactgttttggaaatatttggttaaatgtgtgttaatttggctgtaatggcatttaaagcaaacaaacaaacaaaaaaagctgtgaaaatggccttggagcattatctttagttacttgaagagtttctagtttttttaaaatacagtttatgttaaaataatttttattaatttagagaagacaatcaatgtctgtgagaaaacggactttcttttggattttctttttgtggtcattgtgagtgattgctttttccttttcttagtttcacattcttcctttgttctaaaacttagactgacatctagctttgacaatcatagtatgttttattttcctgagggggaataacttataatgctgtttagttttgtactattggtgtgttggtgaatttttaaactgtgtgctaactgcaataaattatatgaactgagaa

SEQ ID NO: 52 - protein, 522 aa, Homo sapiens
MEFPDHSRHLLQCLSEQRHQGFLCDCTVLVGDAQFRAHRAVLASCSMYFHLFYKDQLDKRDIVHLNSDIVTAPAFALLLEFMYEGKLQFKDLPIEDVLAAASYLHMYDIVKVCKKKLKEKATTEADSTKKEEDASSCSDKVESLSDGSSHIAGDLPSDEDEGEDEKLNILPSKRDLAAEPGNMWMRLPSDSAGIPQAGGEAEPHATAAGKTVASPCSSTESLSQRSVTSVRDSADVDCVLDLSVKSSLSGVENLNSSYFSSQDVLRSNLVQVKVEKEASCDESDVGTNDYDMEHSTVKESVSTNNRVQYEPAHLAPLREDSVLRELDREDKASDDEMMTPESERVQVEGGMESSLLPYVSNILSPAGQIFMCPLCNKVFPSPHILQIHLSTHFREQDGIRSKPAADVNVPTCSLCGKTFSCMYTLKRHERTHSGEKPYTCTQCGKSFQYSHNLSRHAVVHTREKPHACKWCERRFTQSGDLYRHIRKFHCELVNSLSVKSEALSLPTVRDWTLEDSSQELWK

SEQ ID NO: 53 - DNA, 4319 bp, Homo sapiens
gttgtttgctaatttctggactaattatgcaatttcatttttttttcttgattcagccaaatgtgtgtgtgtgtgtttaaactgtgctttctaagcacagtcaggtagcaaaagtaataaaaaggatggttgaacaagttttcttgtatgttccaggatatgtttgggacttttctttgtttattatatgagttgttccctttgaaattaaagctattttgtaggttttgtgggacataatttgataagtagagttaattaaatttcttctggaagagatctaaattcttattcttagtgagagactgtagttaaaggaaggcttttagaacttgggttcaaggaagatggagatgcgtcggaagctctttggcgggggtgaggaagttcagaaagtgtgcattttccttctggcatttaggtcttgtccgtgtgatttggtggtgcttgggtcataagcctgattaaaattcagggacatgtaccacggcggccaaagcggaattaatttttttatatggggactggagcgctgaaaagttgttcctgaccaggctctaatgagaaattcctctctccccaggttatgaagacagtatggagtttccagaccatagtagacatttgctacagtgtctgagcgagcagagacaccagggttttctttgtgactgcactgttctggtgggagatgcccagttccgagcgcaccgagctgtactggcttcatgcagcatgtatttccacctcttttacaaggaccagctggacaaaagagacattgttcatctgaacagcgacattgttacagcccccgctttcgctctcctgcttgaattcatgtatgaagggaaactccagttcaaagacttgcccattgaagacgtgctagcagctgccagttatctccacatgtatgacattgtcaaagtctgcaaaaagaagctgaaagagaaagccaccacggaggcagacagcaccaaaaaggaagaagatgcttcaagttgttcggacaaagtcgagagtctctccgatggcagcagccacatagcaggcgatttgcccagtgatgaagatgaaggagaagatgaaaaattgaacatcctgcccagcaaaagggacttggcggccgagcctgggaacatgtggatgcgattgccctcagactcagcaggcatcccccaggctggcggagaggcagagccacacgccacagcagctggaaaaacagtagccagcccctgcagctcaacagagtctttgtcccagaggtctgtcacctccgtgagggattcggcagatgttgactgtgtgctggacctgtctgtcaagtccagcctttcaggagttgaaaatctgaacagctcttatttctcttcacaggacgtgctgagaagcaacctggtgcaggtgaaggtggagaaagaggcttcctgtgatgagagtgatgttggcactaatgactatgacatggaacatagcactgtgaaagaaagtgtgagcactaataacagggtacagtatgagccggcccatctggctcccctgagggaggactcggtcttgagggagctggaccgggaggacaaagccagtgatgatgagatgatgaccccagagagcgagcgtgtccaggtggagggaggcatggagagcagtctgctcccctacgtctccaacatcctgagccccgcgggccagatcttcatgtgccccctgtgcaacaaggtcttccccagcccccacatcctgcagatccacctgagcacgcacttccgcgagcaggacggcatccgcagcaagcccgccgccgatgtcaacgtgcccacgtgctcgctgtgtgggaagactttctcttgcatgtacaccctcaagcgccacgagaggactcactcgggggagaagccctacacatgcacccagtgcggcaagagcttccagtactcgcacaacctgagccgccatgccgtggtgcacacccgcgagaagccgcacgcctgcaagtggtgcgagcgcaggttcacgcagtccggggacctgtacagacacattcgcaagttccactgtgagttggtgaactccttgtcggtcaaaagcgaagcactgagcttgcctactgtcagagactggaccttagaagatagctctcaagaactttggaaataattttatatatatataaataatatatatatatatacatatatataaatagatctctatatagttgtggtacggtctaaaagcagtcttgtttcctggaaataaaaagttgggatattaacttgtttttgcactttagaatagcatgagaatctcactaatttagcattctgataaaagaaactttagagcaagtcagaatagagaggtgtttttcctttgaggggataggggaagtaagccaataagaaccttttaaacaaatcgtcctgtcacaaaatgctttcatatggcttaattttgtcaacactgcattgtcttttgagctcttttttcccccccaacaaagtttttttgttttttgtttttttttttaagtagaaattccctccagttttattagcctctttatatgtctcaaattgcatgaattttttctggctgttggaaacctgaatgcttttagacccaaatggaaaatttctgaaatgctggattatctatttttaaacaagcagttgacttaaaactttctgtggcaacttctggttttctgacagttcccagtgagagaaatgctgaaagtacactgggatcactgggacactgtcttatgaaggtttgcttgggatgaaaaaggatattgcagcttcagcagtgttgaactgtgtgtttaaaaatgtgaattactgttattgtatactgtaattgattacatgggctgggggggtgtcaaagaacttgacaggttgtgttgatgctcttagttgagtcttgaaaagtaaatattaacgctacagaaatgcatgagtttcaatatattttttgtctttgtttgcattgtataactttaacgagtgagtttaaaattatttaatttccttagaaaaatagcaccatttggaaaaaaaaactggtgttatgaagaacgtaaatgcactgtttttatttttattttatataatttaaattgactttcccactgtctttaagttgaaactgttaagctgaataaaaacttaagctgcaaattgataacttcgctacataacaaggaaaatataaatgtttacaaacagcttaaagatttgcatgtgcagtgtgcatttataacaaacttctaattgcacaaaacccatgccagctcagagtttaggtgtacacatttacccagttgagcgttcttagaataactactgcacaagttgacaataggtcgttctctctttttttttgtttgctccctttttctttttctccccttcctccttaccctccctcccttactctccccccccaccaccaccctccacccccaactcatgaaaagattctatggactgaaaaagccccaggctgaaaggactggactgccttgattgacatggggaagggggttagtagactatgtggattgcggcagcagaggctgcagcctaacgtgtggttttaatgaccagcacgcaaggcaaaagcattttgcacagtgtttgttttcctgtcttgcacttacaaataaggtctatgggagtagcatggaaaacgtttgctgtttttcccttttttttttaattgcttttgtttaaaatttgatcgccttaactactgtaaacatagcctatttttgtgcttaagatactgaatggaaaactccattgtgtgttgctggactgttttggaaatatttggttaaatgtgtgttaatttggctgtaatggcatttaaagcaaacaaacaaacaaaaaaagctgtgaaaatggccttggagcattatctttagttacttgaagagtttctagtttttttaaaatacagtttatgttaaaataatttttattaatttagagaagacaatcaatgtctgtgagaaaacggactttcttttggattttctttttgtggtcattgtgagtgattgctttttccttttcttagtttcacattcttcctttgttctaaaacttagactgacatctagctttgacaatcatagtatgttttattttcctgagggggaataacttataatgctgtttagttttgtactattggtgtgttggtgaatttttaaactgtgtgctaactgcaataaattatatgaactgagaaaaaaaaaaaaaaaaaaaaaaaaaaaaaaaaaaaaaaaaaaaaaaaaa

SEQ ID NO: 54 - protein, 522 aa, Homo sapiens; identical to SEQ ID NO: 52

SEQ ID NO: 55 - DNA, 4030 bp, Homo sapiens
acccggcccggcggctccgcgctcgctccctcgctcgctccctcgctgtgtctggcaggctgcggccgccgctgggctgctgccatggggagccgttgagagtggggccctcttcgctccgccgcgctcctccggctgccagcgggggtgggcgggagggaggggcgggggggagcgggcgacggagccgggattggggggtgggggggccgggagggaggggggcaagagccggaggaagcccaccaagtccggcggcggcggcggcggccgggaggaggaggaggaggaggcggaggacgcggcggcggcggggacgcggtgacttagggccgctccgtgttatgaagacagtatggagtttccagaccatagtagacatttgctacagtgtctgagcgagcagagacaccagggttttctttgtgactgcactgttctggtgggagatgcccagttccgagcgcaccgagctgtactggcttcatgcagcatgtatttccacctcttttacaaggaccagctggacaaaagagacattgttcatctgaacagcgacattgttacagcccccgctttcgctctcctgcttgaattcatgtatgaagggaaactccagttcaaagacttgcccattgaagacgtgctagcagctgccagttatctccacatgtatgacattgtcaaagtctgcaaaaagaagctgaaagagaaagccaccacggaggcagacagcaccaaaaaggaagaagatgcttcaagttgttcggacaaagtcgagagtctctccgatggcagcagccacatagcaggcgatttgcccagtgatgaagatgaaggagaagatgaaaaattgaacatcctgcccagcaaaagggacttggcggccgagcctgggaacatgtggatgcgattgccctcagactcagcaggcatcccccaggctggcggagaggcagagccacacgccacagcagctggaaaaacagtagccagcccctgcagctcaacagagtctttgtcccagaggtctgtcacctccgtgagggattcggcagatgttgactgtgtgctggacctgtctgtcaagtccagcctttcaggagttgaaaatctgaacagctcttatttctcttcacaggacgtgctgagaagcaacctggtgcaggtgaaggtggagaaagaggcttcctgtgatgagagtgatgttggcactaatgactatgacatggaacatagcactgtgaaagaaagtgtgagcactaataacagggtacagtatgagccggcccatctggctcccctgagggaggactcggtcttgagggagctggaccgggaggacaaagccagtgatgatgagatgatgaccccagagagcgagcgtgtccaggtggagggaggcatggagagcagtctgctcccctacgtctccaacatcctgagccccgcgggccagatcttcatgtgccccctgtgcaacaaggtcttccccagcccccacatcctgcagatccacctgagcacgcacttccgcgagcaggacggcatccgcagcaagcccgccgccgatgtcaacgtgcccacgtgctcgctgtgtgggaagactttctcttgcatgtacaccctcaagcgccacgagaggactcactcgggggagaagccctacacatgcacccagtgcggcaagagcttccagtactcgcacaacctgagccgccatgccgtggtgcacacccgcgagaagccgcacgcctgcaagtggtgcgagcgcaggttcacgcagtccggggacctgtacagacacattcgcaagttccactgtgagttggtgaactccttgtcggtcaaaagcgaagcactgagcttgcctactgtcagagactggaccttagaagatagctctcaagaactttggaaataattttatatatatataaataatatatatatatatacatatatataaatagatctctatatagttgtggtacggtctaaaagcagtcttgtttcctggaaataaaaagttgggatattaacttgtttttgcactttagaatagcatgagaatctcactaatttagcattctgataaaagaaactttagagcaagtcagaatagagaggtgtttttcctttgaggggataggggaagtaagccaataagaaccttttaaacaaatcgtcctgtcacaaaatgctttcatatggcttaattttgtcaacactgcattgtcttttgagctcttttttcccccccaacaaagtttttttgttttttgtttttttttttaagtagaaattccctccagttttattagcctctttatatgtctcaaattgcatgaattttttctggctgttggaaacctgaatgcttttagacccaaatggaaaatttctgaaatgctggattatctatttttaaacaagcagttgacttaaaactttctgtggcaacttctggttttctgacagttcccagtgagagaaatgctgaaagtacactgggatcactgggacactgtcttatgaaggtttgcttgggatgaaaaaggatattgcagcttcagcagtgttgaactgtgtgtttaaaaatgtgaattactgttattgtatactgtaattgattacatgggctgggggggtgtcaaagaacttgacaggttgtgttgatgctcttagttgagtcttgaaaagtaaatattaacgctacagaaatgcatgagtttcaatatattttttgtctttgtttgcattgtataactttaacgagtgagtttaaaattatttaatttccttagaaaaataacaccatttggaaaaaaaaactggtgttatgaagaacgtaaatgcactgtttttatttttattttatataatttaaattgactttcccactgtctttaagttgaaactgttaagctgaataaaaacttaagctgcaaattgataacttcgctacataacaaggaaaatataaatgtttacaaacagcttaaagatttgcatgtgcagtgtgcatttataacaaacttctaattgcacaaaacccatgccagctcagagtttaggtgtacacatttacccagttgagcgttcttagaataactactgcacaagttgacaataggtcgttctctctttttttttgtttgctccctttttctttttctccccttcctccttaccctccctcccttactctccccccccaccaccaccctccacccccaactcatgaaaagattctatggactgaaaaagccccaggctgaaaggactggactgccttgattgacatggggaagggggttagtagactatgtggattgcggcagcagaggctgcagcctaacgtgtggttttaatgaccagcacgcaaggcaaaagcattttgcacagtgtttgttttcctgtcttgcacttacaaataaggtctatgggagtagcatggaaaacgtttgctgtttttcccttttttttttaattgcttttgtttaaaatttgatcgccttaactactgtaaacatagcctatttttgtgcttaagatactgaatggaaaactccattgtgtgttgctggactgttttggaaatatttggttaaatgtgtgttaatttggctgtaatggcatttaaagcaaacaaacaaacaaaaaaagctgtgaaaatggccttggagcattatctttagttacttgaagagtttctagtttttttaaaatacagtttatgttaaaataatttttattaatttagagaagacaatcaatgtctgtgagaaaacggactttcttttggattttctttttgtggtcattgtgagtgattgctttttccttttcttagtttcacattcttcctttgttctaaaacttagactgacatctagctttgacaatcatagtatgttttattttcctgagggggaataacttataatgctgtttagttttgtactattggtgtgttggtgaatttttaaactgtgtgctaactgcaataaattatatgaactgagaa